SEQUENCE LISTING (285 sequences)

SEQ ID NO: 1 - protein, 30 aa, synthetic construct
MKKLLLLLVVLLGLAALLLSGCGTSVSAAT

SEQ ID NO: 2 - protein, 30 aa, synthetic construct
MKKLLLLLVVLAGLAALLLSGCGTSVSAAT

SEQ ID NO: 3 - protein, 30 aa, synthetic construct
MKKLLLLLVVLLGLAALLLSGCGTSVSAAK

SEQ ID NO: 4 - protein, 30 aa, synthetic construct
MKKLLILLVVLLGLAALLLSGCGTSVSAAT

SEQ ID NO: 5 - protein, 30 aa, synthetic construct
MKKLLLLLVVLLSLAALLLSGCGTSVSAAK

SEQ ID NO: 6 - protein, 30 aa, synthetic construct
MKKLLLLLVVLAGLAALLLSGAGTSVSAAT

SEQ ID NO: 7 - protein, 30 aa, synthetic construct
MKKLLIGLVVLLGLAALLLSGLGTSVSAAT

SEQ ID NO: 8 - protein, 30 aa, synthetic construct
MKKLLLLLVVLAGLAALLLSGCGTSVSAAD

SEQ ID NO: 9 - protein, 30 aa, synthetic construct
MKKLTLLLVVVLGLAALCLSGCGTSVSADE

SEQ ID NO: 10 - protein, 30 aa, synthetic construct
MHKLLLLLVVLVGLAALLLSGCGTTVSAAT

SEQ ID NO: 11 - protein, 30 aa, synthetic construct
MRKLLLLLVVLLGLAALLLSGAGTPVSAAT

SEQ ID NO: 12 - protein, 30 aa, synthetic construct
MKRLLLGLVGLLGLAILGLSGCGTSVSAQT

SEQ ID NO: 13 - protein, 30 aa, synthetic construct
MKKLLLLLVLVLGLAAMLLSGCGTPTSADT

SEQ ID NO: 14 - protein, 30 aa, synthetic construct
MKKILLLVVVLLGLAALLWSGAGTSVSAST

SEQ ID NO: 15 - protein, 30 aa, synthetic construct
MRRLLLLLVVVLGLAALLLSGCGTSVSAQA

SEQ ID NO: 16 - protein, 30 aa, synthetic construct
MRKLHELLIVLLGISFVLWSGCGTSVSAAG

SEQ ID NO: 17 - protein, 30 aa, synthetic construct
MKKLLLGLVLLLGLAALALTGCGTPVSAAT

SEQ ID NO: 18 - protein, 30 aa, synthetic construct
MKKLLLLLVVLLSVAALLASGCGTFVSAAT

SEQ ID NO: 19 - protein, 30 aa, synthetic construct
MKRLLLKAVVLLSLAALLWSGAGTSVSAAT

SEQ ID NO: 20 - protein, 30 aa, synthetic construct
MKRLLIGAVVLLGLAALLLSGCGTSVSAAT

SEQ ID NO: 21 - protein, 114 aa, synthetic construct
EVQLVESGGGLVQPGGSLRLSCAASGFTFSDYWMYWVRQAPGKGLEWVSEINTNGLITKYPDSVGRFTISRDNAKNTLYLQMNSLRPEDTAVYYCARSPSGFNRGQGTLVTVSS

SEQ ID NO: 22 - protein, 146 aa, synthetic construct
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 23 - protein, 45 aa, synthetic construct
GIINTLQKYYCRVRGGRCAVLSCLPKEEQIGKCSTRGRKCCRRKK

SEQ ID NO: 24 - protein, 177 aa, synthetic construct
HSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 25 - protein, 146 aa, synthetic construct
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 26 - protein, 209 aa, synthetic construct
RELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 27 - protein, 5 aa, synthetic construct
GGGGS

SEQ ID NO: 28 - protein, 15 aa, synthetic construct
GGGGSGGGGSGGGGS

SEQ ID NO: 29 - protein, 5 aa, synthetic construct
  REPEAT 1..5: note = sequence is present n times, wherein n = 1, 2, 3, or 4
GGGGS

SEQ ID NO: 30 - protein, 8 aa, synthetic construct
GGGGGGGG

SEQ ID NO: 31 - protein, 6 aa, synthetic construct
GGGGGG

SEQ ID NO: 32 - protein, 15 aa, synthetic construct
EAAAKEAAAKEAAAK

SEQ ID NO: 33 - protein, 4 aa, synthetic construct
  REPEAT 1..4: note = sequence is present n times, wherein n is 1, 2, or 3
EAAK

SEQ ID NO: 34 - protein, 46 aa, synthetic construct
AEAAAKEAAAKEAAAKEAAAKALEAEAAAKEAAAKEAAAKEAAAKA

SEQ ID NO: 35 - protein, 12 aa, synthetic construct
AEAAAKEAAAKA

SEQ ID NO: 36 - protein, 5 aa, synthetic construct
PAPAP

SEQ ID NO: 37 - protein, 10 aa, synthetic construct
  REPEAT 1..2: note = The sequence may be present n times, wherein n an integer selected from 5 to 17. SEQ ID NO: 37 represents the embodiment where n=10
APAPAPAPAP

SEQ ID NO: 38 - protein, 17 aa, synthetic construct
VSQTSKLTRAETVFPDV

SEQ ID NO: 39 - protein, 6 aa, synthetic construct
PLGLWA

SEQ ID NO: 40 - protein, 6 aa, synthetic construct
RVLAEA

SEQ ID NO: 41 - protein, 10 aa, synthetic construct
EDVVCCSMSY

SEQ ID NO: 42 - protein, 8 aa, synthetic construct
GGIEGRGS

SEQ ID NO: 43 - protein, 10 aa, synthetic construct
TRHRQPRGWE

SEQ ID NO: 44 - protein, 10 aa, synthetic construct
AGNRVRRSVG

SEQ ID NO: 45 - protein, 9 aa, synthetic construct
RRRRRRRRR

SEQ ID NO: 46 - protein, 4 aa, synthetic construct
GFLG

SEQ ID NO: 47 - protein, 30 aa, synthetic construct
MKKLTLLLTVSLGMAGFLLSGCGQPVSAAQ

SEQ ID NO: 48 - protein, 30 aa, synthetic construct
MHKLLMLLVVLLGLAGLSLSGCGSSTSAAT

SEQ ID NO: 49 - protein, 36 aa, synthetic construct
DHYNCISSGGQCLYSACPIFTKIQGTCYRGKAKCCK

SEQ ID NO: 50 - protein, 41 aa, synthetic construct
GIGDPVTCLKSGAICHPVFCPRRYKQIGTCGLPGTKCCKKP

SEQ ID NO: 51 - protein, 15 aa, synthetic construct
RSMRLSFRARGYGWR

SEQ ID NO: 52 - protein, 29 aa, synthetic construct
GLWSTIKQKGKEAAIAAAKAAGQAALGAL

SEQ ID NO: 53 - protein, 75 aa, synthetic construct
ESLQERADEATTQKQSGEDNQDLAISFAGNGLSALRTSGSQARATCYCRTGRCATRESLSGVCEISGRLYRLCCR

SEQ ID NO: 54 - protein, 149 aa, synthetic construct
EEPQRELPSARIRCPKGSKAYGSHCYALFLSPKSWTDADLACQKRPSGNLVSVLSGAEGSFVSSLVKSIGNSYSYVWIGLHDPTQGTEPNGEGWEWSSSDVMNYFAWERNPSTISSPGHCASLSRSTAFLRWKDYNCNVRLPYVCKFTD

SEQ ID NO: 55 - protein, 18 aa, synthetic construct
SWLRDIWDWICEVLSDFK

SEQ ID NO: 56 - protein, 83 aa, synthetic construct
ATMENKVICALVLVSMLALGTLAEAQTETCTVAPRERQNCGFPGVTPSQCANKGCCFDDTVRGVPWCFYPNTIDVPPEEECEF

SEQ ID NO: 57 - protein, 128 aa, synthetic construct
GRRDAQLLAALLVLGLCALAGSEKPSPCQCSRLSPHNRTNCGFPGITSDQCFDNGCCFDSSVTGVPWCFHPLPKQESDQCVMEVSDRRNCGYPGISPEECASRKCCFSNFIFEVPWCFFPKSVEDCHY

SEQ ID NO: 58 - protein, 79 aa, synthetic construct
AARALCMLGLVLALLSSSSAEEYVGLSANQCAVPAKDRVDCGYPHVTPKECNNRGCCFDSRIPGVPWCFKPLQEAECTF

SEQ ID NO: 59 - protein, 259 aa, synthetic construct
TPQNITDLCAEYHNTQIYTLNDKIFSYTESLAGKREMAIITFKNGAIFQVEVPGSQHIDSQKKAIERMKDTLRIAYLTEAKVEKLCVWNNKTPHAIAAISMANGPGPKKRKSVAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 60 - protein, 169 aa, synthetic construct
AVPSSKEELIKAINSNFSLLNKKLESITPQLAFEPLLEGHAKGTTISVANLVSYLIGWGELVLHWHDQEAKGKTIIFPEEGFKWNELGRLAQKFYRDYEDITEYEVLLARLKENKQQLVALIERFSNDELYGKPWYNKWTRGRMIQFNTASPYKNASGRLNKLQKCLAE

SEQ ID NO: 61 - protein, 211 aa, synthetic construct
MTGDHIKVIYFNGRGRAESIRMTLVAAGVNYEDERISFQDWPKIKPTIPGGRLPAVKITDNHGHVKWMVESLAIARYMAKKHHMMGGTEEEYYNVEKLIGQAEDLEHEYYKTLMKPEEEKQKIIKEILNGKVPVLLDIICESLKASTGKLAVGDKVTLADLVLIAVIDHVTDLDKEFLTGKYPEIHKHRENLLASSPRLAKYLSDRAATPF

SEQ ID NO: 62 - protein, 169 aa, synthetic construct
AIPESKSELIEAINKNYASLVKKLAAVPAQRVFQPIMEGHVKGTTMSVAQLVSYLIGWGELVLSWHVKEANGNEIVFPEVGFKWNEMGKLAQKFYLDYQDITDYKVLLQRLENNKNEIIKLIEGFSDDDLYGVGWCEKYTRGRMIQLNTASPYKNATGRLATLLKSIDS

SEQ ID NO: 63 - protein, 168 aa, synthetic construct
IPESKQELIEAINKNYTLLTKKLAAVPEQKAYLPLMEGHAKGTMMSAAQLVSYLIGWGELVLSWHKQEQSGQKIAFPEAGYKWNQLGLLAQKFYRDYQDITDFKQLLSLLETNKKNLISLIDSFSNEELYGSPWHEKYTRGRMIQFNTSSPYKNATGRLNKLLKKIAE

SEQ ID NO: 64 - protein, 411 aa, synthetic construct
AQSEPELKLESVVIVSRHGVRAPTKATQLMQDVTPDAWPTWPVKLGELTPRGGELLAYLGHYWRQRLVADGLLPKCGCPQSGQVAILADVDERTRKTGEAFAAGLAPDCAITVHTQADTSSPDPLFNPLKTGVCQLDNANVTDAILERAGGSLADFTGHYQTAFRELERVLNFPQSNLCLKREKQDESCSLTQALPSELKVSADCVSLTGAVSLASMLTEIFLLQQAQGMPEPGWGRITDSHQWNTLLSLHNAQFDLLQRTPEVARSRATPLLDLIKTALTPHPPQKQAYGVTLPTSVLFLAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSL

SEQ ID NO: 65 - protein, 513 aa, synthetic construct
SMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 66 - protein, 554 aa, synthetic construct
KWWQSSVIYEIYPKSFYDTNNDGIGDLKGVEKKLPYLKELGIDAIWLTPIYLSPQIDNGYDVADYRTVDPMFGNNQDLKELIDRAHRLNIKVILDMVANHTSDQCIWFKESKKSKDNYFSDFYIWKDPKPDGSEPNNWAASFGGSAWTYVPERSQYYLHYYASQQPDLNWENPLVRQYVYDAMRYWKSQGVDGWRLDVITQISKDLTFKDVPTTDSNKKYIPLKNTSGPHMHKFIHELNQQVLKPFDMMSVGEAPNSNIEEILKLTSPERDELNMAFSFEHMRIDKAIGKNGAIAVKKPNFLKLKQVLSKMQYELNSKSWNALYFENHDRSRIPTRWGNDTEYRYESATAFATILHGLKGTPFIFQGEEIGMTNPKYSVDEYEDIDLKNKYQEQVKEKHLLTTDEFLKVEHKIARDNARTPMQWNNKKNAGFTEGKPWYKVNPNFTEINVESDKLSKKSIFRYYQQLITLRHKEDILIKGDYQELLEDDKNIFAYQRSWKNKSWLILVNMTEKDLSLNELHDNLKNYKKIILHNYLNQKLEDILKPYEAYILEK

SEQ ID NO: 67 - protein, 485 aa, synthetic construct
ATTGEAYFEQLLDHHNPEKGTFSQRYWWSTEYWGGPGSPVVLFNPGEVSADGYEGYLTNDTLTGVYAQEIQGAVILIEHRYWGDSSPYEVLNAETLQYLTLDQSILDMTYFAETVKLQFDNSSRSNAQNAPWVMVGGSYSGALTAWTESIAPGTFWAYHATSAPVEAIYDFWQYFYPIQQGMAQNCSKDVSLVAEYVDKIGKNGTAKEQQELKELFGLGAVEHYDDFAAVLPNGPYLWQDNDFVTGYSSFFQFCDAVEGVEAGAAVTPGPEGVGLEKALANYANWFNSTILPNYCASYGYWTDEWSVACFDSYNASSPIFTDTSVGNPVDRQWEWFLCNEPFFWWQDGAPEGTSTIVPRLVSASYWQRQCPLYFPEVNGYTYGSAKGKNSATVNSWTGGWDMTRNTTRLIWTNGQYDPWRDSGVSSTFRPGGPLVSTANEPVQIIPGGFHCSDLYMEDYYANEGVRKVVDNEVKQIKEWVEEYYA

SEQ ID NO: 68 - protein, 472 aa, synthetic construct
RKQLPKDFVIGGATAAYQVEGATKEDGKGRVLWDDFLEKQGRFSPDPAADFYHRYDEDLALAEAYGHQVIRLSIAWSRIFPDGAGAVEPRGVAFYHRLFAACAKHHLIPFVTLHHFDTPERLHAIGDWLSQEMLEDFVEYARFCFEEFPEIKHWITINEPTSMAVQQYTSGTFPPAETGHFDKTFQAEHNQIVAHARIVNLYKSMGLDGEIGIVHALQTPYPYSDSSEDQHAADLQDALENRLYLDGTLAGDYAPKTLALIKEILAANQQPMFKRTDEEMAAIKKAAHQLDFVGVNNYFSKWLRAYHGKSETIHNGDGSKGSSVARLHGIGEEKKPAGIETTDWDWSIYPRGMYDMLMRIHRDYPLVPAIYVTENGIGLKESLPAEVTPNTVIADPKRIDYLKKYLSAIADAIQAGANVKGYFVWSLQDQFSWTNGYSKRYGLFFVDFPTQKRYVKQSAEWLKQVSQTHVIP

SEQ ID NO: 69 - protein, 557 aa, synthetic construct
EKWWKNAVVYQVYPSSYQDSNNDGIGDLPGITKRLDYIKKLGVDIVWLSPIYKSPQVDNGYDISDYRAINPDFGSMEDFDKLLGKAHDLGLKIMMDLVVNHTSDENKWFEESRKSKTNPYRDYYIWRDGNAGKSPNNWGSFFRGPAWKYDEQTGQYYLHLFAPQQPDLNWENPNVRHSVYDMMNWWASKGVDGFRMDVINYISKPEGLPDAQLQADEELGNPEPLVANGHRVHEFLQEMNNKVMSKHRMVTVGEAPSVTTDEAIKYAGLDGKELDMVFQFEHMGLDGNENPVLGHWSDRKASLKDLRSNLTKWQEALEGKAWNSLYWNNHDQPRVVSRFGDDSNEEYRVLSAKMLATMLHLQQGTPFIFEGEEIGMTNSYFKKLSDYVDLDSINTYHQFVDKQHLVGSQTMLKYLAMHSRDNARTPMQWNSTDNGGFSKHEPWEHVNPNYKHINVKQSLDDPNSIFYYYQKLIRLRHELPVITDGKYRLVKGNEDDEAVYAYTRKDENTTLLVILNYTKTTLDRDYQVPDNAELVINNYPDDKGNTLRPYEAKVYKY

SEQ ID NO: 70 - protein, 985 aa, synthetic construct
IKHRLNGFTILEHPDPAKRDLLQDIVTWDDKSLFINGERIMLFSGEVHPFRLPVPSLWLDIFHKIRALGFNCVSFYIDWALLEGKPGDYRAEGIFALEPFFDAAKEAGIYLIARPGSYINAEVSGGGFPGWLQRVNGTLRSSDEPFLKATDNYIANAAAAVAKAQITNGGPVILYQPENEYSGGCCGVKYPDADYMQYVMDQARKADIVVPFISNDASPSGHNAPGSGTSAVDIYGHDSYPLGFDCANPSVWPEGKLPDNFRTLHLEQSPSTPYSLLEFQAGAFDPWGGPGFEKCYALVNHEFSRVFYRNDLSFGVSTFNLYMTFGGTNWGNLGHPGGYTSYDYGSPITETRNVTREKYSDIKLLANFVKASPSYLTATPRNLTTGVYTDTSDLAVTPLIGDSPGSFFVVRHTDYSSQESTSYKLKLPTSAGNLTIPQLEGTLSLNGRDSKIHVVDYNVSGTNIIYSTAEVFTWKKFDGNKVLVLYGGPKEHHELAIASKSNVTIIEGSDSGIVSTRKGSSVIIGWDVSSTRRIVQVGDLRVFLLDRNSAYNYWVPELPTEGTSPGFSTSKTTASSIIVKAGYLLRGAHLDGADLHLTADFNATTPIEVIGAPTGAKNLFVNGEKASHTVDKNGIWSSEVKYAAPEIKLPGLKDLDWKYLDTLPEIKSSYDDSAWVSADLPKTKNTHRPLDTPTSLYSSDYGFHTGYLIYRGHFVANGKESEFFIRTQGGSAFGSSVWLNETYLGSWTGADYAMDGNSTYKLSQLESGKNYVITVVIDNLGLDENWTVGEETMKNPRGILSYKLSGQDASAITWKLTGNLGGEDYQDKVRGPLNEGGLYAERQGFHQPQPPSESWESGSPLEGLSKPGIGFYTAQFDLDLPKGWDVPLYFNFGNNTQAARAQLYVNGYQYGKFTGNVGPQTSFPVPEGILNYRGTNYVALSLWALESDGAKLGSFELSYTTPVLTGYGNVESPEQPKYEQRKGAY

SEQ ID NO: 71 - protein, 722 aa, synthetic construct
DTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCAPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 72 - protein, 397 aa, synthetic construct
MLNPTPADKFSFGLWTVGWPARDQFGKASRPDLDPFDSIRKLKELGAWGFTFHDNDVIPFDATPAERDDLICTLKKTIDETGMVCEMVTTDTFAEPVFKDGAFTSNDRRVRRYGLRKVLRNVDLAAELGATTFVMWGGREGAEYDSSKDLHAALERYAEGIDTVAGYIKSQGYDLRIGLEPKPNEPRGDIFLPTIGHALAFIQRLDNGDVVGLNPETGHEQMATLNYTHGLAQALWSEKLFHIDLNGQNGPMYDQDLVFGHGNLISAFFTVDLLENGFPSKPGAYEGARHFDYKTSRTDHVTGVWDSALANMRMYLILKERALAFRADPEVQEALAYAGVHELAEPTLGADESLESFLADPSVYEGLDLTGPSERNYGYVRLHQLAVEHLTGARGVG

SEQ ID NO: 73 - protein, 297 aa, synthetic construct
MGIELTQMALNRKIAQKRPLENFFQLAEAAGIHQVELRNDMTTSDGSETVIDGMQVAEFQTLKQKYDLQILTINAIQQFNNPAKLNKNRDLLTKLAELSAQIGNQAIIFVPEVNAQDKRTEQQRLDDAVSSLQVFGDILSAYHLTGFIEPLGFRASTMRYPWTALDAINLSGRTEFKLTIDTFHFFLAHLTAEQFKAGVDINRVGLIHLSGIEPIHALREVVDEDRILITERDIMQNIEQVHLFEAMGYRGHYSFEPFSSRLAAETNQQLTQQILASIERLNQPTAVFSTEVTQPVG

SEQ ID NO: 74 - protein, 311 aa, synthetic construct
MKTATRLNAYLPHYDNKIDKVFGELKKVGINYVDINFPQQTIGIPARDMKKMLDENGLRPNGMEMRFEGHYLNGDLGNLNDKIYQDAIEMCKQAVDYCREIGGSMINIWSAHDGFDYSFQIDYLKVWHRIVKACQEIADYAPDIKFSIEYKPYEPRAYAFVDSMGTLGMILKDINRKNFGVLLDYCHMLMKHENPAMGSEIFASRGELFGVHINDGYGVHDDGLMVGTVTPFKTLEFLYYMKKYHFDGMYYFDTAPVIEDPSQETRKNIEMLKYLLNLLDRVGMEKIDKVINENDAIKARDLLLDFIKKVG

SEQ ID NO: 75 - protein, 311 aa, synthetic construct
MKTATRLNAYLPHYNNDINQVFKELKRVGINYVDINFPQQTIGIPATKMKQMLQQNDLCPNGMEMRFEDHYLNGDLSNLDDRIYRDAIEMCKKAADYCHAIGGTMINIWSAHDGFDYSFQIDYLKVWQRIVKACQEIADYAPDIKFSIEYKPYEPRAYAFVDSMGILGMMLKDINRPNFGVLLDYCHMLMKHENPAMGAALFASQKELFGVHINDGYGVHDDGLMVGTVTPFKTLEFLYYMKKYDFDGMYYFDTAPVIENPSQETAKNIEMLKYLLNLLDRIGMDHIETIIQENDAIKARELLLEFMKNVG

SEQ ID NO: 76 - protein, 568 aa, synthetic construct
SEWWKEAVVYQIYPRSFYDANGDGFGDLQGVIQKLDYIKNLGADVIWLSPVFDSPQDDNGYDISDYKNMYEKFGTNEDMFQLIDEVHKRGMKIVMDLVVNHTSDEHAWFAESRKSKDNPYRDYYLWKDPKPDGSEPNNWGSIFSGSAWTYDEGTGQYYLHYFSKKQPDLNWENEAVRREVYDVMRFWMDRGVDGWRMDVIGSISKYTDFPDYETDHSRSYIVGRYHSNGPRLHEFIQEMNREVLSHYDCMTVGEANGSDIEEAKKYTDASRQELNMIFTFEHMDIDKEQNSPNGKWQIKPFDLIALKKTMTRWQTGLMNVGWNTLYFENHDQPRVISRWGNDRKLRKECAKAFATVLHGMKGTPFIYQGEEIGMVNSDMPLEMYDDLEIKNAYRELVVENKTMSEKEFVKAVMIKGRDHARTPMQWDAGKHAGFTAGDPWIPVNSRYQDINVKESLEDQDSIFFYYQKLIQLRKQYKIMIYGDYQLLQENDPQVFSYLREYRGEKLLVVVNLSEEKALFEAPPELIHERWKVLISNYPQERADLKSISLKPYEAVMGISIGSSGGSSG

SEQ ID NO: 77 - protein, 221 aa, synthetic construct
AEGFLTQQTSPDSLSILPPPPAEDSVVFQADKAHYEFGRSLRDANRVRLASEDAYYENFGLAFSDAYGMDISRENTPILYQLLTQVLQDSHDYAVRNAKEYYKRVRPFVIYKDATCTPDKDENMAITGSYPSGHASFGWAVALILAEINPQRKAEILRRGYEFGESRVICGAHWQSDVEAGRLMGASVVAVLHNTPEFTKSLSEAKKEFEELNTPTNELTP

SEQ ID NO: 78 - protein, 443 aa, synthetic construct
ETLDEKKQKIESKQSEVASSIEAKEKELTELQENQSKIEKELKDINDKALDTSNKIEDKKEENDKTKEEIKKLKKEIKETEARIEKRNEILKKRVRSLQESGGSQGYIDVLLGSTSFGDFISRATAVSSIVDADKDLIKQQEQDKAKLEDSEADLNDKLKEVQAALAKLETMQKDLDKQLNEKDKLFDEAKASQKKTAKAISELKSEASELANQKANTEAEQARIKKEQEAAAALIKKQEEAQKASDETQTDDSQTATTESSKASSSDDSSDNSSDNSSNGSSNSSSNGSSSKKSSGSNSNSGGTVISNSGGIEGAISVGSSIVGQSPYKFGGGRTQSDINNRIFDCSSFVRWAYASAGVNLGPVGGTTTDTLVGRGQAVSASEMKRGDLVFFDTYKTNGHVGIYLGNGTFLNDNTSHGVSVDSMSNPYWKAAFKGVVRRVVQ

SEQ ID NO: 79 - protein, 209 aa, synthetic construct; identical to SEQ ID NO: 26

SEQ ID NO: 80 - protein, 217 aa, synthetic construct
RELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIVGSSGGSSG

SEQ ID NO: 81 - protein, 199 aa, synthetic construct
RPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 82 - protein, 207 aa, synthetic construct
RPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLWGSSGGSSG

SEQ ID NO: 83 - protein, 29 aa, synthetic construct
GPFPVIRGPFPVIRFALPEYLKGPFPVIR

SEQ ID NO: 84 - protein, 36 aa, synthetic construct
FALPEYLKGPFPVIRFILLQGVLKFFVAPFPEVFGK

SEQ ID NO: 85 - protein, 36 aa, synthetic construct
FALPEYLKFILLQGVLKFFVAPFPEVFGKGPFPVIR

SEQ ID NO: 86 - protein, 37 aa, synthetic construct
GPFPVIRFILLQGVLKFFVAPFPEVFGKFILLQGVLK

SEQ ID NO: 87 - protein, 249 aa, synthetic construct
RELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIVFALPEYLKFALPEYLKGPFPVIRFILLQGVLKGSSGGSSG

SEQ ID NO: 88 - protein, 282 aa, synthetic construct
RPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLWGPFPVIRFILLQGVLKFFVAPFPEVFGKFILLQGVLKFILLQGVLKFFVAPFPEVFGKFALPEYLKFILLQGVLKGSSGGSSG

SEQ ID NO: 89 - protein, 21 aa, synthetic construct
RGRVSPLMLLLGILVLASVSA

SEQ ID NO: 90 - protein, 151 aa, synthetic construct
RQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRY

SEQ ID NO: 91 - protein, 147 aa, synthetic construct
GPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 92 - protein, 260 aa, synthetic construct
TPQNITDLCAEYHNTQIYTLNDKIFSYTESLAGKREMAIITFKNGAIFQVEVPGSQHIDSQKKAIERMKDTLRIAYLTEAKVEKLCVWNNKTPHAIAAISMANGPGPKKRKSVGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 93 - protein, 57 aa, synthetic construct
AQEPVKGPVSTKPGSCPIILIRCAMLNPPNRCLKDTDCPGIKKCCEGSCGMACFVPQ

SEQ ID NO: 94 - protein, 31 aa, synthetic construct
HAEGTFTSDVSSYLEGQAAKEFIAWLVKGRG

SEQ ID NO: 95 - protein, 39 aa, synthetic construct
HGEGTFTSDLSKQMEEEAVRLFIEWLKNGGPSSGAPPPS

SEQ ID NO: 96 - protein, 21 aa, synthetic construct
SSMKLSFRARAYGFRGPGPQL

SEQ ID NO: 97 - protein, 82 aa, synthetic construct
VPIDIDKTKVQNIHPVESAKIEPPDTGLYYDEYLKQVIDVLETDKHFREKLQKADIEEIKSGRLSKELDLVSHHVRTKLDEL

SEQ ID NO: 98 - protein, 14 aa, synthetic construct
CCEYCCNPACTGCY

SEQ ID NO: 99 - protein, 16 aa, synthetic construct
NDECELCVNVACTGCL

SEQ ID NO: 100 - protein, 16 aa, synthetic construct
NDDCELCVNVACTGCL

SEQ ID NO: 101 - protein, 34 aa, synthetic construct
IKPEAPREDASPEELNRYYASLRHYLNLVTRQRY

SEQ ID NO: 102 - protein, 15 aa, synthetic construct
PGTCEICAYAACTGC

SEQ ID NO: 103 - protein, 59 aa, synthetic construct
STFWAYQPDGDNDPTDYQKYEHTSSPSELLAPGDYKLDYKDHDGDYKDHDIDYKDDDDK

SEQ ID NO: 104 - protein, 120 aa, synthetic construct
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 105 - protein, 260 aa, synthetic construct
LKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 106 - protein, 133 aa, synthetic construct
EVQLVESGGGLVQAGGSLRLSCTASGRTFSSYAMGWFRQTPGKEREFVAAITSSGGSTYYADSVKGRFTISRDNAKSTMYLQMDSLMLDDTSVYYCAADSSRPQYSDSALRRILSLSNSYPYWGQGTQVTVSS

SEQ ID NO: 107 - protein, 123 aa, synthetic construct
EVQLVESGGGLVQAGDSLRLSCAASGRTFSSYRMGWFRQAPGKEREFVAAISRSGASAYYADSVKGRFTCSRDNAKDTVYLQMNKLKPEDTAVYYCAASSRMGVDDPETYGYWGQGTQVTVSS

SEQ ID NO: 108 - protein, 123 aa, synthetic construct
EVQLVESGGGLVQAGGSLRLSCAASGGTFSSYRMGWFRQAPGKEREFVTLISRSGGTTDYTDSVKGRFTISRDNAKNTVSLQMNNLKPADTAVYYCAAAGSMGWNHLRDYDYWGQGTQVTVSS

SEQ ID NO: 109 - protein, 126 aa, synthetic construct
EVQLVESGGGLVQPGGSLRLSCAASGFTLDYLAIGWFRQAPGKEREGVSCVSSSGQYTYYADSVKGRFTISRDNAESTVYLQMNSLKPEDTAVYYCATDPECYRVRGYYNGEYDYWGQGTQVTVSS

SEQ ID NO: 110 - protein, 125 aa, synthetic construct
EVQLVESGGGLVQAGGSLRLSCAASGRTFEGNPMGWFRQASGKKREFVASIDWSGGITSYADSVKGRFTISRDNAKNTVYLQMNSLKPEDTAVYYCAASARFGSGSYYDLMYDYWGQGTQVTVSS

SEQ ID NO: 111 - protein, 33 aa, synthetic construct
HGDGSFSDEMNTILDNLAARDFINWLIQTKITD

SEQ ID NO: 112 - protein, 125 aa, synthetic construct
DVQLVSYSWFRQAISKGGRFTISRDNAKSRAYGSSWGQGTESGGGMGAPGKYKYDAVNTVYLQINSLRLRLADTLVTVSLVQPGEREFSLEGRPEDTAVYYCYEYSGSLRLVVASSCAASGRTFS

SEQ ID NO: 113 - protein, 118 aa, synthetic construct
EVQLVESGGGLVQAGGSLRLSCAASGRTFSSYDMGWYRQAPGKEREFVAIITWNSSTYYADSVKGRFTISRDNAKNTVYLQMNSLKPEDTAIYYCNAQYGLGYAEDYWGQGTQVTVSS

SEQ ID NO: 114 - protein, 128 aa, synthetic construct
QVQLQESGGGSVQAGGSLRLSCVASGSIFSINAMDWYRQAPGKQRELVAGITSGGSTNYGDFVKGRFTISRDNAKNTVYLQMDSLKPEDTAVYYCAAEVGGWGPPRPDYWGHGTQVTVSSGSLEVLFQ

SEQ ID NO: 115 - protein, 127 aa, synthetic construct
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGS

SEQ ID NO: 116 - protein, 133 aa, synthetic construct
GSVQAGGSLRLSCAASGDADSRNCMGWIRQAPGKEREGVAGIYIGGGRTWYAAAVKGRFTISQDNAKNTVYLQMNSLKPEDTAMYYCAIGEIKVVDGKSSCAAEDAGGQGTLVTVSSAHHSEDPGPRGLAAAG

SEQ ID NO: 117 - protein, 120 aa, synthetic construct [sequence omitted]

SEQ ID NO: 118 - protein, 115 aa, synthetic construct
DVQLVESGGGLVQPGGSLKLSCAASGFDFSSHWMYWVRQAPGKELEWLSEINTNGLITHYGDSVKGRFTVSRNNAANKMYLELTRLEPEDTALYYCARNQHGLNKGQGTQVTVSS

SEQ ID NO: 119 - protein, 119 aa, synthetic construct
EVQLLESGGGLVQPGGSLRLSCAASGFTFAHETMVWVRQAPGKGLEWVSHIPPDGQDPFYADSVKGRFTISRDNSKNTLYLQMNSLRAEDTAVYHCALLPKRGPWFDYWGQGTLVTVSS

SEQ ID NO: 120 - protein, 124 aa, synthetic construct
DVQLVESGGGLVQAGGSLRLSCAASGGTFSSIIMAWFRQAPGKEREFVGAVSWSGGTTVYADSVLGRFEISRDSARKSVYLQMNSLKPEDTAVYYCAARPYQKYNWASASYNVWGQGTLVTVSS

SEQ ID NO: 121 - protein, 52 aa, synthetic construct
YRQSMNNFQGLRSFGCRFGTCTVQKLAHQIYQFTDKDKDNVAPRSKISPQGY

SEQ ID NO: 122 - protein, 27 aa, synthetic construct
IPLRGAFINGRWDSQCHRFSNGAIACA

SEQ ID NO: 123 - protein, 335 aa, synthetic construct
RNEEQSGDGSRHSGSRHHEPSTRAGSSRHSQVGQGESAGSKTSRRQGSSVSQDRDSEGHSEDSERRSESASRNHYGSAREQSRHGSRNPRSHQEDRASHGHSAESSRQSGTRHAETSSGGQAASSQEQARSSPGERHGSRHQQSADSSTDSGTGRRQDSSVVGDSGNRGSSGSQASDSEGHSEESDTQSVSAHGQAGPHQQSHQESTRGQSGERSGRSGSFLYQVSTHEQSESAHGRTGPSTGGRQRSRHEQARDSSRHSASQEGQDTIRGHPGSSRGGRQGSHYEQSVDSSGHSGSHHSHTTSQERSDVSRGQSGSRSVSRQTRRMRRMRRMRR

SEQ ID NO: 124 - protein, 9 aa, synthetic construct
LEISSTCDA

SEQ ID NO: 125 - protein, 25 aa, synthetic construct
FCTFFEKHHRKWDILLEKSTGVMEA

SEQ ID NO: 126 - protein, 125 aa, synthetic construct
QVQLQESGGGLVQPGGSLRLSCAASGFTLDYYYIGWFRQAPGKEREAVSCISGSSGSTYYPDSVKGRFTISRDNAKNTVYLQMNSLKPEDTAVYYCATIRSSSWGGCVHYGMDYWGKGTQVTVSS

SEQ ID NO: 127 - protein, 129 aa, synthetic construct
QLQLVETGGGLVQPGGSLRLSCAASGFTFSDYVMTWVRQAPGKGPEWIATINTDGSTMRDDSTKGRFTISRDNAKNTLYLQMTSLKPEDTALYYCARGVISASAIRGAVRGPGTQVTVSSEPKTPKPQP

SEQ ID NO: 128 - protein, 135 aa, synthetic construct
QVQLVESGGGLVQPGGSLRLSCAASGFSLDYYGIGWFRQAPGKERQEVSYISASAKTKLYSDSVKGRFTISRDNAKNAVYLEMNSLKREDTAVYYCARRRFDASASNRWLAADYDYWGQGTQVTVSSEPKTPKPQ

SEQ ID NO: 129 - protein, 123 aa, synthetic construct
QVQLQDSGGGLVQVGDRLSLSCAASGRTFSSYDMAWFRQAPGKEREFVAAITTSEGTWYGDAGKGRFTIARVNAKNTVYLHMNRLKPEDTAVYYCAASNQGGSLQISTNYNYWGQGTQVTVSS

SEQ ID NO: 130 - protein, 126 aa, synthetic construct
EVQLVESGGGLVQPGGSLRLSCAASGSTLDYYAIGWFRQAPGKEREGVSCTSNSGSTYYGGSVKGRFTASRDNAKNTVYLQMNSLRPEDTAVYYCVATIGCATLGGTLDVQRYYYRGQGTQVTVSS

SEQ ID NO: 131 - protein, 122 aa, synthetic construct
DVQLLESGGGLVQPGGSLRLSCAASGFTFGNYDMAWVRQAPGKRPEWVSSIDTGGDITHYADSVKGRFTISRDNAKNTLYLQMNSLRPEDTAVYWCATDEEYALGPNEFDYYGQGTLVTVSS

SEQ ID NO: 132 - protein, 90 aa, synthetic construct
ATGAAAAAATTGTTGTTGTTGTTGGTTGTTTTGGCAGGCTTGGCAGCATTGTTGTTGTCAGGCGCAGGCACCTCAGTTTCAGCAGCAACC

SEQ ID NO: 133 - protein, 90 aa, synthetic construct
ATGAAAAAATTGTTGATTGGCTTGGTTGTTTTGTTGGGCTTGGCAGCATTGTTGTTGTCAGGCTTGGGCACCTCAGTTTCAGCAGCAACC

SEQ ID NO: 134 - protein, 90 aa, synthetic construct
ATGAAAAAAATTTTGTTGTTGGTTGTTGTTTTGTTGGGCTTGGCAGCATTGTTGTGGTCAGGCGCAGGCACCTCAGTTTCAGCATCAACC

SEQ ID NO: 135 - protein, 90 aa, synthetic construct
ATGAAAAAGCTGCTCCTGGGTCTGGTTCTGCTCCTGGGCCTGGCAGCGCTGGCCCTGACCGGTTGTGGCACTCCGGTGAGCGCCGCAACT

SEQ ID NO: 136 - protein, 90 aa, synthetic construct
ATGAAGAAACTGACCCTGCTCCTGACTGTCTCCCTGGGTATGGCAGGTTTCCTCCTGTCCGGTTGCGGCCAGCCTGTTTCCGCGGCCCAG

SEQ ID NO: 137 - protein, 90 aa, synthetic construct
ATGAAGAAACTGCTCCTGCTCCTGGTAGTTCTCCTGTCCGTTGCCGCTCTCCTGGCGAGCGGCTGTGGTACTTTCGTATCCGCAGCAACC

SEQ ID NO: 138 - protein, 90 aa, synthetic construct
ATGAAACGCCTGCTCCTGAAAGCTGTTGTGCTGCTCTCTCTGGCAGCCCTCCTGTGGTCTGGCGCAGGCACCTCCGTGAGCGCTGCCACC

SEQ ID NO: 139 - protein, 90 aa, synthetic construct
ATGCACAAACTCCTGATGCTCCTGGTCGTGCTCCTGGGTCTCGCTGGCCTGTCTCTGAGCGGTTGCGGTAGCTCCACGTCCGCTGCGACC

SEQ ID NO: 140 - protein, 90 aa, synthetic construct
ATGAAGCGCCTGCTCATCGGTGCGGTTGTCCTCCTGGGTCTGGCGGCTCTGCTCCTGTCCGGTTGTGGTACTTCTGTTTCCGCAGCCACG

SEQ ID NO: 141 - protein, 30 aa, synthetic construct
MKKALLLLGVATGLAAMLLSGCGTSVSAAT

SEQ ID NO: 142 - protein, 30 aa, synthetic construct
MKKLLLLLVVLLGLAFLLLSGCGTSTSAQN

SEQ ID NO: 143 - protein, 113 aa, synthetic construct
TPQNITDLCAEYHNTQIYTLNDKIFSYTESLAGKREMAIITFKNGAIFQVEVPGSQHIDSQKKAIERMKDTLRIAYLTEAKVEKLCVWNNKTPHAIAAISMANGPGPKKRKSV

SEQ ID NO: 144 - protein, 30 aa, synthetic construct
MKRLLELLVVVLGLAALLLSGCGTSVSAAS

SEQ ID NO: 145 - protein, 30 aa, synthetic construct
MKKLTLLIVVLLGLAALGLSGCGTSVSAAT

SEQ ID NO: 146 - protein, 30 aa, synthetic construct
MKKLHLLLVVVFGLAALLLAGCGTSVSAAT

SEQ ID NO: 147 - protein, 30 aa, synthetic construct
MKKLLLLIVVLLGLAFLLLSGCGTSVSAAS

SEQ ID NO: 148 - protein, 30 aa, synthetic construct
MKKLLLLLVASLSLAAVLLSGIGTSVSAAT

SEQ ID NO: 149 - protein, 30 aa, synthetic construct
MKKKLLTLVLLLGLAALLLTGCGQSVSAAT

SEQ ID NO: 150 - protein, 30 aa, synthetic construct
MKKLLLLLVVLLGLGGFLLSGCGTSVSAST

SEQ ID NO: 151 - protein, 30 aa, synthetic construct
MKKLIATLVVLLGLAALMLSACGTSVSAAT

SEQ ID NO: 152 - protein, 30 aa, synthetic construct
MKKLHLLLVGLLGLAALLLSGIGTSVSAAT

SEQ ID NO: 153 - protein, 30 aa, synthetic construct
MKKLLLLLLVLLSVAALLLSACGTSVSAAT

SEQ ID NO: 154 - protein, 30 aa, synthetic construct
MKKLLLLLVVLLGLAALLLSGCGTTVCAAT

SEQ ID NO: 155 - protein, 30 aa, synthetic construct
MKKWLLLLVVLLGLGALLLSGCGTSVSAAT

SEQ ID NO: 156 - protein, 30 aa, synthetic construct
MKKLLLLLVVLLGLAFLLLSGCGTTVSAAT

SEQ ID NO: 157 - protein, 30 aa, synthetic construct
MKKLLLKLVVLLGLAAILLSGCGTTVSAAT

SEQ ID NO: 158 - protein, 30 aa, synthetic construct
MKKLLGLLVVALSLAALLFSGVGTSVSAAT

SEQ ID NO: 159 - protein, 30 aa, synthetic construct
MKKLLLLLGVLAGLAALLLSGCGTSVSADT

SEQ ID NO: 160 - protein, 30 aa, synthetic construct
MKKLLLLLVVLLGLTAFLLSGCGTSVSAAT

SEQ ID NO: 161 - protein, 30 aa, synthetic construct
MKKLLLLLVVLLGLAALCLSGCGTSVSASG

SEQ ID NO: 162 - protein, 30 aa, synthetic construct
MKKLLLLLTVLLGLAALWLSGFGTSSSAAT

SEQ ID NO: 163 - protein, 30 aa, synthetic construct
MKKLLLLAVVLLGLSALLLSGCGTSVSAAG

SEQ ID NO: 164 - protein, 30 aa, synthetic construct
MKKLLITLVCAIGLAALLLSGCGTSVSAAT

SEQ ID NO: 165 - protein, 30 aa, synthetic construct
MKKLLLGLVVVLGLAALFLSGCGTSVSAAT

SEQ ID NO: 166 - protein, 30 aa, synthetic construct
MKRLLILLVAVLGLAALLLSGCGTSVSAST

SEQ ID NO: 167 - protein, 30 aa, synthetic construct
MKKALLKLVVLMGLAALLLSGCGTSVSAAS

SEQ ID NO: 168 - protein, 27 aa, synthetic construct
MKKLLLLLVVALGLAALLLSTSTSAST

SEQ ID NO: 169 - protein, 30 aa, synthetic construct
MKRLLLLLTLLLGLAAFLLSGCGTSVSAAN

SEQ ID NO: 170 - protein, 30 aa, synthetic construct
MKKLLLLLVVLTGLAAMLLSSCGTSVSAAT

SEQ ID NO: 171 - protein, 30 aa, synthetic construct
MKKLKALLVVLLGLAALLFSGCGTSVSAAT

SEQ ID NO: 172 - protein, 30 aa, synthetic construct
MKKLLLLLVVLLGLAMLLLSGCGTQVSASS

SEQ ID NO: 173 - protein, 30 aa, synthetic construct
MKKLLALLVVALGLSALLLSGCGTSVSAAT

SEQ ID NO: 174 - protein, 30 aa, synthetic construct
MKKLILLLTVLTGLAALLLSGCGSSVSAAT

SEQ ID NO: 175 - protein, 30 aa, synthetic construct
MKKLLLLLVVALSLAALLLSGCGTSVSAQT

SEQ ID NO: 176 - protein, 30 aa, synthetic construct
MKRLLLLLLVLLGLALLLLSGCGASVSAAT

SEQ ID NO: 177 - protein, 30 aa, synthetic construct
MKKLLLLAVVLLGLAALLASGCGTSTSAAT

SEQ ID NO: 178 - protein, 30 aa, synthetic construct
MKKLLMLLVVLLSIAALLLAGCGTSVSAAT

SEQ ID NO: 179 - protein, 30 aa, synthetic construct
MKKLLFLLVVLLGLAALGLSGVGTSVSAAT

SEQ ID NO: 180 - protein, 30 aa, synthetic construct
MKKLLLGLVVLFGLAALLLSGAGVSVSAST

SEQ ID NO: 181 - protein, 30 aa, synthetic construct
MKKILLLLVGLFGLAALLLSGCGTSVSAAT

SEQ ID NO: 182 - protein, 30 aa, synthetic construct
MKKWLLALVVLLTLAAFLVSGCGTSVSAAT

SEQ ID NO: 183 - protein, 30 aa, synthetic construct
MKRLLLGLVVLIGVTAFLLSGCGTSVSAAT

SEQ ID NO: 184 - protein, 30 aa, synthetic construct
MKKLLLLLVGLLGVAALLLSGCGTSASAAT

SEQ ID NO: 185 - protein, 30 aa, synthetic construct
MKKVLALLVVLLGLAALGLSGCGTSVSAAT

SEQ ID NO: 186 - protein, 30 aa, synthetic construct
MKKLLLLLVCLLGLAALGLSACGTSVSAAT

SEQ ID NO: 187 - protein, 30 aa, synthetic construct
MKKLLILLTLVISLAAFLLSGCGTSVSAAT

SEQ ID NO: 188 - protein, 30 aa, synthetic construct
MKKLLLLLVVVLGLALLLLSGCGTSVHAAS

SEQ ID NO: 189 - protein, 30 aa, synthetic construct
MKKLISLLVVLLGLAALLLSGCGTSDSAAA

SEQ ID NO: 190 - protein, 30 aa, synthetic construct
MKKLLILLVVLLGMAALLLSGCGTSVSADT

SEQ ID NO: 191 - protein, 30 aa, synthetic construct
MKKLLLLLTVALGLAALLLSGLGTSVSAAT

SEQ ID NO: 192 - protein, 30 aa, synthetic construct
MKKLTLGLVVAAGLAALLLSGCGTPTSAAE

SEQ ID NO: 193 - protein, 30 aa, synthetic construct
MKRLLLLLVVVLGLAALLLSGCGTSVHAAT

SEQ ID NO: 194 - protein, 30 aa, synthetic construct
MKRLLELLVVLLGLAALCLSGCGTSVSAAA

SEQ ID NO: 195 - protein, 30 aa, synthetic construct
MKKLLLLAVVLLGLAGLLLSGCGTPVSAAT

SEQ ID NO: 196 - protein, 30 aa, synthetic construct
MKKLLLLVTVLLGLAAMGLSGAGVSVSAST

SEQ ID NO: 197 - protein, 30 aa, synthetic construct
MKKLLALLVVLLGLAALLLSVCGTSVSAAT

SEQ ID NO: 198 - protein, 30 aa, synthetic construct
MKKLLLLLVMLFGLAFLLLSGCGTSVSAST

SEQ ID NO: 199 - protein, 30 aa, synthetic construct
MKKLLLLLVVALGLAAMLLSGCGSSVHAAT

SEQ ID NO: 200 - protein, 30 aa, synthetic construct
MKKLLLLLTVLLSLAALLLSGTGTPVSAAT

SEQ ID NO: 201 - protein, 30 aa, synthetic construct
MKKLLLLLVGLLGLAALLLSGVGTSVHAAT

SEQ ID NO: 202 - protein, 30 aa, synthetic construct
MKRLLILLVVLIGLAALGLSGCGTSVSAAT

SEQ ID NO: 203 - protein, 30 aa, synthetic construct
MKKLLSLIVVLLGLAAMLLSGCGTSVSAAC

SEQ ID NO: 204 - protein, 30 aa, synthetic construct
MKKLLLLLLVLLFMAALLLSGCGTSVSAST

SEQ ID NO: 205 - protein, 30 aa, synthetic construct
MKRLLLLLVLLLGLAAALLTGCGTSVSAAT

SEQ ID NO: 206 - protein, 30 aa, synthetic construct
MKKLLLLLVVLLGVTALLLSGCGKSVSAAT

SEQ ID NO: 207 - protein, 30 aa, synthetic construct
MKKLLLLLVVLLGCAALLLSGCGTPSSAAT

SEQ ID NO: 208 - protein, 30 aa, synthetic construct
MKKLLLLFVVLLGLAALLFSGAGTSVSAAT

SEQ ID NO: 209 - protein, 30 aa, synthetic construct
MKKLLLLLVVLLFVAALGLSGCGTSTSAAT

SEQ ID NO: 210 - protein, 30 aa, synthetic construct
MKKLLLFLVVLLGMSALGLSGCGTSVHAAS

SEQ ID NO: 211 - protein, 30 aa, synthetic construct
MKKLLLLLVLLLGLAAFLLSGCGTPVSAQT

SEQ ID NO: 212 - protein, 30 aa, synthetic construct
MKRLLELLVVLLGLAFLMLSGCGTRVHAAT

SEQ ID NO: 213 - protein, 90 aa, synthetic construct
ATGAAACGCTTGCTTGAGTTATTGGTTGTGGTTCTGGGCCTGGCCGCACTACTGTTGAGTGGCTGCGGCACTAGCGTCAGCGCTGCTTCA

SEQ ID NO: 214 - protein, 90 aa, synthetic construct
ATGAAGAAATTAACGCTCCTTATTGTTGTACTGCTTGGACTCGCGGCCTTGGGACTGAGCGGATGCGGAACATCTGTTTCCGCGGCAACG

SEQ ID NO: 215 - protein, 90 aa, synthetic construct
ATGAAAAAGTTGCATCTATTATTGGTGGTAGTCTTCGGCCTCGCTGCCTTGTTATTGGCGGGCTGTGGGACCTCCGTCAGCGCCGCAACC

SEQ ID NO: 216 - protein, 90 aa, synthetic construct
ATGAAAAAGTTGCTCTTATTGATTGTTGTCCTATTGGGGTTGGCATTTCTCCTTCTATCAGGCTGTGGAACCTCGGTTTCCGCTGCGTCT

SEQ ID NO: 217 - protein, 90 aa, synthetic construct
ATGAAGAAATTGCTTCTGCTATTAGTTGCAAGCCTCTCTCTGGCAGCTGTCTTGCTGTCAGGAATCGGCACGTCTGTTTCGGCCGCCACG

SEQ ID NO: 218 - protein, 90 aa, synthetic construct
ATGAAGAAAAAGTTACTGACATTGGTATTGCTGCTCGGGTTGGCTGCACTTCTGCTAACTGGATGCGGACAATCTGTTAGTGCTGCGACA

SEQ ID NO: 219 - protein, 90 aa, synthetic construct
ATGAAGAAATTGTTATTGCTTTTGGTCGTTTTGCTGGGATTGGGCGGTTTTTTGCTTTCAGGCTGTGGGACGTCAGTAAGTGCCTCAACC

SEQ ID NO: 220 - protein, 90 aa, synthetic construct
ATGAAGAAACTAATTGCCACGTTAGTTGTCCTTTTGGGGCTTGCCGCACTAATGCTGAGCGCTTGTGGAACATCTGTATCCGCAGCAACG

SEQ ID NO: 221 - protein, 90 aa, synthetic construct
ATGAAGAAATTACATCTCCTTTTGGTAGGCCTTTTAGGATTAGCTGCGTTGCTTTTAAGCGGAATCGGCACGTCAGTATCAGCGGCAACG

SEQ ID NO: 222 - protein, 90 aa, synthetic construct
ATGAAGAAACTTTTGTTATTGTTACTCGTCTTATTGAGCGTAGCAGCCTTACTCTTATCGGCGTGCGGGACTTCGGTCAGCGCGGCTACG

SEQ ID NO: 223 - protein, 90 aa, synthetic construct
ATGAAAAAGCTCCTTTTACTGCTTGTAGTGTTGCTTGGACTGGCAGCTCTCCTGCTAAGCGGCTGCGGGACAACCGTTTGCGCGGCAACG

SEQ ID NO: 224 - protein, 90 aa, synthetic construct
ATGAAGAAATGGTTGCTTCTGCTTGTTGTCCTATTGGGTTTGGGTGCTTTACTGTTATCAGGCTGCGGAACTTCAGTATCCGCAGCGACA

SEQ ID NO: 225 - protein, 90 aa, synthetic construct
ATGAAGAAACTACTTTTATTGTTAGTTGTCTTACTGGGCTTAGCTTTTCTGTTGCTATCAGGTTGTGGCACAACCGTTAGCGCCGCCACC

SEQ ID NO: 226 - protein, 90 aa, synthetic construct
ATGAAAAAGTTGCTTCTGAAGTTAGTCGTGTTACTGGGTCTTGCGGCTATCTTACTTTCCGGTTGCGGGACGACCGTTTCAGCTGCCACA

SEQ ID NO: 227 - protein, 90 aa, synthetic construct
ATGAAGAAATTATTGGGACTTTTGGTGGTCGCGTTGTCCTTGGCGGCACTGTTATTTTCAGGTGTAGGGACTTCGGTTTCGGCCGCTACT

SEQ ID NO: 228 - protein, 90 aa, synthetic construct
ATGAAGAAATTGCTTTTATTGCTAGGCGTTTTGGCAGGTTTGGCTGCCTTACTGCTTAGCGGGTGCGGCACCTCCGTCTCGGCTGATACA

SEQ ID NO: 229 - protein, 90 aa, synthetic construct
ATGAAGAAATTGCTCTTGCTTTTAGTAGTTTTACTTGGTTTGACTGCATTTCTGTTAAGCGGGTGTGGTACGTCAGTCTCCGCAGCCACT

SEQ ID NO: 230 - protein, 90 aa, synthetic construct
ATGAAAAAGCTTCTGTTGCTGCTAGTTGTGTTGTTAGGTCTGGCGGCCCTTTGTCTTTCAGGTTGCGGCACTTCCGTCTCGGCATCAGGC

SEQ ID NO: 231 - protein, 90 aa, synthetic construct
ATGAAAAAGCTCTTACTCCTACTTACCGTTCTACTTGGCCTAGCAGCCCTCTGGTTGTCAGGGTTTGGTACATCTTCAAGCGCCGCAACT

SEQ ID NO: 232 - protein, 90 aa, synthetic construct
ATGAAAAAGCTTCTGCTATTGGCCGTGGTATTATTGGGCCTCTCAGCGTTACTGTTGAGTGGATGTGGGACAAGCGTCAGCGCCGCAGGG

SEQ ID NO: 233 - protein, 90 aa, synthetic construct
ATGAAAAAGCTCCTTATAACCCTTGTATGCGCCATCGGCCTTGCGGCCTTGCTCCTGAGCGGCTGTGGCACTAGCGTGTCAGCGGCCACC

SEQ ID NO: 234 - protein, 90 aa, synthetic construct
ATGAAGAAATTATTGTTAGGCTTGGTTGTGGTTCTGGGCCTCGCTGCCCTTTTCTTGAGCGGCTGCGGCACGTCTGTATCAGCAGCTACG

SEQ ID NO: 235 - protein, 90 aa, synthetic construct
ATGAAGAGACTTTTAATCCTTTTAGTAGCCGTCCTAGGGCTAGCTGCGTTACTTTTAAGTGGCTGTGGAACATCGGTAAGCGCCTCGACC

SEQ ID NO: 236 - protein, 90 aa, synthetic construct
ATGAAGAAAGCATTGTTAAAACTGGTAGTTCTGATGGGGCTTGCAGCGCTTCTGTTGAGTGGTTGCGGAACTAGTGTTAGCGCCGCCTCC

SEQ ID NO: 237 - protein, 81 aa, synthetic construct
ATGAAGAAACTCCTACTTTTGCTTGTTGTGGCCTTAGGCTTGGCGGCTCTTCTACTGAGTACATCAACGTCGGCGTCAACC

SEQ ID NO: 238 - protein, 90 aa, synthetic construct
ATGAAACGCTTGTTACTCTTGCTCACGTTGCTTCTAGGCCTTGCTGCCTTCTTATTGTCGGGCTGCGGCACTTCAGTTTCCGCAGCGAAC

SEQ ID NO: 239 - protein, 90 aa, synthetic construct
ATGAAGAAACTTCTGTTATTGCTTGTGGTTCTTACCGGACTTGCTGCAATGCTTCTATCGTCATGCGGCACTTCGGTGTCAGCTGCGACT

SEQ ID NO: 240 - protein, 90 aa, synthetic construct
ATGAAAAAGCTCAAAGCTCTATTAGTAGTGCTACTTGGACTCGCGGCATTATTGTTCAGTGGTTGTGGTACCTCAGTGTCCGCAGCTACC

SEQ ID NO: 241 - protein, 90 aa, synthetic construct
ATGAAAAAGCTGCTTTTGTTATTGGTTGTACTTTTGGGACTTGCTATGCTCTTACTTTCTGGTTGCGGTACTCAAGTCTCAGCTTCCAGC

SEQ ID NO: 242 - protein, 90 aa, synthetic construct
ATGAAGAAACTATTGGCACTTTTAGTTGTCGCGCTAGGTCTTTCCGCTTTGCTTTTGTCTGGTTGTGGTACCAGCGTGTCGGCCGCTACG

SEQ ID NO: 243 - protein, 90 aa, synthetic construct
ATGAAGAAACTAATTCTCCTTTTGACGGTGTTGACGGGCTTAGCAGCGTTATTGCTATCGGGTTGCGGATCCAGTGTTTCCGCAGCCACA

SEQ ID NO: 244 - protein, 90 aa, synthetic construct
ATGAAAAAGTTACTGTTGCTCCTAGTTGTAGCTCTTTCTTTGGCCGCGTTGTTACTTAGCGGCTGTGGCACGTCAGTTAGTGCTCAAACC

SEQ ID NO: 245 - protein, 90 aa, synthetic construct
ATGAAGCGACTTTTGTTACTTTTACTTGTTCTACTTGGACTTGCGTTGCTTTTGCTCTCTGGTTGCGGCGCCTCTGTTTCGGCAGCTACG

SEQ ID NO: 246 - protein, 90 aa, synthetic construct
ATGAAAAAGCTTTTATTGCTTGCTGTGGTTCTTCTGGGGTTAGCCGCACTGTTGGCGAGCGGGTGCGGTACTTCCACTAGTGCTGCAACG

SEQ ID NO: 247 - protein, 90 aa, synthetic construct
ATGAAGAAATTATTGATGTTATTGGTCGTGTTGCTTTCGATTGCTGCGCTGTTGCTAGCCGGCTGTGGCACATCTGTGTCGGCTGCCACG

SEQ ID NO: 248 - protein, 90 aa, synthetic construct
ATGAAAAAGCTACTGTTTCTACTTGTTGTCCTTCTGGGCTTGGCGGCTCTAGGCCTCAGTGGTGTCGGCACATCAGTAAGCGCTGCTACC

SEQ ID NO: 249 - protein, 90 aa, synthetic construct
ATGAAGAAATTACTACTTGGCCTCGTGGTCCTGTTTGGGCTCGCTGCCCTTTTGCTGAGTGGAGCGGGGGTTTCAGTTTCAGCATCCACC

SEQ ID NO: 250 - protein, 90 aa, synthetic construct
ATGAAAAAGATCCTGCTTTTACTGGTCGGGTTGTTTGGTCTAGCTGCCCTTTTGTTATCAGGCTGTGGAACATCAGTGTCTGCCGCAACG

SEQ ID NO: 251 - protein, 90 aa, synthetic construct
ATGAAGAAATGGCTTTTGGCATTGGTTGTGTTGCTAACCCTTGCAGCCTTCTTAGTATCAGGTTGCGGCACAAGCGTGTCAGCTGCGACG

SEQ ID NO: 252 - protein, 90 aa, synthetic construct
ATGAAAAGGCTTTTGCTTGGCCTGGTAGTTCTTATTGGCGTAACTGCGTTTCTGTTATCGGGTTGTGGTACATCCGTCTCCGCCGCCACC

SEQ ID NO: 253 - protein, 90 aa, synthetic construct
ATGAAGAAATTACTTCTGCTCCTTGTAGGCTTGCTGGGCGTAGCTGCCTTGCTTTTGAGTGGTTGCGGGACCTCCGCCTCGGCTGCAACC

SEQ ID NO: 254 - protein, 90 aa, synthetic construct
ATGAAAAAGGTGCTTGCCCTGCTTGTCGTTCTCTTAGGTTTGGCAGCTCTTGGTTTATCAGGATGCGGTACGTCAGTGTCAGCCGCCACG

SEQ ID NO: 255 - protein, 90 aa, synthetic construct
ATGAAAAAGTTACTACTTTTGCTGGTCTGTCTGCTCGGATTGGCGGCCTTAGGCTTGTCCGCGTGCGGCACATCAGTGTCCGCTGCAACT

SEQ ID NO: 256 - protein, 90 aa, synthetic construct
ATGAAGAAACTACTGATCCTTTTAACGTTGGTTATATCCCTGGCGGCCTTCTTGCTGTCAGGATGCGGCACATCAGTTTCCGCTGCCACC

SEQ ID NO: 257 - protein, 90 aa, synthetic construct
ATGAAAAAGCTGTTGCTATTACTTGTAGTTGTCCTAGGACTGGCTTTACTGTTACTGTCAGGTTGTGGAACTTCGGTTCACGCCGCCTCC

SEQ ID NO: 258 - protein, 90 aa, synthetic construct
ATGAAGAAACTGATCAGCTTGCTCGTAGTTTTGTTAGGTTTGGCCGCATTACTATTATCTGGCTGCGGTACGAGTGATTCAGCCGCGGCT

SEQ ID NO: 259 - protein, 90 aa, synthetic construct
ATGAAGAAACTCCTGATCCTGCTCGTAGTTCTCCTTGGCATGGCAGCTTTATTGTTATCTGGTTGTGGGACCTCTGTGTCCGCGGACACC

SEQ ID NO: 260 - protein, 90 aa, synthetic construct
ATGAAGAAACTTTTGCTTCTGCTTACCGTGGCACTAGGCCTGGCGGCACTGTTGCTTAGTGGTCTTGGAACATCCGTCTCCGCCGCCACC

SEQ ID NO: 261 - protein, 90 aa, synthetic construct
ATGAAAAAGTTGACACTTGGCCTTGTGGTAGCGGCCGGCCTCGCGGCATTACTCTTGTCAGGGTGCGGCACACCTACGAGTGCGGCCGAG

SEQ ID NO: 262 - protein, 90 aa, synthetic construct
ATGAAGCGACTGTTGCTTTTGTTAGTAGTCGTATTAGGATTGGCGGCCTTATTGTTAAGTGGTTGCGGAACAAGCGTGCATGCGGCTACA

SEQ ID NO: 263 - protein, 90 aa, synthetic construct
ATGAAACGATTACTTGAGCTTCTAGTAGTGTTGCTTGGTTTAGCCGCATTATGCCTGTCCGGTTGCGGTACAAGTGTATCGGCTGCGGCA

SEQ ID NO: 264 - protein, 90 aa, synthetic construct
ATGAAGAAACTTCTATTATTGGCCGTAGTCTTACTTGGTTTAGCAGGCTTGCTTTTGTCAGGGTGCGGTACACCCGTCTCAGCGGCGACT

SEQ ID NO: 265 - protein, 90 aa, synthetic construct
ATGAAAAAGTTACTGTTGCTGGTTACCGTCTTACTTGGCCTTGCTGCAATGGGTTTGTCCGGAGCCGGTGTCTCGGTCAGTGCTAGCACT

SEQ ID NO: 266 - protein, 90 aa, synthetic construct
ATGAAAAAGCTGTTAGCGCTATTAGTAGTCTTATTGGGTTTAGCGGCCTTACTGTTGTCTGTGTGTGGAACCAGCGTTTCAGCCGCAACC

SEQ ID NO: 267 - protein, 90 aa, synthetic construct
ATGAAGAAATTGCTCTTGCTGCTCGTTATGTTGTTCGGCCTTGCTTTCCTGCTCTTATCAGGCTGTGGAACCTCCGTTTCGGCCTCCACA

SEQ ID NO: 268 - protein, 90 aa, synthetic construct
ATGAAGAAATTGCTCTTGCTACTTGTTGTAGCACTAGGATTGGCGGCCATGCTCTTGTCAGGGTGCGGATCCAGTGTTCACGCAGCTACC

SEQ ID NO: 269 - protein, 90 aa, synthetic construct
ATGAAGAAACTCTTGTTATTGCTTACCGTCTTGTTAAGTTTAGCAGCCTTGCTTTTGTCCGGTACAGGGACTCCCGTTTCAGCCGCTACG

SEQ ID NO: 270 - protein, 90 aa, synthetic construct
ATGAAGAAATTGTTACTACTTTTGGTAGGTTTGCTTGGGTTGGCCGCTTTGCTTTTGTCTGGCGTGGGAACGTCGGTCCACGCGGCCACC

SEQ ID NO: 271 - protein, 90 aa, synthetic construct
ATGAAGCGACTCTTAATTCTATTGGTTGTACTTATAGGTTTGGCAGCCTTAGGCCTGTCTGGCTGCGGTACATCTGTCAGTGCCGCCACG

SEQ ID NO: 272 - protein, 90 aa, synthetic construct
ATGAAGAAATTGCTTAGCTTGATCGTCGTTCTATTAGGACTGGCCGCTATGCTGTTATCTGGATGTGGAACGTCAGTTTCAGCCGCTTGT

SEQ ID NO: 273 - protein, 90 aa, synthetic construct
ATGAAGAAATTGCTATTACTGTTGCTGGTACTTCTGTTTATGGCAGCCCTACTTCTCTCAGGCTGCGGCACCTCAGTGTCCGCGAGTACC

SEQ ID NO: 274 - protein, 90 aa, synthetic construct
ATGAAACGCCTGCTTTTACTCTTGGTCCTTTTGTTAGGACTTGCAGCTGCACTTCTGACCGGATGCGGGACCTCGGTTAGTGCCGCCACC

SEQ ID NO: 275 - protein, 90 aa, synthetic construct
ATGAAGAAATTACTCCTATTGCTTGTTGTACTGTTGGGCGTTACCGCCTTATTGCTTTCCGGCTGCGGTAAATCGGTTTCAGCGGCCACT

SEQ ID NO: 276 - protein, 90 aa, synthetic construct
ATGAAAAAGTTACTTCTGCTTTTGGTCGTTTTGTTAGGTTGTGCCGCACTACTCCTTAGTGGTTGTGGGACACCATCCAGTGCAGCCACT

SEQ ID NO: 277 - protein, 90 aa, synthetic construct
ATGAAGAAATTGCTGCTATTATTTGTTGTACTACTTGGTCTTGCAGCCCTGCTATTTTCAGGTGCAGGCACATCAGTCAGCGCCGCGACC

SEQ ID NO: 278 - protein, 90 aa, synthetic construct
ATGAAAAAGCTTTTATTGTTATTGGTGGTTCTTCTGTTTGTCGCAGCCCTTGGATTGTCAGGATGCGGCACATCTACATCTGCGGCTACT

SEQ ID NO: 279 - protein, 90 aa, synthetic construct
ATGAAGAAATTGTTATTGTTCCTAGTGGTCCTGTTAGGGATGAGTGCTCTCGGTTTGTCTGGATGCGGCACGAGTGTGCATGCCGCGTCG

SEQ ID NO: 280 - protein, 90 aa, synthetic construct
ATGAAAAAGTTATTGCTCTTATTGGTTTTACTCTTGGGTCTTGCCGCATTTCTGTTAAGCGGGTGTGGCACACCAGTCAGTGCCCAGACG

SEQ ID NO: 281 - protein, 90 aa, synthetic construct
ATGAAGAGATTACTCGAGCTCCTGGTCGTGCTTTTAGGCCTGGCGTTCCTGATGTTAAGTGGTTGCGGCACACGCGTACATGCTGCCACA

SEQ ID NO: 282 - protein, 146 aa, synthetic construct
LPVNTRCKLEVSNFQQPYIVNRTFMLAKEASLADNNTDVRLIGEKLFRGVSAKDQCYLMKQVLNFTLEDVLLPQSDRFQPYMQEVVPFLTKLSNQLSSCHISGDDQNIQKNVRRLKETVKKLGESGEIKAIGELDLLFMSLRNACV

SEQ ID NO: 283 - protein, 327 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGSGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 284 - protein, 160 aa, synthetic construct
SPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 285 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

(19) United States
(12) Patent Application Publication
(10) Pub. No.: US 2026/0217810 A1
(43) Pub. Date: Jul. 30, 2026

(54) ARTIFICIAL SECRETION PEPTIDES FOR HETEROLOGOUS PROTEIN PRODUCTION

(71) Applicant: Tenza, Inc., Boston, MA (US)

(72) Inventors: Anik Debnath, Somerville, MA (US); Denise Sirias, Cambridge, MA (US); Devon Stork, Somerville, MA (US)

(21) Appl. No.: 19/144,771

(22) PCT Filed: Dec. 29, 2023
(86) PCT No.: PCT/US2023/086408

§ 371 Date: Jun. 30, 2025

Related U.S. Application Data
(60) Provisional application No. 63/477,959, filed on Dec. 30, 2022.

(51) Int. Cl. C07K 16/24; A61K 35/00; A61K 35/747; C07K 14/00

(52) U.S. Cl. CPC C07K 16/241; A61K 35/747; C07K 14/001; A61K 2035/11; C07K 2319/02

(57) ABSTRACT

Provided herein are pre-protein signal peptides that direct secretion of expressed payload proteins from Lactobacillus bacteria. The pre-protein signal peptides described herein are designed for increased payload secretion over known signal peptides with minimal or reduced effect on bacterial strain fitness. Also provided herein are methods using the pre-protein signal peptides provided herein.

### CROSS REFERENCE TO RELATED APPLICATIONS

This application claims the benefit of U.S. Provisional Application Ser. No. 63/477,959 filed 30 Dec. 2022, which is hereby incorporated by reference in its entirety.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created on 29 Dec. 2023, is named “TNZ-008WO_SL.xml” and is 273,334 bytes in size.

### FIELD

Embodiments provided herein relate generally to signal peptides and more particularly to synthetic pre-protein signal peptides that increase secretion of a recombinant protein in Lactobacillus.

### BACKGROUND

Bacteria are routinely used as hosts to produce proteins for research, therapeutics and industrial purposes. The first step during the secretion of a desired target protein into the growth medium is its transport across the cytoplasmic membrane. In bacteria, two major export pathways are utilized for the transport of proteins across the plasma membrane, the general secretion or Sec pathway and the twin-arginine translocation or Tat pathway. The routing into one of these alternative protein export systems requires the fusion of a Sec- or Tat-specific signal peptide to the amino-terminal end of the desired target protein. Signal peptides are required for the targeting to and membrane translocation by the respective protein translocases, but also have influences on the biosynthesis, the folding kinetics, and the stability of the respective payload proteins. Currently, it is not possible to predict which signal peptide will perform best in the context of a given payload protein and a given bacterial expression host.

Further, while increased secretory production of a payload protein from the use of a signal peptide is desired, there are additional factors that must be taken in consideration when developing a method for manufacturing a particular payload protein. For example, a signal peptide resulting in increased secretion of a recombinant payload protein may be deleterious to the host bacteria. Accordingly, one must identify the beneficial level of increased protein secretion that will also maintain overall host bacterial health, or “fitness”. The embodiments of the present disclosure address these needs and others. In particular, the signal peptides of the present disclosure are optimized to provide increased recombinant payload protein yield over known signal peptides while having minimal impact on the fitness of the host bacteria.

### SUMMARY

In some embodiments, a pre-protein signal peptide is provided. In some embodiments, the pre-protein signal peptide comprises an amino acid sequence having at least 95%, 96%, 97%, 98%, 99%, or 100% identity to an amino acid sequence selected from SEQ ID NO: 6, SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180. In some embodiments, the pre-protein signal peptide comprises an amino acid sequence having at least 95%, 96%, 97%, 98%, 99%, or 100% identity to SEQ ID NO: 6. In some embodiments, the pre-protein signal peptide comprises an amino acid sequence having at least 95%, 96%, 97%, 98%, 99%, or 100% identity to SEQ ID NO: 7. In some embodiments, the pre-protein signal peptide comprises an amino acid sequence having at least 95%, 96%, 97%, 98%, 99%, or 100% identity to SEQ ID NO: 14. In some embodiments, the pre-protein signal peptide comprises an amino acid sequence having at least 95%, 96%, 97%, 98%, 99%, or 100% identity to SEQ ID NO: 17. In some embodiments, the pre-protein signal peptide comprises an amino acid sequence having at least 95%, 96%, 97%, 98%, 99%, or 100% identity to SEQ ID NO: 18. In some embodiments, the pre-protein signal peptide comprises an amino acid sequence having at least 95%, 96%, 97%, 98%, 99%, or 100% identity to SEQ ID NO: 19. In some embodiments, the pre-protein signal peptide comprises an amino acid sequence having at least 95%, 96%, 97%, 98%, 99%, or 100% identity to SEQ ID NO: 20. In some embodiments, the pre-protein signal peptide comprises an amino acid sequence having at least 95%, 96%, 97%, 98%, 99%, or 100% identity to SEQ ID NO: 47. In some embodiments, the pre-protein signal peptide comprises an amino acid sequence having at least 95%, 96%, 97%, 98%, 99%, or 100% identity to SEQ ID NO: 48. In some embodiments, the pre-protein signal peptide comprises an amino acid sequence having at least 95%, 96%, 97%, 98%, 99%, or 100% identity to SEQ ID NO: 148. In some embodiments, the pre-protein signal peptide comprises an amino acid sequence having at least 95%, 96%, 97%, 98%, 99%, or 100% identity to SEQ ID NO: 180.

In some embodiments, a polypeptide is provided. In some embodiments, the polypeptide comprises a formula of X₁-Z₁, wherein X₁ is a pre-protein signal peptide and Z₁ is a payload protein. In some embodiments, X₁ comprises an amino acid sequence having at least 95%, 96%, 97%, 98%, 99%, or 100% identity to an amino acid sequence selected from SEQ ID NO: 6, SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180. In some embodiments, Z₁ is selected from the group consisting of antimicrobial peptides, Catestatin, CRAMP, hBD1, hBD3, β-Cateslytin, Dermaseptin, IL-10, IL-22, ClbS, TIGIT, TFF1, TFF2, TFF3, anti-CCL2 antibody, anti-CCL2 antibody fragments, anti-IL23 antibody, anti-IL23 antibody fragments, anti-TNFα antibody, anti-TNFα antibody fragments, GLP2, IGF1, leptin, and casein. I

n some embodiments, X₁ comprises an amino acid sequence of SEQ ID NO: 6, and Z₁ comprises an amino acid sequence of SEQ ID NO: 21, SEQ ID NO: 118, SEQ ID NO: 119, SEQ ID NO: 120, or a fragment thereof.

In some embodiments, X₁ comprises an amino acid sequence of SEQ ID NO: 7, and Z₁ comprises an amino acid sequence of SEQ ID NO: 21, SEQ ID NO: 118, SEQ ID NO: 119, SEQ ID NO: 120, or a fragment thereof.

In some embodiments, X₁ comprises an amino acid sequence of SEQ ID NO: 14, and Z₁ comprises an amino acid sequence of SEQ ID NO: 21, SEQ ID NO: 118, SEQ ID NO: 119, SEQ ID NO: 120, or a fragment thereof.

In some embodiments, X₁ comprises an amino acid sequence of SEQ ID NO: 6, and Z₁ comprises an amino acid sequence of SEQ ID NO: 25.

In some embodiments, X₁ comprises an amino acid sequence of SEQ ID NO: 7, and Z₁ comprises an amino acid sequence of SEQ ID NO: 25.

In some embodiments, X₁ comprises an amino acid sequence of SEQ ID NO: 14, and Z₁ comprises an amino acid sequence of SEQ ID NO: 25.

In some embodiments, X₁ comprises an amino acid sequence of SEQ ID NO: 17, and Z₁ comprises an amino acid sequence of SEQ ID NO: 25.

In some embodiments, X₁ comprises an amino acid sequence of SEQ ID NO: 18, and Z₁ comprises an amino acid sequence of SEQ ID NO: 25.

In some embodiments, X₁ comprises an amino acid sequence of SEQ ID NO: 19, and Z₁ comprises an amino acid sequence of SEQ ID NO: 25.

In some embodiments, X₁ comprises an amino acid sequence of SEQ ID NO: 47, and Z₁ comprises an amino acid sequence of SEQ ID NO: 25.

In some embodiments, X₁ comprises an amino acid sequence of SEQ ID NO: 48, and Z₁ comprises an amino acid sequence of SEQ ID NO: 25.

In some embodiments, X₁ comprises an amino acid sequence of SEQ ID NO: 148, and Z₁ comprises an amino acid sequence of SEQ ID NO: 25.

In some embodiments, X₁ comprises an amino acid sequence of SEQ ID NO: 180, and Z₁ comprises an amino acid sequence of SEQ ID NO: 25.

In some embodiments, X₁ comprises an amino acid sequence of SEQ ID NO: 20, and Z₁ comprises an amino acid sequence of SEQ ID NO: 24, SEQ ID NO: 283, SEQ ID NO: 284, or SEQ ID NO: 285.

In some embodiments, X₁ comprises an amino acid sequence of SEQ ID NO: 7, and Z₁ comprises an amino acid sequence of SEQ ID NO: 22, SEQ ID NO: 59, or SEQ ID NO: 282.

In some embodiments, X₁ comprises an amino acid sequence of SEQ ID NO: 14, and Z₁ comprises an amino acid sequence of SEQ ID NO: 22, SEQ ID NO: 59, or SEQ ID NO: 282.

In some embodiments, X₁ comprises an amino acid sequence of SEQ ID NO: 148, and Z₁ comprises an amino acid sequence of SEQ ID NO: 22, SEQ ID NO: 59, or SEQ ID NO: 282.

In some embodiments, X₁ comprises an amino acid sequence of SEQ ID NO: 180, and Z₁ comprises an amino acid sequence of SEQ ID NO: 22, SEQ ID NO: 59, or SEQ ID NO: 282.

In some embodiments, a nucleic acid molecule is provided. In some embodiments, the nucleic acid molecule encodes for a signal peptide as provided for herein.

In some embodiments, a nucleic acid molecule is provided. In some embodiments, the nucleic acid molecule encodes for a polypeptide as provided for herein.

In some embodiments, a bacterium is provided. In some embodiments, the bacterium comprises a heterologous nucleic acid molecule encoding for a polypeptide as provided for herein.

In some embodiments, a pharmaceutical composition is provided. In some embodiments, the pharmaceutical composition comprises a bacterium as provided for herein and a pharmaceutically acceptable carrier.

In some embodiments, a method for producing a protein is provided. In some embodiments, the method comprises transfecting a bacterium with a nucleic acid molecule encoding for a polypeptide as provided for herein to produce a bacterium comprising the nucleic acid molecule; and culturing the bacteria comprising the nucleic acid molecule under conditions sufficient to grow the bacteria, wherein the bacteria expresses and secretes the payload protein.

In some embodiments, a method for producing a protein in a subject is provided. In some embodiments, the method comprises administering to the subject a bacteria as provided for herein or a pharmaceutical composition as provided for herein, wherein the bacteria are live and wherein the live bacteria produce the protein in the subject.

In some embodiments, a method for treating a disease or disorder in a subject in need thereof is provided. In some embodiments, the method comprises administering to the subject a therapeutically effective amount of a bacteria as provided for herein or a pharmaceutical composition as provided for herein.

In some embodiments, a method for treating inflammation in a subject in need thereof is provided. In some embodiments, the method comprises administering to the subject a therapeutically effective amount of a bacteria as provided for herein or a pharmaceutical composition as provided for herein.

In some embodiments, a method for treating inflammation in a subject in need thereof is provided. In some embodiments, the method comprises administering to the subject a therapeutically effective amount of a bacteria comprising a heterologous nucleic acid molecule encoding for a polypeptide having the formula X₁-Z₁, wherein X₁ comprises an amino acid sequence of SEQ ID NO: 6, SEQ ID NO: 7, SEQ ID NO: 14, or SEQ ID NO: 20 and Z₁ comprises an amino acid sequence of SEQ ID NO: 21, SEQ ID NO: 24, SEQ ID NO: 118, SEQ ID NO: 119, or SEQ ID NO: 120.

In some embodiments, a method for treating obesity in a subject in need thereof is provided. In some embodiments, the method comprises administering to the subject a therapeutically effective amount of a bacteria as provided for herein or a pharmaceutical composition as provided for herein.

In some embodiments, a method for treating obesity in a subject in need thereof is provided. In some embodiments, the method comprises administering to the subject a therapeutically effective amount of a bacteria comprising a heterologous nucleic acid molecule encoding for a polypeptide having the formula X₁-Z₁, wherein, X₁ comprises an amino acid sequence of SEQ ID NO: 17, SEQ ID NO: 18, SEQ ID NO: 19, SEQ ID NO: 47, or SEQ ID NO: 48 and Z₁ comprises an amino acid sequence of SEQ ID NO: 25.

### BRIEF DESCRIPTION OF THE DRAWINGS

FIG. 1 illustrates the influence of a panel of unique signal peptides (B-P) on anti-TNFα antibody secretion and on bacteria strain doubling time as compared to a known signal peptide (A). Dotted line represents doubling time of untransfected wild-type bacteria. Data points for peptide I and peptide O are overlapping.

FIG. 2 illustrates the influence of signal peptides F (SEQ ID NO: 6), G (SEQ ID NO: 7), and N (SEQ ID NO: 14) on the secretion of various payload proteins and bacterial strain doubling time as compared to a known signal peptide, A (SEQ ID NO: 1).

FIG. 3 illustrates the distribution of leptin secretion from a library of signal peptides.

FIG. 4 illustrates the influence of lead candidate signal peptides on leptin secretion and on bacterial strain doubling time as compared to a known signal peptide, A (SEQ ID NO: 1).

FIG. 5 illustrates the distribution of IL-10 secretion from a library of signal peptides.

FIGS. 6A and 6B illustrate the influence of SEQ ID NO: 20 on IL-10 secretion and on bacterial strain doubling time as compared to a known signal peptide, SEQ ID NO: 1. FIG. 6A illustrates the comparison of IL-10 secretion. FIG. 6B illustrates the comparison of change in doubling time as compared to wild-type untransfected bacteria.

FIG. 7 illustrates the distribution of mouse IL-22 secretion from a library of signal peptides.

FIG. 8 illustrates the influence of signal peptides A2 (SEQ ID NO: 148) and D2 (SEQ ID NO: 180) on mouse IL-22 secretion and on bacterial strain doubling time as compared to signal peptide G (SEQ ID NO: 7). The dotted line represents doubling time of the WT bacterial strain.

FIGS. 9A and 9B illustrate the influence of signal peptides A2 (SEQ ID NO: 148) and D2 (SEQ ID NO: 180) on secretion of additional protein targets and on bacterial strain doubling time. FIG. 9A illustrates human IL-22 secretion as compared to signal peptide G (SEQ ID NO: 7). FIG. 9B illustrates leptin secretion as compared to signal peptide lib-10 (SEQ ID NO: 19) and signal peptide A (SEQ ID NO: 1).

### DETAILED DESCRIPTION

The present disclosure presents a solution to the aforementioned challenges by providing new, synthetic signal peptides that direct secretion of expressed proteins or peptides in Bacillus bacteria. The disclosed signal peptides overcome performance variability challenges posed by previously characterized and native signal peptides and may be used to generate and facilitate secretion of any protein or peptide from bacteria.

The disclosed synthetic pre-protein signal peptides increase secretion of any recombinant protein in Lactobacillus bacteria. The use of synthetic pre-protein signal peptide may further improve secretion of a payload protein, for example, through facilitating translocation across the cytoplasmic membrane. Advantageously, the signal peptides disclosed herein have been generated and optimized to promote secretion of payload proteins from Lactobacillus bacteria. Use of the disclosed synthetic pre-protein signal peptides may be used to achieve increased secretion of the desired payload to any bacteria-compatible environment, such as in therapeutics, agriculture, or food products.

Before the present compositions and methods are described, it is to be understood that the scope of the invention is not limited to the particular processes, compositions, or methodologies described herein, as these may vary. It is also to be understood that the terminology used in the description is for the purpose of describing the particular versions or embodiments only, and is not intended to limit the scope of the present invention. Unless defined otherwise, all technical and scientific terms used herein have the same meanings as commonly understood by one of ordinary skill in the art. Although any methods and materials similar or equivalent to those described herein can be used in the practice or testing of embodiments of the methods and systems disclosed herein, the preferred methods, devices, and materials are now described.

### Definitions

The following explanations of terms and methods are provided to better describe the present disclosure and to guide those of ordinary skill in the art in the practice of the present disclosure.

As used herein, “comprising” means “including” and the singular forms “a” or “an” or “the” include plural references unless the context clearly dictates otherwise. For example, reference to “comprising a therapeutic agent” includes one or a plurality of such therapeutic agents. The term “or” refers to a single element of stated alternative elements, unless the context clearly indicates otherwise. For example, the phrase “A or B” refers to A alone or B alone. The phrase “A, B, or a combination thereof” refers to A alone, B alone, or a combination of A and B. Similarly, “one or more of A and B” refers to A, B, or a combination of both A and B. The phrase “A and B” refers to a combination of A and B. Furthermore, the various elements, features and steps discussed herein, as well as other known equivalents for each such element, feature or step, can be mixed and matched by one of ordinary skill in this art to perform methods in accordance with principles described herein. Among the various elements, features, and steps some will be specifically included and others specifically excluded in particular examples.

Unless otherwise defined, all technical and scientific terms used herein have the same meaning as commonly understood to one of ordinary skill in the art. Although methods and materials similar or equivalent to those described herein can be used in the practice or testing of the present disclosure, suitable methods and materials are described below. The materials, methods, and examples are illustrative only and not intended to be limiting. All references cited herein are incorporated by reference in their entirety.

In some examples, the numbers expressing quantities of ingredients, properties such as molecular weight, reaction conditions, and so forth, used to describe and claim certain embodiments are to be understood as being modified in some instances by the term “about” or “approximately.” For example, “about” or “approximately” can indicate +/−5% variation of the value it describes. Accordingly, in some embodiments, the numerical parameters set forth herein are approximations that can vary depending upon the desired properties for a particular embodiment. Notwithstanding that the numerical ranges and parameters setting forth the broad scope of some examples are approximations, the numerical values set forth in the specific examples are reported as precisely as practicable. The recitation of ranges of values herein is merely intended to serve as a shorthand method of referring individually to each separate value falling within the range.

To facilitate review of the various embodiments of this disclosure, the following explanations of specific terms are provided:

As used herein, “bacteria” (plural) and “bacterium” (singular) refer to a unicellular prokaryotic microorganism. Bacteria cells are generally surrounded by two protective coatings: an outer cell wall and an inner cell membrane. Bacteria may be classified according to the Gram stain, which identifies bacteria by the composition of their cell walls. Gram-positive bacteria do not have an outer membrane whereas Gram-negative bacteria do not. Bacteria generally reproduce by binary fission, where a parent cell makes a copy of its DNA and grows larger by doubling its cellular content. The cell then splits apart, pushing the extra cellular content out, creating two daughter cells. Some bacteria utilize other processes, such as budding. In some embodiments, the bacteria are wild-type natural isolates of bacteria. In some embodiments, the bacteria are laboratory strains of bacteria that have undergone domestication processes of mutagenesis and selection. As used herein, “bacteria” refers to any wild type or laboratory strain of bacteria known.

As used herein, “Lactobacillus bacteria” refer to a genus of gram-positive, aerotolerant anaerobes or microaerophilic, rod-shaped, non-spore-forming bacteria that constitute a significant component of the human and animal microbiota. Examples of Lactobacillus bacteria include, but are not limited to, Lactobacillus acetotolerans, Lactobacillus acidifarinae, Lactobacillus acidipiscis, Lactobacillus acidophilus, Lactobacillus agilis, Lactobacillus algidus, Lactobacillus alimentarius, Lactobacillus alvei, Lactobacillus alvi, Lactobacillus amylolyticus, Lactobacillus amylophilus, Lactobacillus amylotrophicus, Lactobacillus amylovorus, Lactobacillus animalis, Lactobacillus animata, Lactobacillus antri, Lactobacillus apinorum, Lactobacillus apis, Lactobacillus apodemi, Lactobacillus aquaticus, Lactobacillus aviarius, Lactobacillus backii, Lactobacillus bifermentans, Lactobacillus bombi, Lactobacillus bombicola, Lactobacillus brantae, Lactobacillus brevis, Lactobacillus brevisimilis, Lactobacillus buchneri, Lactobacillus cacaonum, Lactobacillus camelliae, Lactobacillus capillatus, Lactobacillus casei, Lactobacillus paracasei, Lactobacillus zeae, Lactobacillus catenefornis, Lactobacillus ceti, Lactobacillus coleohominis, Lactobacillus colini, Lactobacillus collinoides, Lactobacillus composti, Lactobacillus concavus, Lactobacillus coryniformis, Lactobacillus crispatus, Lactobacillus crustorum, Lactobacillus curieae, Lactobacillus curvatus, Lactobacillus delbrueckii, Lactobacillus dextrinicus, Lactobacillus diolivorans, Lactobacillus equi, Lactobacillus equicursoris, Lactobacillus equigenerosi, Lactobacillus fabifermentans, Lactobacillus faecis, Lactobacillus faeni, Lactobacillus farciminis, Lactobacillus farraginis, Lactobacillus fermentum, Lactobacillus floricola, Lactobacillus florum, Lactobacillus formosensis, Lactobacillus fornicalis, Lactobacillus fructivorans, Lactobacillus frumenti, Lactobacillus fuchuensis, Lactobacillus furfuricola, Lactobacillus futsaii, Lactobacillus gallinarum, Lactobacillus gasseri, Lactobacillus gastricus, Lactobacillus ghanensis, Lactobacillus gigeriorum, Lactobacillus ginsenosidimutans, Lactobacillus gorillae, Lactobacillus graminis, Lactobacillus guizhouensis, Lactobacillus halophilus, Lactobacillus hammesii, Lactobacillus hamsteri, Lactobacillus harbinensis, Lactobacillus hayakitensis, Lactobacillus heilongjiangensis, Lactobacillus helsingborgensis, Lactobacillus helveticus, Lactobacillus herbarum, Lactobacillus heterohiochii, Lactobacillus hilgardii, Lactobacillus hokkaidonensis, Lactobacillus hominis, Lactobacillus homohiochii, Lactobacillus hordei, Lactobacillus iatae, Lactobacillus iners, Lactobacillus ingluviei, Lactobacillus insectis, Lactobacillus insicii, Lactobacillus intermedius, Lactobacillus intestinalis, Lactobacillus iwatensis, Lactobacillus ixorae, Lactobacillus japonicus, Lactobacillus jensenii, Lactobacillus johnsonii, Lactobacillus kalixensis, Lactobacillus kefiranofaciens, Lactobacillus kefiri, Lactobacillus kimbladii, Lactobacillus kimchicus, Lactobacillus kimchiensis, Lactobacillus kisonensis, Lactobacillus kitasatonis, Lactobacillus koreensis, Lactobacillus kullabergensis, Lactobacillus kunkeei, Lactobacillus larvae, Lactobacillus leichmannii, Lactobacillus letivazi, Lactobacillus lindneri, Lactobacillus malefermentans, Lactobacillus mali, Lactobacillus manihotivorans, Lactobacillus mellifer, Lactobacillus mellis, Lactobacillus melliventris, Lactobacillus micheneri, Lactobacillus mindensis, Lactobacillus mixtipabuli, Lactobacillus mobilis, Lactobacillus modestisalitolerans, Lactobacillus mucosae, Lactobacillus mudanjiangensis, Lactobacillus murinus, Lactobacillus nagelii, Lactobacillus namurensis, Lactobacillus nantensis, Lactobacillus nasuensis, Lactobacillus nenjiangensis, Lactobacillus nodensis, Lactobacillus odoratitofui, Lactobacillus oeni, Lactobacillus oligofermentans, Lactobacillus ori, Lactobacillus oryzae, Lactobacillus otakiensis, Lactobacillus ozensis, Lactobacillus panis, Lactobacillus pantheris, Lactobacillus parabrevis, Lactobacillus parabuchneri, Lactobacillus paracollinoides, Lactobacillus parafarraginis, Lactobacillus parakefiri, Lactobacillus paralimentarius, Lactobacillus paraplantarum, Lactobacillus pasteurii, Lactobacillus paucivorans, Lactobacillus pentosus, Lactobacillus perolens, Lactobacillus plajomi, Lactobacillus plantarum, Lactobacillus pobuzihii, Lactobacillus pontis, Lactobacillus porcinae, Lactobacillus psittaci, Lactobacillus rapi, Lactobacillus rennanquilfy, Lactobacillus rennini, Lactobacillus reuteri, Lactobacillus rhamnosus, Lactobacillus rodentium, Lactobacillus rogosae, Lactobacillus rossiae, Lactobacillus ruminis, Lactobacillus saerimneri, Lactobacillus sakei, Lactobacillus salivarius, Lactobacillus sanfranciscensis, Lactobacillus saniviri, Lactobacillus satsumensis, Lactobacillus secaliphilus, Lactobacillus selangorensis, Lactobacillus senioris, Lactobacillus senmaizukei, Lactobacillus sharpeae, Lactobacillus shenzhenensis, Lactobacillus sicerae, Lactobacillus silagei, Lactobacillus siliginis, Lactobacillus similis, Lactobacillus songhuajiangensis, Lactobacillus spicheri, Lactobacillus sucicola, Lactobacillus suebicus, Lactobacillus sunkii, Lactobacillus taiwanensis, Lactobacillus thailandensis, Lactobacillus tucceti, Lactobacillus ultunensis, Lactobacillus uvarum, Lactobacillus vaccinostercus, Lactobacillus vaginalis, Lactobacillus vermiforme, Lactobacillus vespulae, Lactobacillus vini, Lactobacillus wasatchensis, Lactobacillus xiangfangensis, Lactobacillus yonginensis, or Lactobacillus zymae. It is to be understood that, as used herein, “Lactobacillus bacteria” also refers to and encompasses the species of Lactobacillus that were recently reclassified into alternate genera. These include, but are not limited to, Atopobium (e.g., A. deltae), Carnobacterium (e.g., C. alterfunditum), Weissella (e.g., W. beninensis), Oenococcus (e.g., O. oeni), Leuconostoc (e.g., L. carnosum), Pediococcus (e.g., P. acidilactici), Paralactobacillus (e.g., P. selangorensis), Holzapfelia (e.g., H. floricola), Amylolactobacillus (e.g., A. amylophilus), Bombilactobacillus (e.g., B. mellifer), Companilactobacillus (e.g., C. alimentarius), Lapidilactobacillus (e.g., L. concavus), Agrilactobacillus (e.g., A. composti), Schleiferilactobacillus (e.g., S. perolens), Loigolactobacillus (e.g., L. coryniformis), Lacticaseibacillus (e.g., L. casei), Latilactobacillus (e.g., L. sakei), Dellaglioa (e.g., D. algidus), Liquorilactobacillus (e.g., L. mail), Ligilactobacillus (e.g., L. salivarius), Lactiplantibacillus (e.g., L. plantarum), Furfurilactobacillus (e.g., F. rossiae), Paucilactobacillus (e.g., P. vaccinostercus), Limosilactobacillus (e.g., L. fermentum), Fructilactobacillus (e.g., F. fructivorans), Acetilactobacillus (e.g., A. jinshani), Apilactobacillus (e.g., A. kunkeei), Levilactobacillus (L. brevis), Secundilactobacillus (e.g., S. collinoides), and Lentilactobacillus (e.g., L. buchneri). It is to be understood that the examples of recently reclassified Lactobacillus are provided for clarity purposes only and are not meant to be limiting in any way. Thus, for example, Lactiplantibacillus is not limited to only L. plantarum, but rather comprises any species of Lactiplantibacillus known or yet to be known or currently classified or yet to be classified as Lactiplantibacillus. Thus, in referring to Lactiplantibacillus, species would include, but not be limited to, L. argentoratensis, L. daoliensis, L. daowaiensis, L. dongliensis, L. fabifermentans, L. garii, L. herbarum, L. modestisalitolerans, L. mudanjiangensis, L. paraplantarum, L. pentosus, L. pingfangensis, L. plajomi, L. plantarum, L. songbeiensis, and L. xiangfangensis. Such understanding is also applicable to all genera recited above. In some embodiments, the Lactobacillus bacteria are wild-type natural isolates of Lactobacillus. In some embodiments, the Lactobacillus bacteria are laboratory strains of Lactobacillus that have undergone domestication processes of mutagenesis and selection. As used herein, “Lactobacillus bacteria” refers to any wild type strain or laboratory strain of Lactobacillus bacteria known. Further, in referring to any specific Lactobacillus species, the recitation of the species also includes any wild type strain or laboratory strain of the Lactobacillus species know.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, “fitness” refers to a bacteria's ability to survive, grow, or be otherwise metabolically active in different environments. Fitness is generally measured via several methods including quantification of biofilm growth, survival in water, resistance to drying, and measurement of growth rate (e.g. doubling time). In the context of the present disclosure, fitness is typically measured via measurement of growth rate. Further, in the context of the present disclosure, the identity of a particular signal peptide, payload protein, or both are factors which can affect the bacterial strain fitness. If the use of a particular signal peptide or signal peptide and payload protein combination results in an increase in bacterial doubling time, the bacteria are considered less fit. Similarly, if the use of a particular signal peptide or signal peptide and payload protein combination results in a decrease in bacterial doubling time (i.e. the bacteria double at a quicker rate), the bacteria are considered to be more fit. Change in the bacterial growth rate can be an indicator of how the bacteria may fair in a harsher environment, e.g. the acidic environment of the gut. If a particular signal peptide or signal peptide and payload protein combination results in an increase in bacterial doubling time, that may indicate that use of that combination would be deleterious in a harsher growth environment. On the contrary, if a particular signal peptide or signal peptide and payload protein combination results in a decrease in bacterial doubling time, that may indicate that use of that combination would not affect or may positively affect the survivability of the bacteria in the harsher growth environment.

As used herein, “fitness cost” refers to the change in bacterial growth rate as a result of using a particular signal peptide or signal peptide and payload protein combination. If the use of a particular signal peptide or signal peptide and payload protein combination results in an increase in bacterial doubling time, then the particular combination is considered to have a negative fitness cost. Similarly, if the use of a particular signal peptide or signal peptide and payload protein combination results in a decrease in bacterial doubling time (i.e. the bacteria double at a quicker rate), then the particular combination is considered to have a positive fitness cost. In the context of the present disclosure, an optimal signal peptide is one that has a balance between increased protein secretion and fitness cost.

As used herein, “genetically modified” or any grammatical variation thereof, refers to a practice of introducing a nucleic acid into a bacterial cell that encodes to promote the expression of a recombinant protein therein. A nucleic acid may be DNA, mRNA, tRNA, or rRNA. A nucleic acid is composed of nucleotide monomers, each triplet of monomers (a codon) encoding for either a triplet of RNA nucleotide monomers (if the nucleic acid is DNA) or an amino acid (if the nucleic acid is RNA). DNA also comprises one or more promoter regions, which indicate where transcription of the DNA should start. mRNA also comprises a ribosome binding site, which indicates where translation of the mRNA should start as well as one or more stop codons, which indicates where mRNA translation should end.

In any embodiment or aspect disclosed herein, a nucleic acid encoding for a recombinant protein, as disclosed herein, may be introduced into a bacterial cell using any method known to those skilled in the art for such introduction. Such methods include transfection, transformation, transduction, infection (e.g., viral transduction), injection, microinjection, gene gun, nucleofection, nanoparticle bombardment, transformation, conjugation, by application of the nucleic acid in a gel, oil, or cream, by electroporation, using lipid-based transfection reagents, or by any other suitable transfection method. One of skill in the art will readily understand and adapt such methods using readily identifiable literature sources.

As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection (e.g., using commercially available reagents such as, for example, LIPOFECTIN® (Invitrogen Corp., San Diego, CA), LIPOFECTAMINE® (Invitrogen), FUGENER (Roche Applied Science, Basel, Switzerland), JETPEI™ (Polyplus-transfection Inc., New York, NY), EFFECTENE® (Qiagen, Valencia, CA), DREAMFECT™ (OZ Biosciences, France) and the like), or electroporation (e.g., in vivo electroporation). Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

Methods and materials of non-viral delivery of nucleic acids to cells further include biolistics, virosomes, liposomes, immunoliposomes, polycation or lipid-nucleic acid conjugates, naked DNA, artificial virions, and agent-enhanced uptake of DNA. Lipofection is described in U.S. Pat. Nos. 5,049,386, 4,946,787; and 4,897,355 and lipofection reagents are sold commercially (e.g., TRANSFECTAM™ and LIPOFECTIN™). Cationic and neutral lipids that are suitable for efficient receptor-recognition lipofection of polynucleotides include those disclosed in WO91/17424 and WO 91/16024.

The methods described herein comprise generating a recombinant protein within a microorganism host. As used herein, recombinant describes a protein or nucleic acid that may or may not be naturally found in or produced by the host. For example, in some embodiments the host may be a specific Lactobacillus species and the recombinant protein being expressed may also be found naturally within the specific Lactobacillus species. In another example, the host may be a specific Lactobacillus species and the recombinant protein being expressed may not be found naturally within the specific Lactobacillus species, for example a protein from a different Lactobacillus species or from a different organism altogether.

As used herein, “payload protein” or “protein of interest” refers to the protein that will be generated by the host and chaperoned through the secretory pathway into the extracellular space, facilitated by the presence of a synthetic signal peptide. Upon secretion into the extracellular space, all, some, or none of the synthetic signal peptide may be fused to the payload protein. Optionally, a payload protein still being attached partially or fully to the synthetic signal peptide may be further processed, for example, to remove the remaining signal peptide. A payload protein may be any protein known or yet to be known, for example, an enzyme, enzyme inhibitor, growth factor, hormone, antibody, antigen, vaccine, a therapeutic agent, or any combination thereof. More specific examples follow herein below. A payload protein secreted by the various genetically modified bacteria disclosed herein, which are interchangeably referred to as “engineered bacteria”, may be provided to a subject in a pharmaceutical composition. Additionally or alternatively, the engineered bacteria itself may be provided to a subject in a pharmaceutical composition.

In some embodiments, the payload protein or protein of interest comprises an antibody. “Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g., IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

In some embodiments, the payload protein or protein of interest comprises an antibody fragment. “Antibody fragment”, “antigen binding fragment” or “antigen binding domain” refer to a portion of an immunoglobulin molecule that binds an antigen. Antigen binding fragments may be synthetic, enzymatically obtainable or genetically engineered polypeptides and include the VH, the VL, the VH and the VL, Fab, F(ab′) 2, Fd and Fv fragments, domain antibodies (dAb) consisting of one VH domain or one VL domain, shark variable IgNAR domains, camelized VH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3. VH and VL domains may be linked together via a synthetic linker to form various types of single chain antibody designs where the VH/VL domains may pair intramolecularly, or intermolecularly in those cases when the VH and VL domains are expressed by separate single chain antibody constructs, to form a monovalent antigen binding site, such as single chain Fv (scFv) or diabody. Other antibody fragments, or antigen binding fragments are known in the art, and any such fragment is applicable to the present disclosure.

The methods described herein comprise generating a recombinant protein within a bacterial host. As used herein, a “recombinant protein” comprises a payload protein and a synthetic signal peptide fused directly or indirectly thereto. As used herein, a signal peptide is any protein or peptide fused directly or indirectly to the N-terminus of a payload protein that facilitates the extracellular secretion of the payload protein after it is generated.

The methods disclosed herein utilize a synthetic signal peptide to increase extracellular secretion of a payload protein by a bacterium. As used herein, a “synthetic signal peptide” refers to a signal peptide whose sequence is generated as provided for herein and is made recombinantly. The recombinantly produced signal peptide can be referred to as a “synthetic signal peptide” or simply as a “signal peptide”. In some embodiments, the signal peptide may comprise a synthetic pre-protein signal peptide. As highlighted previously, the term synthetic in this context refers to a recombinantly produced pre-protein signal peptide whose sequence is generated as provided for herein. Hereafter, the pre-signal peptide may be referred to as a “synthetic” pre signal peptide, or simply as a pre-protein signal peptide.

A pre-protein signal peptide (synthetic or native) comprises 10 to 50 amino acids, which are appended either directly to the N-terminus of a payload protein or indirectly (e.g., using one or more spacers) to the N-terminus of a payload protein.

A synthetic pre-protein signal peptide may be appended to an adjacent amino acid via a bond to the N-terminal amino acid of the adjacent amino acid, for example, by a peptide bond, a peptide spacer (e.g., LEISSTCDA, represented by SEQ ID NO: 124) or a membrane-associating/lipidophilic alpha-helical peptide signal peptide (e.g., MISTIC, represented by SEQ ID NO: 125).

The chemical makeup of a peptide will be described herein by a series of amino acid single letter abbreviations or an “amino acid sequence(s)” or “sequence(s),” which are conventional and known to those in the art. While reference sequences will be explicitly disclosed, in any aspect and embodiment, a reference sequence may be modified to include conservative amino acid substitutions, as well as variants and fragments, while maintaining the characteristics and functionality of the reference sequence.

As used herein, “substantially identical” or “substantially similar” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison. These proteins may include homologs, orthologues, allelic variants and functional mutants. Typically, 50% identity or more between two polypeptide sequences is considered to be an indication of functional equivalence. Identity between polypeptides is preferably determined by the Smith-Waterman homology search algorithm as implemented in the MPSRCH program (Oxford Molecular), using an affine gap search with parameters gap open penalty-12 and gap extension penalty=1

Sequence identity can be measured/determined using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e3 and e100 indicating a closely related sequence. In some embodiments, sequence identity is determined by using BLAST with the default settings.

These proteins may, compared to the disclosed proteins, include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, Substitution of single amino acids within these families does not have a major effect on the biological activity. The proteins may have one or more (e.g. 1, 2, 3, 4, 5, 6, 7, 8, 9, 10, etc.) single amino acid deletions relative to the disclosed protein sequences. The proteins may also include one or more (e.g. 1, 2, 3, 4, 5, 6, 7, 8, 9, 10, etc.) insertions (e.g. each of 1, 2, 3, 4 or 5 amino acids) relative to the disclosed protein sequences.

The compositions disclosed herein may be provided to a subject in a variety of ways through administration of the composition to the subject. As used herein, administer or administration means to provide or the providing of a composition to a subject. Oral administration, as used herein, refers to delivery of an active agent through the mouth. Topical administration, as used herein, refers to the delivery of an active agent to a body surface, such as the skin, a mucosal membrane (e.g., nasal membrane, vaginal membrane, buccal membrane, or the like).

The various compositions disclosed herein may be useful in treating a number of diseases, for example, cancer. As used herein, cancer refers to a condition characterized by unregulated cell growth. Examples of cancer include, but are not limited to, squamous cell cancer, small-cell lung cancer, non-small cell lung cancer, lung adenocarcinoma, lung squamous cell carcinoma, gastrointestinal cancer, Hodgkin's and non-Hodgkin's lymphoma, pancreatic cancer, glioblastoma, cervical cancer, colon cancer, colorectal cancer, endometrial or uterine carcinoma, kidney cancer such as renal cell carcinoma and Wilms' tumors, basal cell carcinoma, melanoma, prostate cancer, and esophageal cancer.

The various compositions disclosed herein may also be useful in treating other indications. For example, the compositions disclosed herein may be useful for treating one or more of infections, functional gut disorders, allergies, short bowel syndrome, chronic idiopathic constipation, COPD, inflammation, chronic inflammation, colorectal cancer, periodontits, oral mucositis, infection, pathogenic E coli, inflammatory bowel disease, ulcerative colitis, Crohn's disease, type 2 diabetes, obesity, MERS-COV, SARS-COV, Bovine viral diarrhea, hypercholesterolemia, influenza, atopic dermatitis, or any disease or disorder that may derive from any of the diseases or disorders recited herein. Accordingly, it is to be understood that the recitation of a disease or disorder is not limiting to said recited disease or disorder, but rather also encompasses any disease or disorder deriving therefrom. For example, inflammation and chronic inflammation have been linked to a number of diseases or disorders. Therefore, the compositions provided for herein which may be used to treat inflammation or chronic inflammation may also be useful in treating the diseases or disorders which derive from inflammation or chronic inflammation.

The various compositions disclosed herein may comprise one or more drugs, biologics, or active agents, which are used interchangeably herein and refer to a chemical substance or compound that induces a desired pharmacological or physiological effect, and includes agents that are therapeutically effective, prophylactically effective, or cosmetically effective, i.e. the payload. “Drug,” “biologic,” and “active agent” include any pharmaceutically acceptable, pharmacologically active derivatives and analogs of those drugs, biologics, and active agents specifically mentioned herein, including, but not limited to, salts, esters, amides, prodrugs, active metabolites, inclusion complexes, analogs, and the like. Suitable drugs, biologics, and active agents may include, but are not limited to, alcohol deterrents; amino acids; ammonia detoxicants; anabolic agents; analeptic agents; analgesic agents; androgenic agents; anesthetic agents; anorectic compounds; anorexic agents; antagonists; anti-allergic agents; anti-amebic agents; anti-anemic agents; anti-anginal agents; anti-anxiety agents; anti-arthritic agents; anti-atherosclerotic agents; anti-bacterial agents; anti-cancer agents, including antineoplastic drugs, and anti-cancer supplementary potentiating agents; anticholinergics; anticholelithogenic agents; anti-coagulants; anti-coccidal agents; anti-convulsants; anti-depressants; anti-diabetic agents; anti-diarrheals; anti-diuretics; antidotes; anti-dyskinetics agents; anti-emetic agents; anti-epileptic agents; anti-estrogen agents; anti-fibrinolytic agents; anti-fungal agents; anti-glaucoma agents; anti-hemophilic agents; anti-hemorrhagic agents; antihistamines; anti-hyperlipidemic agents; anti-hyperlipoproteinemic agents; antihypertensive agents; anti-hypotensives; anti-infective agents such as antibiotics and antiviral agents; anti-inflammatory agents, both steroidal and non-steroidal; anti-keratinizing agents; anti-malarial agents; antimicrobial agents; anti-migraine agents; anti-mitotic agents; anti-mycotic agents; antinauseants; antineoplastic agents; anti-neutropenic agents; anti-obsessional agents; anti-parasitic agents; antiparkinsonism drugs; anti-pneumocystic agents; anti-proliferative agents; anti-prostatic hypertrophy drugs; anti-protozoal agents; antipruritics; anti-psoriatic agents; antipsychotics; antipyretics; antispasmodics; anti-rheumatic agents; anti-schistosomal agents; anti-seborrheic agents; anti-spasmodic agents; anti-thrombotic agents; anti-tubercular agents; antitussive agents; anti-ulcerative agents; anti-urolithic agents; antiviral agents; GERD medications, anxiolytics; appetite suppressants; attention deficit disorder (ADD) and attention deficit hyperactivity disorder (ADHD) drugs; bacteriostatic and bactericidal agents; benign prostatic hyperplasia therapy agents; blood glucose regulators; bone resorption inhibitors; bronchodilators; carbonic anhydrase inhibitors; cardiovascular preparations including anti-anginal agents, anti-arrhythmic agents, beta-blockers, calcium channel blockers, cardiac depressants, cardiovascular agents, cardioprotectants, and cardiotonic agents; central nervous system (CNS) agents; central nervous system stimulants; choleretic agents; cholinergic agents; cholinergic agonists; cholinesterase deactivators; coccidiostat agents; cognition adjuvants and cognition enhancers; cough and cold preparations, including decongestants; depressants; diagnostic aids; diuretics; dopaminergic agents; ectoparasiticides; emetic agents; enzymes which inhibit the formation of plaque, calculus or dental caries; enzyme inhibitors; estrogens; fibrinolytic agents; fluoride anticavity/antidecay agents; free oxygen radical scavengers; gastrointestinal motility agents; genetic materials; glucocorticoids; gonad-stimulating principles; hemostatic agents; herbal remedies; histamine H2 receptor antagonists; hormones; hormonolytics; hypnotics; hypocholesterolemic agents; hypoglycemic agents; hypolipidemic agents; hypotensive agents; immunizing agents; immunomodulators; immunoregulators; immunostimulants; immunosuppressants; impotence therapy adjuncts; inhibitors; keratolytic agents; leukotriene inhibitors; liver disorder treatments; metal chelators such as ethylenediaminetetraacetic acid, tetrasodium salt; mitotic inhibitors; mood regulators; mucolytics; mucosal protective agents; muscle relaxants; mydriatic agents; narcotic antagonists; neuroleptic agents; neuromuscular blocking agents; neuroprotective agents; nicotine; NMDA antagonists; non-hormonal sterol derivatives; nutritional agents, such as vitamins, essential amino acids and fatty acids; ophthalmic drugs such as antiglaucoma agents; oxytocic agents; pain relieving agents; parasympatholytics; peptide drugs; plasminogen activators; platelet activating factor antagonists; platelet aggregation inhibitors; post-stroke and post-head trauma treatments; potentiators; progestins; prostaglandins; prostate growth inhibitors; proteolytic enzymes as wound cleansing agents; prothyrotropin agents; psychostimulants; psychotropic agents; radioactive agents; regulators; relaxants; repartitioning agents; scabicides; sclerosing agents; sedatives; sedative-hypnotic agents; selective adenosine Al antagonists; serotonin antagonists; serotonin inhibitors; serotonin receptor antagonists; steroids, including progestogens, estrogens, corticosteroids, androgens and anabolic agents; smoking cessation agents; stimulants; suppressants; sympathomimetics; synergists; thyroid hormones; thyroid inhibitors; thyromimetic agents; tranquilizers; tooth desensitizing agents; tooth whitening agents such as peroxides, metal chlorites, perborates, percarbonates, peroxyacids, and combinations thereof; unstable angina agents; uricosuric agents; vasoconstrictors; vasodilators including general coronary, peripheral and cerebral; vulnerary agents; wound healing agents; xanthine oxidase inhibitors; and the like.

The various compositions disclosed herein may comprise a therapeutically effective amount of a drug, biologic, or active agent. Therapeutically effective amount refers to an amount of a drug, biologic, or active agent (alone or with one or more other active agents) sufficient to induce a desired response, such as to prevent, treat, reduce and/or ameliorate a condition. A therapeutically effective amount of an active agent, alone or with one or more other active agents, can be determined in many different ways, such as assaying for a reduction in of one or more signs or symptoms associated with the condition in the subject or measuring the level of one or more molecules associated with the condition to be treated.

The term “subject” includes living organisms, including those in which an immune response can be elicited (e.g., mammals). As used herein, the term “subject” or “patient” or “individual” may be used interchangeably. A “subject”, as used herein, may be a human or non-human mammal. Non-human mammals include, for example, livestock and pets, such as ovine, bovine, porcine, canine, non-human primates, feline and murine mammals. In some embodiments, the subject is human.

As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “in need thereof” means that the subject (animal or mammal) has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

The term “therapeutic” as used herein means a treatment and/or prophylaxis. A therapeutic effect is obtained by suppression, remission, or eradication of a disease state.

To “treat” a disease as the term is used herein, means to reduce the frequency or severity of at least one sign or symptom of a disease or disorder experienced by a subject.

Ranges: throughout this disclosure, various aspects of the embodiments can be presented in a range format. It should be understood that the description in range format is merely for convenience and brevity and should not be construed as an inflexible limitation. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. Unless otherwise explicitly stated to the contrary, a range that is disclosed also includes the endpoints of the range.

### Synthetic Pre-Protein Signal Peptides

Synthetic pre-protein signal peptides that increase secretion of a payload protein from bacteria are provided herein. Table 1 and Table 2 below lists various amino acid and polynucleotide sequences, respectively, that will be referred to herein.

TABLE 1

| SEQ ID | | |
|---|---|---|
| NO: | Sequence | Description |
| 1 | MKKLLLLLVVLLGLAALLLSGCGTSVSAAT | Reference signal peptide |
| 2 | MKKLLLLLVVLAGLAALLLSGCGTSVSAAT | Pre-Protein signal peptide |
| 3 | MKKLLLLLVVLLGLAALLLSGCGTSVSAAK | Pre-Protein signal peptide |
| 4 | MKKLLILLVVLLGLAALLLSGCGTSVSAAT | Pre-Protein signal peptide |
| 5 | MKKLLLLLVVLLSLAALLLSGCGTSVSAAK | Pre-Protein signal peptide |
| 6 | MKKLLLLLVVLAGLAALLLSGAGTSVSAAT | Pre-Protein signal peptide |
| 7 | MKKLLIGLVVLLGLAALLLSGLGTSVSAAT | Pre-Protein signal peptide |
| 8 | MKKLLLLLVVLAGLAALLLSGCGTSVSAAD | Pre-Protein signal peptide |
| 9 | MKKLTLLLVVVLGLAALCLSGCGISVSADE | Pre-Protein signal peptide |
| 10 | MHKLLLLLVVLVGLAALLLSGCGTTVSAAT | Pre-Protein signal peptide |
| 11 | MRKLLLLLVVLLGLAALLLSGAGTPVSAAT | Pre-Protein signal peptide |
| 12 | MKRLLLGLVGLLGLAILGLSGCGTSVSAQT | Pre-Protein signal peptide |
| 13 | MKKLLLLLVLVLGLAAMLLSGCGTPTSADT | Pre-Protein signal peptide |
| 14 | MKKILLLVVVLLGLAALLWSGAGTSVSAST | Pre-Protein signal peptide |
| 15 | MRRLLLLLVVVLGLAALLLSGCGTSVSAQA | Pre-Protein signal peptide |
| 16 | MRKLHELLIVLLGISFVLWSGCGTSVSAAG | Pre-Protein signal peptide |
| 17 | MKKLLLGLVLLLGLAALALTGCGTPVSAAT | Pre-Protein signal peptide |
| 18 | MKKLLLLLVVLLSVAALLASGCGTFVSAAT | Pre-Protein signal peptide |
| 19 | MKRLLLKAVVLLSLAALLWSGAGTSVSAAT | Pre-Protein signal peptide |
| 20 | MKRLLIGAVVLLGLAALLLSGCGTSVSAAT | Pre-Protein signal peptide |
| 47 | MKKLTLLLTVSLGMAGFLLSGCGQPVSAAQ | Pre-Protein signal peptide |
| 48 | MHKLLMLLVVLLGLAGLSLSGCGSSTSAAT | Pre-Protein signal peptide |
| 144 | MKRLLELLVVVLGLAALLLSGCGTSVSAAS | Pre-Protein signal peptide |
| 145 | MKKLTLLIVVLLGLAALGLSGCGTSVSAAT | Pre-Protein signal peptide |
| 146 | MKKLHLLLVVVFGLAALLLAGCGTSVSAAT | Pre-Protein signal peptide |
| 147 | MKKLLLLIVVLLGLAFLLLSGCGTSVSAAS | Pre-Protein signal peptide |
| 148 | MKKLLLLLVASLSLAAVLLSGIGTSVSAAT | Pre-Protein signal peptide |
| 149 | MKKKLLTLVLLLGLAALLLTGCGQSVSAAT | Pre-Protein signal peptide |
| 150 | MKKLLLLLVVLLGLGGFLLSGCGTSVSAST | Pre-Protein signal peptide |
| 151 | MKKLIATLVVLLGLAALMLSACGTSVSAAT | Pre-Protein signal peptide |
| 152 | MKKLHLLLVGLLGLAALLLSGIGTSVSAAT | Pre-Protein signal peptide |
| 153 | MKKLLLLLLVLLSVAALLLSACGTSVSAAT | Pre-Protein signal peptide |
| 154 | MKKLLLLLVVLLGLAALLLSGCGTTVCAAT | Pre-Protein signal peptide |
| 155 | MKKWLLLLVVLLGLGALLLSGCGTSVSAAT | Pre-Protein signal peptide |
| 156 | MKKLLLLLVVLLGLAFLLLSGCGTTVSAAT | Pre-Protein signal peptide |
| 157 | MKKLLLKLVVLLGLAAILLSGCGTTVSAAT | Pre-Protein signal peptide |
| 158 | MKKLLGLLVVALSLAALLESGVGTSVSAAT | Pre-Protein signal peptide |
| 159 | MKKLLLLLGVLAGLAALLLSGCGTSVSADT | Pre-Protein signal peptide |
| 160 | MKKLLLLLVVLLGLTAFLLSGCGTSVSAAT | Pre-Protein signal peptide |
| 161 | MKKLLLLLVVLLGLAALCLSGCGTSVSASG | Pre-Protein signal peptide |
| 162 | MKKLLLLLTVLLGLAALWLSGFGTSSSAAT | Pre-Protein signal peptide |
| 163 | MKKLLLLAVVLLGLSALLLSGCGTSVSAAG | Pre-Protein signal peptide |
| 164 | MKKLLITLVCAIGLAALLLSGCGTSVSAAT | Pre-Protein signal peptide |
| 165 | MKKLLLGLVVVLGLAALFLSGCGTSVSAAT | Pre-Protein signal peptide |
| 166 | MKRLLILLVAVLGLAALLLSGCGTSVSAST | Pre-Protein signal peptide |
| 167 | MKKALLKLVVLMGLAALLLSGCGTSVSAAS | Pre-Protein signal peptide |
| 168 | MKKLLLLLVVALGLAALLLSTSTSAST | Pre-Protein signal peptide |
| 169 | MKRLLLLLTLLLGLAAFLLSGCGTSVSAAN | Pre-Protein signal peptide |
| 170 | MKKLLLLLVVLTGLAAMLLSSCGTSVSAAT | Pre-Protein signal peptide |
| 171 | MKKLKALLVVLLGLAALLFSGCGTSVSAAT | Pre-Protein signal peptide |
| 172 | MKKLLLLLVVLLGLAMLLLSGCGTQVSASS | Pre-Protein signal peptide |
| 173 | MKKLLALLVVALGLSALLLSGCGTSVSAAT | Pre-Protein signal peptide |
| 174 | MKKLILLLTVLTGLAALLLSGCGSSVSAAT | Pre-Protein signal peptide |
| 175 | MKKLLLLLVVALSLAALLLSGCGTSVSAQT | Pre-Protein signal peptide |
| 176 | MKRLLLLLLVLLGLALLLLSGCGASVSAAT | Pre-Protein signal peptide |
| 177 | MKKLLLLAVVLLGLAALLASGCGTSTSAAT | Pre-Protein signal peptide |
| 178 | MKKLLMLLVVLLSIAALLLAGCGTSVSAAT | Pre-Protein signal peptide |
| 179 | MKKLLFLLVVLLGLAALGLSGVGTSVSAAT | Pre-Protein signal peptide |
| 180 | MKKLLLGLVVLFGLAALLLSGAGVSVSAST | Pre-Protein signal peptide |
| 181 | MKKILLLLVGLFGLAALLLSGCGTSVSAAT | Pre-Protein signal peptide |
| 182 | MKKWLLALVVLLTLAAFLVSGCGTSVSAAT | Pre-Protein signal peptide |
| 183 | MKRLLLGLVVLIGVTAFLLSGCGTSVSAAT | Pre-Protein signal peptide |
| 184 | MKKLLLLLVGLLGVAALLLSGCGTSASAAT | Pre-Protein signal peptide |
| 185 | MKKVLALLVVLLGLAALGLSGCGTSVSAAT | Pre-Protein signal peptide |
| 186 | MKKLLLLLVCLLGLAALGLSACGTSVSAAT | Pre-Protein signal peptide |
| 187 | MKKLLILLTLVISLAAFLLSGCGTSVSAAT | Pre-Protein signal peptide |
| 188 | MKKLLLLLVVVLGLALLLLSGCGTSVHAAS | Pre-Protein signal peptide |
| 189 | MKKLISLLVVLLGLAALLLSGCGTSDSAAA | Pre-Protein signal peptide |
| 190 | MKKLLILLVVLLGMAALLLSGCGTSVSADT | Pre-Protein signal peptide |
| 191 | MKKLLLLLTVALGLAALLLSGLGTSVSAAT | Pre-Protein signal peptide |
| 192 | MKKLTLGLVVAAGLAALLLSGCGTPTSAAE | Pre-Protein signal peptide |
| 193 | MKRLLLLLVVVLGLAALLLSGCGTSVHAAT | Pre-Protein signal peptide |
| 194 | MKRLLELLVVLLGLAALCLSGCGTSVSAAA | Pre-Protein signal peptide |
| 195 | MKKLLLLAVVLLGLAGLLLSGCGTPVSAAT | Pre-Protein signal peptide |
| 196 | MKKLLLLVTVLLGLAAMGLSGAGVSVSAST | Pre-Protein signal peptide |
| 197 | MKKLLALLVVLLGLAALLLSVCGTSVSAAT | Pre-Protein signal peptide |
| 198 | MKKLLLLLVMLFGLAFLLLSGCGTSVSAST | Pre-Protein signal peptide |
| 199 | MKKLLLLLVVALGLAAMLLSGCGSSVHAAT | Pre-Protein signal peptide |
| 200 | MKKLLLLLTVLLSLAALLLSGTGTPVSAAT | Pre-Protein signal peptide |
| 201 | MKKLLLLLVGLLGLAALLLSGVGTSVHAAT | Pre-Protein signal peptide |
| 202 | MKRLLILLVVLIGLAALGLSGCGTSVSAAT | Pre-Protein signal peptide |
| 203 | MKKLLSLIVVLLGLAAMLLSGCGTSVSAAC | Pre-Protein signal peptide |
| 204 | MKKLLLLLLVLLFMAALLLSGCGTSVSAST | Pre-Protein signal peptide |
| 205 | MKRLLLLLVLLLGLAAALLTGCGTSVSAAT | Pre-Protein signal peptide |
| 206 | MKKLLLLLVVLLGVTALLLSGCGKSVSAAT | Pre-Protein signal peptide |
| 207 | MKKLLLLLVVLLGCAALLLSGCGTPSSAAT | Pre-Protein signal peptide |
| 208 | MKKLLLLFVVLLGLAALLESGAGTSVSAAT | Pre-Protein signal peptide |
| 209 | MKKLLLLLVVLLFVAALGLSGCGTSTSAAT | Pre-Protein signal peptide |
| 210 | MKKLLLFLVVLLGMSALGLSGCGTSVHAAS | Pre-Protein signal peptide |
| 211 | MKKLLLLLVLLLGLAAFLLSGCGTPVSAQT | Pre-Protein signal peptide |
| 212 | MKRLLELLVVLLGLAFLMLSGCGTRVHAAT | Pre-Protein signal peptide |

TABLE 2

| SEQ ID | | |
|---|---|---|
| NO: | Sequence | Description |
| 132 | ATGAAAAAATTGTTGTTGTTGTTGGTTGTT | Nucleotide sequence for |
| | TTGGCAGGCTTGGCAGCATTGTTGTTGTCA | SEQ ID NO: 6 |
| | GGCGCAGGCACCTCAGTTTCAGCAGCAACC | |
| 133 | ATGAAAAAATTGTTGATTGGCTTGGTTGTT | Nucleotide sequence for |
| | TTGTTGGGCTTGGCAGCATTGTTGTTGTCA | SEQ ID NO: 7 |
| | GGCTTGGGCACCTCAGTTTCAGCAGCAACC | |
| 134 | ATGAAAAAAATTTTGTTGTTGGTTGTTGTT | Nucleotide sequence for |
| | TTGTTGGGCTTGGCAGCATTGTTGTGGTCA | SEQ ID NO: 14 |
| | GGCGCAGGCACCTCAGTTTCAGCATCAACC | |
| 135 | ATGAAAAAGCTGCTCCTGGGTCTGGTTCTG | Nucleotide sequence for |
| | CTCCTGGGCCTGGCAGCGCTGGCCCTGACC | SEQ ID NO: 17 |
| | GGTTGTGGCACTCCGGTGAGCGCCGCAACT | |
| 136 | ATGAAGAAACTGACCCTGCTCCTGACTGTC | Nucleotide sequence for |
| | TCCCTGGGTATGGCAGGTTTCCTCCTGTCC | SEQ ID NO: 47 |
| | GGTTGCGGCCAGCCTGTTTCCGCGGCCCAG | |
| 137 | ATGAAGAAACTGCTCCTGCTCCTGGTAGTT | Nucleotide sequence for |
| | CTCCTGTCCGTTGCCGCTCTCCTGGCGAGC | SEQ ID NO: 18 |
| | GGCTGTGGTACTTTCGTATCCGCAGCAACC | |
| 138 | ATGAAACGCCTGCTCCTGAAAGCTGTTGTG | Nucleotide sequence for |
| | CTGCTCTCTCTGGCAGCCCTCCTGTGGTCT | SEQ ID NO: 19 |
| | GGCGCAGGCACCTCCGTGAGCGCTGCCACC | |
| 139 | ATGCACAAACTCCTGATGCTCCTGGTCGTG | Nucleotide sequence for |
| | CTCCTGGGTCTCGCTGGCCTGTCTCTGAGC | SEQ ID NO: 48 |
| | GGTTGCGGTAGCTCCACGTCCGCTGCGACC | |
| 140 | ATGAAGCGCCTGCTCATCGGTGCGGTTGTC | Nucleotide sequence for |
| | CTCCTGGGTCTGGCGGCTCTGCTCCTGTCC | SEQ ID NO: 20 |
| | GGTTGTGGTACTTCTGTTTCCGCAGCCACG | |
| 213 | ATGAAACGCTTGCTTGAGTTATTGGTTGTG | Nucleotide sequence for |
| | GTTCTGGGCCTGGCCGCACTACTGTTGAGT | SEQ ID NO: 144 |
| | GGCTGCGGCACTAGCGTCAGCGCTGCTTCA | |
| 214 | ATGAAGAAATTAACGCTCCTTATTGTTGTA | Nucleotide sequence for |
| | CTGCTTGGACTCGCGGCCTTGGGACTGAGC | SEQ ID NO: 145 |
| | GGATGCGGAACATCTGTTTCCGCGGCAACG | |
| 215 | ATGAAAAAGTTGCATCTATTATTGGTGGTA | Nucleotide sequence for |
| | GTCTTCGGCCTCGCTGCCTIGTTATTGGCG | SEQ ID NO: 146 |
| | GGCTGTGGGACCTCCGTCAGCGCCGCAACC | |
| 216 | ATGAAAAAGTTGCTCTTATTGATTGTTGTC | Nucleotide sequence for |
| | CTATTGGGGTTGGCATTTCTCCTTCTATCA | SEQ ID NO: 147 |
| | GGCTGTGGAACCTCGGTTTCCGCTGCGTCT | |
| 217 | ATGAAGAAATTGCTTCTGCTATTAGTTGCA | Nucleotide sequence for |
| | AGCCTCTCTCTGGCAGCTGTCTTGCTGTCA | SEQ ID NO: 148 |
| | GGAATCGGCACGTCTGTTTCGGCCGCCACG | |
| 218 | ATGAAGAAAAAGTTACTGACATTGGTATTG | Nucleotide sequence for |
| | CTGCTCGGGTTGGCTGCACTTCTGCTAACT | SEQ ID NO: 149 |
| | GGATGCGGACAATCTGTTAGTGCTGCGACA | |
| 219 | ATGAAGAAATTGTTATTGCTTTTGGTCGTT | Nucleotide sequence for |
| | TTGCTGGGATTGGGCGGTTTTTTGCTTTCA | SEQ ID NO: 150 |
| | GGCTGTGGGACGTCAGTAAGTGCCTCAACC | |
| 220 | ATGAAGAAACTAATTGCCACGTTAGTTGTC | Nucleotide sequence for |
| | CTTTTGGGGCTTGCCGCACTAATGCTGAGC | SEQ ID NO: 151 |
| | GCTTGTGGAACATCTGTATCCGCAGCAACG | |
| 221 | ATGAAGAAATTACATCTCCTTTTGGTAGGC | Nucleotide sequence for |
| | CTTTTAGGATTAGCTGCGTTGCTTTTAAGC | SEQ ID NO: 152 |
| | GGAATCGGCACGTCAGTATCAGCGGCAACG | |
| 222 | ATGAAGAAACTTTTGTTATTGTTACTCGTC | Nucleotide sequence for |
| | TTATTGAGCGTAGCAGCCTTACTCTTATCG | SEQ ID NO: 153 |
| | GCGTGCGGGACTTCGGTCAGCGCGGCTACG | |
| 223 | ATGAAAAAGCTCCTTTTACTGCTTGTAGTG | Nucleotide sequence for |
| | TTGCTTGGACTGGCAGCTCTCCTGCTAAGC | SEQ ID NO: 154 |
| | GGCTGCGGGACAACCGTTTGCGCGGCAACG | |
| 224 | ATGAAGAAATGGTTGCTTCTGCTTGTTGTC | Nucleotide sequence for |
| | CTATTGGGTTTGGGTGCTTTACTGTTATCA | SEQ ID NO: 155 |
| | GGCTGCGGAACTTCAGTATCCGCAGCGACA | |
| 225 | ATGAAGAAACTACTTTTATTGTTAGTTGTC | Nucleotide sequence for |
| | TTACTGGGCTTAGCTTTTCTGTTGCTATCA | SEQ ID NO: 156 |
| | GGTTGTGGCACAACCGTTAGCGCCGCCACC | |
| 226 | ATGAAAAAGTTGCTTCTGAAGTTAGTCGTG | Nucleotide sequence for |
| | TTACTGGGTCTTGCGGCTATCTTACTTTCC | SEQ ID NO: 157 |
| | GGTTGCGGGACGACCGTTTCAGCTGCCACA | |
| 227 | ATGAAGAAATTATTGGGACTTTTGGTGGTC | Nucleotide sequence for |
| | GCGTTGTCCTTGGCGGCACTGTTATTTTCA | SEQ ID NO: 158 |
| | GGTGTAGGGACTTCGGTTTCGGCCGCTACT | |
| 228 | ATGAAGAAATTGCTTTTATTGCTAGGCGTT | Nucleotide sequence for |
| | TTGGCAGGTTTGGCTGCCTTACTGCTTAGC | SEQ ID NO: 159 |
| | GGGTGCGGCACCTCCGTCTCGGCTGATACA | |
| 229 | ATGAAGAAATTGCTCTTGCTTTTAGTAGTT | Nucleotide sequence for |
| | TTACTTGGTTTGACTGCATTTCTGTTAAGC | SEQ ID NO: 160 |
| | GGGTGTGGTACGTCAGTCTCCGCAGCCACT | |
| 230 | ATGAAAAAGCTTCTGTTGCTGCTAGTTGTG | Nucleotide sequence for |
| | TTGTTAGGTCTGGCGGCCCTTTGTCTTTCA | SEQ ID NO: 161 |
| | GGTTGCGGCACTTCCGTCTCGGCATCAGGC | |
| 231 | ATGAAAAAGCTCTTACTCCTACTTACCGTT | Nucleotide sequence for |
| | CTACTTGGCCTAGCAGCCCTCTGGTTGTCA | SEQ ID NO: 162 |
| | GGGTTTGGTACATCTTCAAGCGCCGCAACT | |
| 232 | ATGAAAAAGCTTCTGCTATTGGCCGTGGTA | Nucleotide sequence for |
| | TTATTGGGCCTCTCAGCGTTACTGTTGAGT | SEQ ID NO: 163 |
| | GGATGTGGGACAAGCGTCAGCGCCGCAGGG | |
| 233 | ATGAAAAAGCTCCTTATAACCCTTGTATGC | Nucleotide sequence for |
| | GCCATCGGCCTTGCGGCCTTGCTCCTGAGC | SEQ ID NO: 164 |
| | GGCTGTGGCACTAGCGTGTCAGCGGCCACC | |
| 234 | ATGAAGAAATTATTGTTAGGCTTGGTTGTG | Nucleotide sequence for |
| | GTTCTGGGCCTCGCTGCCCTTTTCTTGAGC | SEQ ID NO: 165 |
| | GGCTGCGGCACGTCTGTATCAGCAGCTACG | |
| 235 | ATGAAGAGACTTTTAATCCTTTTAGTAGCC | Nucleotide sequence for |
| | GTCCTAGGGCTAGCTGCGTTACTTTTAAGT | SEQ ID NO: 166 |
| | GGCTGTGGAACATCGGTAAGCGCCTCGACC | |
| 236 | ATGAAGAAAGCATTGTTAAAACTGGTAGTT | Nucleotide sequence for |
| | CTGATGGGGCTTGCAGCGCTTCTGTTGAGT | SEQ ID NO: 167 |
| | GGTTGCGGAACTAGTGTTAGCGCCGCCTCC | |
| 237 | ATGAAGAAACTCCTACTTTTGCTTGTTGTG | Nucleotide sequence for |
| | GCCTTAGGCTTGGCGGCTCTTCTACTGAGT | SEQ ID NO: 168 |
| | ACATCAACGTCGGCGTCAACC | |
| 238 | ATGAAACGCTTGTTACTCTTGCTCACGTTG | Nucleotide sequence for |
| | CTTCTAGGCCTTGCTGCCTTCTTATTGTCG | SEQ ID NO: 169 |
| | GGCTGCGGCACTTCAGTTTCCGCAGCGAAC | |
| 239 | ATGAAGAAACTTCTGTTATTGCTTGTGGTT | Nucleotide sequence for |
| | CTTACCGGACTTGCTGCAATGCTTCTATCG | SEQ ID NO: 170 |
| | TCATGCGGCACTTCGGTGTCAGCTGCGACT | |
| 240 | ATGAAAAAGCTCAAAGCTCTATTAGTAGTG | Nucleotide sequence for |
| | CTACTTGGACTCGCGGCATTATTGTTCAGT | SEQ ID NO: 171 |
| | GGTTGTGGTACCTCAGTGTCCGCAGCTACC | |
| 241 | ATGAAAAAGCTGCTTTTGTTATTGGTTGTA | Nucleotide sequence for |
| | CTTTTGGGACTTGCTATGCTCTTACTTTCT | SEQ ID NO: 172 |
| | GGTTGCGGTACTCAAGTCTCAGCTTCCAGC | |
| 242 | ATGAAGAAACTATTGGCACTTTTAGTIGTC | Nucleotide sequence for |
| | GCGCTAGGTCTTTCCGCTTTGCTTTTGTCT | SEQ ID NO: 173 |
| | GGTTGTGGTACCAGCGTGTCGGCCGCTACG | |
| 243 | ATGAAGAAACTAATTCTCCTTTTGACGGTG | Nucleotide sequence for |
| | TTGACGGGCTTAGCAGCGTTATTGCTATCG | SEQ ID NO: 174 |
| | GGTTGCGGATCCAGTGTTTCCGCAGCCACA | |
| 244 | ATGAAAAAGTTACTGTTGCTCCTAGTTGTA | Nucleotide sequence for |
| | GCTCTTTCTTTGGCCGCGTTGTTACTTAGC | SEQ ID NO: 175 |
| | GGCTGTGGCACGTCAGTTAGTGCTCAAACC | |
| 245 | ATGAAGCGACTTTTGTTACTTTTACTTGTT | Nucleotide sequence for |
| | CTACTTGGACTTGCGTTGCTTTTGCTCTCT | SEQ ID NO: 176 |
| | GGTTGCGGCGCCTCTGTTTCGGCAGCTACG | |
| 246 | ATGAAAAAGCTTTTATTGCTTGCTGTGGTT | Nucleotide sequence for |
| | CTTCTGGGGTTAGCCGCACTGTTGGCGAGC | SEQ ID NO: 177 |
| | GGGTGCGGTACTTCCACTAGTGCTGCAACG | |
| 247 | ATGAAGAAATTATTGATGTTATTGGTCGTG | Nucleotide sequence for |
| | TTGCTTTCGATTGCTGCGCTGTTGCTAGCC | SEQ ID NO: 178 |
| | GGCTGTGGCACATCTGTGTCGGCTGCCACG | |
| 248 | ATGAAAAAGCTACTGTTTCTACTTGTTGTC | Nucleotide sequence for |
| | CTTCTGGGCTTGGCGGCTCTAGGCCTCAGT | SEQ ID NO: 179 |
| | GGTGTCGGCACATCAGTAAGCGCTGCTACC | |
| 249 | ATGAAGAAATTACTACTTGGCCTCGTGGTC | Nucleotide sequence for |
| | CTGTTTGGGCTCGCTGCCCTTTTGCTGAGT | SEQ ID NO: 180 |
| | GGAGCGGGGGTTTCAGTTTCAGCATCCACC | |
| 250 | ATGAAAAAGATCCTGCTTTTACTGGTCGGG | Nucleotide sequence for |
| | TTGTTTGGTCTAGCTGCCCTTTTGTTATCA | SEQ ID NO: 181 |
| | GGCTGTGGAACATCAGTGTCTGCCGCAACG | |
| 251 | ATGAAGAAATGGCTTTTGGCATTGGTTGTG | Nucleotide sequence for |
| | TTGCTAACCCTTGCAGCCTTCTTAGTATCA | SEQ ID NO: 182 |
| | GGTTGCGGCACAAGCGTGTCAGCTGCGACG | |
| 252 | ATGAAAAGGCTTTTGCTTGGCCTGGTAGIT | Nucleotide sequence for |
| | CTTATTGGCGTAACTGCGTTTCTGTTATCG | SEQ ID NO: 183 |
| | GGTTGTGGTACATCCGTCTCCGCCGCCACC | |
| 253 | ATGAAGAAATTACTTCTGCTCCTTGTAGGC | Nucleotide sequence for |
| | TTGCTGGGCGTAGCTGCCTTGCTTTTGAGT | SEQ ID NO: 184 |
| | GGTTGCGGGACCTCCGCCTCGGCTGCAACC | |
| 254 | ATGAAAAAGGTGCTTGCCCTGCTTGTCGTT | Nucleotide sequence for |
| | CTCTTAGGTTTGGCAGCTCTTGGTTTATCA | SEQ ID NO: 185 |
| | GGATGCGGTACGTCAGTGTCAGCCGCCACG | |
| 255 | ATGAAAAAGTTACTACTTTTGCTGGTCTGT | Nucleotide sequence for |
| | CTGCTCGGATTGGCGGCCTTAGGCTTGTCC | SEQ ID NO: 186 |
| | GCGTGCGGCACATCAGTGTCCGCTGCAACT | |
| 256 | ATGAAGAAACTACTGATCCTTTTAACGTTG | Nucleotide sequence for |
| | GTTATATCCCTGGCGGCCTTCTTGCTGTCA | SEQ ID NO: 187 |
| | GGATGCGGCACATCAGTTTCCGCTGCCACC | |
| 257 | ATGAAAAAGCTGTTGCTATTACTTGTAGTT | Nucleotide sequence for |
| | GTCCTAGGACTGGCTTTACTGTTACTGTCA | SEQ ID NO: 188 |
| | GGTTGTGGAACTTCGGTTCACGCCGCCTCC | |
| 258 | ATGAAGAAACTGATCAGCTTGCTCGTAGTT | Nucleotide sequence for |
| | TTGTTAGGTTTGGCCGCATTACTATTATCT | SEQ ID NO: 189 |
| | GGCTGCGGTACGAGTGATTCAGCCGCGGCT | |
| 259 | ATGAAGAAACTCCTGATCCTGCTCGTAGTT | Nucleotide sequence for |
| | CTCCTTGGCATGGCAGCTTTATTGTTATCT | SEQ ID NO: 190 |
| | GGTTGTGGGACCTCTGTGTCCGCGGACACC | |
| 260 | ATGAAGAAACTTTTGCTTCTGCTTACCGTG | Nucleotide sequence for |
| | GCACTAGGCCTGGCGGCACTGTTGCTTAGT | SEQ ID NO: 191 |
| | GGTCTTGGAACATCCGTCTCCGCCGCCACC | |
| 261 | ATGAAAAAGTTGACACTTGGCCTTGTGGTA | Nucleotide sequence for |
| | GCGGCCGGCCTCGCGGCATTACTCTTGTCA | SEQ ID NO: 192 |
| | GGGTGCGGCACACCTACGAGTGCGGCCGAG | |
| 262 | ATGAAGCGACTGTTGCTTTTGTTAGTAGTC | Nucleotide sequence for |
| | GTATTAGGATTGGCGGCCTTATTGTTAAGT | SEQ ID NO: 193 |
| | GGTTGCGGAACAAGCGTGCATGCGGCTACA | |
| 263 | ATGAAACGATTACTTGAGCTTCTAGTAGTG | Nucleotide sequence for |
| | TTGCTTGGTTTAGCCGCATTATGCCTGTCC | SEQ ID NO: 194 |
| | GGTTGCGGTACAAGTGTATCGGCTGCGGCA | |
| 264 | ATGAAGAAACTTCTATTATTGGCCGTAGTC | Nucleotide sequence for |
| | TTACTTGGTTTAGCAGGCTTGCTTTTGTCA | SEQ ID NO: 195 |
| | GGGTGCGGTACACCCGTCTCAGCGGCGACT | |
| 265 | ATGAAAAAGTTACTGTTGCTGGTTACCGTC | Nucleotide sequence for |
| | TTACTTGGCCTTGCTGCAATGGGTTTGTCC | SEQ ID NO: 196 |
| | GGAGCCGGTGTCTCGGTCAGTGCTAGCACT | |
| 266 | ATGAAAAAGCTGTTAGCGCTATTAGTAGTC | Nucleotide sequence for |
| | TTATTGGGTTTAGCGGCCTTACTGTTGTCT | SEQ ID NO: 197 |
| | GTGTGTGGAACCAGCGTTTCAGCCGCAACC | |
| 267 | ATGAAGAAATTGCTCTTGCTGCTCGTTATG | Nucleotide sequence for |
| | TTGTTCGGCCTTGCTTTCCTGCTCTTATCA | SEQ ID NO: 198 |
| | GGCTGTGGAACCTCCGTTTCGGCCTCCACA | |
| 268 | ATGAAGAAATTGCTCTTGCTACTTGTTGTA | Nucleotide sequence for |
| | GCACTAGGATTGGCGGCCATGCTCTTGTCA | SEQ ID NO: 199 |
| | GGGTGCGGATCCAGTGTTCACGCAGCTACC | |
| 269 | ATGAAGAAACTCTTGTTATTGCTTACCGTC | Nucleotide sequence for |
| | TTGTTAAGTTTAGCAGCCTTGCTTTTGTCC | SEQ ID NO: 200 |
| | GGTACAGGGACTCCCGTTTCAGCCGCTACG | |
| 270 | ATGAAGAAATTGTTACTACTTTTGGTAGGT | Nucleotide sequence for |
| | TTGCTTGGGTTGGCCGCTTTGCTTTTGTCT | SEQ ID NO: 201 |
| | GGCGTGGGAACGTCGGTCCACGCGGCCACC | |
| 271 | ATGAAGCGACTCTTAATTCTATTGGTTGTA | Nucleotide sequence for |
| | CTTATAGGTTTGGCAGCCTTAGGCCTGTCT | SEQ ID NO: 202 |
| | GGCTGCGGTACATCTGTCAGTGCCGCCACG | |
| 272 | ATGAAGAAATTGCTTAGCTTGATCGTCGTT | Nucleotide sequence for |
| | CTATTAGGACTGGCCGCTATGCTGTTATCT | SEQ ID NO: 203 |
| | GGATGTGGAACGTCAGTTTCAGCCGCTTGT | |
| 273 | ATGAAGAAATTGCTATTACTGTTGCTGGTA | Nucleotide sequence for |
| | CTTCTGTTTATGGCAGCCCTACTICTCTCA | SEQ ID NO: 204 |
| | GGCTGCGGCACCTCAGTGTCCGCGAGTACC | |
| 274 | ATGAAACGCCTGCTTTTACTCTTGGTCCTT | Nucleotide sequence for |
| | TTGTTAGGACTTGCAGCTGCACTICTGACC | SEQ ID NO: 205 |
| | GGATGCGGGACCTCGGTTAGTGCCGCCACC | |
| 275 | ATGAAGAAATTACTCCTATTGCTTGTTGTA | Nucleotide sequence for |
| | CTGTTGGGCGTTACCGCCTTATTGCTTTCC | SEQ ID NO: 206 |
| | GGCTGCGGTAAATCGGTTTCAGCGGCCACT | |
| 276 | ATGAAAAAGTTACTTCTGCTTTTGGTCGTT | Nucleotide sequence for |
| | TTGTTAGGTTGTGCCGCACTACTCCTTAGT | SEQ ID NO: 207 |
| | GGTTGTGGGACACCATCCAGTGCAGCCACT | |
| 277 | ATGAAGAAATTGCTGCTATTATTIGTTGTA | Nucleotide sequence for |
| | CTACTTGGTCTTGCAGCCCTGCTATTTTCA | SEQ ID NO: 208 |
| | GGTGCAGGCACATCAGTCAGCGCCGCGACC | |
| 278 | ATGAAAAAGCTTTTATTGTTATTGGTGGTT | Nucleotide sequence for |
| | CTTCTGTTTGTCGCAGCCCTTGGATTGTCA | SEQ ID NO: 209 |
| | GGATGCGGCACATCTACATCTGCGGCTACT | |
| 279 | ATGAAGAAATTGTTATTGTTCCTAGTGGTC | Nucleotide sequence for |
| | CTGTTAGGGATGAGTGCTCTCGGTTTGTCT | SEQ ID NO: 210 |
| | GGATGCGGCACGAGTGTGCATGCCGCGTCG | |
| 280 | ATGAAAAAGTTATTGCTCTTATTGGTTTTA | Nucleotide sequence for |
| | CTCTTGGGTCTTGCCGCATTTCTGTTAAGC | SEQ ID NO: 211 |
| | GGGTGTGGCACACCAGTCAGTGCCCAGACG | |
| 281 | ATGAAGAGATTACTCGAGCTCCTGGTCGTG | Nucleotide sequence for |
| | CTTTTAGGCCTGGCGTTCCTGATGTTAAGT | SEQ ID NO: 212 |
| | GGTTGCGGCACACGCGTACATGCTGCCACA | |

The synthetic pre-protein signal peptides disclosed herein are optimized for use in Lactobacillus bacteria and any bacteria within the Lactobacillus genus can be used to express a payload protein as provided for herein. Particular examples of suitable bacteria species are provided herein below to exemplify the particular synthetic signal peptides that have been developed.

In some embodiments, a pre-protein signal peptide is provided. In some embodiments, the pre-protein signal peptide comprises an amino acid sequence having at least 80%, 85%, 90%, 91%, 92%, 93%, 94%, 95%, 96%, 97%, 98%, 99%, or 100% identity to an amino acid sequence as provided in Table 1. In some embodiments, the pre-protein signal peptide comprises an amino acid sequence having at least 90%, 91%, 92%, 93%, 94%, 95%, 96%, 97%, 98%, 99%, or 100% identity to an amino acid sequence as provided in Table 1. In some embodiments, the pre-protein signal peptide comprises an amino acid sequence having at least 95%, 96%, 97%, 98%, 99%, or 100% identity to an amino acid sequence as provided in Table 1. In some embodiments, the pre-protein signal peptide comprises an amino acid sequence having at least 95% identity to an amino acid sequence as provided in Table 1. In some embodiments, the pre-protein signal peptide comprises an amino acid sequence having at least 98% identity to an amino acid sequence as provided in Table 1. In some embodiments, the pre-protein signal peptide comprises an amino acid sequence as provided in Table 1.

In some embodiments, the pre-protein signal peptide comprises an amino acid sequence having at least 80%, 85%, 90%, 91%, 92%, 93%, 94%, 95%, 96%, 97%, 98%, 99%, or 100% identity to an amino acid sequence selected from SEQ ID NO: 6. SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180. In some embodiments, the pre-protein signal peptide comprises an amino acid sequence having at least 98% identity to an amino acid sequence selected from SEQ ID NO: 6. SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180. In some embodiments, the pre-protein signal peptide comprises an amino acid sequence selected from SEQ ID NO: 6. SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180.

In some embodiments, the pre-protein signal peptide comprises an amino acid sequence having at least 80%, 85%, 90%, 91%, 92%, 93%, 94%, 95%, 96%, 97%, 98%, 99%, or 100% identity to an amino acid sequence selected from SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, or SEQ ID NO: 212 In some embodiments, the pre-protein signal peptide comprises an amino acid sequence having at least 98% identity to an amino acid sequence selected from SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, or SEQ ID NO: 212. In some embodiments, the pre-protein signal peptide comprises an amino acid sequence selected from SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, or SEQ ID NO: 212.

In some embodiments, the pre-protein signal peptide comprises an amino acid sequence having at least 80%, 85%, 90%, 91%, 92%, 93%, 94%, 95%, 96%, 97%, 98%, 99%, or 100% identity to the amino acid sequence of SEQ ID NO: 6. In some embodiments, the pre-protein signal peptide comprises an amino acid sequence having at least 98% identity to the amino acid sequence of SEQ ID NO: 6. In some embodiments, the pre-protein signal peptide comprises the amino acid sequence of SEQ ID NO: 6.

In some embodiments, the pre-protein signal peptide comprises an amino acid sequence having at least 80%, 85%, 90%, 91%, 92%, 93%, 94%, 95%, 96%, 97%, 98%, 99%, or 100% identity to the amino acid sequence of SEQ ID NO: 7. In some embodiments, the pre-protein signal peptide comprises an amino acid sequence having at least 98% identity to the amino acid sequence of SEQ ID NO: 7. In some embodiments, the pre-protein signal peptide comprises the amino acid sequence of SEQ ID NO: 7.

In some embodiments, the pre-protein signal peptide comprises an amino acid sequence having at least 80%, 85%, 90%, 91%, 92%, 93%, 94%, 95%, 96%, 97%, 98%, 99%, or 100% identity to the amino acid sequence of SEQ ID NO: 14. In some embodiments, the pre-protein signal peptide comprises an amino acid sequence having at least 98% identity to the amino acid sequence of SEQ ID NO: 14. In some embodiments, the pre-protein signal peptide comprises the amino acid sequence of SEQ ID NO: 14.

In some embodiments, the pre-protein signal peptide comprises an amino acid sequence having at least 80%, 85%, 90%, 91%, 92%, 93%, 94%, 95%, 96%, 97%, 98%, 99%, or 100% identity to the amino acid sequence of SEQ ID NO: 17. In some embodiments, the pre-protein signal peptide comprises an amino acid sequence having at least 98% identity to the amino acid sequence of SEQ ID NO: 17. In some embodiments, the pre-protein signal peptide comprises the amino acid sequence of SEQ ID NO: 17.

In some embodiments, the pre-protein signal peptide comprises an amino acid sequence having at least 80%, 85%, 90%, 91%, 92%, 93%, 94%, 95%, 96%, 97%, 98%, 99%, or 100% identity to the amino acid sequence of SEQ ID NO: 18. In some embodiments, the pre-protein signal peptide comprises an amino acid sequence having at least 98% identity to the amino acid sequence of SEQ ID NO: 18. In some embodiments, the pre-protein signal peptide comprises the amino acid sequence of SEQ ID NO: 18.

In some embodiments, the pre-protein signal peptide comprises an amino acid sequence having at least 80%, 85%, 90%, 91%, 92%, 93%, 94%, 95%, 96%, 97%, 98%, 99%, or 100% identity to the amino acid sequence of SEQ ID NO: 19. In some embodiments, the pre-protein signal peptide comprises an amino acid sequence having at least 98% identity to the amino acid sequence of SEQ ID NO: 19. In some embodiments, the pre-protein signal peptide comprises the amino acid sequence of SEQ ID NO: 19.

In some embodiments, the pre-protein signal peptide comprises an amino acid sequence having at least 80%, 85%, 90%, 91%, 92%, 93%, 94%, 95%, 96%, 97%, 98%, 99%, or 100% identity to the amino acid sequence of SEQ ID NO: 20. In some embodiments, the pre-protein signal peptide comprises an amino acid sequence having at least 98% identity to the amino acid sequence of SEQ ID NO: 20. In some embodiments, the pre-protein signal peptide comprises the amino acid sequence of SEQ ID NO: 20.

In some embodiments, the pre-protein signal peptide comprises an amino acid sequence having at least 80%, 85%, 90%, 91%, 92%, 93%, 94%, 95%, 96%, 97%, 98%, 99%, or 100% identity to the amino acid sequence of SEQ ID NO: 47. In some embodiments, the pre-protein signal peptide comprises an amino acid sequence having at least 98% identity to the amino acid sequence of SEQ ID NO: 47. In some embodiments, the pre-protein signal peptide comprises the amino acid sequence of SEQ ID NO: 47.

In some embodiments, the pre-protein signal peptide comprises an amino acid sequence having at least 80%, 85%, 90%, 91%, 92%, 93%, 94%, 95%, 96%, 97%, 98%, 99%, or 100% identity to the amino acid sequence of SEQ ID NO: 48. In some embodiments, the pre-protein signal peptide comprises an amino acid sequence having at least 98% identity to the amino acid sequence of SEQ ID NO: 48. In some embodiments, the pre-protein signal peptide comprises the amino acid sequence of SEQ ID NO: 48.

In some embodiments, the pre-protein signal peptide comprises an amino acid sequence having at least 80%, 85%, 90%, 91%, 92%, 93%, 94%, 95%, 96%, 97%, 98%, 99%, or 100% identity to the amino acid sequence of SEQ ID NO: 148. In some embodiments, the pre-protein signal peptide comprises an amino acid sequence having at least 98% identity to the amino acid sequence of SEQ ID NO: 148. In some embodiments, the pre-protein signal peptide comprises the amino acid sequence of SEQ ID NO: 148.

In some embodiments, the pre-protein signal peptide comprises an amino acid sequence having at least 80%, 85%, 90%, 91%, 92%, 93%, 94%, 95%, 96%, 97%, 98%, 99%, or 100% identity to the amino acid sequence of SEQ ID NO: 180. In some embodiments, the pre-protein signal peptide comprises an amino acid sequence having at least 98% identity to the amino acid sequence of SEQ ID NO: 180. In some embodiments, the pre-protein signal peptide comprises the amino acid sequence of SEQ ID NO: 180.

### Polypeptides

In some embodiments, a polypeptide is provided. In some embodiments, the polypeptide comprises a formula of X₁-Z₁, wherein X₁ is a pre-protein signal peptide and Z₁ is a payload protein.

In some embodiments, X₁ comprises a pre-protein signal peptide as provided for herein. In some embodiments, X₁ comprises an amino acid sequence having at least 80%, 85%, 90%, 91%, 92%, 93%, 94%, 95%, 96%, 97%, 98%, 99%, or 100% identity to an amino acid sequence as provided in Table 1. In some embodiments, X₁ comprises an amino acid sequence having at least 98% identity to an amino acid sequence as provided in Table 1. In some embodiments, X₁ comprises an amino acid sequence as provided in Table 1. In some embodiments, X₁ comprises an amino acid sequence having at least 80%, 85%, 90%, 91%, 92%, 93%, 94%, 95%, 96%, 97%, 98%, 99%, or 100% identity to an amino acid sequence selected from SEQ ID NO: 6. SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180. In some embodiments, X₁ comprises an amino acid sequence having at least 98% identity to an amino acid sequence selected from SEQ ID NO: 6. SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180. In some embodiments, X₁ comprises an amino acid sequence selected from SEQ ID NO: 6. SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 6. In some embodiments, X₁ comprises an amino acid sequence having at least 98% identity to the amino acid sequence of SEQ ID NO: 6. In some embodiments, X₁ comprises the amino acid sequence of SEQ ID NO: 6.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 7. In some embodiments, X₁ comprises an amino acid sequence having at least 98% identity to the amino acid sequence of SEQ ID NO: 7. In some embodiments, X₁ comprises the amino acid sequence of SEQ ID NO: 7.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 14. In some embodiments, X₁ comprises an amino acid sequence having at least 98% identity to the amino acid sequence of SEQ ID NO: 14. In some embodiments, X₁ comprises the amino acid sequence of SEQ ID NO: 14.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 17. In some embodiments, X₁ comprises an amino acid sequence having at least 98% identity to the amino acid sequence of SEQ ID NO: 17. In some embodiments, X₁ comprises the amino acid sequence of SEQ ID NO: 17.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 18. In some embodiments, X₁ comprises an amino acid sequence having at least 98% identity to the amino acid sequence of SEQ ID NO: 18. In some embodiments, X₁ comprises the amino acid sequence of SEQ ID NO: 18.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 19. In some embodiments, X₁ comprises an amino acid sequence having at least 98% identity to the amino acid sequence of SEQ ID NO: 19. In some embodiments, X₁ comprises the amino acid sequence of SEQ ID NO: 19.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 20. In some embodiments, X₁ comprises an amino acid sequence having at least 98% identity to the amino acid sequence of SEQ ID NO: 20. In some embodiments, X₁ comprises the amino acid sequence of SEQ ID NO: 20.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 47. In some embodiments, X₁ comprises an amino acid sequence having at least 98% identity to the amino acid sequence of SEQ ID NO: 47. In some embodiments, X₁ comprises the amino acid sequence of SEQ ID NO: 47.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 48. In some embodiments, X₁ comprises an amino acid sequence having at least 98% identity to the amino acid sequence of SEQ ID NO: 48. In some embodiments, X₁ comprises the amino acid sequence of SEQ ID NO: 48.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 148. In some embodiments, X₁ comprises an amino acid sequence having at least 98% identity to the amino acid sequence of SEQ ID NO: 148. In some embodiments, X₁ comprises the amino acid sequence of SEQ ID NO: 148.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 180. In some embodiments, X₁ comprises an amino acid sequence having at least 98% identity to the amino acid sequence of SEQ ID NO: 180. In some embodiments, X₁ comprises the amino acid sequence of SEQ ID NO: 180.

In some embodiments, Z₁ is a protein. In some embodiments, Z₁ is a therapeutic protein. In some embodiments, Z₁ is a payload protein selected from an antimicrobial peptide, a cytokine, an enzyme, a food protein, a growth factor, a hormone, an immunoreceptor, a mucolytic, an antibody, a nanobody, or a peptide. In some embodiments Z₁ is a payload protein selected from the group consisting of antimicrobial peptides, Catestatin, CRAMP, hBD1, hBD3, β-Cateslytin, Dermaseptin, IL-10, IL-22, ClbS, TIGIT, TFF1, TFF2, TFF3, anti-CCL2 antibody, anti-CCL2 antibody fragments, anti-IL23 antibody, anti-IL23 antibody fragments, anti-TNFα antibody, anti-TNFα antibody fragments, GLP2, IGF1, leptin, and casein. In some embodiments Z₁ is a payload protein selected from the group consisting of anti-TNFα antibody, anti-TNFα antibody fragment, IL-10, IL-22, hBD3, leptin, and beta-casein. In some embodiments Z₁ is an anti-TNFα antibody. In some embodiments Z₁ is an anti-TNFα antibody fragment. In some embodiments Z₁ is IL-10. In some embodiments Z₁ is IL-22. In some embodiments Z₁ is hBD3. In some embodiments Z₁ is leptin. In some embodiments Z₁ is beta-casein. In some embodiments, Z₁ is a protein comprising an amino acid sequence as recited in Table 3 below.

TABLE 3

| SEQ ID NO | Z₁ | Sequence |
|---|---|---|
| 21 | anti-TNFα | EVQLVESGGGLVQPGGSLRLSCAASGFTFSDYWMYWVRQA |
| | | PGKGLEWVSEINTNGLITKYPDSVGRFTISRDNAKNTLYLQM |
| | | NSLRPEDTAVYYCARSPSGFNRGQGTLVTVSS |
| 22 | IL-22 | APISSHCRLDKSNFQQPYITNRTFMLAKEASLADNNTDVRLI |
| | | GEKLFHGVSMSERCYLMKQVLNFTLEEVLFPQSDRFQPYMQ |
| | | EVVPFLARLSNRLSTCHIEGDDLHIQRNVQKLKDTVKKLGES |
| | | GEIKAIGELDLLFMSLRNACI |
| 23 | hBD3 | GIINTLQKYYCRVRGGRCAVLSCLPKEEQIGKCSTRGRKCCR |
| | | RKK |
| 24 | IL-10 | HSSALLCCLVLLTGVRASPGQGTQSENSCTHFPGNLPNMLRD |
| | | LRDAFSRVKTFFQMKDQLDNLLLKESLLEDFKGYLGCQALS |
| | | EMIQFYLEEVMPQAENQDPDIKAHVNSLGENLKTLRLRLRR |
| | | CHRFLPCENKSKAVEQVKNAFNKLQEKGIYKAMSEFDIFINY |
| | | IEAYMTMKIRN |
| 25 | Leptin | VPIQKVQDDTKTLIKTIVTRINDISHTQSVSSKQKVTGLDFIPG |
| | | LHPILTLSKMDQTLAVYQQILTSMPSRNVIQISNDLENLRDLL |
| | | HVLAFSKSCHLPWASGLETLDSLGGVLEASGYSTEVVALSRL |
| | | QGSLQDMLWQLDLSPGC |
| 26 | beta-casein | RELEELNVPGEIVESLSSSEESITRINKKIEKFQSEEQQQTEDEL |
| | | QDKIHPFAQTQSLVYPFPGPIPNSLPQNIPPLTQTPVVVPPFLQ |
| | | PEVMGVSKVKEAMAPKHKEMPFPKYPVEPFTESQSLTLTDV |
| | | ENLHLPLPLLQSWMHQPHQPLPPTVMFPPQSVLSLSQSKVLP |
| | | VPQKAVPYPQRDMPIQAFLLYQEPVLGPVRGPFPIIV |
| 49 | hBD1 | DHYNCISSGGQCLYSACPIFTKIQGTCYRGKAKCCK |
| 50 | hBD2 | GIGDPVTCLKSGAICHPVFCPRRYKQIGTCGLPGTKCCKKP |
| 51 | b-Cateslytin | RSMRLSFRARGYGWR |
| | (Bovine) | |
| 52 | Dermaseptin | GLWSTIKQKGKEAAIAAAKAAGQAALGAL |
| | (Xenopus) | |
| 53 | HD5 | ESLQERADEATTQKQSGEDNQDLAISFAGNGLSALRTSGSQA |
| | | RATCYCRTGRCATRESLSGVCEISGRLYRLCCR |
| 54 | Reg3a | EEPQRELPSARIRCPKGSKAYGSHCYALFLSPKSWTDADLAC |
| | | QKRPSGNLVSVLSGAEGSFVSSLVKSIGNSYSYVWIGLHDPT |
| | | QGTEPNGEGWEWSSSDVMNYFAWERNPSTISSPGHCASLSR |
| | | STAFLRWKDYNCNVRLPYVCKFTD |
| 55 | C5a | SWLRDIWDWICEVLSDFK |
| 56 | TFF1 | ATMENKVICALVLVSMLALGTLAEAQTETCTVAPRERQNCG |
| | | FPGVTPSQCANKGCCFDDTVRGVPWCFYPNTIDVPPEEECEF |
| 57 | TFF2 | GRRDAQLLAALLVLGLCALAGSEKPSPCQCSRLSPHNRTNC |
| | | GFPGITSDQCFDNGCCFDSSVTGVPWCFHPLPKQESDQCVME |
| | | VSDRRNCGYPGISPEECASRKCCFSNFIFEVPWCFFPKSVEDC |
| | | HY |
| 58 | TFF3 | AARALCMLGLVLALLSSSSAEEYVGLSANQCAVPAKDRVDC |
| | | GYPHVTPKECNNRGCCFDSRIPGVPWCFKPLQEAECTF |
| 59 | CTB-IL-22 | TPQNITDLCAEYHNTQIYTLNDKIFSYTESLAGKREMAIITFK |
| | | NGAIFQVEVPGSQHIDSQKKAIERMKDTLRIAYLTEAKVEKL |
| | | CVWNNKTPHAIAAISMANGPGPKKRKSVAPISSHCRLDKSNF |
| | | QQPYITNRTFMLAKEASLADNNTDVRLIGEKLFHGVSMSERC |
| | | YLMKQVLNFTLEEVLFPQSDRFQPYMQEVVPFLARLSNRLST |
| | | CHIEGDDLHIQRNVQKLKDTVKKLGESGEIKAIGELDLLFMS |
| | | LRNACI |
| 60 | ClbS | AVPSSKEELIKAINSNFSLLNKKLESITPQLAFEPLLEGIIAKGT |
| | | TISVANLVSYLIGWGELVLHWHDQEAKGKTIIFPEEGFKWNE |
| | | LGRLAQKFYRDYEDITEYEVLLARLKENKQQLVALIERFSND |
| | | ELYGKPWYNKWTRGRMIQFNTASPYKNASGRLNKLQKCLA |
| | | E |
| 61 | shP28GST | MTGDHIKVIYFNGRGRAESIRMTLVAAGVNYEDERISFQDW |
| | | PKIKPTIPGGRLPAVKITDNHGHVKWMVESLAIARYMAKKH |
| | | HMMGGTEEEYYNVEKLIGQAEDLEHEYYKTLMKPEEEKQKI |
| | | IKEILNGKVPVLLDIICESLKASTGKLAVGDKVTLADLVLIAVI |
| | | DHVTDLDKEFLTGKYPEIHKHRENLLASSPRLAKYLSDRAAT |
| | | PF |
| 62 | ClbS | AIPESKSELIEAINKNYASLVKKLAAVPAQRVFQPIMEGHVK |
| | (Gilliamella | GTTMSVAQLVSYLIGWGELVLSWHVKEANGNEIVFPEVGFK |
| | apicola) | WNEMGKLAQKFYLDYQDITDYKVLLQRLENNKNEIIKLIEGF |
| | | SDDDLYGVGWCEKYTRGRMIQLNTASPYKNATGRLATLLK |
| | | SIDS |
| 63 | ClbS | IPESKQELIEAINKNYTLLTKKLAAVPEQKAYLPLMEGHAKG |
| | (Snodgrassella | TMMSAAQLVSYLIGWGELVLSWHKQEQSGQKIAFPEAGYK |
| | alvi) | WNQLGLLAQKFYRDYQDITDFKQLLSLLETNKKNLISLIDSFS |
| | | NEELYGSPWHEKYTRGRMIQFNTSSPYKNATGRLNKLLKKI |
| | | AE |
| 64 | Phytase | AQSEPELKLESVVIVSRHGVRAPTKATQLMQDVTPDAWPTW |
| | | PVKLGELTPRGGELLAYLGHYWRQRLVADGLLPKCGCPQSG |
| | | QVAILADVDERTRKTGEAFAAGLAPDCAITVHTQADTSSPDP |
| | | LFNPLKTGVCQLDNANVTDAILERAGGSLADFTGHYQTAFR |
| | | ELERVLNFPQSNLCLKREKQDESCSLTQALPSELKVSADCVS |
| | | LTGAVSLASMLTEIFLLQQAQGMPEPGWGRITDSIIQWNTLL |
| | | SLHNAQFDLLQRTPEVARSRATPLLDLIKTALTPHPPQKQAY |
| | | GVTLPTSVLFLAGHDTNLANLGGALELNWTLPGQPDNTPPG |
| | | GELVFERWRRLSDNSQWIQVSLVFQTLQQMRDKTPLSLNTP |
| | | PGEVKLTLAGCEERNAQGMCSLAGFTQIVNEARIPACSL |
| 65 | SUC2 | SMTNETSDRPLVHFTPNKGWMNDPNGLWYDEKDAKWHLY |
| | | FQYNPNDTVWGTPLFWGHATSDDLTNWEDQPIAIAPKRNDS |
| | | GAFSGSMVVDYNNTSGFFNDTIDPRQRCVAIWTYNTPESEEQ |
| | | YISYSLDGGYTFTEYQKNPVLAANSTQFRDPKVFWYEPSQK |
| | | WIMTAAKSQDYKIEIYSSDDLKSWKLESAFANEGFLGYQYE |
| | | CPGLIEVPTEQDPSKSYWVMFISINPGAPAGGSFNQYFVGSFN |
| | | GTHFEAFDNQSRVVDFGKDYYALQTFFNTDPTYGSALGIAW |
| | | ASNWEYSAFVPTNPWRSSMSLVRKFSLNTEYQANPETELINL |
| | | KAEPILNISNAGPWSRFATNTTLTKANSYNVDLSNSTGTLEFE |
| | | LVYAVNTTQTISKSVFADLSLWFKGLEDPEEYLRMGFEVSAS |
| | | SFFLDRGNSKVKFVKENPYFTNRMSVNNQPFKSENDLSYYK |
| | | VYGLLDQNILELYFNDGDVVSTNTYFMTTGNALGSVNMTTG |
| | | VDNLFYIDKFQVREVK |
| 66 | SUIS | KWWQSSVIYEIYPKSFYDTNNDGIGDLKGVEKKLPYLKELGI |
| | | DAIWLTPIYLSPQIDNGYDVADYRTVDPMFGNNQDLKELIDR |
| | | AHRLNIKVILDMVANHTSDQCIWFKESKKSKDNYFSDFYIW |
| | | KDPKPDGSEPNNWAASFGGSAWTYVPERSQYYLHYYASQQ |
| | | PDLNWENPLVRQYVYDAMRYWKSQGVDGWRLDVITQISKD |
| | | LTFKDVPTTDSNKKYIPLKNTSGPHMHKFIHELNQQVLKPFD |
| | | MMSVGEAPNSNIEEILKLTSPERDELNMAFSFEHMRIDKAIG |
| | | KNGAIAVKKPNFLKLKQVLSKMQYELNSKSWNALYFENHD |
| | | RSRIPTRWGNDTEYRYESATAFATILHGLKGTPFIFQGEEIGM |
| | | TNPKYSVDEYEDIDLKNKYQEQVKEKHLLTTDEFLKVEHKI |
| | | ARDNARTPMQWNNKKNAGFTEGKPWYKVNPNFTEINVESD |
| | | KLSKKSIFRYYQQLITLRHKEDILIKGDYQELLEDDKNIFAYQ |
| | | RSWKNKSWLILVNMTEKDLSLNELHDNLKNYKKIILHNYLN |
| | | QKLEDILKPYEAYILEK |
| 67 | AnPep | ATTGEAYFEQLLDHHNPEKGTFSQRYWWSTEYWGGPGSPV |
| | | VLFNPGEVSADGYEGYLTNDTLTGVYAQEIQGAVILIEHRY |
| | | WGDSSPYEVLNAETLQYLTLDQSILDMTYFAETVKLQFDNSS |
| | | RSNAQNAPWVMVGGSYSGALTAWTESIAPGTFWAYHATSA |
| | | PVEAIYDFWQYFYPIQQGMAQNCSKDVSLVAEYVDKIGKNG |
| | | TAKEQQELKELFGLGAVEHYDDFAAVLPNGPYLWQDNDFV |
| | | TGYSSFFQFCDAVEGVEAGAAVTPGPEGVGLEKALANYAN |
| | | WFNSTILPNYCASYGYWTDEWSVACFDSYNASSPIFTDTSVG |
| | | NPVDRQWEWFLCNEPFFWWQDGAPEGTSTIVPRLVSASYW |
| | | QRQCPLYFPEVNGYTYGSAKGKNSATVNSWTGGWDMTRNT |
| | | TRLIWTNGQYDPWRDSGVSSTFRPGGPLVSTANEPVQUIPGGF |
| | | HCSDLYMEDYYANEGVRKVVDNEVKQIKEWVEEYYA |
| 68 | Beta- | RKQLPKDFVIGGATAAYQVEGATKEDGKGRVLWDDFLEKQ |
| | galactosidase | GRFSPDPAADFYHRYDEDLALAEAYGHQVIRLSIAWSRIFPD |
| | (lactobacillus) | GAGAVEPRGVAFYHRLFAACAKHHLIPFVTLHHFDTPERLH |
| | | AIGDWLSQEMLEDFVEYARFCFEEFPEIKHWITINEPTSMAV |
| | | QQYTSGTFPPAETGHFDKTFQAEHNQIVAHARIVNLYKSMG |
| | | LDGEIGIVHALQTPYPYSDSSEDQHAADLQDALENRLYLDGT |
| | | LAGDYAPKTLALIKEILAANQQPMFKRTDEEMAAIKKAAHQ |
| | | LDFVGVNNYFSKWLRAYHGKSETIHNGDGSKGSSVARLHGI |
| | | GEEKKPAGIETTDWDWSIYPRGMYDMLMRIHRDYPLVPAIY |
| | | VTENGIGLKESLPAEVTPNTVIADPKRIDYLKKYLSAIADAIQ |
| | | AGANVKGYFVWSLQDQFSWTNGYSKRYGLFFVDFPTQKRY |
| | | VKQSAEWLKQVSQTHVIP |
| 69 | LACPL Exo- | EKWWKNAVVYQVYPSSYQDSNNDGIGDLPGITKRLDYIKKL |
| | alfa-1,4- | GVDIVWLSPIYKSPQVDNGYDISDYRAINPDFGSMEDFDKLL |
| | glucosidase | GKAHDLGLKIMMDLVVNHTSDENKWFEESRKSKTNPYRDY |
| | | YIWRDGNAGKSPNNWGSFFRGPAWKYDEQTGQYYLHLFAP |
| | | QQPDLNWENPNVRHSVYDMMNWWASKGVDGFRMDVINYI |
| | | SKPEGLPDAQLQADEELGNPEPLVANGHRVHEFLQEMNNKV |
| | | MSKHRMVTVGEAPSVTTDEAIKYAGLDGKELDMVFQFEHM |
| | | GLDGNENPVLGHWSDRKASLKDLRSNLTKWQEALEGKAW |
| | | NSLYWNNHDQPRVVSRFGDDSNEEYRVLSAKMLATMLHLQ |
| | | QGTPFIFEGEEIGMTNSYFKKLSDYVDLDSINTYHQFVDKQH |
| | | LVGSQTMLKYLAMHSRDNARTPMQWNSTDNGGFSKHEPW |
| | | EHVNPNYKHINVKQSLDDPNSIFYYYQKLIRLRHELPVITDG |
| | | KYRLVKGNEDDEAVYAYTRKDENTTLLVILNYTKTTLDRDY |
| | | QVPDNAELVINNYPDDKGNTLRPYEAKVYKY |
| 70 | Beta- | IKHRLNGFTILEHPDPAKRDLLQDIVTWDDKSLFINGERIMLF |
| | galactosidase | SGEVHPFRLPVPSLWLDIFHKIRALGFNCVSFYIDWALLEGKP |
| | (yeast) | GDYRAEGIFALEPFFDAAKEAGIYLIARPGSYINAEVSGGGFP |
| | | GWLQRVNGTLRSSDEPFLKATDNYIANAAAAVAKAQITNGG |
| | | PVILYQPENEYSGGCCGVKYPDADYMQYVMDQARKADIVV |
| | | PFISNDASPSGHNAPGSGTSAVDIYGHDSYPLGFDCANPSVW |
| | | PEGKLPDNFRTLHLEQSPSTPYSLLEFQAGAFDPWGGPGFEK |
| | | CYALVNHEFSRVFYRNDLSFGVSTFNLYMTFGGTNWGNLG |
| | | HPGGYTSYDYGSPITETRNVTREKYSDIKLLANFVKASPSYLT |
| | | ATPRNLTTGVYTDTSDLAVTPLIGDSPGSFFVVRHTDYSSQES |
| | | TSYKLKLPTSAGNLTIPQLEGTLSLNGRDSKIHVVDYNVSGT |
| | | NIIYSTAEVFTWKKFDGNKVLVLYGGPKEHHELAIASKSNVT |
| | | IIEGSDSGIVSTRKGSSVIIGWDVSSTRRIVQVGDLRVFLLDRN |
| | | SAYNYWVPELPTEGTSPGFSTSKTTASSIIVKAGYLLRGAHLD |
| | | GADLIILTADFNATTPIEVIGAPTGAKNLFVNGEKASHTVDK |
| | | NGIWSSEVKYAAPEIKLPGLKDLDWKYLDTLPEIKSSYDDSA |
| | | WVSADLPKTKNTHRPLDTPTSLYSSDYGFHTGYLIYRGHFVA |
| | | NGKESEFFIRTQGGSAFGSSVWLNETYLGSWTGADYAMDGN |
| | | STYKLSQLESGKNYVITVVIDNLGLDENWTVGEETMKNPRGI |
| | | LSYKLSGQDASAITWKLTGNLGGEDYQDKVRGPLNEGGLY |
| | | AERQGFHQPQPPSESWESGSPLEGLSKPGIGFYTAQFDLDLPK |
| | | GWDVPLYFNFGNNTQAARAQLYVNGYQYGKFTGNVGPQTS |
| | | FPVPEGILNYRGTNYVALSLWALESDGAKLGSFELSYTTPVL |
| | | TGYGNVESPEQPKYEQRKGAY |
| 71 | ACE2 | DTIEEQAKTFLDKFNHEAEDLFYQSSLASWNYNTNITEENVQ |
| | | NMNNAGDKWSAFLKEQSTLAQMYPLQEIQNLTVKLQLQAL |
| | | QQNGSSVLSEDKSKRLNTILNTMSTIYSTGKVCNPDNPQECL |
| | | LLEPGLNEIMANSLDYNERLWAWESWRSEVGKQLRPLYEEY |
| | | VVLKNEMARANHYEDYGDYWRGDYEVNGVDGYDYSRGQ |
| | | LIEDVEHTFEEIKPLYEHLHAYVRAKLMNAYPSYISPIGCLPA |
| | | HLLGDMWGRFWTNLYSLTVPFGQKPNIDVTDAMVDQAWD |
| | | AQRIFKEAEKFFVSVGLPNMTQGFWENSMLTDPGNVQKAVC |
| | | APTAWDLGKGDFRILMCTKVTMDDFLTAHHEMGHIQYDMA |
| | | YAAQPFLLRNGANEGFHEAVGEIMSLSAATPKHLKSIGLLSP |
| | | DFQEDNETEINFLLKQALTIVGTLPFTYMLEKWRWMVFKGEI |
| | | PKDQWMKKWWEMKREIVGVVEPVPHDETYCDPASLFHVSN |
| | | DYSFIRYYTRTLYQFQFQEALCQAAKHEGPLHKCDISNSTEA |
| | | GQKLFNMLRLGKSEPWTLALENVVGAKNMNVRPLLNYFEP |
| | | LFTWLKDQNKNSFVGWSTDWSPYADQSIKVRISLKSALGDK |
| | | AYEWNDNEMYLFRSSVAYAMRQYFLKVKNQMILFGEEDVR |
| | | VANLKPRISFNFFVTAPKNVSDIIPRTEVEKAIRMSRSRINDAF |
| | | RLNDNSLEFLGIQPTLGPPNQPPVS |
| 72 | Xylose | MLNPTPADKFSFGLWTVGWPARDQFGKASRPDLDPFDSIRK |
| | Isomerase | LKELGAWGFTFHDNDVIPFDATPAERDDLICTLKKTIDETGM |
| | (RainFlu) | VCEMVTTDTFAEPVFKDGAFTSNDRRVRRYGLRKVLRNVDL |
| | | AAELGATTFVMWGGREGAEYDSSKDLHAALERYAEGIDTV |
| | | AGYIKSQGYDLRIGLEPKPNEPRGDIFLPTIGHALAFIQRLDN |
| | | GDVVGLNPETGHEQMATLNYTHGLAQALWSEKLFHIDLNG |
| | | QNGPMYDQDLVFGHGNLISAFFTVDLLENGFPSKPGAYEGA |
| | | RHFDYKTSRTDHVTGVWDSALANMRMYLILKERALAFRAD |
| | | PEVQEALAYAGVHELAEPTLGADESLESFLADPSVYEGLDLT |
| | | GPSERNYGYVRLHQLAVEHLTGARGVG |
| 73 | Xylose | MGIELTQMALNRKIAQKRPLENFFQLAEAAGIHQVELRNDM |
| | Isomerase | TTSDGSETVIDGMQVAEFQTLKQKYDLQILTINAIQQFNNPA |
| | | KLNKNRDLLTKLAELSAQIGNQAIIFVPEVNAQDKRTEQQRL |
| | | DDAVSSLQVFGDILSAYHLTGFIEPLGFRASTMRYPWTALDA |
| | | INLSGRTEFKLTIDTFIIFFLAIILTAEQFKAGVDINRVGLIIILS |
| | | GIEPIHALREVVDEDRILITERDIMQNIEQVHLFEAMGYRGHY |
| | | SFEPFSSRLAAETNQQLTQQILASIERLNQPTAVFSTEVTQPV |
| | | G |
| 74 | Xylose | MKTATRLNAYLPHYDNKIDKVFGELKKVGINYVDINFPQQTI |
| | Isomerase | GIPARDMKKMLDENGLRPNGMEMRFEGHYLNGDLGNLND |
| | | KIYQDAIEMCKQAVDYCREIGGSMINIWSAHDGFDYSFQIDY |
| | | LKVWHRIVKACQEIADYAPDIKFSIEYKPYEPRAYAFVDSMG |
| | | TLGMILKDINRKNFGVLLDYCHMLMKHENPAMGSEIFASRG |
| | | ELFGVHINDGYGVHDDGLMVGTVTPFKTLEFLYYMKKYHF |
| | | DGMYYFDTAPVIEDPSQETRKNIEMLKYLLNLLDRVGMEKI |
| | | DKVINENDAIKARDLLLDFIKKVG |
| 75 | Xylose | MKTATRLNAYLPHYNNDINQVFKELKRVGINYVDINFPQQTI |
| | Isomerase | GIPATKMKQMLQQNDLCPNGMEMRFEDHYLNGDLSNLDDR |
| | | IYRDAIEMCKKAADYCHAIGGTMINIWSAHDGFDYSFQIDYL |
| | | KVWQRIVKACQEIADYAPDIKFSIEYKPYEPRAYAFVDSMGI |
| | | LGMMLKDINRPNFGVLLDYCHMLMKHENPAMGAALFASQ |
| | | KELFGVHINDGYGVHDDGLMVGTVTPFKTLEFLYYMKKYD |
| | | FDGMYYFDTAPVIENPSQETAKNIEMLKYLLNLLDRIGMDHI |
| | | ETIIQENDAIKARELLLEFMKNVG |
| 76 | MalL | SEWWKEAVVYQIYPRSFYDANGDGFGDLQGVIQKLDYIKNL |
| | | GADVIWLSPVFDSPQDDNGYDISDYKNMYEKFGTNEDMFQL |
| | | IDEVHKRGMKIVMDLVVNHTSDEHAWFAESRKSKDNPYRD |
| | | YYLWKDPKPDGSEPNNWGSIFSGSAWTYDEGTGQYYLHYFS |
| | | KKQPDLNWENEAVRREVYDVMRFWMDRGVDGWRMDVIG |
| | | SISKYTDFPDYETDHSRSYIVGRYHSNGPRLHEFIQEMNREVL |
| | | SHYDCMTVGEANGSDIEEAKKYTDASRQELNMIFTFEHMDI |
| | | DKEQNSPNGKWQIKPFDLIALKKTMTRWQTGLMNVGWNTL |
| | | YFENHDQPRVISRWGNDRKLRKECAKAFATVLHGMKGTPFI |
| | | YQGEEIGMVNSDMPLEMYDDLEIKNAYRELVVENKTMSEK |
| | | EFVKAVMIKGRDHARTPMQWDAGKHAGFTAGDPWIPVNSR |
| | | YQDINVKESLEDQDSIFFYYQKLIQLRKQYKIMIYGDYQLLQ |
| | | ENDPQVFSYLREYRGEKLLVVVNLSEEKALFEAPPELIHERW |
| | | KVLISNYPQERADLKSISLKPYEAVMGISIGSSGGSSG |
| 77 | apyrase | AEGFLTQQTSPDSLSILPPPPAEDSVVFQADKAHYEFGRSLRD |
| | | ANRVRLASEDAYYENFGLAFSDAYGMDISRENTPILYQLLTQ |
| | | VLQDSHDYAVRNAKEYYKRVRPFVIYKDATCTPDKDENMAI |
| | | TGSYPSGHASFGWAVALILAEINPQRKAEILRRGYEFGESRVI |
| | | CGAHWQSDVEAGRLMGASVVAVLHNTPEFTKSLSEAKKEF |
| | | EELNTPTNELTP |
| 78 | DL- | ETLDEKKQKIESKQSEVASSIEAKEKELTELQENQSKIEKELK |
| | endopeptidase | DINDKALDTSNKIEDKKEENDKTKEEIKKLKKEIKETEARIEK |
| | | RNEILKKRVRSLQESGGSQGYIDVLLGSTSFGDFISRATAVSSI |
| | | VDADKDLIKQQEQDKAKLEDSEADLNDKLKEVQAALAKLE |
| | | TMQKDLDKQLNEKDKLFDEAKASQKKTAKAISELKSEASEL |
| | | ANQKANTEAEQARIKKEQEAAAALIKKQEEAQKASDETQTD |
| | | DSQTATTESSKASSSDDSSDNSSDNSSNGSSNSSSNGSSSKKS |
| | | SGSNSNSGGTVISNSGGIEGAISVGSSIVGQSPYKFGGGRTQS |
| | | DINNRIFDCSSFVRWAYASAGVNLGPVGGTTTDTLVGRGQA |
| | | VSASEMKRGDLVFFDTYKTNGHVGIYLGNGTFLNDNTSHGV |
| | | SVDSMSNPYWKAAFKGVVRRVVQ |
| 79 | beta-casein- | RELEELNVPGEIVESLSSSEESITRINKKIEKFQSEEQQQTEDEL |
| | CSN2 | QDKIHPFAQTQSLVYPFPGPIPNSLPQNIPPLTQTPVVVPPFLQ |
| | | PEVMGVSKVKEAMAPKHKEMPFPKYPVEPFTESQSLTLTDV |
| | | ENLHLPLPLLQSWMHQPHQPLPPTVMFPPQSVLSLSQSKVLP |
| | | VPQKAVPYPQRDMPIQAFLLYQEPVLGPVRGPFPIIV |
| 80 | beta-casein- | RELEELNVPGEIVESLSSSEESITRINKKIEKFQSEEQQQTEDEL |
| | CSN2 | QDKIHPFAQTQSLVYPFPGPIPNSLPQNIPPLTQTPVVVPPFLQ |
| | | PEVMGVSKVKEAMAPKHKEMPFPKYPVEPFTESQSLTLTDV |
| | | ENLHLPLPLLQSWMHQPHQPLPPTVMFPPQSVLSLSQSKVLP |
| | | VPQKAVPYPQRDMPIQAFLLYQEPVLGPVRGPFPIIVGSSGGS |
| | | SG |
| 81 | alpha-casein- | RPKHPIKHQGLPQEVLNENLLRFFVAPFPEVFGKEKVNELSK |
| | CSN1S1 | DIGSESTEDQAMEDIKQMEAESISSSEEIVPNSVEQKHIQKED |
| | | VPSERYLGYLEQLLRLKKYKVPQLEIVPNSAEERLHSMKEGI |
| | | HAQQKEPMIGVNQELAYFYPELFRQFYQLDAYPSGAWYYVP |
| | | LGTQYTDAPSFSDIPNPIGSENSEKTTMPLW |
| 82 | alpha-casein- | RPKHPIKHQGLPQEVLNENLLRFFVAPFPEVFGKEKVNELSK |
| | CSN1S1 | DIGSESTEDQAMEDIKQMEAESISSSEEIVPNSVEQKHIQKED |
| | | VPSERYLGYLEQLLRLKKYKVPQLEIVPNSAEERLIISMKEGI |
| | | HAQQKEPMIGVNQELAYFYPELFRQFYQLDAYPSGAWYYVP |
| | | LGTQYTDAPSFSDIPNPIGSENSEKTTMPLWGSSGGSSG |
| 83 | alpha-casein- | GPFPVIRGPFPVIRFALPEYLKGPFPVIR |
| | derived- | |
| | peptide-1 | |
| 84 | alpha-casein- | FALPEYLKGPFPVIRFILLQGVLKFFVAPFPEVFGK |
| | derived- | |
| | peptide-2 | |
| 85 | alpha-casein- | FALPEYLKFILLQGVLKFFVAPFPEVFGKGPFPVIR |
| | derived- | |
| | peptide-8 | |
| 86 | alpha-casein- | GPFPVIRFILLQGVLKFFVAPFPEVFGKFILLQGVLK |
| | derived- | |
| | peptide-15 | |
| 87 | beta-casein- | RELEELNVPGEIVESLSSSEESITRINKKIEKFQSEEQQQTEDEL |
| | CSN2 plus | QDKIHPFAQTQSLVYPFPGPIPNSLPQNIPPLTQTPVVVPPFLQ |
| | peptide 3 | PEVMGVSKVKEAMAPKHKEMPFPKYPVEPFTESQSLTLTDV |
| | | ENLHLPLPLLQSWMHQPHQPLPPTVMFPPQSVLSLSQSKVLP |
| | | VPQKAVPYPQRDMPIQAFLLYQEPVLGPVRGPFPIIVFALPEY |
| | | LKFALPEYLKGPFPVIRFILLQGVLKGSSGGSSG |
| 88 | alpha-casein- | RPKHPIKHQGLPQEVLNENLLRFFVAPFPEVFGKEKVNELSK |
| | CSN1S1 plus | DIGSESTEDQAMEDIKQMEAESISSSEEIVPNSVEQKHIQKED |
| | peptide 7 | VPSERYLGYLEQLLRLKKYKVPQLEIVPNSAEERLHSMKEGI |
| | | HAQQKEPMIGVNQELAYFYPELFRQFYQLDAYPSGAWYYVP |
| | | LGTQYTDAPSFSDIPNPIGSENSEKTTMPLWGPFPVIRFILLQG |
| | | VLKFFVAPFPEVFGKFILLQGVLKFILLQGVLKFFVAPFPEVF |
| | | GKFALPEYLKFILLQGVLKGSSGGSSG |
| 89 | Ara h1 | RGRVSPLMLLLGILVLASVSA |
| 90 | Ara h2 | RQQWELQGDRRCQSQLERANLRPCEQHLMQKIQRDEDSYG |
| | | RDPYSPSQDPYSPSQDPDRRDPYSPSPYDRRGAGSSQHQERC |
| | | CNELNEFENNQRCMCEALQQIMENQSDRLQGRQQEQQFKRE |
| | | LRNLPQQCGLRAPQRCDLEVESGGRDRY |
| 91 | IGF1 | GPETLCGAELVDALQFVCGDRGFYFNKPTGYGSSSRRAPQT |
| | | GIVDECCFRSCDLRRLEMYCAPLKPAKSARSVRAQRHTDMP |
| | | KTQKYQPPSTNKNTKSQRRKGWPKTHPGGEQKEGTEASLQI |
| | | RGKKKEQRREIGSRNAECRGKKGK |
| 92 | CTB-IGF1 | TPQNITDLCAEYHNTQIYTLNDKIFSYTESLAGKREMAIITFK |
| | | NGAIFQVEVPGSQHIDSQKKAIERMKDTLRIAYLTEAKVEKL |
| | | CVWNNKTPHAIAAISMANGPGPKKRKSVGPETLCGAELVDA |
| | | LQFVCGDRGFYFNKPTGYGSSSRRAPQTGIVDECCFRSCDLR |
| | | RLEMYCAPLKPAKSARSVRAQRHTDMPKTQKYQPPSTNKNT |
| | | KSQRRKGWPKTHPGGEQKEGTEASLQIRGKKKEQRREIGSR |
| | | NAECRGKKGK |
| 93 | Elafin | AQEPVKGPVSTKPGSCPIILIRCAMLNPPNRCLKDTDCPGIKK |
| | | CCEGSCGMACFVPQ |
| 94 | GLP1 | HAEGTFTSDVSSYLEGQAAKEFIAWLVKGRG |
| 95 | Exenatide-4 | HGEGTFTSDLSKQMEEEAVRLFIEWLKNGGPSSGAPPPS |
| 96 | Catestatin | SSMKLSFRARAYGFRGPGPQL |
| 97 | Nesfatin | VPIDIDKTKVQNIHPVESAKIEPPDTGLYYDEYLKQVIDVLET |
| | | DKHFREKLQKADIEEIKSGRLSKELDLVSHHVRTKLDEL |
| 98 | Linaclotide | CCEYCCNPACTGCY |
| 99 | Plecanatide | NDECELCVNVACTGCL |
| 100 | Uroguanylin | NDDCELCVNVACTGCL |
| 101 | PYY | IKPEAPREDASPEELNRYYASLRHYLNLVTRQRY |
| 102 | Guanylin | PGTCEICAYAACTGC |
| 103 | LCRF | STFWAYQPDGDNDPTDYQKYEHTSSPSELLAPGDYKLDYKD |
| | | HDGDYKDHDIDYKDDDDK |
| 104 | TIGIT | MMTGTIETTGNISAEKGGSIILQCHLSSTTAQVTQVNWEQQD |
| | | QLLAICNADLGWHISPSFKDRVAPGPGLGLTLQSLTVNDTGE |
| | | YFCIYHTYPDGTYTGRIFLEVLESSVAEHGARFQIP |
| 105 | Dnase I | LKIAAFNIQTFGETKMSNATLVSYIVQILSRYDIALVQEVRDS |
| | | HLTAVGKLLDNLNQDAPDTYHYVVSEPLGRNSYKERYLFVY |
| | | RPDQVSAVDSYYYDDGCEPCGNDTFNREPAIVRFFSRFTEVR |
| | | EFAIVPLHAAPGDAVAEIDALYDVYLDVQEKWGLEDVMLM |
| | | GDFNAGCSYVRPSQWSSIRLWTSPTFQWLIPDSADTTATPTII |
| | | CAYDRIVVAGMLLRGAVVPDSALPENFQAAYGLSDQLAQAI |
| | | SDHYPVEVMLK |
| 106 | anti-EGFR- | EVQLVESGGGLVQAGGSLRLSCTASGRTFSSYAMGWFRQTP |
| | IIIa5 | GKEREFVAAITSSGGSTYYADSVKGRFTISRDNAKSTMYLQ |
| | | MDSLMLDDTSVYYCAADSSRPQYSDSALRRILSLSNSYPYW |
| | | GQGTQVTVSS |
| 107 | anti-CCL2 | EVQLVESGGGLVQAGDSLRLSCAASGRTFSSYRMGWFRQAP |
| | (8e10) | GKEREFVAAISRSGASAYYADSVKGRFTCSRDNAKDTVYLQ |
| | | MNKLKPEDTAVYYCAASSRMGVDDPETYGYWGQGTQVTV |
| | | SS |
| 108 | anti-CCL2 | EVQLVESGGGLVQAGGSLRLSCAASGGTFSSYRMGWFRQAP |
| | (8e3) | GKEREFVTLISRSGGTTDYTDSVKGRFTISRDNAKNTVSLQM |
| | | NNLKPADTAVYYCAAAGSMGWNHLRDYDYWGQGTQVTV |
| | | SS |
| 109 | anti-IL-23 | EVQLVESGGGLVQPGGSLRLSCAASGFTLDYLAIGWFRQAP |
| | (37D5) | GKEREGVSCVSSSGQYTYYADSVKGRFTISRDNAESTVYLQ |
| | | MNSLKPEDTAVYYCATDPECYRVRGYYNGEYDYWGQGTQ |
| | | VTVSS |
| 110 | anti-IL-23 | EVQLVESGGGLVQAGGSLRLSCAASGRTFEGNPMGWFRQAS |
| | (80D10) | GKKREFVASIDWSGGITSYADSVKGRFTISRDNAKNTVYLQ |
| | | MNSLKPEDTAVYYCAASARFGSGSYYDLMYDYWGQGTQV |
| | | TVSS |
| 111 | GLP2 | HGDGSFSDEMNTILDNLAARDFINWLIQTKITD |
| | (teduglutide) | |
| 112 | anti- | DVQLVSYSWFRQAISKGGRFTISRDNAKSRAYGSSWGQGTE |
| | VEGFBII038 | SGGGMGAPGKYKYDAVNTVYLQINSLRLRLADTLVTVSLV |
| | | QPGEREFSLEGRPEDTAVYYCYEYSGSLRLVVASSCAASGRT |
| | | FS |
| 113 | anti-IL6R | EVQLVESGGGLVQAGGSLRLSCAASGRTFSSYDMGWYRQA |
| | (vobarilizumab) | PGKEREFVAIITWNSSTYYADSVKGRFTISRDNAKNTVYLQM |
| | | NSLKPEDTAIYYCNAQYGLGYAEDYWGQGTQVTVSS |
| 114 | anti-MERS | QVQLQESGGGSVQAGGSLRLSCVASGSIFSINAMDWYRQAP |
| | | GKQRELVAGITSGGSTNYGDFVKGRFTISRDNAKNTVYLQM |
| | | DSLKPEDTAVYYCAAEVGGWGPPRPDYWGHGTQVTVSSGS |
| | | LEVLFQ |
| 115 | anti-SARS | QVQLQESGGGLVQAGGSLRLSCAASGRTFSEYAMGWFRQA |
| | | PGKEREFVATISWSGGSTYYTDSVKGRFTISRDNAKNTVYLQ |
| | | MNSLKPDDTAVYYCAAAGLGTVVSEWDYDYDYWGQGTQV |
| | | TVSSGS |
| 116 | anti-BVD | GSVQAGGSLRLSCAASGDADSRNCMGWIRQAPGKEREGVA |
| | | GIYIGGGRTWYAAAVKGRFTISQDNAKNTVYLQMNSLKPED |
| | | TAMYYCAIGEIKVVDGKSSCAAEDAGGQGTLVTVSSAHHSE |
| | | DPGPRGLAAAG |
| 117 | Anti- | DVQLVESGGGLVQPGGSLRLSCAASGSIFSIYAMGWYRQAP |
| | Norovirus | GKQRELVASISSGGGTNYADSVKGRFTISGDNAKNTVYLQM |
| | | NSLKPEDTAVYYCKREDYSAYAPPSGSRGRGTQVTVSS |
| 118 | anti-TNFa | DVQLVESGGGLVQPGGSLKLSCAASGFDFSSHWMYWVRQA |
| | (V565) | PGKELEWLSEINTNGLITHYGDSVKGRFTVSRNNAANKMYL |
| | | ELTRLEPEDTALYYCARNQHGLNKGQGTQVTVSS |
| 119 | anti-TNFR1 | EVQLLESGGGLVQPGGSLRLSCAASGFTFAHETMVWVRQAP |
| | (GSK1995057) | GKGLEWVSHIPPDGQDPFYADSVKGRFTISRDNSKNTLYLQ |
| | | MNSLRAEDTAVYHCALLPKRGPWFDYWGQGTLVTVSS |
| 120 | anti-TNFa | DVQLVESGGGLVQAGGSLRLSCAASGGTFSSIIMAWFRQAP |
| | | GKEREFVGAVSWSGGTTVYADSVLGRFEISRDSARKSVYLQ |
| | | MNSLKPEDTAVYYCAARPYQKYNWASASYNVWGQGTLVT |
| | | VSS |
| 121 | Adrenomedullin | YRQSMNNFQGLRSFGCRFGTCTVQKLAHQIYQFTDKDKDNV |
| | | APRSKISPQGY |
| 122 | Urumin (frog) | IPLRGAFINGRWDSQCHRFSNGAIACA |
| 123 | FLG | RNEEQSGDGSRHSGSRHHEPSTRAGSSRHSQVGQGESAGSKT |
| | | SRRQGSSVSQDRDSEGHSEDSERRSESASRNHYGSAREQSRH |
| | | GSRNPRSHQEDRASHGHSAESSRQSGTRHAETSSGGQAASSQ |
| | | EQARSSPGERHGSRHQQSADSSTDSGTGRRQDSSVVGDSGN |
| | | RGSSGSQASDSEGHSEESDTQSVSAHGQAGPHQQSHQESTRG |
| | | QSGERSGRSGSFLYQVSTHEQSESAHGRTGPSTGGRQRSRHE |
| | | QARDSSRHSASQEGQDTIRGHPGSSRGGRQGSHYEQSVDSSG |
| | | HSGSHHSHTTSQERSDVSRGQSGSRSVSRQTRRMRRMRRMR |
| | | R |
| 124 | Anti-RSV | QVQLQESGGGLVQPGGSLRLSCAASGFTLDYYYIGWFRQAP |
| | | GKEREAVSCISGSSGSTYYPDSVKGRFTISRDNAKNTVYLQM |
| | | NSLKPEDTAVYYCATIRSSSWGGCVHYGMDYWGKGTQVTV |
| | | SS |
| 127 | Anti-TcdA | QLQLVETGGGLVQPGGSLRLSCAASGFTFSDYVMTWVRQAP |
| | | GKGPEWIATINTDGSTMRDDSTKGRFTISRDNAKNTLYLQM |
| | | TSLKPEDTALYYCARGVISASAIRGAVRGPGTQVTVSSEPKTP |
| | | KPQP |
| 128 | Anti-TcdB | QVQLVESGGGLVQPGGSLRLSCAASGFSLDYYGIGWFRQAP |
| | | GKERQEVSYISASAKTKLYSDSVKGRFTISRDNAKNAVYLE |
| | | MNSLKREDTAVYYCARRRFDASASNRWLAADYDYWGQGT |
| | | QVTVSSEPKTPKPQ |
| 129 | Anti-PCSK9 | QVQLQDSGGGLVQVGDRLSLSCAASGRTFSSYDMAWFRQA |
| | | PGKEREFVAAITTSEGTWYGDAGKGRFTIARVNAKNTVYLH |
| | | MNRLKPEDTAVYYCAASNQGGSLQISTNYNYWGQGTQVTV |
| | | SS |
| 130 | Anti- | EVQLVESGGGLVQPGGSLRLSCAASGSTLDYYAIGWFRQAP |
| | Pseudomonas | GKEREGVSCTSNSGSTYYGGSVKGRFTASRDNAKNTVYLQ |
| | Toxin | MNSLRPEDTAVYYCVATIGCATLGGTLDVQRYYYRGQGTQ |
| | | VTVSS |
| 131 | Anti-IgE | DVQLLESGGGLVQPGGSLRLSCAASGFTFGNYDMAWVRQA |
| | | PGKRPEWVSSIDTGGDITHYADSVKGRFTISRDNAKNTLYLQ |
| | | MNSLRPEDTAVYWCATDEEYALGPNEFDYYGQGTLVTVSS |
| 282 | Mouse IL-22 | LPVNTRCKLEVSNFQQPYIVNRTFMLAKEASLADNNTDVRLI |
| | | GEKLFRGVSAKDQCYLMKQVLNFTLEDVLLPQSDRFQPYM |
| | | QEVVPFLTKLSNQLSSCHISGDDQNIQKNVRRLKETVKKLGE |
| | | SGEIKAIGELDLLFMSLRNACV |
| 283 | IL-10 Dimer | SPGQGTQSENSCTHFPGNLPNMLRDLRDAFSRVKTFFQMKD |
| | | QLDNLLLKESLLEDFKGYLGCQALSEMIQFYLEEVMPQAEN |
| | | QDPDIKAHVNSLGENLKTLRLRLRRCHRFLPCENKSKAVEQ |
| | | VKNAFNKLQEKGIYKAMSEFDIFINYIEAYMTMKIRNGGGSG |
| | | GGSPGQGTQSENSCTHFPGNLPNMLRDLRDAFSRVKTFFQM |
| | | KDQLDNLLLKESLLEDFKGYLGCQALSEMIQFYLEEVMPQA |
| | | ENQDPDIKAHVNSLGENLKTLRLRLRRCHRFLPCENKSKAVE |
| | | QVKNAFNKLQEKGIYKAMSEFDIFINYIEAYMTMKIRN |
| 284 | IL-10 Biased | SPGQGTQSENSCTHFPGYLPNMLRDLRDAFSRVKTFFQMKD |
| | | QLDNLLLKESLLEDFKGYLGCQALSEMIQFYLEEVMPQAEN |
| | | QDPDIKAHVQSLGENLKDLRLWLRRCHRFLPCENKSKAVEQ |
| | | VKNAFNKLQEKGIYKAMSEFDIFINYIEAYMTMKIRN |
| 285 | IL-10 WT no | SPGQGTQSENSCTHFPGNLPNMLRDLRDAFSRVKTFFQMKD |
| | N-term tag | QLDNLLLKESLLEDFKGYLGCQALSEMIQFYLEEVMPQAEN |
| | | QDPDIKAHVNSLGENLKTLRLRLRRCHRFLPCENKSKAVEQ |
| | | VKNAFNKLQEKGIYKAMSEFDIFINYIEAYMTMKIRN |

It is to be understood that the sequences provided in Table 3 are exemplary and are not meant to be limiting in any way. For example, SEQ ID NO: 59 and SEQ ID NO: 92 each contain the subsequence of:

(SEQ ID NO: 143)
TPQNITDLCAEYHNTQIYTLNDKIFSYTESLAGKREMAIITFKNGAIFQV
EVPGSQHIDSQKKAIERMKDTLRIAYLTEAKVEKLCVWNNKTPHAIAAIS
MANGPGPKKRKSV

which promotes epithelial cell uptake. In some embodiments, any sequence provided in Table 2 may further comprise the sequence of SEQ ID NO: 143 at the N terminus of the payload protein.

In some embodiments, the amino acid sequence of Z₁ is substantially similar to an amino acid sequence provided in Table 3. In some embodiments, the amino acid sequence of Z₁ is a fragment of an amino acid sequence provided in Table 3. In some embodiments, the amino acid sequence of Z₁ is substantially similar to an amino acid sequence provided in Table 3, or is a fragment of an amino acid sequence provided in Table 3, and further comprises the sequence of SEQ ID NO: 143 on the N terminus of the payload protein. In some embodiments, the amino acid sequence of Z₁ has at least 80%, 85%, 90%, 91%, 92%, 93%, 94%, 95%, 96%, 97%, 98%, 99%, or 100% identity to an amino acid sequence provided in Table 3. Further, it is to be understood that the identity of Z₁ may also comprise an amino acid sequence that is a homolog of a sequence provided in Table 3. For example, Table 3 may provide an amino acid sequence corresponding to a human amino acid sequence. It is to be understood that the present embodiments are not limited to the human amino acid sequence provided, but also may comprise the amino acid sequence of a homologous protein from, for example, non-human primates, mouse, rat, dog, horse, sheep, goat, etc.

In some embodiments, the polypeptide comprises a formula of X₁-Z₁, wherein X₁ is a pre-protein signal peptide comprising an amino acid sequence having at least 80%, 85%, 90%, 91%, 92%, 93%, 94%, 95%, 96%, 97%, 98%, 99%, or 100% identity to an amino acid sequence provided in Table 1 and Z₁ is a payload protein having at least 80%, 85%, 90%, 91%, 92%, 93%, 94%, 95%, 96%, 97%, 98%, 99%, or 100% identity to an amino acid sequence provided in Table 3. In some embodiments, X₁ is as provided for herein and Z₁ is as provided for herein.

In some embodiments, the polypeptide comprises a formula of X₁-Z₁, wherein X₁ is a pre-protein signal peptide as provided for herein and Z₁ is a vaccine antigen. In some embodiments, the vaccine antigen is a bacterial antigen or a viral antigen. In some embodiments, the vaccine antigen is a bacterial antigen. In some embodiments, the bacterial antigen is a protein from a pathogenic bacteria. In some embodiments, the bacterial antigen is from Streptococcus agalactiae GBS, Borrelia burgdorferi, Colstridium tetani, Erysipelothrix rhusiopathiae, Helicobacter pylori, Salmonella enterica, Streptococcus mutans, Streptococcus pyogens, Streptococcus pneumonia, or Listeria monocytogenes. In some embodiments, the bacterial antigen comprises Pili proteins, outer surface protein A of Borellia burgdoferi (OspA), tetanus toxin (TTFC), surface protective antigen A (SpaA), Urease B (UreB), Cag12, flagellin (FliC), surface protein antigen of S. mutans serotype c (PAc), M6, pneumococcal surface adhesion A (PsaA), pneumococcal protective protein A (PppA), Listeriolysin O (LLO), or a fragment thereof. In some embodiments, the polypeptide comprises a formula of X₁-Z₁, wherein X₁ is a pre-protein signal peptide as provided for herein and Z₁ is a bacterial vaccine antigen comprising Pili, OspA, TTFC, SpaA, UreB, Cag12, FliC, PAc, M6, PsaA, PppA, LLO, or a fragment thereof.

In some embodiments, the vaccine antigen is a viral antigen. In some embodiments, the viral antigen is from bovine coronavirus, coronavirus, Dengue virus serotype 2, HIV-1, HPV-16, avian influenza, infectious bursal disease virus (IBDV), chicken anemia virus (CAV), porcine parvovirus, rotavirus, or Hepatitis B virus. In some embodiments, the viral antigen comprises NSP4, SARS glycoprotein, SARS spike glycoprotein S, Dengue virus envelope glycoprotein domain III (EDIII), V3 of HIV-1 gp120 glycoprotein, HPV-16 E7 protein, HPV-16 E6 protein, HPV-16 L1 protein, HA envelope protein of H5N1 avian influenza, VP2 of IBDV, VP3 of IBDV, VP1 of CAV, VP2 of porcine parvovirus, VP7 of rotavirus, PreS domain of Hepatitis B, or PreSa domain of Hepatitis B. In some embodiments, the polypeptide comprises a formula of X₁-Z₁, wherein X₁ is a pre-protein signal peptide as provided for herein and Z₁ is a viral antigen comprising, NSP4, SARS glycoprotein, SARS spike glycoprotein S, Dengue virus envelope glycoprotein domain III (EDIII), V3 of HIV-1 gp120 glycoprotein, HPV-16 E7 protein, HPV-16 E6 protein, HPV-16 L1 protein, HA envelope protein of H5N1 avian influenza, VP2 of IBDV, VP3 of IBDV, VP1 of CAV, VP2 of porcine parvovirus, VP7 of rotavirus, PreS domain of Hepatitis B, or PreSa domain of Hepatitis B.

In some embodiments, the polypeptide comprises a formula of X₁-Z₁, wherein X₁ is a pre-protein signal peptide as provided for herein and Z₁ is an anti-microbial peptide. In some embodiments, the anti-microbial peptide is any anti-microbial peptide. In some embodiments, the anti-microbial peptide is hBD1, hBD2, hBD3, b-Cateslytin, Dermaseptin, HD5, Reg3a, or C5a. In some embodiments, the anti-microbial peptide comprises an amino acid sequence having at least 80%, 85%, 90%, 91%, 92%, 93%, 94%, 95%, 96%, 97%, 98%, 99%, or 100% to an amino acid sequence selected from SEQ ID NO: 23, SEQ ID NO: 49, SEQ ID NO: 50, SEQ ID NO: 51, SEQ ID NO: 52, SEQ ID NO: 53, SEQ ID NO: 54, or SEQ ID NO: 55. In some embodiments, the polypeptide comprises a formula of X₁-Z₁, wherein X₁ is a pre-protein signal peptide as provided for herein and Z₁ is an anti-microbial peptide comprising an amino acid sequence having at least 80%, 85%, 90%, 91%, 92%, 93%, 94%, 95%, 96%, 97%, 98%, 99%, or 100% to an amino acid sequence selected from SEQ ID NO: 23, SEQ ID NO: 49, SEQ ID NO: 50, SEQ ID NO: 51, SEQ ID NO: 52, SEQ ID NO: 53, SEQ ID NO: 54, or SEQ ID NO: 55.

In some embodiments, the polypeptide comprises a formula of X₁-Z₁, wherein X₁ is a pre-protein signal peptide as provided for herein and Z₁ is an enzyme. In some embodiments, the enzyme is any enzyme. In some embodiments, the enzyme is Clbs, shP28GST, Clbs (Gilliamella apicola), Clbs (Snodgrassella alvi), Phytase, SUC2, SUIS, AnPep, beta-galactosidase (Lactobacillus), LACPL Exo-alpha-1,4-glucosidase, beta-galactosidase (yeast), ACE2, Xylose isomerase (RainFlu), Xylose isomerase (Lactobacillus), MaIL, apyrase, or DL-endopeptidase. In some embodiments, the enzyme comprises an amino acid sequence having at least 80%, 85%, 90%, 91%, 92%, 93%, 94%, 95%, 96%, 97%, 98%, 99%, or 100% to an amino acid sequence selected from SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, or SEQ ID NO: 78. In some embodiments, the polypeptide comprises a formula of X₁-Z₁, wherein X₁ is a pre-protein signal peptide as provided for herein and Z₁ is an enzyme comprising an amino acid sequence having at least 80%, 85%, 90%, 91%, 92%, 93%, 94%, 95%, 96%, 97%, 98%, 99%, or 100% to an amino acid sequence selected from SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, or SEQ ID NO: 78.

In some embodiments, the polypeptide comprises a formula of X₁-Z₁, wherein X₁ is a pre-protein signal peptide as provided for herein and Z₁ is a food protein. In some embodiments, the food protein is any food protein. In some embodiments, the food protein is beta-casein, alpha-casein, alpha casein derived peptides, Ara h1, or Ara h2. In some embodiments, the food protein comprises an amino acid sequence having at least 80%, 85%, 90%, 91%, 92%, 93%, 94%, 95%, 96%, 97%, 98%, 99%, or 100% to an amino acid sequence selected from SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 89, or SEQ ID NO: 90. In some embodiments, the polypeptide comprises a formula of X₁-Z₁, wherein X₁ is a pre-protein signal peptide as provided for herein and Z₁ is a food protein comprising an amino acid sequence having at least 80%, 85%, 90%, 91%, 92%, 93%, 94%, 95%, 96%, 97%, 98%, 99%, or 100% to an amino acid sequence selected from SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 89, or SEQ ID NO: 90.

In some embodiments, the polypeptide comprises a formula of X₁-Z₁, wherein X₁ is a pre-protein signal peptide as provided for herein and Z₁ is a growth factor. In some embodiments, the growth factor is any growth factor. In some embodiments, the growth factor is selected from IGF1, CTB-IGF1, or Elafin. In some embodiments, the growth factor comprises an amino acid sequence having at least 80%, 85%, 90%, 91%, 92%, 93%, 94%, 95%, 96%, 97%, 98%, 99%, or 100% to an amino acid sequence selected from SEQ ID NO: 91, SEQ ID NO: 92, or SEQ ID NO: 93. In some embodiments, the polypeptide comprises a formula of X₁-Z₁, wherein X₁ is a pre-protein signal peptide as provided for herein and Z₁ is a growth factor comprising an amino acid sequence having at least 80%, 85%, 90%, 91%, 92%, 93%, 94%, 95%, 96%, 97%, 98%, 99%, or 100% to an amino acid sequence selected from SEQ ID NO: 91, SEQ ID NO: 92, or SEQ ID NO: 93.

In some embodiments, the polypeptide comprises a formula of X₁-Z₁, wherein X₁ is a pre-protein signal peptide as provided for herein and Z₁ is an immunoreceptor. In some embodiments, the immunoreceptor is any immunoreceptor. In some embodiments, the immunoreceptor is TIGIT. In some embodiments, the immunoreceptor comprises an amino acid sequence having at least 80%, 85%, 90%, 91%, 92%, 93%, 94%, 95%, 96%, 97%, 98%, 99%, or 100% to an amino acid sequence of SEQ ID NO: 104. In some embodiments, the polypeptide comprises a formula of X₁-Z₁, wherein X₁ is a pre-protein signal peptide as provided for herein and Z₁ is an immunoreceptor comprising an amino acid sequence having at least 80%, 85%, 90%, 91%, 92%, 93%, 94%, 95%, 96%, 97%, 98%, 99%, or 100% to an amino acid sequence of SEQ ID NO: 104.

In some embodiments, the polypeptide comprises a formula of X₁-Z₁, wherein X₁ is a pre-protein signal peptide as provided for herein and Z₁ is a mucolytic. In some embodiments, the mucolytic is any mucolytic. In some embodiments, the mucolytic is DNAse1. In some embodiments, the mucolytic comprises an amino acid sequence having at least 80%, 85%, 90%, 91%, 92%, 93%, 94%, 95%, 96%, 97%, 98%, 99%, or 100% to an amino acid sequence of SEQ ID NO: 105. In some embodiments, the polypeptide comprises a formula of X₁-Z₁, wherein X₁ is a pre-protein signal peptide as provided for herein and Z₁ is a mucolytic comprising an amino acid sequence having at least 80%, 85%, 90%, 91%, 92%, 93%, 94%, 95%, 96%, 97%, 98%, 99%, or 100% to an amino acid sequence of SEQ ID NO: 105.

In some embodiments, the polypeptide comprises a formula of X₁-Z₁, wherein X₁ is a pre-protein signal peptide as provided for herein and Z₁ is a peptide. In some embodiments, the peptide is any peptide. In some embodiments, the peptide is selected from andrenomedullin or urumin. In some embodiments, the peptide comprises an amino acid sequence having at least 80%, 85%, 90%, 91%, 92%, 93%, 94%, 95%, 96%, 97%, 98%, 99%, or 100% to an amino acid sequence of SEQ ID NO: 121 or SEQ ID NO: 122. In some embodiments, the polypeptide comprises a formula of X₁-Z₁, wherein X₁ is a pre-protein signal peptide as provided for herein and Z₁ is a peptide comprising an amino acid sequence having at least 80%, 85%, 90%, 91%, 92%, 93%, 94%, 95%, 96%, 97%, 98%, 99%, or 100% to an amino acid sequence of SEQ ID NO: 121 or SEQ ID NO: 122.

In some embodiments, the polypeptide comprises a formula of X₁-Z₁, wherein X₁ is a pre-protein signal peptide as provided for herein and Z₁ is a filament-associated protein. In some embodiments, the filament-associated protein is any filament-associate protein. In some embodiments, the filament-associated protein is filaggrin. In some embodiments, the filament-associated protein comprises an amino acid sequence having at least 80%, 85%, 90%, 91%, 92%, 93%, 94%, 95%, 96%, 97%, 98%, 99%, or 100% to an amino acid sequence of SEQ ID NO: 123. In some embodiments, the polypeptide comprises a formula of X₁-Z₁, wherein X₁ is a pre-protein signal peptide as provided for herein and Z₁ is a filament-associated protein comprising an amino acid sequence having at least 80%, 85%, 90%, 91%, 92%, 93%, 94%, 95%, 96%, 97%, 98%, 99%, or 100% to an amino acid sequence of SEQ ID NO: 123.

In some embodiments, the polypeptide comprises a formula of X₁-Z₁, wherein X₁ comprises an amino acid having at least 80%, 85%, 90%, 91%, 92%, 93%, 94%, 95%, 96%, 97%, 98%, 99%, or 100% identity to an amino acid sequence of SEQ ID NO: 20, and Z₁ comprises a lectin. In some embodiments, the lectin is any lectin. In some embodiments, the lectin is TFF1, TFF2, or TFF3. In some embodiments, the cytokine comprises an amino acid sequence having at least 80%, 85%, 90%, 91%, 92%, 93%, 94%, 95%, 96%, 97%, 98%, 99%, or 100% to an amino acid sequence selected from SEQ ID NO: 56, SEQ ID NO: 57, or SEQ ID NO: 58. In some embodiments, the polypeptide comprises a formula of X₁-Z₁, wherein X₁ is a pre-protein signal peptide as provided for herein and Z₁ comprises a lectin comprising an amino acid sequence having at least 80%, 85%, 90%, 91%, 92%, 93%, 94%, 95%, 96%, 97%, 98%, 99%, or 100% to an amino acid sequence selected from SEQ ID NO: 56, SEQ ID NO: 57, or SEQ ID NO: 58.

In some embodiments, the polypeptide comprises a formula of X₁-Z₁, wherein X₁ comprises an amino acid having at least 80%, 85%, 90%, 91%, 92%, 93%, 94%, 95%, 96%, 97%, 98%, 99%, or 100% identity to an amino acid sequence selected from SEQ ID NO: 6, SEQ ID NO: 7, or SEQ ID NO: 14, and Z₁ comprises a nanobody. In some embodiments, the nanobody can be any nanobody. In some embodiments, the nanobody binds to a target protein, wherein the target protein can be any protein. In some embodiments, the nanobody binds to a target protein, wherein the target protein is TNFα, CCL2, IL23, EGFR-III, GLP2, VEGFB, ILR6, MERS, SARS, BVD, Norovirus, RSV, TcdA, TcdB, PCSK9, Pseudomonas toxin, or IgE. In some embodiments, the nanobody comprises an amino acid sequence having at least 80%, 85%, 90%, 91%, 92%, 93%, 94%, 95%, 96%, 97%, 98%, 99%, or 100% to an amino acid sequence selected from SEQ ID NO: 21, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 126, SEQ ID NO: 127, SEQ ID NO: 128, SEQ ID NO: 129, SEQ ID NO: 130, or SEQ ID NO: 131. In some embodiments, the polypeptide comprises a formula of X₁-Z₁, wherein X₁ comprises an amino acid having at least 80%, 85%, 90%, 91%, 92%, 93%, 94%, 95%, 96%, 97%, 98%, 99%, or 100% identity to an amino acid sequence selected from SEQ ID NO: 6, SEQ ID NO: 7, or SEQ ID NO: 14, and Z₁ comprises a nanobody comprising an amino acid sequence having at least 80%, 85%, 90%, 91%, 92%, 93%, 94%, 95%, 96%, 97%, 98%, 99%, or 100% to an amino acid sequence selected from SEQ ID NO: 21, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 126, SEQ ID NO: 127, SEQ ID NO: 128, SEQ ID NO: 129, SEQ ID NO: 130, or SEQ ID NO: 131.

In some embodiments, the polypeptide comprises a formula of X₁-Z₁, wherein X₁ comprises an amino acid having at least 80%, 85%, 90%, 91%, 92%, 93%, 94%, 95%, 96%, 97%, 98%, 99%, or 100% identity to an amino acid sequence selected from SEQ ID NO: 6, SEQ ID NO: 7, SEQ ID NO: 14, SEQ ID NO: 17, SEQ ID NO: 18, SEQ ID NO: 19, SEQ ID NO: 47, SEQ ID NO: 48, SEQ ID NO: 148, or SEQ ID NO: 180, and Z₁ comprises a hormone. In some embodiments, the hormone is any hormone. In some embodiments, the hormone is GLP1, Exenatide-4, Catestatin, Leptin, Nesfatin, Linaclotide, Plecanatide, Uroguanylin, PYY, Guanylin, or LCRF. In some embodiments, the hormone comprises an amino acid sequence having at least 80%, 85%, 90%, 91%, 92%, 93%, 94%, 95%, 96%, 97%, 98%, 99%, or 100% to an amino acid sequence selected from SEQ ID NO: 25, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, or SEQ ID NO: 103. In some embodiments, the polypeptide comprises a formula of X₁-Z₁, wherein X₁ comprises an amino acid having at least 80%, 85%, 90%, 91%, 92%, 93%, 94%, 95%, 96%, 97%, 98%, 99%, or 100% identity to an amino acid sequence selected from SEQ ID NO: 6, SEQ ID NO: 7, SEQ ID NO: 14, SEQ ID NO: 17, SEQ ID NO: 18, SEQ ID NO: 19, SEQ ID NO: 47, SEQ ID NO: 48, SEQ ID NO: 148, or SEQ ID NO: 180, and Z₁ comprises a hormone comprising an amino acid sequence having at least 80%, 85%, 90%, 91%, 92%, 93%, 94%, 95%, 96%, 97%, 98%, 99%, or 100% to an amino acid sequence selected from SEQ ID NO: 25, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, or SEQ ID NO: 103.

In some embodiments, the polypeptide comprises a formula of X₁-Z₁, wherein X₁ comprises an amino acid having at least 80%, 85%, 90%, 91%, 92%, 93%, 94%, 95%, 96%, 97%, 98%, 99%, or 100% identity to an amino acid sequence of SEQ ID NO: 7, SEQ ID NO: 14, SEQ ID NO: 20, SEQ ID NO: 148, or SEQ ID NO: 180, and Z₁ comprises a cytokine. In some embodiments, the cytokine is any cytokine. In some embodiments, the cytokine is IL-10, IL-22, or CTB-IL-22. In some embodiments, the cytokine comprises an amino acid sequence having at least 80%, 85%, 90%, 91%, 92%, 93%, 94%, 95%, 96%, 97%, 98%, 99%, or 100% to an amino acid sequence selected from SEQ ID NO: 22, SEQ ID NO: 24, SEQ ID NO: 59, SEQ ID NO: 282, SEQ ID NO: 283, SEQ ID NO: 284, or SEQ ID NO: 285. In some embodiments, the polypeptide comprises a formula of X₁-Z₁, wherein X₁ comprises an amino acid having at least 80%, 85%, 90%, 91%, 92%, 93%, 94%, 95%, 96%, 97%, 98%, 99%, or 100% identity to an amino acid sequence of SEQ ID NO: 7, SEQ ID NO: 14, SEQ ID NO: 20, SEQ ID NO: 148, or SEQ ID NO: 180, and Z₁ comprises a cytokine comprising an amino acid sequence having at least 80%, 85%, 90%, 91%, 92%, 93%, 94%, 95%, 96%, 97%, 98%, 99%, or 100% to an amino acid sequence selected from SEQ ID NO: 22, SEQ ID NO: 24, SEQ ID NO: 59, SEQ ID NO: 282, SEQ ID NO: 283, SEQ ID NO: 284, or SEQ ID NO: 285.

In some embodiments, the polypeptide comprises a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 6 and Z₁ comprises an anti-TNFα antibody, or an anti-TNFα antibody fragment. In some embodiments, the polypeptide comprises a formula of X₁-Z₁, wherein X₁ comprises an amino acid sequence having at least 98% identity to the amino acid sequence of SEQ ID NO: 6 and Z₁ comprises an anti-TNFα antibody, or an anti-TNFα antibody fragment. In some embodiments, the polypeptide comprises a formula of X₁-Z₁, wherein X₁ comprises an amino acid sequence having the amino acid sequence of SEQ ID NO: 6 and Z₁ comprises an anti-TNFα antibody, or an anti-TNFα antibody fragment. In some embodiments, Z₁ comprises an anti-TNFα antibody. In some embodiments, Z₁ comprises an anti-TNFα antibody fragment. In some embodiments, the anti-TNFα antibody is as provided for herein. In some embodiments, the anti-TNFα antibody fragment is a fragment of an anti-TNFα antibody as provided for herein.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 6 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, or SEQ ID NO: 120, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 6 and Z₁ comprises the amino acid of SEQ ID NO: 21, SEQ ID NO: 118, SEQ ID NO: 119, or SEQ ID NO: 120, or fragment thereof. In some embodiments, X₁ comprises an amino acid sequence having at least 98% identity to the amino acid sequence of SEQ ID NO: 6 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, or SEQ ID NO: 120, or fragment thereof. In some embodiments, X₁ comprises an amino acid sequence having at least 98% identity to the amino acid sequence of SEQ ID NO: 6 and Z₁ comprises the amino acid of SEQ ID NO: 21, SEQ ID NO: 118, SEQ ID NO: 119, or SEQ ID NO: 120, or fragment thereof. In some embodiments, X₁ comprises the amino acid sequence of SEQ ID NO: 6 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, or SEQ ID NO: 120, or fragment thereof. In some embodiments, X₁ comprises the amino acid sequence of SEQ ID NO: 6 and Z₁ comprises the amino acid of SEQ ID NO: 21, SEQ ID NO: 118, SEQ ID NO: 119, or SEQ ID NO: 120, or fragment thereof. In some embodiments, X₁ comprises the amino acid sequence of SEQ ID NO: 6 and Z₁ comprises the amino acid of SEQ ID NO: 21, or fragment thereof. In some embodiments, X₁ comprises the amino acid sequence of SEQ ID NO: 6 and Z₁ comprises the amino acid of SEQ ID NO: 118, or fragment thereof. In some embodiments, X₁ comprises the amino acid sequence of SEQ ID NO: 6 and Z₁ comprises the amino acid of SEQ ID NO: 119, or fragment thereof. In some embodiments, X₁ comprises the amino acid sequence of SEQ ID NO: 6 and Z₁ comprises the amino acid of SEQ ID NO: 120, or fragment thereof.

In some embodiments, the polypeptide comprises a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 7 and Z₁ comprises an anti-TNFα antibody, or an anti-TNFα antibody fragment. In some embodiments, the polypeptide comprises a formula of X₁-Z₁, wherein X₁ comprises an amino acid sequence having at least 98% identity to the amino acid sequence of SEQ ID NO: 7 and Z₁ comprises an anti-TNFα antibody, or an anti-TNFα antibody fragment. In some embodiments, the polypeptide comprises a formula of X₁-Z₁, wherein X₁ comprises an amino acid sequence having the amino acid sequence of SEQ ID NO: 7 and Z₁ comprises an anti-TNFα antibody, or an anti-TNFα antibody fragment. In some embodiments, Z₁ comprises an anti-TNFα antibody. In some embodiments, Z₁ comprises an anti-TNFα antibody fragment. In some embodiments, the anti-TNFα antibody is as provided for herein. In some embodiments, the anti-TNFα antibody fragment is a fragment of an anti-TNFα antibody as provided for herein.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 7 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, or SEQ ID NO: 120, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 7 and Z₁ comprises the amino acid of SEQ ID NO: 21, SEQ ID NO: 118, SEQ ID NO: 119, or SEQ ID NO: 120, or fragment thereof. In some embodiments, X₁ comprises an amino acid sequence having at least 98% identity to the amino acid sequence of SEQ ID NO: 7 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, or SEQ ID NO: 120, or fragment thereof. In some embodiments, X₁ comprises an amino acid sequence having at least 98% identity to the amino acid sequence of SEQ ID NO: 7 and Z₁ comprises the amino acid of SEQ ID NO: 21, SEQ ID NO: 118, SEQ ID NO: 119, or SEQ ID NO: 120, or fragment thereof. In some embodiments, X₁ comprises an amino acid sequence of SEQ ID NO: 7 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, or SEQ ID NO: 120, or fragment thereof. In some embodiments, X₁ comprises the amino acid sequence of SEQ ID NO: 7 and Z₁ comprises the amino acid of SEQ ID NO: 21, SEQ ID NO: 118, SEQ ID NO: 119, or SEQ ID NO: 120, or fragment thereof. In some embodiments, X₁ comprises the amino acid sequence of SEQ ID NO: 7 and Z₁ comprises the amino acid of SEQ ID NO: 21, or fragment thereof. In some embodiments, X₁ comprises the amino acid sequence of SEQ ID NO: 7 and Z₁ comprises the amino acid of SEQ ID NO: 118, or fragment thereof. In some embodiments, X₁ comprises the amino acid sequence of SEQ ID NO: 7 and Z₁ comprises the amino acid of SEQ ID NO: 119, or fragment thereof. In some embodiments, X₁ comprises the amino acid sequence of SEQ ID NO: 7 and Z₁ comprises the amino acid of SEQ ID NO: 120, or fragment thereof.

In some embodiments, the polypeptide comprises a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 14 and Z₁ comprises an anti-TNFα antibody, or an anti-TNFα antibody fragment. In some embodiments, the polypeptide comprises a formula of X₁-Z₁, wherein X₁ comprises an amino acid sequence having at least 98% identity to the amino acid sequence of SEQ ID NO: 14 and Z₁ comprises an anti-TNFα antibody, or an anti-TNFα antibody fragment. In some embodiments, the polypeptide comprises a formula of X₁-Z₁, wherein X₁ comprises an amino acid sequence having the amino acid sequence of SEQ ID NO: 14 and Z₁ comprises an anti-TNFα antibody, or an anti-TNFα antibody fragment. In some embodiments, Z₁ comprises an anti-TNFα antibody. In some embodiments, Z₁ comprises an anti-TNFα antibody fragment. In some embodiments, the anti-TNFα antibody is as provided for herein. In some embodiments, the anti-TNFα antibody fragment is a fragment of an anti-TNFα antibody as provided for herein.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 14 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, or SEQ ID NO: 120, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 14 and Z₁ comprises an amino acid sequence of SEQ ID NO: 21, SEQ ID NO: 118, SEQ ID NO: 119, or SEQ ID NO: 120, or fragment thereof. In some embodiments, X₁ comprises an amino acid sequence having at least 98% identity to the amino acid sequence of SEQ ID NO: 14 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, or SEQ ID NO: 120, or fragment thereof. In some embodiments, X₁ comprises an amino acid sequence having at least 98% identity to the amino acid sequence of SEQ ID NO: 14 and Z₁ comprises an amino acid sequence of SEQ ID NO: 21, SEQ ID NO: 118, SEQ ID NO: 119, or SEQ ID NO: 120, or fragment thereof. In some embodiments, X₁ comprises an amino acid sequence of SEQ ID NO: 14 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, or SEQ ID NO: 120, or fragment thereof. In some embodiments, X₁ comprises the amino acid sequence of SEQ ID NO: 14 and Z₁ comprises the amino acid of SEQ ID NO: 21, SEQ ID NO: 118, SEQ ID NO: 119, or SEQ ID NO: 120, or fragment thereof. In some embodiments, X₁ comprises the amino acid sequence of SEQ ID NO: 14 and Z₁ comprises the amino acid of SEQ ID NO: 21, or fragment thereof. In some embodiments, X₁ comprises the amino acid sequence of SEQ ID NO: 14 and Z₁ comprises the amino acid of SEQ ID NO: 118, or fragment thereof. In some embodiments, X₁ comprises the amino acid sequence of SEQ ID NO: 14 and Z₁ comprises the amino acid of SEQ ID NO: 119, or fragment thereof. In some embodiments, X₁ comprises the amino acid sequence of SEQ ID NO: 14 and Z₁ comprises the amino acid of SEQ ID NO: 120, or fragment thereof.

In some embodiments, the polypeptide comprises a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 17 and Z₁ comprises leptin, or a fragment thereof. In some embodiments, the polypeptide comprises a formula of X₁-Z₁, wherein X₁ comprises an amino acid sequence having at least 98% identity to the amino acid sequence of SEQ ID NO: 17 and Z₁ comprises leptin, or a fragment thereof. In some embodiments, the polypeptide comprises a formula of X₁-Z₁, wherein X₁ comprises an amino acid sequence having the amino acid sequence of SEQ ID NO: 17 and Z₁ comprises leptin, or a fragment thereof. In some embodiments, Z₁ comprises leptin. In some embodiments, Z₁ comprises a fragment of leptin.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 17 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 17 and Z₁ comprises an amino acid sequence of SEQ ID NO: 25, or fragment thereof. In some embodiments, X₁ comprises an amino acid sequence having at least 98% identity to the amino acid sequence of SEQ ID NO: 17 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 98% identity to the amino acid sequence of SEQ ID NO: 17 and Z₁ comprises an amino acid sequence of SEQ ID NO: 25, or fragment thereof. In some embodiments, X₁ comprises the amino acid sequence of SEQ ID NO: 17 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises the amino acid sequence of SEQ ID NO: 17 and Z₁ comprises the amino acid of SEQ ID NO: 25, or fragment thereof.

In some embodiments, the polypeptide comprises a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 18 and Z₁ comprises leptin, or a fragment thereof. In some embodiments, the polypeptide comprises a formula of X₁-Z₁, wherein X₁ comprises an amino acid sequence having at least 98% identity to the amino acid sequence of SEQ ID NO: 18 and Z₁ comprises leptin, or a fragment thereof. In some embodiments, the polypeptide comprises a formula of X₁-Z₁, wherein X₁ comprises an amino acid sequence having the amino acid sequence of SEQ ID NO: 18 and Z₁ comprises leptin, or a fragment thereof. In some embodiments, Z₁ comprises leptin. In some embodiments, Z₁ comprises a fragment of leptin.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 18 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 18 and Z₁ comprises an amino acid sequence of SEQ ID NO: 25, or fragment thereof. In some embodiments, X₁ comprises an amino acid sequence having at least 98% identity to the amino acid sequence of SEQ ID NO: 18 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 98% identity to the amino acid sequence of SEQ ID NO: 18 and Z₁ comprises an amino acid sequence of SEQ ID NO: 25, or fragment thereof. In some embodiments, X₁ comprises an amino acid sequence of SEQ ID NO: 18 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises the amino acid sequence of SEQ ID NO: 18 and Z₁ comprises the amino acid of SEQ ID NO: 25, or fragment thereof.

In some embodiments, the polypeptide comprises a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 19 and Z₁ comprises leptin, or a fragment thereof. In some embodiments, the polypeptide comprises a formula of X₁-Z₁, wherein X₁ comprises an amino acid sequence having at least 98% identity to the amino acid sequence of SEQ ID NO: 19 and Z₁ comprises leptin, or a fragment thereof. In some embodiments, the polypeptide comprises a formula of X₁-Z₁, wherein X₁ comprises an amino acid sequence having the amino acid sequence of SEQ ID NO: 19 and Z₁ comprises leptin, or a fragment thereof. In some embodiments, Z₁ comprises leptin. In some embodiments, Z₁ comprises a fragment of leptin.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 19 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 19 and Z₁ comprises an amino acid sequence of SEQ ID NO: 25, or fragment thereof. In some embodiments, X₁ comprises an amino acid sequence having at least 98% identity to the amino acid sequence of SEQ ID NO: 19 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 98% identity to the amino acid sequence of SEQ ID NO: 19 and Z₁ comprises an amino acid sequence of SEQ ID NO: 25, or fragment thereof. In some embodiments, X₁ comprises an amino acid sequence of SEQ ID NO: 19 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises the amino acid sequence of SEQ ID NO: 19 and Z₁ comprises the amino acid of SEQ ID NO: 25, or fragment thereof.

In some embodiments, the polypeptide comprises a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 47 and Z₁ comprises leptin, or a fragment thereof. In some embodiments, the polypeptide comprises a formula of X₁-Z₁, wherein X₁ comprises an amino acid sequence having at least 98% identity to the amino acid sequence of SEQ ID NO: 47 and Z₁ comprises leptin, or a fragment thereof. In some embodiments, the polypeptide comprises a formula of X₁-Z₁, wherein X₁ comprises an amino acid sequence having the amino acid sequence of SEQ ID NO: 47 and Z₁ comprises leptin, or a fragment thereof. In some embodiments, Z₁ comprises leptin. In some embodiments, Z₁ comprises a fragment of leptin.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 47 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 47 and Z₁ comprises an amino acid sequence of SEQ ID NO: 25, or fragment thereof. In some embodiments, X₁ comprises an amino acid sequence having at least 98% identity to the amino acid sequence of SEQ ID NO: 47 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 98% identity to the amino acid sequence of SEQ ID NO: 47 and Z₁ comprises an amino acid sequence of SEQ ID NO: 25, or fragment thereof. In some embodiments, X₁ comprises an amino acid sequence of SEQ ID NO: 47 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises the amino acid sequence of SEQ ID NO: 47 and Z₁ comprises the amino acid of SEQ ID NO: 25, or fragment thereof.

In some embodiments, the polypeptide comprises a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 48 and Z₁ comprises leptin, or a fragment thereof. In some embodiments, the polypeptide comprises a formula of X₁-Z₁, wherein X₁ comprises an amino acid sequence having at least 98% identity to the amino acid sequence of SEQ ID NO: 48 and Z₁ comprises leptin, or a fragment thereof. In some embodiments, the polypeptide comprises a formula of X₁-Z₁, wherein X₁ comprises an amino acid sequence having the amino acid sequence of SEQ ID NO: 48 and Z₁ comprises leptin, or a fragment thereof. In some embodiments, Z₁ comprises leptin. In some embodiments, Z₁ comprises a fragment of leptin.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 48 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 48 and Z₁ comprises an amino acid sequence of SEQ ID NO: 25, or fragment thereof. In some embodiments, X₁ comprises an amino acid sequence having at least 98% identity to the amino acid sequence of SEQ ID NO: 48 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 98% identity to the amino acid sequence of SEQ ID NO: 48 and Z₁ comprises an amino acid sequence of SEQ ID NO: 25, or fragment thereof. In some embodiments, X₁ comprises an amino acid sequence of SEQ ID NO: 48 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises the amino acid sequence of SEQ ID NO: 48 and Z₁ comprises the amino acid of SEQ ID NO: 25, or fragment thereof.

In some embodiments, the polypeptide comprises a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 148 and Z₁ comprises leptin, or a fragment thereof. In some embodiments, the polypeptide comprises a formula of X₁-Z₁, wherein X₁ comprises an amino acid sequence having at least 98% identity to the amino acid sequence of SEQ ID NO: 148 and Z₁ comprises leptin, or a fragment thereof. In some embodiments, the polypeptide comprises a formula of X₁-Z₁, wherein X₁ comprises an amino acid sequence having the amino acid sequence of SEQ ID NO: 148 and Z₁ comprises leptin, or a fragment thereof. In some embodiments, Z₁ comprises leptin. In some embodiments, Z₁ comprises a fragment of leptin.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 148 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 148 and Z₁ comprises an amino acid sequence of SEQ ID NO: 25, or fragment thereof. In some embodiments, X₁ comprises an amino acid sequence having at least 98% identity to the amino acid sequence of SEQ ID NO: 148 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 98% identity to the amino acid sequence of SEQ ID NO: 148 and Z₁ comprises an amino acid sequence of SEQ ID NO: 25, or fragment thereof. In some embodiments, X₁ comprises an amino acid sequence of SEQ ID NO: 148 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises the amino acid sequence of SEQ ID NO: 148 and Z₁ comprises the amino acid of SEQ ID NO: 25, or fragment thereof.

In some embodiments, the polypeptide comprises a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 180 and Z₁ comprises leptin, or a fragment thereof. In some embodiments, the polypeptide comprises a formula of X₁-Z₁, wherein X₁ comprises an amino acid sequence having at least 98% identity to the amino acid sequence of SEQ ID NO: 180 and Z₁ comprises leptin, or a fragment thereof. In some embodiments, the polypeptide comprises a formula of X₁-Z₁, wherein X₁ comprises an amino acid sequence having the amino acid sequence of SEQ ID NO: 180 and Z₁ comprises leptin, or a fragment thereof. In some embodiments, Z₁ comprises leptin. In some embodiments, Z₁ comprises a fragment of leptin.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 180 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 180 and Z₁ comprises an amino acid sequence of SEQ ID NO: 25, or fragment thereof. In some embodiments, X₁ comprises an amino acid sequence having at least 98% identity to the amino acid sequence of SEQ ID NO: 180 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 98% identity to the amino acid sequence of SEQ ID NO: 180 and Z₁ comprises an amino acid sequence of SEQ ID NO: 25, or fragment thereof. In some embodiments, X₁ comprises an amino acid sequence of SEQ ID NO: 180 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises the amino acid sequence of SEQ ID NO: 180 and Z₁ comprises the amino acid of SEQ ID NO: 25, or fragment thereof.

In some embodiments, the polypeptide comprises a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 20 and Z₁ comprises IL-10, or a fragment thereof. In some embodiments, the polypeptide comprises a formula of X₁-Z₁, wherein X₁ comprises an amino acid sequence having at least 98% identity to the amino acid sequence of SEQ ID NO: 20 and Z₁ comprises IL-10, or a fragment thereof. In some embodiments, the polypeptide comprises a formula of X₁-Z₁, wherein X₁ comprises an amino acid sequence having the amino acid sequence of SEQ ID NO: 20 and Z₁ comprises IL-10, or a fragment thereof. In some embodiments, Z₁ comprises IL-10. In some embodiments, Z₁ comprises a fragment of IL-10.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 20 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 24, SEQ ID NO: 283, SEQ ID NO: 284, or SEQ ID NO: 285, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 20 and Z₁ comprises an amino acid sequence of SEQ ID NO: 24, SEQ ID NO: 283, SEQ ID NO: 284, or SEQ ID NO: 285, or fragment thereof. In some embodiments, X₁ comprises an amino acid sequence having at least 98% identity to the amino acid sequence of SEQ ID NO: 20 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 24, SEQ ID NO: 283, SEQ ID NO: 284, or SEQ ID NO: 285, or fragment thereof. In some embodiments, X₁ comprises an amino acid sequence having at least 98% identity to the amino acid sequence of SEQ ID NO: 20 and Z₁ comprises an amino acid sequence of SEQ ID NO: 24, SEQ ID NO: 283, SEQ ID NO: 284, or SEQ ID NO: 285, or fragment thereof. In some embodiments, X₁ comprises an amino acid sequence of SEQ ID NO: 20 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 24, SEQ ID NO: 283, SEQ ID NO: 284, or SEQ ID NO: 285, or fragment thereof. In some embodiments, X₁ comprises the amino acid sequence of SEQ ID NO: 20 and Z₁ comprises the amino acid of SEQ ID NO: 24, SEQ ID NO: 283, SEQ ID NO: 284, or SEQ ID NO: 285, or fragment thereof. In some embodiments, X₁ comprises the amino acid sequence of SEQ ID NO: 20 and Z₁ comprises the amino acid sequence of SEQ ID NO: 24. In some embodiments, X₁ comprises the amino acid sequence of SEQ ID NO: 20 and Z₁ comprises the amino acid sequence of SEQ ID NO: 283. In some embodiments, X₁ comprises the amino acid sequence of SEQ ID NO: 20 and Z₁ comprises the amino acid sequence of SEQ ID NO: 284. In some embodiments, X₁ comprises the amino acid sequence of SEQ ID NO: 20 and Z₁ comprises the amino acid sequence of SEQ ID NO: 285.

In some embodiments, the polypeptide comprises a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 7 and Z₁ comprises IL-22, or a fragment thereof. In some embodiments, the polypeptide comprises a formula of X₁-Z₁, wherein X₁ comprises an amino acid sequence having at least 98% identity to the amino acid sequence of SEQ ID NO: 7 and Z₁ comprises IL-22, or a fragment thereof. In some embodiments, the polypeptide comprises a formula of X₁-Z₁, wherein X₁ comprises an amino acid sequence having the amino acid sequence of SEQ ID NO: 7 and Z₁ comprises IL-22, or a fragment thereof. In some embodiments, Z₁ comprises IL-22. In some embodiments, Z₁ comprises a fragment of IL-22.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 7 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 7 and Z₁ comprises an amino acid sequence of SEQ ID NO: 22, SEQ ID NO: 59, or SEQ ID NO: 282, or fragment thereof. In some embodiments, X₁ comprises an amino acid sequence having at least 98% identity to the amino acid sequence of SEQ ID NO: 7 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises an amino acid sequence having at least 98% identity to the amino acid sequence of SEQ ID NO: 7 and Z₁ comprises an amino acid sequence of SEQ ID NO: 22, SEQ ID NO: 59, or SEQ ID NO: 282, or fragment thereof. In some embodiments, X₁ comprises an amino acid of SEQ ID NO: 7 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises the amino acid sequence of SEQ ID NO: 7 and Z₁ comprises the amino acid of SEQ ID NO: 22, SEQ ID NO: 59, or SEQ ID NO: 282, or fragment thereof. In some embodiments, X₁ comprises the amino acid sequence of SEQ ID NO: 7 and Z₁ comprises the amino acid of SEQ ID NO: 22, or fragment thereof. In some embodiments, X₁ comprises the amino acid sequence of SEQ ID NO: 7 and Z₁ comprises the amino acid of SEQ ID NO: 59, or fragment thereof. In some embodiments, X₁ comprises the amino acid sequence of SEQ ID NO: 7 and Z₁ comprises the amino acid of SEQ ID NO: 282, or fragment thereof.

In some embodiments, the polypeptide comprises a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 14 and Z₁ comprises IL-22, or a fragment thereof. In some embodiments, the polypeptide comprises a formula of X₁-Z₁, wherein X₁ comprises an amino acid sequence having at least 98% identity to the amino acid sequence of SEQ ID NO: 14 and Z₁ comprises IL-22, or a fragment thereof. In some embodiments, the polypeptide comprises a formula of X₁-Z₁, wherein X₁ comprises an amino acid sequence having the amino acid sequence of SEQ ID NO: 14 and Z₁ comprises IL-22, or a fragment thereof. In some embodiments, Z₁ comprises IL-22. In some embodiments, Z₁ comprises a fragment of IL-22.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 14 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 14 and Z₁ comprises an amino acid sequence of SEQ ID NO: 22, SEQ ID NO: 59, or SEQ ID NO: 282, or fragment thereof. In some embodiments, X₁ comprises an amino acid sequence having at least 98% identity to the amino acid sequence of SEQ ID NO: 14 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises an amino acid sequence having at least 98% identity to the amino acid sequence of SEQ ID NO: 14 and Z₁ comprises an amino acid sequence of SEQ ID NO: 22, SEQ ID NO: 59, or SEQ ID NO: 282, or fragment thereof. In some embodiments, X₁ comprises an amino acid sequence of SEQ ID NO: 14 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises the amino acid sequence of SEQ ID NO: 14 and Z₁ comprises the amino acid of SEQ ID NO: 22, SEQ ID NO: 59, or SEQ ID NO: 282, or fragment thereof. In some embodiments, X₁ comprises the amino acid sequence of SEQ ID NO: 14 and Z₁ comprises the amino acid of SEQ ID NO: 22, or fragment thereof. In some embodiments, X₁ comprises the amino acid sequence of SEQ ID NO: 14 and Z₁ comprises the amino acid of SEQ ID NO: 59, or fragment thereof. In some embodiments, X₁ comprises the amino acid sequence of SEQ ID NO: 14 and Z₁ comprises the amino acid of SEQ ID NO: 282, or fragment thereof.

In some embodiments, the polypeptide comprises a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 148 and Z₁ comprises IL-22, or a fragment thereof. In some embodiments, the polypeptide comprises a formula of X₁-Z₁, wherein X₁ comprises an amino acid sequence having at least 98% identity to the amino acid sequence of SEQ ID NO: 148 and Z₁ comprises IL-22, or a fragment thereof. In some embodiments, the polypeptide comprises a formula of X₁-Z₁, wherein X₁ comprises an amino acid sequence having the amino acid sequence of SEQ ID NO: 148 and Z₁ comprises IL-22, or a fragment thereof. In some embodiments, Z₁ comprises IL-22. In some embodiments, Z₁ comprises a fragment of IL-22.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 148 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 148 and Z₁ comprises an amino acid sequence of SEQ ID NO: 22, SEQ ID NO: 59, or SEQ ID NO: 282, or fragment thereof. In some embodiments, X₁ comprises an amino acid sequence having at least 98% identity to the amino acid sequence of SEQ ID NO: 148 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises an amino acid sequence having at least 98% identity to the amino acid sequence of SEQ ID NO: 148 and Z₁ comprises an amino acid of SEQ ID NO: 22, SEQ ID NO: 59, or SEQ ID NO: 282, or fragment thereof. In some embodiments, X₁ comprises an amino acid sequence of SEQ ID NO: 148 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises the amino acid sequence of SEQ ID NO: 148 and Z₁ comprises the amino acid of SEQ ID NO: 22, SEQ ID NO: 59, or SEQ ID NO: 282, or fragment thereof. In some embodiments, X₁ comprises the amino acid sequence of SEQ ID NO: 148 and Z₁ comprises the amino acid of SEQ ID NO: 22, or fragment thereof. In some embodiments, X₁ comprises the amino acid sequence of SEQ ID NO: 148 and Z₁ comprises the amino acid of SEQ ID NO: 59, or fragment thereof. In some embodiments, X₁ comprises the amino acid sequence of SEQ ID NO: 148 and Z₁ comprises the amino acid of SEQ ID NO: 282, or fragment thereof.

In some embodiments, the polypeptide comprises a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 180 and Z₁ comprises IL-22, or a fragment thereof. In some embodiments, the polypeptide comprises a formula of X₁-Z₁, wherein X₁ comprises an amino acid sequence having at least 98% identity to the amino acid sequence of SEQ ID NO: 180 and Z₁ comprises IL-22, or a fragment thereof. In some embodiments, the polypeptide comprises a formula of X₁-Z₁, wherein X₁ comprises an amino acid sequence having the amino acid sequence of SEQ ID NO: 180 and Z₁ comprises IL-22, or a fragment thereof. In some embodiments, Z₁ comprises IL-22. In some embodiments, Z₁ comprises a fragment of IL-22.

In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 180 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 180 and Z₁ comprises an amino acid sequence of SEQ ID NO: 22, SEQ ID NO: 59, or SEQ ID NO: 282, or fragment thereof. In some embodiments, X₁ comprises an amino acid sequence having at least 98% identity to the amino acid sequence of SEQ ID NO: 180 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises an amino acid sequence having at least 98% identity to the amino acid sequence of SEQ ID NO: 180 and Z₁ comprises an amino acid sequence of SEQ ID NO: 22, SEQ ID NO: 59, or SEQ ID NO: 282, or fragment thereof. In some embodiments, X₁ comprises an amino acid sequence of SEQ ID NO: 180 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises the amino acid sequence of SEQ ID NO: 180 and Z₁ comprises the amino acid of SEQ ID NO: 22, SEQ ID NO: 59, or SEQ ID NO: 282, or fragment thereof. In some embodiments, X₁ comprises the amino acid sequence of SEQ ID NO: 180 and Z₁ comprises the amino acid of SEQ ID NO: 22, or fragment thereof. In some embodiments, X₁ comprises the amino acid sequence of SEQ ID NO: 180 and Z₁ comprises the amino acid of SEQ ID NO: 59, or fragment thereof. In some embodiments, X₁ comprises the amino acid sequence of SEQ ID NO: 180 and Z₁ comprises the amino acid of SEQ ID NO: 282, or fragment thereof.

In some embodiments, a polypeptide is provided. In some embodiments, the polypeptide comprises a formula of X₁-Z₁ wherein X₁ is a pre-protein signal peptide comprising the amino acid sequence of SEQ ID NO: 6; and Z₁ is a payload protein comprising an anti-TNFα antibody or an anti-TNFα antibody fragment. In some embodiments, Z₁ comprises an amino acid sequence selected from SEQ ID NO: 21, SEQ ID NO: 118, SEQ ID NO: 119, or SEQ ID NO: 120. In some embodiments, X₁ comprises the amino acid sequence of SEQ ID NO: 6, and Z₁ comprises the amino acid sequence of SEQ ID NO: 21. In some embodiments, X₁ comprises the amino acid sequence of SEQ ID NO: 6, and Z₁ comprises the amino acid sequence of SEQ ID NO: 118. In some embodiments, X₁ comprises the amino acid sequence of SEQ ID NO: 6, and Z₁ comprises the amino acid sequence of SEQ ID NO: 119. In some embodiments, X₁ comprises the amino acid sequence of SEQ ID NO: 6, and Z₁ comprises the amino acid sequence of SEQ ID NO: 120.

In some embodiments, a polypeptide is provided. In some embodiments, the polypeptide comprises a formula of X₁-Z₁ wherein X₁ is a pre-protein signal peptide comprising the amino acid sequence of SEQ ID NO: 7; and Z₁ is a payload protein comprising an anti-TNFα antibody or an anti-TNFα antibody fragment. In some embodiments, Z₁ comprises an amino acid sequence selected from SEQ ID NO: 21, SEQ ID NO: 118, SEQ ID NO: 119, or SEQ ID NO: 120. In some embodiments, X₁ comprises the amino acid sequence of SEQ ID NO: 7, and Z₁ comprises the amino acid sequence of SEQ ID NO: 21. In some embodiments, X₁ comprises the amino acid sequence of SEQ ID NO: 7, and Z₁ comprises the amino acid sequence of SEQ ID NO: 118. In some embodiments, X₁ comprises the amino acid sequence of SEQ ID NO: 7, and Z₁ comprises the amino acid sequence of SEQ ID NO: 119. In some embodiments, X₁ comprises the amino acid sequence of SEQ ID NO: 7, and Z₁ comprises the amino acid sequence of SEQ ID NO: 120.

In some embodiments, a polypeptide is provided. In some embodiments, the polypeptide comprises a formula of X₁-Z₁ wherein X₁ is a pre-protein signal peptide comprising the amino acid sequence of SEQ ID NO: 14; and Z₁ is a payload protein comprising an anti-TNFα antibody or an anti-TNFα antibody fragment. In some embodiments, Z₁ comprises an amino acid sequence selected from SEQ ID NO: 21, SEQ ID NO: 118, SEQ ID NO: 119, or SEQ ID NO: 120. In some embodiments, X₁ comprises the amino acid sequence of SEQ ID NO: 14, and Z₁ comprises the amino acid sequence of SEQ ID NO: 21. In some embodiments, X₁ comprises the amino acid sequence of SEQ ID NO: 14, and Z₁ comprises the amino acid sequence of SEQ ID NO: 118. In some embodiments, X₁ comprises the amino acid sequence of SEQ ID NO: 14, and Z₁ comprises the amino acid sequence of SEQ ID NO: 119. In some embodiments, X₁ comprises the amino acid sequence of SEQ ID NO: 14, and Z₁ comprises the amino acid sequence of SEQ ID NO: 120.

In some embodiments, a polypeptide is provided. In some embodiments, the polypeptide comprises a formula of X₁-Z₁ wherein X₁ is a pre-protein signal peptide comprising the amino acid sequence of SEQ ID NO: 6; and Z₁ is a payload protein comprising leptin. In some embodiments, Z₁ comprises the amino acid sequence of SEQ ID NO: 25. In some embodiments, X₁ comprises the amino acid sequence of SEQ ID NO: 6, and Z₁ comprises the amino acid sequence of SEQ ID NO: 25.

In some embodiments, a polypeptide is provided. In some embodiments, the polypeptide comprises a formula of X₁-Z₁ wherein X₁ is a pre-protein signal peptide comprising the amino acid sequence of SEQ ID NO: 7; and Z₁ is a payload protein comprising leptin. In some embodiments, Z₁ comprises the amino acid sequence of SEQ ID NO: 25. In some embodiments, X₁ comprises the amino acid sequence of SEQ ID NO: 7, and Z₁ comprises the amino acid sequence of SEQ ID NO: 25.

In some embodiments, a polypeptide is provided. In some embodiments, the polypeptide comprises a formula of X₁-Z₁ wherein X₁ is a pre-protein signal peptide comprising the amino acid sequence of SEQ ID NO: 14; and Z₁ is a payload protein comprising leptin. In some embodiments, Z₁ comprises the amino acid sequence of SEQ ID NO: 25. In some embodiments, X₁ comprises the amino acid sequence of SEQ ID NO: 14, and Z₁ comprises the amino acid sequence of SEQ ID NO: 25.

In some embodiments, a polypeptide is provided. In some embodiments, the polypeptide comprises a formula of X₁-Z₁ wherein X₁ is a pre-protein signal peptide comprising the amino acid sequence of SEQ ID NO: 17; and Z₁ is a payload protein comprising leptin. In some embodiments, Z₁ comprises the amino acid sequence of SEQ ID NO: 25. In some embodiments, X₁ comprises the amino acid sequence of SEQ ID NO: 17, and Z₁ comprises the amino acid sequence of SEQ ID NO: 25.

In some embodiments, a polypeptide is provided. In some embodiments, the polypeptide comprises a formula of X₁-Z₁ wherein X₁ is a pre-protein signal peptide comprising the amino acid sequence of SEQ ID NO: 18; and Z₁ is a payload protein comprising leptin. In some embodiments, Z₁ comprises the amino acid sequence of SEQ ID NO: 25. In some embodiments, X₁ comprises the amino acid sequence of SEQ ID NO: 18, and Z₁ comprises the amino acid sequence of SEQ ID NO: 25.

In some embodiments, a polypeptide is provided. In some embodiments, the polypeptide comprises a formula of X₁-Z₁ wherein X₁ is a pre-protein signal peptide comprising the amino acid sequence of SEQ ID NO: 19; and Z₁ is a payload protein comprising leptin. In some embodiments, Z₁ comprises the amino acid sequence of SEQ ID NO: 25. In some embodiments, X₁ comprises the amino acid sequence of SEQ ID NO: 19, and Z₁ comprises the amino acid sequence of SEQ ID NO: 25.

In some embodiments, a polypeptide is provided. In some embodiments, the polypeptide comprises a formula of X₁-Z₁ wherein X₁ is a pre-protein signal peptide comprising the amino acid sequence of SEQ ID NO: 47; and Z₁ is a payload protein comprising leptin. In some embodiments, Z₁ comprises the amino acid sequence of SEQ ID NO: 25. In some embodiments, X₁ comprises the amino acid sequence of SEQ ID NO: 47, and Z₁ comprises the amino acid sequence of SEQ ID NO: 25.

In some embodiments, a polypeptide is provided. In some embodiments, the polypeptide comprises a formula of X₁-Z₁ wherein X₁ is a pre-protein signal peptide comprising the amino acid sequence of SEQ ID NO: 48; and Z₁ is a payload protein comprising leptin. In some embodiments, Z₁ comprises the amino acid sequence of SEQ ID NO: 25. In some embodiments, X₁ comprises the amino acid sequence of SEQ ID NO: 48, and Z₁ comprises the amino acid sequence of SEQ ID NO: 25.

In some embodiments, a polypeptide is provided. In some embodiments, the polypeptide comprises a formula of X₁-Z₁ wherein X₁ is a pre-protein signal peptide comprising the amino acid sequence of SEQ ID NO: 148; and Z₁ is a payload protein comprising leptin. In some embodiments, Z₁ comprises the amino acid sequence of SEQ ID NO: 25. In some embodiments, X₁ comprises the amino acid sequence of SEQ ID NO: 148, and Z₁ comprises the amino acid sequence of SEQ ID NO: 25.

In some embodiments, a polypeptide is provided. In some embodiments, the polypeptide comprises a formula of X₁-Z₁ wherein X₁ is a pre-protein signal peptide comprising the amino acid sequence of SEQ ID NO: 180; and Z₁ is a payload protein comprising leptin. In some embodiments, Z₁ comprises the amino acid sequence of SEQ ID NO: 25. In some embodiments, X₁ comprises the amino acid sequence of SEQ ID NO: 180, and Z₁ comprises the amino acid sequence of SEQ ID NO: 25.

In some embodiments, a polypeptide is provided. In some embodiments, the polypeptide comprises a formula of X₁-Z₁ wherein X₁ is a pre-protein signal peptide comprising the amino acid sequence of SEQ ID NO: 20; and Z₁ is a payload protein comprising IL-10. In some embodiments, Z₁ comprises the amino acid sequence of SEQ ID NO: 24, SEQ ID NO: 283, SEQ ID NO: 284, or SEQ ID NO: 285. In some embodiments, X₁ comprises the amino acid sequence of SEQ ID NO: 20, and Z₁ comprises the amino acid sequence of SEQ ID NO: 24. In some embodiments, X₁ comprises the amino acid sequence of SEQ ID NO: 20, and Z₁ comprises the amino acid sequence of SEQ ID NO: 283. In some embodiments, X₁ comprises the amino acid sequence of SEQ ID NO: 20, and Z₁ comprises the amino acid sequence of SEQ ID NO: 284. In some embodiments, X₁ comprises the amino acid sequence of SEQ ID NO: 20, and Z₁ comprises the amino acid sequence of SEQ ID NO: 285.

In some embodiments, a polypeptide is provided. In some embodiments, the polypeptide comprises a formula of X₁-Z₁ wherein X₁ is a pre-protein signal peptide comprising the amino acid sequence of SEQ ID NO: 7; and Z₁ is a payload protein comprising IL-22. In some embodiments, Z₁ comprises the amino acid sequence of SEQ ID NO: 22, SEQ ID NO: 59, or SEQ ID NO: 282. In some embodiments, X₁ comprises the amino acid sequence of SEQ ID NO: 7, and Z₁ comprises the amino acid sequence of SEQ ID NO: 22. In some embodiments, X₁ comprises the amino acid sequence of SEQ ID NO: 7, and Z₁ comprises the amino acid sequence of SEQ ID NO: 59. In some embodiments, X₁ comprises the amino acid sequence of SEQ ID NO: 7, and Z₁ comprises the amino acid sequence of SEQ ID NO: 282.

In some embodiments, a polypeptide is provided. In some embodiments, the comprising the amino acid sequence of SEQ ID NO: 14; and Z₁ is a payload protein comprising IL-22. In some embodiments, Z₁ comprises the amino acid sequence of SEQ ID NO: 22, SEQ ID NO: 59, or SEQ ID NO: 282. In some embodiments, X₁ comprises the amino acid sequence of SEQ ID NO: 14, and Z₁ comprises the amino acid sequence of SEQ ID NO: 22. In some embodiments, X₁ comprises the amino acid sequence of SEQ ID NO: 14, and Z₁ comprises the amino acid sequence of SEQ ID NO: 59. In some embodiments, X₁ comprises the amino acid sequence of SEQ ID NO: 14, and Z₁ comprises the amino acid sequence of SEQ ID NO: 282.

In some embodiments, a polypeptide is provided. In some embodiments, the polypeptide comprises a formula of X₁-Z₁ wherein X₁ is a pre-protein signal peptide comprising the amino acid sequence of SEQ ID NO: 148; and Z₁ is a payload protein comprising IL-22. In some embodiments, Z₁ comprises the amino acid sequence of SEQ ID NO: 22, SEQ ID NO: 59, or SEQ ID NO: 282. In some embodiments, X₁ comprises the amino acid sequence of SEQ ID NO: 148, and Z₁ comprises the amino acid sequence of SEQ ID NO: 22. In some embodiments, X₁ comprises the amino acid sequence of SEQ ID NO: 148, and Z₁ comprises the amino acid sequence of SEQ ID NO: 59. In some embodiments, X₁ comprises the amino acid sequence of SEQ ID NO: 148, and Z₁ comprises the amino acid sequence of SEQ ID NO: 282.

In some embodiments, a polypeptide is provided. In some embodiments, the polypeptide comprises a formula of X₁-Z₁ wherein X₁ is a pre-protein signal peptide comprising the amino acid sequence of SEQ ID NO: 180; and Z₁ is a payload protein comprising IL-22. In some embodiments, Z₁ comprises the amino acid sequence of SEQ ID NO: 22, SEQ ID NO: 59, or SEQ ID NO: 282. In some embodiments, X₁ comprises the amino acid sequence of SEQ ID NO: 180, and Z₁ comprises the amino acid sequence of SEQ ID NO: 22. In some embodiments, X₁ comprises the amino acid sequence of SEQ ID NO: 180, and Z₁ comprises the amino acid sequence of SEQ ID NO: 59. In some embodiments, X₁ comprises the amino acid sequence of SEQ ID NO: 180, and Z₁ comprises the amino acid sequence of SEQ ID NO: 282.

In some embodiments, a polypeptide is provided. In some embodiments, the polypeptide comprises a formula of X₁-Z₁ wherein X₁ is a pre-protein signal peptide comprising the amino acid sequence of SEQ ID NO: 144; and Z₁ is a payload protein comprising IL-22. In some embodiments, the IL-22 is as provided for herein. In some embodiments, X₁ comprises the amino acid sequence of SEQ ID NO: 144; and Z₁ comprises the amino acid sequence of SEQ ID NO: 22. In some embodiments, a polypeptide is provided. In some embodiments, the polypeptide comprises a formula of X₁-Z₁ wherein X₁ is a pre-protein signal peptide comprising the amino acid sequence of SEQ ID NO: 145; and Z₁ is a payload protein comprising IL-22. In some embodiments, the IL-22 is as provided for herein. In some embodiments, X₁ comprises the amino acid sequence of SEQ ID NO: 145; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 146; and Z₁ is a payload protein comprising IL-22. In some embodiments, the IL-22 is as provided for herein. In some embodiments, X₁ comprises the amino acid sequence of SEQ ID NO: 146; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 147; and Z₁ is a payload protein comprising IL-22. In some embodiments, the IL-22 is as provided for herein. In some embodiments, X₁ comprises the amino acid sequence of SEQ ID NO: 147; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 149; and Z₁ is a payload protein comprising IL-22. In some embodiments, the IL-22 is as provided for herein. In some embodiments, X₁ comprises the amino acid sequence of SEQ ID NO: 149; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 150; and Z₁ is a payload protein comprising IL-22. In some embodiments, the IL-22 is as provided for herein. In some embodiments, X₁ comprises the amino acid sequence of SEQ ID NO: 150; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 151; and Z₁ is a payload protein comprising IL-22. In some embodiments, the IL-22 is as provided for herein. In some embodiments, X₁ comprises the amino acid sequence of SEQ ID NO: 151; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 152; and Z₁ is a payload protein comprising IL-22. In some embodiments, the IL-22 is as provided for herein. In some embodiments, X₁ comprises the amino acid sequence of SEQ ID NO: 152; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 153; and Z₁ is a payload protein comprising IL-22. In some embodiments, the IL-22 is as provided for herein. In some embodiments, X₁ comprises the amino acid sequence of SEQ ID NO: 153; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 154; and Z₁ is a payload protein comprising IL-22. In some embodiments, the IL-22 is as provided for herein. In some embodiments, X₁ comprises the amino acid sequence of SEQ ID NO: 154; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 155; and Z₁ is a payload protein comprising IL-22. In some embodiments, the IL-22 is as provided for herein. In some embodiments, X₁ comprises the amino acid sequence of SEQ ID NO: 155; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 156; and Z₁ is a payload protein comprising IL-22. In some embodiments, the IL-22 is as provided for herein. In some embodiments, X₁ comprises the amino acid sequence of SEQ ID NO: 156; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 157; and Z₁ is a payload protein comprising IL-22. In some embodiments, the IL-22 is as provided for herein. In some embodiments, X₁ comprises the amino acid sequence of SEQ ID NO: 157; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 158; and Z₁ is a payload protein comprising IL-22. In some embodiments, the IL-22 is as provided for herein. In some embodiments, X₁ comprises the amino acid sequence of SEQ ID NO: 158; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 159; and Z₁ is a payload protein comprising IL-22. In some embodiments, the IL-22 is as provided for herein. In some embodiments, X₁ comprises the amino acid sequence of SEQ ID NO: 159; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 160; and Z₁ is a payload protein comprising IL-22. In some embodiments, the IL-22 is as provided for herein. In some embodiments, X₁ comprises the amino acid sequence of SEQ ID NO: 160; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 161; and Z₁ is a payload protein comprising IL-22. In some embodiments, the IL-22 is as provided for herein. In some embodiments, X₁ comprises the amino acid sequence of SEQ ID NO: 161; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 162; and Z₁ is a payload protein comprising IL-22. In some embodiments, the IL-22 is as provided for herein. In some embodiments, X₁ comprises the amino acid sequence of SEQ ID NO: 162; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 163; and Z₁ is a payload protein comprising IL-22. In some embodiments, the IL-22 is as provided for herein. In some embodiments, X₁ comprises the amino acid sequence of SEQ ID NO: 163; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 164; and Z₁ is a payload protein comprising IL-22. In some embodiments, the IL-22 is as provided for herein. In some embodiments, X₁ comprises the amino acid sequence of SEQ ID NO: 164; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 165; and Z₁ is a payload protein comprising IL-22. In some embodiments, the IL-22 is as provided for herein. In some embodiments, X₁ comprises the amino acid sequence of SEQ ID NO: 165; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 166; and Z₁ is a payload protein comprising IL-22. In some embodiments, the IL-22 is as provided for herein. In some embodiments, X₁ comprises the amino acid sequence of SEQ ID NO: 166; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 167; and Z₁ is a payload protein comprising IL-22. In some embodiments, the IL-22 is as provided for herein. In some embodiments, X₁ comprises the amino acid sequence of SEQ ID NO: 167; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 168; and Z₁ is a payload protein comprising IL-22. In some embodiments, the IL-22 is as provided for herein. In some embodiments, X₁ comprises the amino acid sequence of SEQ ID NO: 168; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 169; and Z₁ is a payload protein comprising IL-22. In some embodiments, the IL-22 is as provided for herein. In some embodiments, X₁ comprises the amino acid sequence of SEQ ID NO: 169; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 170; and Z₁ is a payload protein comprising IL-22. In some embodiments, the IL-22 is as provided for herein. In some embodiments, X₁ comprises the amino acid sequence of SEQ ID NO: 170; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 171; and Z₁ is a payload protein comprising IL-22. In some embodiments, the IL-22 is as provided for herein. In some embodiments, X₁ comprises the amino acid sequence of SEQ ID NO: 171; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 172; and Z₁ is a payload protein comprising IL-22. In some embodiments, the IL-22 is as provided for herein. In some embodiments, X₁ comprises the amino acid sequence of SEQ ID NO: 172; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 173; and Z₁ is a payload protein comprising IL-22. In some embodiments, the IL-22 is as provided for herein. In some embodiments, X₁ comprises the amino acid sequence of SEQ ID NO: 173; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 174; and Z₁ is a payload protein comprising IL-22. In some embodiments, the IL-22 is as provided for herein. In some embodiments, X₁ comprises the amino acid sequence of SEQ ID NO: 174; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 175; and Z₁ is a payload protein comprising IL-22. In some embodiments, the IL-22 is as provided for herein. In some embodiments, X₁ comprises the amino acid sequence of SEQ ID NO: 175; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 176; and Z₁ is a payload protein comprising IL-22. In some embodiments, the IL-22 is as provided for herein. In some embodiments, X₁ comprises the amino acid sequence of SEQ ID NO: 176; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 177; and Z₁ is a payload protein comprising IL-22. In some embodiments, the IL-22 is as provided for herein. In some embodiments, X₁ comprises the amino acid sequence of SEQ ID NO: 177; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 178; and Z₁ is a payload protein comprising IL-22. In some embodiments, the IL-22 is as provided for herein. In some embodiments, X₁ comprises the amino acid sequence of SEQ ID NO: 178; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 179; and Z₁ is a payload protein comprising IL-22. In some embodiments, the IL-22 is as provided for herein. In some embodiments, X₁ comprises the amino acid sequence of SEQ ID NO: 179; and Z₁ comprises the amino acid sequence of SEQ ID NO: 22.

In some embodiments, a polypeptide is provided. In some embodiments, the comprising the amino acid sequence of SEQ ID NO: 181; and Z₁ is a payload protein comprising IL-22. In some embodiments, the IL-22 is as provided for herein. In some embodiments, X₁ comprises the amino acid sequence of SEQ ID NO: 181; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 182; and Z₁ is a payload protein comprising IL-22. In some embodiments, the IL-22 is as provided for herein. In some embodiments, X₁ comprises the amino acid sequence of SEQ ID NO: 182; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 183; and Z₁ is a payload protein comprising IL-22. In some embodiments, the IL-22 is as provided for herein. In some embodiments, X₁ comprises the amino acid sequence of SEQ ID NO: 183; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 184; and Z₁ is a payload protein comprising IL-22. In some embodiments, the IL-22 is as provided for herein. In some embodiments, X₁ comprises the amino acid sequence of SEQ ID NO: 184; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 185; and Z₁ is a payload protein comprising IL-22. In some embodiments, the IL-22 is as provided for herein. In some embodiments, X₁ comprises the amino acid sequence of SEQ ID NO: 185; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 186; and Z₁ is a payload protein comprising IL-22. In some embodiments, the IL-22 is as provided for herein. In some embodiments, X₁ comprises the amino acid sequence of SEQ ID NO: 186; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 187; and Z₁ is a payload protein comprising IL-22. In some embodiments, the IL-22 is as provided for herein. In some embodiments, X₁ comprises the amino acid sequence of SEQ ID NO: 187; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 188; and Z₁ is a payload protein comprising IL-22. In some embodiments, the IL-22 is as provided for herein. In some embodiments, X₁ comprises the amino acid sequence of SEQ ID NO: 188; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 189; and Z₁ is a payload protein comprising IL-22. In some embodiments, the IL-22 is as provided for herein. In some embodiments, X₁ comprises the amino acid sequence of SEQ ID NO: 189; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 190; and Z₁ is a payload protein comprising IL-22. In some embodiments, the IL-22 is as provided for herein. In some embodiments, X₁ comprises the amino acid sequence of SEQ ID NO: 190; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 191; and Z₁ is a payload protein comprising IL-22. In some embodiments, the IL-22 is as provided for herein. In some embodiments, X₁ comprises the amino acid sequence of SEQ ID NO: 191; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 192; and Z₁ is a payload protein comprising IL-22. In some embodiments, the IL-22 is as provided for herein. In some embodiments, X₁ comprises the amino acid sequence of SEQ ID NO: 192; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 193; and Z₁ is a payload protein comprising IL-22. In some embodiments, the IL-22 is as provided for herein. In some embodiments, X₁ comprises the amino acid sequence of SEQ ID NO: 193; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 194; and Z₁ is a payload protein comprising IL-22. In some embodiments, the IL-22 is as provided for herein. In some embodiments, X₁ comprises the amino acid sequence of SEQ ID NO: 194; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 195; and Z₁ is a payload protein comprising IL-22. In some embodiments, the IL-22 is as provided for herein. In some embodiments, X₁ comprises the amino acid sequence of SEQ ID NO: 195; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 196; and Z₁ is a payload protein comprising IL-22. In some embodiments, the IL-22 is as provided for herein. In some embodiments, X₁ comprises the amino acid sequence of SEQ ID NO: 196; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 197; and Z₁ is a payload protein comprising IL-22. In some embodiments, the IL-22 is as provided for herein. In some embodiments, X₁ comprises the amino acid sequence of SEQ ID NO: 197; and Z₁ comprises the amino acid sequence of SEQ ID NO: 22.

In some embodiments, a polypeptide is provided. In some embodiments, the comprising the amino acid sequence of SEQ ID NO: 198; and Z₁ is a payload protein comprising IL-22. In some embodiments, the IL-22 is as provided for herein. In some embodiments, X₁ comprises the amino acid sequence of SEQ ID NO: 198; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 199; and Z₁ is a payload protein comprising IL-22. In some embodiments, the IL-22 is as provided for herein. In some embodiments, X₁ comprises the amino acid sequence of SEQ ID NO: 199; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 200; and Z₁ is a payload protein comprising IL-22. In some embodiments, the IL-22 is as provided for herein. In some embodiments, X₁ comprises the amino acid sequence of SEQ ID NO: 200; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 201; and Z₁ is a payload protein comprising IL-22. In some embodiments, the IL-22 is as provided for herein. In some embodiments, X₁ comprises the amino acid sequence of SEQ ID NO: 201; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 202; and Z₁ is a payload protein comprising IL-22. In some embodiments, the IL-22 is as provided for herein. In some embodiments, X₁ comprises the amino acid sequence of SEQ ID NO: 202; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 203; and Z₁ is a payload protein comprising IL-22. In some embodiments, the IL-22 is as provided for herein. In some embodiments, X₁ comprises the amino acid sequence of SEQ ID NO: 203; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 204; and Z₁ is a payload protein comprising IL-22. In some embodiments, the IL-22 is as provided for herein. In some embodiments, X₁ comprises the amino acid sequence of SEQ ID NO: 204; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 205; and Z₁ is a payload protein comprising IL-22. In some embodiments, the IL-22 is as provided for herein. In some embodiments, X₁ comprises the amino acid sequence of SEQ ID NO: 205; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 206; and Z₁ is a payload protein comprising IL-22. In some embodiments, the IL-22 is as provided for herein. In some embodiments, X₁ comprises the amino acid sequence of SEQ ID NO: 206; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 207; and Z₁ is a payload protein comprising IL-22. In some embodiments, the IL-22 is as provided for herein. In some embodiments, X₁ comprises the amino acid sequence of SEQ ID NO: 207; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 208; and Z₁ is a payload protein comprising IL-22. In some embodiments, the IL-22 is as provided for herein. In some embodiments, X₁ comprises the amino acid sequence of SEQ ID NO: 208; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 209; and Z₁ is a payload protein comprising IL-22. In some embodiments, the IL-22 is as provided for herein. In some embodiments, X₁ comprises the amino acid sequence of SEQ ID NO: 209; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 210; and Z₁ is a payload protein comprising IL-22. In some embodiments, the IL-22 is as provided for herein. In some embodiments, X₁ comprises the amino acid sequence of SEQ ID NO: 210; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 211; and Z₁ is a payload protein comprising IL-22. In some embodiments, the IL-22 is as provided for herein. In some embodiments, X₁ comprises the amino acid sequence of SEQ ID NO: 211; and Z₁ comprises the amino acid sequence of SEQ ID NO: 22.

In some embodiments, a polypeptide is provided. In some embodiments, the polypeptide comprises a formula of X₁-Z₁ wherein X₁ is a pre-protein signal peptide comprising the amino acid sequence of SEQ ID NO: 212; and Z₁ is a payload protein comprising IL-22. In some embodiments, the IL-22 is as provided for herein. In some embodiments, X₁ comprises the amino acid sequence of SEQ ID NO: 212; and Z₁ comprises the amino acid sequence of SEQ ID NO: 22.

In some embodiments, X₁ and Z₁ are not linked by a linker polypeptide. In some embodiments, X₁ and Z₁ are linked by a linker polypeptide. In some embodiments, the linker polypeptide may be any appropriate linker polypeptide. In some embodiments, the linker polypeptide is selected from the group consisting of a flexible peptide linker, a rigid peptide linker, a cleavable peptide linker, or a non-cleavable peptide linker. In some embodiments, the linker polypeptide is a flexible peptide linker. In some embodiments, the linker polypeptide is a rigid peptide linker. In some embodiments, the linker polypeptide is a cleavable peptide linker. In some embodiments, the linker polypeptide is a non-cleavable peptide linker. Non-limiting exemplary linker polypeptides are provided in Table 4 below:

TABLE 4
Exemplary Linker polypeptides

| Type | Sequence | SEQ ID NO |
|---|---|---|
| Flexible | GGGGS | 27 |
| Flexible | (GGGGS)₃ | 28 |
| Flexible | (GGGGS)ₙ (n = 1, 2, 3, 4) | 29 |
| Flexible | (Gly)₈ | 30 |
| Flexible | (Gly)₆ | 31 |
| Rigid | (EAAAK)₃ | 32 |
| Rigid | (EAAK)ₙ (n = 1-3) | 33 |
| Rigid | A(EAAAK)₄ALEA(EAAAK)₄A | 34 |
| Rigid | AEAAAKEAAAKA | 35 |
| Rigid | PAPAP | 36 |
| Rigid | (Ala-Pro)ₙ (10-34 aa) | 37 |
| Cleavable | Disulfide | NA |
| Cleavable | VSQTSKLTRAETVFPDV | 38 |
| Cleavable | PLGLWA | 39 |
| Cleavable | RVLAEA | 40 |
| Cleavable | EDVVCCSMSY | 41 |
| Cleavable | GGIEGRGS | 42 |
| Cleavable | TRHRQPRGWE | 43 |
| Cleavable | AGNRVRRSVG | 44 |
| Cleavable | RRRRRRRRR | 45 |
| Cleavable | GFLG | 46 |
| Dipeptide | LE | NA |

### Nucleic Acid Molecules

In some embodiments, nucleic acid molecules are provided. In some embodiments, the nucleic acid molecules encode for the signal peptides provided for herein. In some embodiments, the nucleic acid molecules encode for the polypeptides comprising a formula of X₁-Z₁ as provided for herein.

In some embodiments, the nucleic acid molecule encoding for a signal peptide as provided for herein comprises a nucleic acid sequence as provided in Table 2. It is to be understood that the above recited sequence identifiers are exemplary only, and are not meant to be limiting in any way. Due to the degenerate nature of codons, those of skill in the art will readily understand that alternative nucleic acid molecules may encode for the same signal peptides. Accordingly, in some embodiments, the nucleic acid molecule encoding for a signal peptide as provided for herein comprises a sequence having at least 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100% identity to a nucleic acid sequence as provided in Table 2. In some embodiments, the nucleic acid molecule encoding for a signal peptide as provided for herein comprises a nucleic acid sequence of SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281. In some embodiments, the nucleic acid molecule encoding for a signal peptide as provided for herein comprises a sequence having at least 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100% identity to a nucleic acid sequence of SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

### Bacterium

In some embodiments, a bacterium is provided. In some embodiments, the bacterium comprises a nucleic acid molecule as provided for herein. In some embodiments, the bacterium comprises a nucleic acid molecule encoding for a polypeptide comprising a formula of X₁-Z₁, wherein X₁ is a pre-protein signal peptide as provided for herein and Z₁ is a payload protein as provided for herein. In some embodiments, X₁ comprises an amino acid sequence having at least 80%, 85%, 90%, 91%, 92%, 93%, 94%, 95%, 96%, 97%, 98%, 99%, or 100% identity to an amino acid sequence as provided in Table 1. In some embodiments, X₁ comprises an amino acid sequence having at least 98% identity to an amino acid sequence as provided in Table 1. In some embodiments, X₁ comprises an amino acid sequence as provided in Table 1. In some embodiments, X₁ comprises an amino acid sequence having at least 80%, 85%, 90%, 91%, 92%, 93%, 94%, 95%, 96%, 97%, 98%, 99%, or 100% identity to an amino acid sequence selected from SEQ ID NO: 6. SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180. In some embodiments, X₁ comprises an amino acid sequence having at least 98% identity to an amino acid sequence selected from SEQ ID NO: 6. SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180. In some embodiments, X₁ comprises an amino acid sequence selected from SEQ ID NO: 6. SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180.

In some embodiments, the polypeptide comprising the formula X₁-Z₁ is as provided for herein.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 6. In some embodiments, X₁ comprises an amino acid sequence having at least 98% identity to the amino acid sequence of SEQ ID NO: 6. In some embodiments, X₁ comprises the amino acid sequence of SEQ ID NO: 6.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 7. In some embodiments, X₁ comprises an amino acid sequence having at least 98% identity to the amino acid sequence of SEQ ID NO: 7. In some embodiments, X₁ comprises the amino acid sequence of SEQ ID NO: 7.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 14. In some embodiments, X₁ comprises an amino acid sequence having at least 98% identity to the amino acid sequence of SEQ ID NO: 14. In some embodiments, X₁ comprises the amino acid sequence of SEQ ID NO: 14.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 17. In some embodiments, X₁ comprises an amino acid sequence having at least 98% identity to the amino acid sequence of SEQ ID NO: 17. In some embodiments, X₁ comprises the amino acid sequence of SEQ ID NO: 17.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 18. In some embodiments, X₁ comprises an amino acid sequence having at least 98% identity to the amino acid sequence of SEQ ID NO: 18. In some embodiments, X₁ comprises the amino acid sequence of SEQ ID NO: 18.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 19. In some embodiments, X₁ comprises an amino acid sequence having at least 98% identity to the amino acid sequence of SEQ ID NO: 19. In some embodiments, X₁ comprises the amino acid sequence of SEQ ID NO: 19.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 20. In some embodiments, X₁ comprises an amino acid sequence having at least 98% identity to the amino acid sequence of SEQ ID NO: 20. In some embodiments, X₁ comprises the amino acid sequence of SEQ ID NO: 20.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 47. In some embodiments, X₁ comprises an amino acid sequence having at least 98% identity to the amino acid sequence of SEQ ID NO: 47. In some embodiments, X₁ comprises the amino acid sequence of SEQ ID NO: 47.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 48. In some embodiments, X₁ comprises an amino acid sequence having at least 98% identity to the amino acid sequence of SEQ ID NO: 48. In some embodiments, X₁ comprises the amino acid sequence of SEQ ID NO: 48.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 148. In some embodiments, X₁ comprises an amino acid sequence having at least 98% identity to the amino acid sequence of SEQ ID NO: 148. In some embodiments, X₁ comprises the amino acid sequence of SEQ ID NO: 148.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 180. In some embodiments, X₁ comprises an amino acid sequence having at least 98% identity to the amino acid sequence of SEQ ID NO: 180. In some embodiments, X₁ comprises the amino acid sequence of SEQ ID NO: 180.

In some embodiments, the bacterium comprises a nucleic acid molecule encoding for a polypeptide comprising a formula of X₁-Z₁, wherein Z₁ comprises an amino acid sequence having at least 80%, 85%, 90%, 91%, 92%, 93%, 94%, 95%, 96%, 97%, 98%, 99%, or 100% identity to an amino acid sequence as provided in Table 3. In some embodiments, the bacterium comprises a nucleic acid molecule encoding for a polypeptide comprising a formula of X₁-Z₁, wherein Z₁ comprises an amino acid sequence having at least 98% identity to an amino acid sequence as provided in Table 3. In some embodiments, the bacterium comprises a nucleic acid molecule encoding for a polypeptide comprising a formula of X₁-Z₁, wherein Z₁ comprises an amino acid sequence as provided in Table 3.

In some embodiments, the bacterium comprises a nucleic acid molecule encoding for a polypeptide comprising a formula of X₁-Z₁, wherein Z₁ is a therapeutic protein. In some embodiments Z₁ is a payload protein selected from the group consisting of antimicrobial peptides, Catestatin, CRAMP, hBD1, hBD3, β-Cateslytin, Dermaseptin, IL-10, IL-22, ClbS, TIGIT, TFF1, TFF2, TFF3, anti-CCL2 antibody, anti-CCL2 antibody fragments, anti-IL23 antibody, anti-IL23 antibody fragments, anti-TNFα antibody, anti-TNFα antibody fragments, GLP2, IGF1, leptin, and casein. In some embodiments Z₁ is a payload protein selected from the group consisting of anti-TNFα antibody, anti-TNFα antibody fragment, IL-10, IL-22, hBD3, leptin, and beta-casein. In some embodiments Z₁ is an anti-TNFα antibody. In some embodiments Z₁ is an anti-TNFα antibody fragment. In some embodiments Z₁ is IL-10. In some embodiments Z₁ is IL-22. In some embodiments Z₁ is hBD3. In some embodiments Z₁ is leptin. In some embodiments Z₁ is beta-casein.

In some embodiments, the bacterium comprises a nucleic acid molecule encoding for a polypeptide comprising a formula of X₁-Z₁, wherein X₁ is a pre-protein signal peptide as provided for herein and Z₁ is an anti-microbial peptide. In some embodiments, the anti-microbial peptide is any anti-microbial peptide. In some embodiments, the anti-microbial peptide is hBD1, hBD2, hBD3, b-Cateslytin, Dermaseptin, HD5, Reg3a, or C5a. In some embodiments, the anti-microbial peptide comprises an amino acid sequence having at least 80%, 85%, 90%, 91%, 92%, 93%, 94%, 95%, 96%, 97%, 98%, 99%, or 100% to an amino acid sequence selected from SEQ ID NO: 23, SEQ ID NO: 49, SEQ ID NO: 50, SEQ ID NO:
51, SEQ ID NO: 52, SEQ ID NO: 53, SEQ ID NO: 54, or SEQ ID NO: 55. In some embodiments, the bacterium comprises a nucleic acid molecule encoding for a polypeptide comprising a formula of X₁-Z₁, wherein X₁ is a pre-protein signal peptide as provided for herein and Z₁ is an anti-microbial peptide comprising an amino acid sequence having at least 80%, 85%, 90%, 91%, 92%, 93%, 94%, 95%, 96%, 97%, 98%, 99%, or 100% to an amino acid sequence selected from SEQ ID NO: 23, SEQ ID NO: 49, SEQ ID NO: 50, SEQ ID NO: 51, SEQ ID NO: 52, SEQ ID NO: 53, SEQ ID NO: 54, or SEQ ID NO: 55.

In some embodiments, the bacterium comprises a nucleic acid molecule encoding for a polypeptide comprising a formula of X₁-Z₁, wherein X₁ is a pre-protein signal peptide as provided for herein and Z₁ is an enzyme. In some embodiments, the enzyme is any enzyme. In some embodiments, the enzyme is Clbs, shP28GST, Clbs (Gilliamella apicola), Clbs (Snodgrassella alvi), Phytase, SUC2, SUIS, AnPep, beta-galactosidase (Lactobacillus), LACPL Exo-alpha-1,4-glucosidase, beta-galactosidase (yeast), ACE2, Xylose isomerase (RainFlu), Xylose isomerase (Lactobacillus), MaIL, apyrase, or DL-endopeptidase. In some embodiments, the enzyme comprises an amino acid sequence having at least 80%, 85%, 90%, 91%, 92%, 93%, 94%, 95%, 96%, 97%, 98%, 99%, or 100% to an amino acid sequence selected from SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, or SEQ ID NO: 78. In some embodiments, the bacterium comprises a nucleic acid molecule encoding for a polypeptide comprising a formula of X₁-Z₁, wherein X₁ is a pre-protein signal peptide as provided for herein and Z₁ is an enzyme comprising an amino acid sequence having at least 80%, 85%, 90%, 91%, 92%, 93%, 94%, 95%, 96%, 97%, 98%, 99%, or 100% to an amino acid sequence selected from SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, or SEQ ID NO: 78.

In some embodiments, the bacterium comprises a nucleic acid molecule encoding for a polypeptide comprising a formula of X₁-Z₁, wherein X₁ is a pre-protein signal peptide as provided for herein and Z₁ is a food protein. In some embodiments, the food protein is any food protein. In some embodiments, the food protein is beta-casein, alpha-casein, alpha casein derived peptides, Ara h1, or Ara h2. In some embodiments, the food protein comprises an amino acid sequence having at least 80%, 85%, 90%, 91%, 92%, 93%, 94%, 95%, 96%, 97%, 98%, 99%, or 100% to an amino acid sequence selected from SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 89, or SEQ ID NO: 90. In some embodiments, the bacterium comprises a nucleic acid molecule encoding for a polypeptide comprising a formula of X₁-Z₁, wherein X₁ is a pre-protein signal peptide as provided for herein and Z₁ is a food protein comprising an amino acid sequence having at least 80%, 85%, 90%, 91%, 92%, 93%, 94%, 95%, 96%, 97%, 98%, 99%, or 100% to an amino acid sequence selected from SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 89, or SEQ ID NO: 90.

In some embodiments, the bacterium comprises a nucleic acid molecule encoding for a polypeptide comprising a formula of X₁-Z₁, wherein X₁ is a pre-protein signal peptide as provided for herein and Z₁ is a growth factor. In some embodiments, the growth factor is any growth factor. In some embodiments, the growth factor is selected from IGF1, CTB-IGF1, or Elafin. In some embodiments, the growth factor comprises an amino acid sequence having at least 80%, 85%, 90%, 91%, 92%, 93%, 94%, 95%, 96%, 97%, 98%, 99%, or 100% to an amino acid sequence selected from SEQ ID NO: 91, SEQ ID NO: 92, or SEQ ID NO: 93. In some embodiments, the bacterium comprises a nucleic acid molecule encoding for a polypeptide comprising a formula of X₁-Z₁, wherein X₁ is a pre-protein signal peptide as provided for herein and Z₁ is a growth factor comprising an amino acid sequence having at least 80%, 85%, 90%, 91%, 92%, 93%, 94%, 95%, 96%, 97%, 98%, 99%, or 100% to an amino acid sequence selected from SEQ ID NO: 91, SEQ ID NO: 92, or SEQ ID NO: 93.

In some embodiments, the bacterium comprises a nucleic acid molecule encoding for a polypeptide comprising a formula of X₁-Z₁, wherein X₁ is a pre-protein signal peptide as provided for herein and Z₁ is an immunoreceptor. In some embodiments, the immunoreceptor is any immunoreceptor. In some embodiments, the immunoreceptor is TIGIT. In some embodiments, the immunoreceptor comprises an amino acid sequence having at least 80%, 85%, 90%, 91%, 92%, 93%, 94%, 95%, 96%, 97%, 98%, 99%, or 100% to an amino acid sequence of SEQ ID NO: 104. In some embodiments, the bacterium comprises a nucleic acid molecule encoding for a polypeptide comprising a formula of X₁-Z₁, wherein X₁ is a pre-protein signal peptide as provided for herein and Z₁ is an immunoreceptor comprising an amino acid sequence having at least 80%, 85%, 90%, 91%, 92%, 93%, 94%, 95%, 96%, 97%, 98%, 99%, or 100% to an amino acid sequence of SEQ ID NO: 104.

In some embodiments, the bacterium comprises a nucleic acid molecule encoding for a polypeptide comprising a formula of X₁-Z₁, wherein X₁ is a pre-protein signal peptide as provided for herein and Z₁ is a mucolytic. In some embodiments, the mucolytic is any mucolytic. In some embodiments, the mucolytic is DNAse1. In some embodiments, the mucolytic comprises an amino acid sequence having at least 80%, 85%, 90%, 91%, 92%, 93%, 94%, 95%, 96%, 97%, 98%, 99%, or 100% to an amino acid sequence of SEQ ID NO: 105. In some embodiments, the bacterium comprises a nucleic acid molecule encoding for a polypeptide comprising of X₁-Z₁, wherein X₁ is a pre-protein signal peptide as provided for herein and Z₁ is a mucolytic comprising an amino acid sequence having at least 80%, 85%, 90%, 91%, 92%, 93%, 94%, 95%, 96%, 97%, 98%, 99%, or 100% to an amino acid sequence of SEQ ID NO: 105.

In some embodiments, the bacterium comprises a nucleic acid molecule encoding for a polypeptide comprising a formula of X₁-Z₁, wherein X₁ is a pre-protein signal peptide as provided for herein and Z₁ is a peptide. In some embodiments, the peptide is any peptide. In some embodiments, the peptide is selected from andrenomedullin or urumin. In some embodiments, the peptide comprises an amino acid sequence having at least 80%, 85%, 90%, 91%, 92%, 93%, 94%, 95%, 96%, 97%, 98%, 99%, or 100% to an amino acid sequence of SEQ ID NO: 121 or SEQ ID NO: 122. In some embodiments, the bacterium comprises a nucleic acid molecule encoding for a polypeptide comprising a formula of X₁-Z₁, wherein X₁ is a pre-protein signal peptide as provided for herein and Z₁ is a peptide comprising an amino acid sequence having at least 80%, 85%, 90%, 91%, 92%, 93%, 94%, 95%, 96%, 97%, 98%, 99%, or 100% to an amino acid sequence of SEQ ID NO: 121 or SEQ ID NO: 122.

In some embodiments, the bacterium comprises a nucleic acid molecule encoding for a polypeptide comprising a formula of X₁-Z₁, wherein X₁ is a pre-protein signal peptide as provided for herein and Z₁ is a filament-associated protein. In some embodiments, the filament-associated protein is any filament-associate protein. In some embodiments, the filament-associated protein is filaggrin. In some embodiments, the filament-associated protein comprises an amino acid sequence having at least 80%, 85%, 90%, 91%, 92%, 93%, 94%, 95%, 96%, 97%, 98%, 99%, or 100% to an amino acid sequence of SEQ ID NO: 123. In some embodiments, the bacterium comprises a nucleic acid molecule encoding for a polypeptide comprising a formula of X₁-Z₁, wherein X₁ is a pre-protein signal peptide as provided for herein and Z₁ is a filament-associated protein comprising an amino acid sequence having at least 80%, 85%, 90%, 91%, 92%, 93%, 94%, 95%, 96%, 97%, 98%, 99%, or 100% to an amino acid sequence of SEQ ID NO: 123.

In some embodiments, the bacterium comprises a nucleic acid molecule encoding for a polypeptide comprising a formula of X₁-Z₁, wherein X₁ comprises an amino acid having at least 80%, 85%, 90%, 91%, 92%, 93%, 94%, 95%, 96%, 97%, 98%, 99%, or 100% identity to an amino acid sequence selected from SEQ ID NO: 6, SEQ ID NO: 7, or SEQ ID NO: 14, and Z₁ comprises a nanobody. In some embodiments, the nanobody can be any nanobody. In some embodiments, the nanobody binds to a target protein, wherein the target protein can be any protein. In some embodiments, the nanobody binds to a target protein, wherein the target protein is TNFα, CCL2, IL23, EGFR-III, GLP2, VEGFB, ILR6, MERS, SARS, BVD, Norovirus, RSV, TcdA, TcdB, PCSK9, Pseudomonas toxin, or IgE. In some embodiments, the nanobody comprises an amino acid sequence having at least 80%, 85%, 90%, 91%, 92%, 93%, 94%, 95%, 96%, 97%, 98%, 99%, or 100% to an amino acid sequence selected from SEQ ID NO: 21, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 126, SEQ ID NO: 127, SEQ ID NO: 128, SEQ ID NO: 129, SEQ ID NO: 130, or SEQ ID NO: 131. In some embodiments, the bacterium comprises a nucleic acid molecule encoding for a polypeptide comprising a formula of X₁-Z₁, wherein X₁ comprises an amino acid having at least 80%, 85%, 90%, 91%, 92%, 93%, 94%, 95%, 96%, 97%, 98%, 99%, or 100% identity to an amino acid sequence selected from SEQ ID NO: 6, SEQ ID NO: 7, or SEQ ID NO: 14, and Z₁ comprises a nanobody comprising an amino acid sequence having at least 80%, 85%, 90%, 91%, 92%, 93%, 94%, 95%, 96%, 97%, 98%, 99%, or 100% to an amino acid sequence selected from SEQ ID NO: 21, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 126, SEQ ID NO: 127, SEQ ID NO: 128, SEQ ID NO: 129, SEQ ID NO: 130, or SEQ ID NO: 131.

In some embodiments, the bacterium comprises a nucleic acid molecule encoding for a polypeptide comprising a formula of X₁-Z₁, wherein X₁ comprises an amino acid having at least 80%, 85%, 90%, 91%, 92%, 93%, 94%, 95%, 96%, 97%, 98%, 99%, or 100% identity to an amino acid sequence selected from SEQ ID NO: 17, SEQ ID NO: 18, SEQ ID NO: 19, SEQ ID NO: 47, or SEQ ID NO: 48, and Z₁ comprises a hormone. In some embodiments, the hormone is any hormone. In some embodiments, the hormone is GLP1, Exenatide-4, Catestatin, Leptin, Nesfatin, Linaclotide, Plecanatide, Uroguanylin, PYY, Guanylin, or LCRF. In some embodiments, the hormone comprises an amino acid sequence having at least 80%, 85%, 90%, 91%, 92%, 93%, 94%, 95%, 96%, 97%, 98%, 99%, or 100% to an amino acid sequence selected from SEQ ID NO: 25, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, or SEQ ID NO: 103. In some embodiments, the bacterium comprises a nucleic acid molecule encoding for a polypeptide comprising a formula of X₁-Z₁, wherein X₁ comprises an amino acid having at least 80%, 85%, 90%, 91%, 92%, 93%, 94%, 95%, 96%, 97%, 98%, 99%, or 100% identity to an amino acid sequence selected from SEQ ID NO: 17, SEQ ID NO: 18, SEQ ID NO: 19, SEQ ID NO: 47, or SEQ ID NO: 48, and Z₁ comprises a hormone comprising an amino acid sequence having at least 80%, 85%, 90%, 91%, 92%, 93%, 94%, 95%, 96%, 97%, 98%, 99%, or 100% to an amino acid sequence selected from SEQ ID NO: 25, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, or SEQ ID NO: 103.

In some embodiments, the bacterium comprises a nucleic acid molecule encoding for a polypeptide comprising a formula of X₁-Z₁, wherein X₁ comprises an amino acid having at least 80%, 85%, 90%, 91%, 92%, 93%, 94%, 95%, 96%, 97%, 98%, 99%, or 100% identity to an amino acid sequence of SEQ ID NO: 7, SEQ ID NO: 14, SEQ ID NO: 20, SEQ ID NO: 148, or SEQ ID NO: 180 and Z₁ comprises a cytokine. In some embodiments, the cytokine is any cytokine. In some embodiments, the cytokine is IL-10, TFF1, TFF2, TFF3, IL-22, or CTB-IL-22. In some embodiments, the cytokine comprises an amino acid sequence having at least 80%, 85%, 90%, 91%, 92%, 93%, 94%, 95%, 96%, 97%, 98%, 99%, or 100% to an amino acid sequence selected from SEQ ID NO: 22, SEQ ID NO: 24, SEQ ID NO: 56, SEQ ID NO: 57, SEQ ID NO: 58, SEQ ID NO: 59, SEQ ID NO: 282, SEQ ID NO: 283, SEQ ID NO: 284, or SEQ ID NO: 285. In some embodiments, the bacterium comprises a nucleic acid molecule encoding for a polypeptide comprising a formula of X₁-Z₁, wherein X₁ comprises an amino acid having at least 80%, 85%, 90%, 91%, 92%, 93%, 94%, 95%, 96%, 97%, 98%, 99%, or 100% identity to an amino acid sequence of SEQ ID NO: 7, SEQ ID NO: 14, SEQ ID NO: 20, SEQ ID NO: 148, or SEQ ID NO: 180 and Z₁ comprises a cytokine comprising an amino acid sequence having at least 80%, 85%, 90%, 91%, 92%, 93%, 94%, 95%, 96%, 97%, 98%, 99%, or 100% to an amino acid sequence selected from SEQ ID NO: 22, SEQ ID NO: 24, SEQ ID NO: 56, SEQ ID NO: 57, SEQ ID NO: 58, SEQ ID NO: 59, SEQ ID NO: 282, SEQ ID NO: 283, SEQ ID NO: 284, or SEQ ID NO: 285.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 6 and Z₁ comprises an anti-TNFα antibody, or an anti-TNFα antibody fragment. In some embodiments, the polypeptide comprises a formula of X₁-Z₁, wherein X₁ comprises an amino acid sequence having at least 98% identity to the amino acid sequence of SEQ ID NO: 6 and Z₁ comprises an anti-TNFαantibody, or an anti-TNFα antibody fragment. In some embodiments, the polypeptide comprises a formula of X₁-Z₁, wherein X₁ comprises an amino acid sequence having the amino acid sequence of SEQ ID NO: 6 and Z₁ comprises an anti-TNFα antibody, or an anti-TNFαantibody fragment. In some embodiments, Z₁ comprises an anti-TNFα antibody. In some embodiments, Z₁ comprises an anti-TNFα antibody fragment. In some embodiments, the anti-TNFα antibody is as provided for herein. In some embodiments, the anti-TNFα antibody fragment is a fragment of an anti-TNFα antibody as provided for herein.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 6 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, or SEQ ID NO: 120, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 6 and Z₁ comprises an amino acid sequence of SEQ ID NO: 21, SEQ ID NO: 118, SEQ ID NO: 119, or SEQ ID NO: 120, or fragment thereof. In some embodiments, X₁ comprises an amino acid sequence having at least 98% identity to the amino acid sequence of SEQ ID NO: 6 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, or SEQ ID NO: 120, or fragment thereof. In some embodiments, X₁ comprises an amino acid sequence having at least 98% identity to the amino acid sequence of SEQ ID NO: 6 and Z₁ comprises an amino acid sequence of SEQ ID NO: 21, SEQ ID NO: 118, SEQ ID NO: 119, or SEQ ID NO: 120, or fragment thereof. In some embodiments, X₁ comprises an amino acid sequence of SEQ ID NO: 6 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, or SEQ ID NO: 120, or fragment thereof. In some embodiments, X₁ comprises the amino acid sequence of SEQ ID NO: 6 and Z₁ comprises the amino acid of SEQ ID NO: 21, SEQ ID NO: 118, SEQ ID NO: 119, or SEQ ID NO: 120, or fragment thereof. In some embodiments, X₁ comprises the amino acid sequence of SEQ ID NO: 6 and Z₁ comprises the amino acid of SEQ ID NO: 21, or fragment thereof. In some embodiments, X₁ comprises the amino acid sequence of SEQ ID NO: 6 and Z₁ comprises the amino acid of SEQ ID NO: 118, or fragment thereof. In some embodiments, X₁ comprises the amino acid sequence of SEQ ID NO: 6 and Z₁ comprises the amino acid of SEQ ID NO: 119, or fragment thereof. In some embodiments, X₁ comprises the amino acid sequence of SEQ ID NO: 6 and Z₁ comprises the amino acid of SEQ ID NO: 120, or fragment thereof.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 7 and Z₁ comprises an anti-TNFα antibody, or an anti-TNFα antibody fragment. In some embodiments, the polypeptide comprises a formula of X₁-Z₁, wherein X₁ comprises an amino acid sequence having at least 98% identity to the amino acid sequence of SEQ ID NO: 7 and Z₁ comprises an anti-TNFαantibody, or an anti-TNFα antibody fragment. In some embodiments, the polypeptide comprises a formula of X₁-Z₁, wherein X₁ comprises an amino acid sequence having the amino acid sequence of SEQ ID NO: 7 and Z₁ comprises an anti-TNFα antibody, or an anti-TNFαantibody fragment. In some embodiments, Z₁ comprises an anti-TNFα antibody. In some embodiments, Z₁ comprises an anti-TNFα antibody fragment. In some embodiments, the anti-TNFα antibody is as provided for herein. In some embodiments, the anti-TNFα antibody fragment is a fragment of an anti-TNFα antibody as provided for herein.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 7 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, or SEQ ID NO: 120, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 7 and Z₁ comprises an amino acid sequence of SEQ ID NO: 21, SEQ ID NO: 118, SEQ ID NO: 119, or SEQ ID NO: 120, or fragment thereof. In some embodiments, X₁ comprises an amino acid sequence having at least 98% identity to the amino acid sequence of SEQ ID NO: 7 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, or SEQ ID NO: 120, or fragment thereof. In some embodiments, X₁ comprises an amino acid sequence having at least 98% identity to the amino acid sequence of SEQ ID NO: 7 and Z₁ comprises an amino acid sequence of SEQ ID NO: 21, SEQ ID NO: 118, SEQ ID NO: 119, or SEQ ID NO: 120, or fragment thereof. In some embodiments, X₁ comprises an amino acid sequence of SEQ ID NO: 7 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, or SEQ ID NO: 120, or fragment thereof. In some embodiments, X₁ comprises the amino acid sequence of SEQ ID NO: 7 and Z₁ comprises the amino acid of SEQ ID NO: 21, SEQ ID NO: 118, SEQ ID NO: 119, or SEQ ID NO: 120, or fragment thereof. In some embodiments, X₁ comprises the amino acid sequence of SEQ ID NO: 7 and Z₁ comprises the amino acid of SEQ ID NO: 21, or fragment thereof. In some embodiments, X₁ comprises the amino acid sequence of SEQ ID NO: 7 and Z₁ comprises the amino acid of SEQ ID NO: 118, or fragment thereof. In some embodiments, X₁ comprises the amino acid sequence of SEQ ID NO: 7 and Z₁ comprises the amino acid of SEQ ID NO: 119, or fragment thereof. In some embodiments, X₁ comprises the amino acid sequence of SEQ ID NO: 7 and Z₁ comprises the amino acid of SEQ ID NO: 120, or fragment thereof.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 14 and Z₁ comprises an anti-TNFα antibody, or an anti-TNFα antibody fragment. In some embodiments, the polypeptide comprises a formula of X₁-Z₁, wherein X₁ comprises an amino acid sequence having at least 98% identity to the amino acid sequence of SEQ ID NO: 14 and Z₁ comprises an anti-TNFα antibody, or an anti-TNFα antibody fragment. In some embodiments, the polypeptide comprises a formula of X₁-Z₁, wherein X₁ comprises an amino acid sequence having the amino acid sequence of SEQ ID NO: 14 and Z₁ comprises an anti-TNFα antibody, or an anti-TNFα antibody fragment. In some embodiments, Z₁ comprises an anti-TNFαantibody. In some embodiments, Z₁ comprises an anti-TNFα antibody fragment. In some embodiments, the anti-TNFα antibody is as provided for herein. In some embodiments, the anti-TNFα antibody fragment is a fragment of an anti-TNFα antibody as provided for herein.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 14 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, or SEQ ID NO: 120, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 14 and Z₁ comprises an amino acid sequence of SEQ ID NO: 21, SEQ ID NO: 118, SEQ ID NO: 119, or SEQ ID NO: 120, or fragment thereof. In some embodiments, X₁ comprises an amino acid sequence having at least 98% identity to the amino acid sequence of SEQ ID NO: 14 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, or SEQ ID NO: 120, or fragment thereof. In some embodiments, X₁ comprises an amino acid sequence having at least 98% identity to the amino acid sequence of SEQ ID NO: 14 and Z₁ comprises an amino acid sequence of SEQ ID NO: 21, SEQ ID NO: 118, SEQ ID NO: 119, or SEQ ID NO: 120, or fragment thereof. In some embodiments, X₁ comprises an amino acid sequence of SEQ ID NO: 14 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, or SEQ ID NO: 120, or fragment thereof. In some embodiments, X₁ comprises the amino acid sequence of SEQ ID NO: 14 and Z₁ comprises the amino acid of SEQ ID NO: 21, SEQ ID NO: 118, SEQ ID NO: 119, or SEQ ID NO: 120, or fragment thereof. In some embodiments, X₁ comprises the amino acid sequence of SEQ ID NO: 14 and Z₁ comprises the amino acid of SEQ ID NO: 21, or fragment thereof. In some embodiments, X₁ comprises the amino acid sequence of SEQ ID NO: 14 and Z₁ comprises the amino acid of SEQ ID NO: 118, or fragment thereof. In some embodiments, X₁ comprises the amino acid sequence of SEQ ID NO: 14 and Z₁ comprises the amino acid of SEQ ID NO: 119, or fragment thereof. In some embodiments, X₁ comprises the amino acid sequence of SEQ ID NO: 14 and Z₁ comprises the amino acid of SEQ ID NO: 120, or fragment thereof.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 17 and Z₁ comprises leptin, or a fragment thereof. In some embodiments, the polypeptide comprises a formula of X₁-Z₁, wherein X₁ comprises an amino acid sequence having at least 98% identity to the amino acid sequence of SEQ ID NO: 17 and Z₁ comprises leptin, or a fragment thereof. In some embodiments, the polypeptide comprises a formula of X₁-Z₁, wherein X₁ comprises an amino acid sequence having the amino acid sequence of SEQ ID NO: 17 and Z₁ comprises leptin, or a fragment thereof. In some embodiments, Z₁ comprises leptin. In some embodiments, Z₁ comprises a fragment of leptin.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 17 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 17 and Z₁ comprises an amino acid sequence of SEQ ID NO: 25, or fragment thereof. In some embodiments, X₁ comprises an amino acid sequence having at least 98% identity to the amino acid sequence of SEQ ID NO: 17 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 98% identity to the amino acid sequence of SEQ ID NO: 17 and Z₁ comprises an amino acid sequence of SEQ ID NO: 25, or fragment thereof. In some embodiments, X₁ comprises an amino acid sequence of SEQ ID NO: 17 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises the amino acid sequence of SEQ ID NO: 17 and Z₁ comprises the amino acid of SEQ ID NO: 25, or fragment thereof.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 18 and Z₁ comprises leptin, or a fragment thereof. In some embodiments, the polypeptide comprises a formula of X₁-Z₁, wherein X₁ comprises an amino acid sequence having at least 98% identity to the amino acid sequence of SEQ ID NO: 18 and Z₁ comprises leptin, or a fragment thereof. In some embodiments, the polypeptide comprises a formula of X₁-Z₁, wherein X₁ comprises an amino acid sequence having the amino acid sequence of SEQ ID NO: 18 and Z₁ comprises leptin, or a fragment thereof. In some embodiments, Z₁ comprises leptin. In some embodiments, Z₁ comprises a fragment of leptin.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 18 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 18 and Z₁ comprises an amino acid sequence of SEQ ID NO: 25, or fragment thereof. In some embodiments, X₁ comprises an amino acid sequence having at least 98% identity to the amino acid sequence of SEQ ID NO: 18 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 98% identity to the amino acid sequence of SEQ ID NO: 18 and Z₁ comprises an amino acid sequence of SEQ ID NO: 25, or fragment thereof. In some embodiments, X₁ comprises an amino acid sequence of SEQ ID NO: 18 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises the amino acid sequence of SEQ ID NO: 18 and Z₁ comprises the amino acid of SEQ ID NO: 25, or fragment thereof.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 19 and Z₁ comprises leptin, or a fragment thereof. In some embodiments, the polypeptide comprises a formula of X₁-Z₁, wherein X₁ comprises an amino acid sequence having at least 98% identity to the amino acid sequence of SEQ ID NO: 19 and Z₁ comprises leptin, or a fragment thereof. In some embodiments, the polypeptide comprises a formula of X₁-Z₁, wherein X₁ comprises an amino acid sequence having the amino acid sequence of SEQ ID NO: 19 and Z₁ comprises leptin, or a fragment thereof. In some embodiments, Z₁ comprises leptin. In some embodiments, Z₁ comprises a fragment of leptin.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 19 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 19 and Z₁ comprises an amino acid sequence of SEQ ID NO: 25, or fragment thereof. In some embodiments, X₁ comprises an amino acid sequence having at least 98% identity to the amino acid sequence of SEQ ID NO: 19 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 98% identity to the amino acid sequence of SEQ ID NO: 19 and Z₁ comprises an amino acid sequence of SEQ ID NO: 25, or fragment thereof. In some embodiments, X₁ comprises an amino acid sequence of SEQ ID NO: 19 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises the amino acid sequence of SEQ ID NO: 19 and Z₁ comprises the amino acid of SEQ ID NO: 25, or fragment thereof.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 47 and Z₁ comprises leptin, or a fragment thereof. In some embodiments, the polypeptide comprises a formula of X₁-Z₁, wherein X₁ comprises an amino acid sequence having at least 98% identity to the amino acid sequence of SEQ ID NO: 47 and Z₁ comprises leptin, or a fragment thereof. In some embodiments, the polypeptide comprises a formula of X₁-Z₁, wherein X₁ comprises an amino acid sequence having the amino acid sequence of SEQ ID NO: 47 and Z₁ comprises leptin, or a fragment thereof. In some embodiments, Z₁ comprises leptin. In some embodiments, Z₁ comprises a fragment of leptin.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 47 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 47 and Z₁ comprises an amino acid sequence of SEQ ID NO: 25, or fragment thereof. In some embodiments, X₁ comprises an amino acid sequence having at least 98% identity to the amino acid sequence of SEQ ID NO: 47 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 98% identity to the amino acid sequence of SEQ ID NO: 47 and Z₁ comprises an amino acid sequence of SEQ ID NO: 25, or fragment thereof. In some embodiments, X₁ comprises an amino acid of SEQ ID NO: 47 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises the amino acid sequence of SEQ ID NO: 47 and Z₁ comprises the amino acid of SEQ ID NO: 25, or fragment thereof.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 48 and Z₁ comprises leptin, or a fragment thereof. In some embodiments, the polypeptide comprises a formula of X₁-Z₁, wherein X₁ comprises an amino acid sequence having at least 98% identity to the amino acid sequence of SEQ ID NO: 48 and Z₁ comprises leptin, or a fragment thereof. In some embodiments, the polypeptide comprises a formula of X₁-Z₁, wherein X₁ comprises an amino acid sequence having the amino acid sequence of SEQ ID NO: 48 and Z₁ comprises leptin, or a fragment thereof. In some embodiments, Z₁ comprises leptin. In some embodiments, Z₁ comprises a fragment of leptin.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 48 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 48 and Z₁ comprises an amino acid sequence of SEQ ID NO: 25, or fragment thereof. In some embodiments, X₁ comprises an amino acid sequence having at least 98% identity to the amino acid sequence of SEQ ID NO: 48 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 98% identity to the amino acid sequence of SEQ ID NO: 48 and Z₁ comprises an amino acid sequence of SEQ ID NO: 25, or fragment thereof. In some embodiments, X₁ comprises an amino acid sequence of SEQ ID NO: 48 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises the amino acid sequence of SEQ ID NO: 48 and Z₁ comprises the amino acid of SEQ ID NO: 25, or fragment thereof.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 148 and Z₁ comprises leptin, or a fragment thereof. In some embodiments, the polypeptide comprises a formula of X₁-Z₁, wherein X₁ comprises an amino acid sequence having at least 98% identity to the amino acid sequence of SEQ ID NO: 148 and Z₁ comprises leptin, or a fragment thereof. In some embodiments, the polypeptide comprises a formula of X₁-Z₁, wherein X₁ comprises an amino acid sequence having the amino acid sequence of SEQ ID NO: 148 and Z₁ comprises leptin, or a fragment thereof. In some embodiments, Z₁ comprises leptin. In some embodiments, Z₁ comprises a fragment of leptin.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 148 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 148 and Z₁ comprises an amino acid sequence of SEQ ID NO: 25, or fragment thereof. In some embodiments, X₁ comprises an amino acid sequence having at least 98% identity to the amino acid sequence of SEQ ID NO: 148 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 98% identity to the amino acid sequence of SEQ ID NO: 148 and Z₁ comprises an amino acid sequence of SEQ ID NO: 25, or fragment thereof. In some embodiments, X₁ comprises an amino acid sequence of SEQ ID NO: 148 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises the amino acid sequence of SEQ ID NO: 148 and Z₁ comprises the amino acid of SEQ ID NO: 25, or fragment thereof.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 180 and Z₁ comprises leptin, or a fragment thereof. In some embodiments, the polypeptide comprises a formula of X₁-Z₁, wherein X₁ comprises an amino acid sequence having at least 98% identity to the amino acid sequence of SEQ ID NO: 180 and Z₁ comprises leptin, or a fragment thereof. In some embodiments, the polypeptide comprises a formula of X₁-Z₁, wherein X₁ comprises an amino acid sequence having the amino acid sequence of SEQ ID NO: 180 and Z₁ comprises leptin, or a fragment thereof. In some embodiments, Z₁ comprises leptin. In some embodiments, Z₁ comprises a fragment of leptin.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 180 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 180 and Z₁ comprises an amino acid sequence of SEQ ID NO: 25, or fragment thereof. In some embodiments, X₁ comprises an amino acid sequence having at least 98% identity to the amino acid sequence of SEQ ID NO: 180 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises an amino acid sequence having at least 98% identity to the amino acid sequence of SEQ ID NO: 180 and Z₁ comprises an amino acid sequence of SEQ ID NO: 25, or fragment thereof. In some embodiments, X₁ comprises an amino acid sequence of SEQ ID NO: 180 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25, or fragment thereof. In some embodiments, X₁ comprises the amino acid sequence of SEQ ID NO: 180 and Z₁ comprises the amino acid of SEQ ID NO: 25, or fragment thereof.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 20 and Z₁ comprises IL-10, or a fragment thereof. In some embodiments, the polypeptide comprises a formula of X₁-Z₁, wherein X₁ comprises an amino acid sequence having at least 98% identity to the amino acid sequence of SEQ ID NO: 20 and Z₁ comprises IL-10, or a fragment thereof. In some embodiments, the polypeptide comprises a formula of X₁-Z₁, wherein X₁ comprises an amino acid sequence having the amino acid sequence of SEQ ID NO: 20 and Z₁ comprises IL-10, or a fragment thereof. In some embodiments, Z₁ comprises IL-10. In some embodiments, Z₁ comprises a fragment of IL-10.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 20 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 24, SEQ ID NO: 283, SEQ ID NO: 284, or SEQ ID NO: 285, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 20 and Z₁ comprises an amino acid sequence of SEQ ID NO: 24, SEQ ID NO: 283, SEQ ID NO: 284, or SEQ ID NO: 285, or fragment thereof. In some embodiments, X₁ comprises an amino acid sequence having at least 98% identity to the amino acid sequence of SEQ ID NO: 20 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 24, SEQ ID NO: 283, SEQ ID NO: 284, or SEQ ID NO: 285, or fragment thereof. In some embodiments, X₁ comprises an amino acid sequence having at least 98% identity to the amino acid sequence of SEQ ID NO: 20 and Z₁ comprises an amino acid sequence of SEQ ID NO: 24, SEQ ID NO: 283, SEQ ID NO: 284, or SEQ ID NO: 285, or fragment thereof. In some embodiments, X₁ comprises an amino acid sequence of SEQ ID NO: 20 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 24, SEQ ID NO: 283, SEQ ID NO: 284, or SEQ ID NO: 285, or fragment thereof. In some embodiments, X₁ comprises the amino acid sequence of SEQ ID NO: 20 and Z₁ comprises the amino acid of SEQ ID NO: 24, SEQ ID NO: 283, SEQ ID NO: 284, or SEQ ID NO: 285, or fragment thereof. In some embodiments, X₁ comprises the amino acid sequence of SEQ ID NO: 20 and Z₁ comprises the amino acid sequence of SEQ ID NO: 24. In some embodiments, X₁ comprises the amino acid sequence of SEQ ID NO: 20 and Z₁ comprises the amino acid sequence of SEQ ID NO: 283. In some embodiments, X₁ comprises the amino acid sequence of SEQ ID NO: 20 and Z₁ comprises the amino acid sequence of SEQ ID NO: 284. In some embodiments, X₁ comprises the amino acid sequence of SEQ ID NO: 20 and Z₁ comprises the amino acid sequence of SEQ ID NO: 285.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 7 and Z₁ comprises IL-22, or a fragment thereof. In some embodiments, the polypeptide comprises a formula of X₁-Z₁, wherein X₁ comprises an amino acid sequence having at least 98% identity to the amino acid sequence of SEQ ID NO: 7 and Z₁ comprises IL-22, or a fragment thereof. In some embodiments, the polypeptide comprises a formula of X₁-Z₁, wherein X₁ comprises an amino acid sequence having the amino acid sequence of SEQ ID NO: 7 and Z₁ comprises IL-22, or a fragment thereof. In some embodiments, Z₁ comprises IL-22. In some embodiments, Z₁ comprises a fragment of IL-22.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 7 and Z comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 7 and Z₁ comprises an amino acid sequence of SEQ ID NO: 22, SEQ ID NO: 59, or SEQ ID NO: 282, or fragment thereof. In some embodiments, X₁ comprises an amino acid sequence having at least 98% identity to the amino acid sequence of SEQ ID NO: 7 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises an amino acid sequence having at least 98% identity to the amino acid sequence of SEQ ID NO: 7 and Z₁ comprises an amino acid sequence of SEQ ID NO: 22, SEQ ID NO: 59, or SEQ ID NO: 282, or fragment thereof. In some embodiments, X₁ comprises an amino acid sequence of SEQ ID NO: 7 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises the amino acid sequence of SEQ ID NO: 7 and Z₁ comprises the amino acid of SEQ ID NO: 22, SEQ ID NO: 59, or SEQ ID NO: 282, or fragment thereof. In some embodiments, X₁ comprises the amino acid sequence of SEQ ID NO: 7 and Z₁ comprises the amino acid sequence of SEQ ID NO: 22. In some embodiments, X₁ comprises the amino acid sequence of SEQ ID NO: 7 and Z₁ comprises the amino acid sequence of SEQ ID NO: 59. In some embodiments, X₁ comprises the amino acid sequence of SEQ ID NO: 7 and Z₁ comprises the amino acid sequence of SEQ ID NO: 282.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 14 and Z₁ comprises IL-22, or a fragment thereof. In some embodiments, the polypeptide comprises a formula of X₁-Z₁, wherein X₁ comprises an amino acid sequence having at least 98% identity to the amino acid sequence of SEQ ID NO: 14 and Z₁ comprises IL-22, or a fragment thereof. In some embodiments, the polypeptide comprises a formula of X₁-Z₁, wherein X₁ comprises an amino acid sequence having the amino acid sequence of SEQ ID NO: 14 and Z₁ comprises IL-22, or a fragment thereof. In some embodiments, Z₁ comprises IL-22. In some embodiments, Z₁ comprises a fragment of IL-22.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 14 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 14 and Z₁ comprises an amino acid sequence of SEQ ID NO: 22, SEQ ID NO: 59, or SEQ ID NO: 282, or fragment thereof. In some embodiments, X₁ comprises an amino acid sequence having at least 98% identity to the amino acid sequence of SEQ ID NO: 14 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises an amino acid sequence having at least 98% identity to the amino acid sequence of SEQ ID NO: 14 and Z₁ comprises an amino acid sequence of SEQ ID NO: 22, SEQ ID NO: 59, or SEQ ID NO: 282, or fragment thereof. In some embodiments, X₁ comprises an amino acid sequence of SEQ ID NO: 14 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises the amino acid sequence of SEQ ID NO: 14 and Z₁ comprises the amino acid of SEQ ID NO: 22, SEQ ID NO: 59, or SEQ ID NO: 282, or fragment thereof. In some embodiments, X₁ comprises the amino acid sequence of SEQ ID NO: 14 and Z₁ comprises the amino acid sequence of SEQ ID NO: 22. In some embodiments, X₁ comprises the amino acid sequence of SEQ ID NO: 14 and Z₁ comprises the amino acid sequence of SEQ ID NO: 59. In some embodiments, X₁ comprises the amino acid sequence of SEQ ID NO: 14 and Z₁ comprises the amino acid sequence of SEQ ID NO: 282.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 148 and Z₁ comprises IL-22, or a fragment thereof. In some embodiments, the polypeptide comprises a formula of X₁-Z₁, wherein X₁ comprises an amino acid sequence having at least 98% identity to the amino acid sequence of SEQ ID NO: 148 and Z₁ comprises IL-22, or a fragment thereof. In some embodiments, the polypeptide comprises a formula of X₁-Z₁, wherein X₁ comprises an amino acid sequence having the amino acid sequence of SEQ ID NO: 148 and Z₁ comprises IL-22, or a fragment thereof. In some embodiments, Z₁ comprises IL-22. In some embodiments, Z₁ comprises a fragment of IL-22.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 148 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 148 and Z₁ comprises an amino acid sequence of SEQ ID NO: 22, SEQ ID NO: 59, or SEQ ID NO: 282, or fragment thereof. In some embodiments, X₁ comprises an amino acid sequence having at least 98% identity to the amino acid sequence of SEQ ID NO: 148 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises an amino acid sequence having at least 98% identity to the amino acid sequence of SEQ ID NO: 148 and Z₁ comprises an amino acid sequence of SEQ ID NO: 22, SEQ ID NO: 59, or SEQ ID NO: 282, or fragment thereof. In some embodiments, X₁ comprises an amino acid sequence of SEQ ID NO: 148 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises the amino acid sequence of SEQ ID NO: 148 and Z₁ comprises the amino acid of SEQ ID NO: 22, SEQ ID NO: 59, or SEQ ID NO: 282, or fragment thereof. In some embodiments, X₁ comprises the amino acid sequence of SEQ ID NO: 148 and Z₁ comprises the amino acid sequence of SEQ ID NO: 22. In some embodiments, X₁ comprises the amino acid sequence of SEQ ID NO: 148 and Z₁ comprises the amino acid sequence of SEQ ID NO: 59. In some embodiments, X₁ comprises the amino acid sequence of SEQ ID NO: 148 and Z₁ comprises the amino acid sequence of SEQ ID NO: 282.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 180 and Z₁ comprises IL-22, or a fragment thereof. In some embodiments, the polypeptide comprises a formula of X₁-Z₁, wherein X₁ comprises an amino acid sequence having at least 98% identity to the amino acid sequence of SEQ ID NO: 180 and Z₁ comprises IL-22, or a fragment thereof. In some embodiments, the polypeptide comprises a formula of X₁-Z₁, wherein X₁ comprises an amino acid sequence having the amino acid sequence of SEQ ID NO: 180 and Z₁ comprises IL-22, or a fragment thereof. In some embodiments, Z₁ comprises IL-22. In some embodiments, Z₁ comprises a fragment of IL-22.

In some embodiments, the bacterium comprises a nucleic acid molecule encoding for a polypeptide comprising a formula of X₁-Z₁, wherein X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 180 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 180 and Z₁ comprises an amino acid sequence of SEQ ID NO: 22, SEQ ID NO: 59, or SEQ ID NO: 282, or fragment thereof. In some embodiments, X₁ comprises an amino acid sequence having at least 98% identity to the amino acid sequence of SEQ ID NO: 180 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises an amino acid sequence having at least 98% identity to the amino acid sequence of SEQ ID NO: 180 and Z₁ comprises an amino acid sequence of SEQ ID NO: 22, SEQ ID NO: 59, or SEQ ID NO: 282, or fragment thereof. In some embodiments, X₁ comprises an amino acid sequence of SEQ ID NO: 180 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282, or fragment thereof. In some embodiments, X₁ comprises the amino acid sequence of SEQ ID NO: 180 and Z₁ comprises the amino acid of SEQ ID NO: 22, SEQ ID NO: 59, OR SEQ ID NO: 282, or fragment thereof. In some embodiments, X₁ comprises the amino acid sequence of SEQ ID NO: 180 and Z₁ comprises the amino acid sequence of SEQ ID NO: 22. In some embodiments, X₁ comprises the amino acid sequence of SEQ ID NO: 180 and Z₁ comprises the amino acid sequence of SEQ ID NO: 59. In some embodiments, X₁ comprises the amino acid sequence of SEQ ID NO: 180 and Z₁ comprises the amino acid sequence of SEQ ID NO: 282.

In some embodiments, the bacterium is a bacterium as provided for herein. In some embodiments, the bacterium is a Lactobacillus bacterium as provided for herein. In some embodiments, the Lactobacillus is Lactobacillus acetotolerans, Lactobacillus acidifarinae, Lactobacillus acidipiscis, Lactobacillus acidophilus, Lactobacillus agilis, Lactobacillus algidus, Lactobacillus alimentarius, Lactobacillus alvei, Lactobacillus alvi, Lactobacillus amylolyticus, Lactobacillus amylophilus, Lactobacillus amylotrophicus, Lactobacillus amylovorus, Lactobacillus animalis, Lactobacillus animata, Lactobacillus antri, Lactobacillus apinorum, Lactobacillus apis, Lactobacillus apodemi, Lactobacillus aquaticus, Lactobacillus aviarius, Lactobacillus backii, Lactobacillus bifermentans, Lactobacillus bombi, Lactobacillus bombicola, Lactobacillus brantae, Lactobacillus brevis, Lactobacillus brevisimilis, Lactobacillus buchneri, Lactobacillus cacaonum, Lactobacillus camelliae, Lactobacillus capillatus, Lactobacillus casei, Lactobacillus paracasei, Lactobacillus zeae, Lactobacillus catenefornis, Lactobacillus ceti, Lactobacillus coleohominis, Lactobacillus colini, Lactobacillus collinoides, Lactobacillus composti, Lactobacillus concavus, Lactobacillus coryniformis, Lactobacillus crispatus, Lactobacillus crustorum, Lactobacillus curieae, Lactobacillus curvatus, Lactobacillus delbrueckii, Lactobacillus dextrinicus, Lactobacillus diolivorans, Lactobacillus equi, Lactobacillus equicursoris, Lactobacillus equigenerosi, Lactobacillus fabifermentans, Lactobacillus faecis, Lactobacillus faeni, Lactobacillus farciminis, Lactobacillus farraginis, Lactobacillus fermentum, Lactobacillus floricola, Lactobacillus florum, Lactobacillus formosensis, Lactobacillus fornicalis, Lactobacillus fructivorans, Lactobacillus frumenti, Lactobacillus fuchuensis, Lactobacillus furfuricola, Lactobacillus futsaii, Lactobacillus gallinarum, Lactobacillus gasseri, Lactobacillus gastricus, Lactobacillus ghanensis, Lactobacillus gigeriorum, Lactobacillus ginsenosidimutans, Lactobacillus gorillae, Lactobacillus graminis, Lactobacillus guizhouensis, Lactobacillus halophilus, Lactobacillus hammesii, Lactobacillus hamsteri, Lactobacillus harbinensis, Lactobacillus hayakitensis, Lactobacillus heilongjiangensis, Lactobacillus helsingborgensis, Lactobacillus helveticus, Lactobacillus herbarum, Lactobacillus heterohiochii, Lactobacillus hilgardii, Lactobacillus hokkaidonensis, Lactobacillus hominis, Lactobacillus homohiochii, Lactobacillus hordei, Lactobacillus iatae, Lactobacillus iners, Lactobacillus ingluviei, Lactobacillus insectis, Lactobacillus insicii, Lactobacillus intermedius, Lactobacillus intestinalis, Lactobacillus iwatensis, Lactobacillus ixorae, Lactobacillus japonicus, Lactobacillus jensenii, Lactobacillus johnsonii, Lactobacillus kalixensis, Lactobacillus kefiranofaciens, Lactobacillus kefiri, Lactobacillus kimbladii, Lactobacillus kimchicus, Lactobacillus kimchiensis, Lactobacillus kisonensis, Lactobacillus kitasatonis, Lactobacillus koreensis, Lactobacillus kullabergensis, Lactobacillus kunkeei, Lactobacillus larvae, Lactobacillus leichmannii, Lactobacillus letivazi, Lactobacillus lindneri, Lactobacillus malefermentans, Lactobacillus mali, Lactobacillus manihotivorans, Lactobacillus mellifer, Lactobacillus mellis, Lactobacillus melliventris, Lactobacillus micheneri, Lactobacillus mindensis, Lactobacillus mixtipabuli, Lactobacillus mobilis, Lactobacillus modestisalitolerans, Lactobacillus mucosae, Lactobacillus mudanjiangensis, Lactobacillus murinus, Lactobacillus nagelii, Lactobacillus namurensis, Lactobacillus nantensis, Lactobacillus nasuensis, Lactobacillus nenjiangensis, Lactobacillus nodensis, Lactobacillus odoratitofui, Lactobacillus oeni, Lactobacillus oligofermentans, Lactobacillus ori, Lactobacillus oryzae, Lactobacillus otakiensis, Lactobacillus ozensis, Lactobacillus panis, Lactobacillus pantheris, Lactobacillus parabrevis, Lactobacillus parabuchneri, Lactobacillus paracollinoides, Lactobacillus parafarraginis, Lactobacillus parakefiri, Lactobacillus paralimentarius, Lactobacillus paraplantarum, Lactobacillus pasteurii, Lactobacillus paucivorans, Lactobacillus pentosus, Lactobacillus perolens, Lactobacillus plajomi, Lactobacillus plantarum, Lactobacillus pobuzihii, Lactobacillus pontis, Lactobacillus porcinae, Lactobacillus psittaci, Lactobacillus rapi, Lactobacillus rennanquilfy, Lactobacillus rennini, Lactobacillus reuteri, Lactobacillus rhamnosus, Lactobacillus rodentium, Lactobacillus rogosae, Lactobacillus rossiae, Lactobacillus ruminis, Lactobacillus saerimneri, Lactobacillus sakei, Lactobacillus salivarius, Lactobacillus sanfranciscensis, Lactobacillus saniviri, Lactobacillus satsumensis, Lactobacillus secaliphilus, Lactobacillus selangorensis, Lactobacillus senioris, Lactobacillus senmaizukei, Lactobacillus sharpeae, Lactobacillus shenzhenensis, Lactobacillus sicerae, Lactobacillus silagei, Lactobacillus siliginis, Lactobacillus similis, Lactobacillus songhuajiangensis, Lactobacillus spicheri, Lactobacillus sucicola, Lactobacillus suebicus, Lactobacillus sunkii, Lactobacillus taiwanensis, Lactobacillus thailandensis, Lactobacillus tucceti, Lactobacillus ultunensis, Lactobacillus uvarum, Lactobacillus vaccinostercus, Lactobacillus vaginalis, Lactobacillus vermiforme, Lactobacillus vespulae, Lactobacillus vini, Lactobacillus wasatchensis, Lactobacillus xiangfangensis, Lactobacillus yonginensis, Lactobacillus zymae, Atopobium (e.g., A. deltae), Carnobacterium (e.g., C. alterfunditum), Weissella (e.g., W. beninensis), Oenococcus (e.g., O. oeni), Leuconostoc (e.g., L. carnosum), Pediococcus (e.g., P. acidilactici), Paralactobacillus (e.g., P. selangorensis), Holzapfelia (e.g., H. floricola), Amylolactobacillus (e.g., A. amylophilus), Bombilactobacillus (e.g., B. mellifer), Companilactobacillus (e.g., C. alimentarius), Lapidilactobacillus (e.g., L. concavus), Agrilactobacillus (e.g., A. composti), Schleiferilactobacillus (e.g., S. perolens), Loigolactobacillus (e.g., L. coryniformis), Lacticaseibacillus (e.g., L. casei), Latilactobacillus (e.g., L. sakei), Dellaglioa (e.g., D. algidus), Liquorilactobacillus (e.g., L. mail), Ligilactobacillus (e.g., L. salivarius), Lactiplantibacillus (e.g., L. plantarum), Furfurilactobacillus (e.g., F. rossiae), Paucilactobacillus (e.g., P. vaccinostercus), Limosilactobacillus (e.g., L. fermentum), Fructilactobacillus (e.g., F. fructivorans), Acetilactobacillus (e.g., A. jinshani), Apilactobacillus (e.g., A. kunkeei), Levilactobacillus (L. brevis), Secundilactobacillus (e.g., S. collinoides), or Lentilactobacillus (e.g., L. buchneri). In some embodiments, the bacterium is a Lactobacillus bacterium selected from L. gasseri, L. jensenii, L. crispatus, L. rhamnosus, L. casei, L. paracasei, L. acidophilus, L. delbruckii, L. johnsonii, L. fermentum, L. reuteri, and L. salivarus. In some embodiments, the Lactobacillus bacterium is L. gasseri. In some embodiments, the Lactobacillus bacterium is L. jensenii. In some embodiments, the Lactobacillus bacterium is L. crispatus. In some embodiments, the Lactobacillus bacterium is L. rhamnosus. In some embodiments, the Lactobacillus bacterium is L. casei. In some embodiments, the Lactobacillus bacterium is L. paracasei. In some embodiments, the Lactobacillus bacterium is L. acidophilus. In some embodiments, the Lactobacillus bacterium is L. delbruckii. In some embodiments, the Lactobacillus bacterium is L. johnsonii. In some embodiments, the Lactobacillus bacterium is L. fermentum. In some embodiments, the Lactobacillus bacterium is L. reuteri. In some embodiments, the Lactobacillus bacterium is L. salivarus. In some embodiments, the Lactobacillus bacterium is selected from L. gasseri and L. rhamnosus. In some embodiments, the Lactobacillus bacterium is L. gasseri. In some embodiments, the Lactobacillus bacterium is L. rhamnosus.

In some embodiments, a L. gasseri bacterium is provided. In some embodiments, the L. gasseri bacterium comprises a nucleic acid molecule as provided for herein. In some embodiments, the L. gasseri bacterium comprises a nucleic acid molecule encoding for a polypeptide comprising a formula of X₁-Z₁, wherein X₁ is a pre-protein signal peptide as provided for herein and Z₁ is a payload protein as provided for herein.

In some embodiments, a L. jensenii bacterium is provided. In some embodiments, the L. jensenii bacterium comprises a nucleic acid molecule as provided for herein. In some embodiments, the L. jensenii bacterium comprises a nucleic acid molecule encoding for a polypeptide comprising a formula of X₁-Z₁, wherein X₁ is a pre-protein signal peptide as provided for herein and Z₁ is a payload protein as provided for herein.

In some embodiments, a L. crispatus bacterium is provided. In some embodiments, the L. crispatus bacterium comprises a nucleic acid molecule as provided for herein. In some embodiments, the L. crispatus bacterium comprises a nucleic acid molecule encoding for a polypeptide comprising a formula of X₁-Z₁, wherein X₁ is a pre-protein signal peptide as provided for herein and Z₁ is a payload protein as provided for herein.

In some embodiments, a L. rhamnosus bacterium is provided. In some embodiments, the L. rhamnosus bacterium comprises a nucleic acid molecule as provided for herein. In some embodiments, L. rhamnosus bacterium comprises a nucleic acid molecule encoding for a polypeptide comprising a formula of X₁-Z₁, wherein X₁ is a pre-protein signal peptide as provided for herein and Z₁ is a payload protein as provided for herein.

In some embodiments, a L. casei bacterium is provided. In some embodiments, the L. casei bacterium comprises a nucleic acid molecule as provided for herein. In some embodiments, the L. casei bacterium comprises a nucleic acid molecule encoding for a polypeptide comprising a formula of X₁-Z₁, wherein X₁ is a pre-protein signal peptide as provided for herein and Z₁ is a payload protein as provided for herein.

In some embodiments, a L. paracasei bacterium is provided. In some embodiments, the L. paracasei bacterium comprises a nucleic acid molecule as provided for herein. In some embodiments, the L. paracasei bacterium comprises a nucleic acid molecule encoding for a polypeptide comprising a formula of X₁-Z₁, wherein X₁ is a pre-protein signal peptide as provided for herein and Z₁ is a payload protein as provided for herein.

In some embodiments, a L. acidophilus bacterium is provided. In some embodiments, the L. acidophilus bacterium comprises a nucleic acid molecule as provided for herein. In some embodiments, the L. acidophilus bacterium comprises a nucleic acid molecule encoding for a polypeptide comprising a formula of X₁-Z₁, wherein X₁ is a pre-protein signal peptide as provided for herein and Z₁ is a payload protein as provided for herein.

In some embodiments, a L. delbruckii bacterium is provided. In some embodiments, the L. delbruckii bacterium comprises a nucleic acid molecule as provided for herein. In some embodiments, the L. delbruckii bacterium comprises a nucleic acid molecule encoding for a polypeptide comprising a formula of X₁-Z₁, wherein X₁ is a pre-protein signal peptide as provided for herein and Z₁ is a payload protein as provided for herein.

In some embodiments, a L. johnsonii bacterium is provided. In some embodiments, the L. johnsonii bacterium comprises a nucleic acid molecule as provided for herein. In some embodiments, the L. johnsonii bacterium comprises a nucleic acid molecule encoding for a polypeptide comprising a formula of X₁-Z₁, wherein X₁ is a pre-protein signal peptide as provided for herein and Z₁ is a payload protein as provided for herein.

In some embodiments, a L. fermentum bacterium is provided. In some embodiments, the L. fermentum bacterium comprises a nucleic acid molecule as provided for herein. In some embodiments, the L. fermentum bacterium comprises a nucleic acid molecule encoding for a polypeptide comprising a formula of X₁-Z₁, wherein X₁ is a pre-protein signal peptide as provided for herein and Z₁ is a payload protein as provided for herein.

In some embodiments, a L. reuteri bacterium is provided. In some embodiments, the L. reuteri bacterium comprises a nucleic acid molecule as provided for herein. In some embodiments, the L. reuteri bacterium comprises a nucleic acid molecule encoding for a polypeptide comprising a formula of X₁-Z₁, wherein X₁ is a pre-protein signal peptide as provided for herein and Z₁ is a payload protein as provided for herein.

In some embodiments, a L. salivarus bacterium is provided. In some embodiments, the L. salivarus bacterium comprises a nucleic acid molecule as provided for herein. In some embodiments, the L. salivarus bacterium comprises a nucleic acid molecule encoding for a polypeptide comprising a formula of X₁-Z₁, wherein X₁ is a pre-protein signal peptide as provided for herein and Z₁ is a payload protein as provided for herein.

### Compositions

In another aspect, the present embodiments provide compositions, e.g., pharmaceutically acceptable compositions, which include the engineered microorganisms as provided for herein and which can, for example, be formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, local, topical, spinal or epidermal administration (e.g. by injection or infusion).

In any of the embodiments provided for herein, the pharmaceutical composition or composition comprises an engineered microorganism as provided for herein. In some embodiments, the engineered microorganism is any engineered microorganism as provided for herein. In some embodiments, the engineered microorganism is a Lactobacillus species as provided for herein. In some embodiments, the Lactobacillus is selected from L. gasseri, L. jensenii, L. crispatus, L. rhamnosus, L. casei, L. paracasei, L. acidophilus, L. delbruckii, L. johnsonii, L. fermentum, L. reuteri, and L. salivarus. In some embodiments, the Lactobacillus bacterium is L. gasseri. In some embodiments, the Lactobacillus bacterium is L. jensenii. In some embodiments, the Lactobacillus bacterium is L. crispatus. In some embodiments, the Lactobacillus bacterium is L. rhamnosus. In some embodiments, the Lactobacillus bacterium is L. casei. In some embodiments, the Lactobacillus bacterium is L. paracasei. In some embodiments, the Lactobacillus bacterium is L. acidophilus. In some embodiments, the Lactobacillus bacterium is L. delbruckii. In some embodiments, the Lactobacillus bacterium is L. johnsonii. In some embodiments, the Lactobacillus bacterium is L. fermentum. In some embodiments, the Lactobacillus bacterium is L. reuteri. In some embodiments, the Lactobacillus bacterium is L. salivarus. In some embodiments, the Lactobacillus bacterium is selected from L. gasseri and L. rhamnosus. In some embodiments, the Lactobacillus bacterium is L. gasseri. In some embodiments, the Lactobacillus bacterium is L. rhamnosus.

In some embodiments, the Lactobacillus bacterium of the composition comprises a nucleic acid molecule as provided for herein. In some embodiments, the Lactobacillus bacterium of the composition comprises a nucleic acid molecule encoding for a polypeptide comprising a formula of X₁-Z₁, wherein X₁ is a pre-protein signal peptide as provided for herein and Z₁ is a payload protein as provided for herein. In some embodiments, the L. gasseri bacterium of the composition comprises a nucleic acid molecule as provided for herein. In some embodiments, the L. gasseri bacterium of the composition comprises a nucleic acid molecule encoding for a polypeptide comprising a formula of X₁-Z₁, wherein X₁ is a pre-protein signal peptide as provided for herein and Z₁ is a payload protein as provided for herein. In some embodiments, the L. rhamnosus bacterium of the composition comprises a nucleic acid molecule as provided for herein. In some embodiments, the L. thamnosus bacterium of the composition comprises a nucleic acid molecule encoding for a polypeptide comprising a formula of X₁-Z₁, wherein X₁ is a pre-protein signal peptide as provided for herein and Z₁ is a payload protein as provided for herein. In some embodiments, X₁ comprises an amino acid sequence having at least 80%, 85%, 90%, 91%, 92%, 93%, 94%, 95%, 96%, 97%, 98%, 99%, or 100% identity to an amino acid sequence selected from SEQ ID NO: 6. SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180 as provided for herein. In some embodiments Z₁ is a payload protein selected from the group consisting of antimicrobial peptides, Catestatin, CRAMP, hBD1, hBD3, β-Cateslytin, Dermaseptin, IL-10, IL-22, ClbS, TIGIT, TFF1, TFF2, TFF3, anti-CCL2 antibody, anti-CCL2 antibody fragments, anti-IL23 antibody, anti-IL23 antibody fragments, anti-TNFα antibody, anti-TNFα antibody fragments, GLP2, IGF1, leptin, and casein as provided for herein. In some embodiments Z₁ is a payload protein selected from the group consisting of anti-TNFα antibody, anti-TNFαantibody fragment, IL-10, IL-22, hBD3, leptin, and beta-casein. In some embodiments Z₁ is an anti-TNFα antibody. In some embodiments Z₁ is an anti-TNFα antibody fragment. In some embodiments Z₁ is IL-10. In some embodiments Z₁ is IL-22. In some embodiments Z₁ is hBD3. In some embodiments Z₁ is leptin. In some embodiments Z₁ is beta-casein.

In some embodiments, the composition further comprises one or more pharmaceutically acceptable carriers or excipients. In some embodiments, the one or more pharmaceutically acceptable carriers or excipients include, but are not limited to, carriers, solvents, co-solvents, emulsifiers, lubricants, disintegrants, binders, fillers, glidants, rheology agents, solubilizers, antimicrobials, antioxidants, preservatives, colorants, flavor agents, emollients, pH modifiers, and the like. Specific carriers, solvents, co-solvents, emulsifiers, lubricants, disintegrants, binders, fillers, glidants, rheology agents, solubilizers, antimicrobials, antioxidants, preservatives, colorants, flavor agents, emollients, pH modifiers, and the like are well known in the art and any such specific carriers, solvents, co-solvents, emulsifiers, lubricants, disintegrants, binders, fillers, glidants, rheology agents, solubilizers, antimicrobials, antioxidants, preservatives, colorants, flavor agents, emollients, pH modifiers, and the like are within the scope of the present disclosure.

Pharmaceutically acceptable carriers useful in this disclosure are those conventionally known in the art. The nature of the carrier can depend on the particular mode of administration being employed. For instance, oral applications usually include pharmaceutically and physiologically acceptable fluids such as water, physiological saline, balanced salt solutions, aqueous dextrose, glycerol, or the like, as a vehicle. In addition to biologically-neutral carriers, oral compositions may also contain auxiliary substances, such as wetting or emulsifying agents, preservatives, and pH buffering agents, and the like. Exemplary pH modifiers include acids (e.g., acetic acid, adipic acid, carbonic acid, citric acid, fumaric acid, phosphoric acid, sorbic acid, succinic acid, tartaric acid), bases (e.g., magnesium oxide, tribasic potassium phosphate), and pharmaceutically acceptable salts thereof.

In some embodiments, the composition may further comprise one or more cosmetically acceptable excipients. The cosmetically acceptable excipient may be in addition to or in lieu of the pharmaceutically acceptable carriers or excipients. In some embodiments, the cosmetically acceptable excipient is selected from the group consisting of a preservative, an antioxidant, a chelator, a filler, a film former, a fragrance, a pH adjuster, and combinations thereof. In some embodiments, the cosmetically acceptable excipient is selected from the group consisting of hydroxyacetophenone, ethylhexylglycerin, sodium phytate, tromethamine, lactic acid, disodium phosphate, phenoxyethanol, xylose, HDI/trimethylol hexyllactone crosspolymer, lactose, ethylhexylglycerine, disodium EDTA, mineral salts, glucose, caprylyl glycol, silica, hexylene glycol, sodium benzoate, fragrance/parfum, BHT, hydrated silica, silica dimethyl silylate, sodium citrate, citric acid, lactic acid, sodium hydroxide, VP/polycarbamyl polygylcol ester, hydrolyzed sesame protein PG-propyl methylsilanediol, glucose, silica, tocopherol, limonene, linalool, isohexadecane, and combinations thereof. In some embodiments, the cosmetically acceptable excipient contains more than one excipient selected from hydroxyacetophenone, ethylhexylglycerin, sodium phytate, tromethamine, lactic acid, disodium phosphate, phenoxyethanol, xylose, HDI/trimethylol hexyllactone crosspolymer, lactose, ethylhexylglycerine, disodium EDTA, mineral salts, glucose, caprylyl glycol, silica, hexylene glycol, sodium benzoate, fragrance/parfum, BHT, hydrated silica, silica dimethyl silylate, sodium citrate, citric acid, lactic acid, sodium hydroxide, VP/polycarbamyl polygylcol ester, hydrolyzed sesame protein PG-propyl methylsilanediol, glucose, silica, tocopherol, limonene, linalool, or isohexadecane.

The compositions may be in a variety of forms. These include, for example, liquid, semi-solid and solid dosage forms, such as liquid solutions (e.g., injectable and infusible solutions), dispersions or suspensions, liposomes and suppositories. The form depends on the intended mode of administration and therapeutic application. Typical compositions are in the form of injectable or infusible solutions. In an embodiment the mode of administration is parenteral (e.g., intravenous, subcutaneous, intraperitoneal, intramuscular). In an embodiment, the composition is administered by intravenous infusion or injection. In some embodiments, the composition is administered by intramuscular or subcutaneous injection. In some embodiments, the composition is administered locally, e.g., by injection, or topical application, to a target site. For example, the pharmaceutical compositions can be lyophilized and reconstituted for use prior to administration to the patient.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high therapeutic molecule concentration. Sterile injectable solutions can be prepared by incorporating the active compound (i.e., therapeutic molecule) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation can be vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

As will be appreciated by the skilled artisan, the route and/or mode of administration will vary depending upon the desired results. In certain embodiments, the engineered microorganism may be prepared with a carrier that will protect the engineered microorganism against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In some embodiments, the pharmaceutical composition can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer composition by other than parenteral administration, it may be necessary to coat the composition with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

The therapeutically effective amount of engineered microorganism may be measured or specified in colony forming units (CFUs) and may be any amount, such as from about 100 CFUs to 1020 CFUs, about 103 to 1015 CFUs, 104 to 1010 CFUs, or about 102 to about 108 CFUs. In some embodiments, the therapeutically effective amount of engineered microorganism is from about 100 CFUs to about 1020 CFUs. In some embodiments, the therapeutically effective amount of engineered microorganism is from about 103 to about 1015 CFUs. In some embodiments, the therapeutically effective amount of engineered microorganism is from about 100 CFUs, about 103 CFUs, or about 104 CFUs to about 108 CFUs, about 1010 CFUs, about 1015 CFUs, or about 1020 CFUs. In some embodiments, the therapeutically effective amount of engineered microorganism is any amount of CFU that falls within any of the above ranges. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions may include a “therapeutically effective amount” or a “prophylactically effective amount” of the compositions or engineered microorganisms. A “therapeutically effective amount” refers to an amount effective, at dosages and for periods of time necessary, to achieve the desired therapeutic result. A therapeutically effective amount of a therapeutic molecule may vary according to factors such as the disease state, age, and weight of the individual, and the ability of the engineered microorganism to elicit a desired response in the individual. A therapeutically effective amount is also one in which any toxic or detrimental effects of an engineered microorganism is outweighed by the therapeutically beneficial effects. A “therapeutically effective dosage” preferably inhibits a measurable parameter, e.g., the percent content of a vaginal dysbiosis causing microbe, by at least about 20%, by at least about 40%, by at least about 60%, and by at least about 80% relative to untreated subjects. The ability of a composition or engineered microorganism to inhibit a measurable parameter, e.g., the percent content of a vaginal dysbiosis causing microbe, can be evaluated in an animal model system predictive of efficacy in reducing the percent content of a vaginal dysbiosis causing microbe. Alternatively, this property of a composition or engineered microorganism can be evaluated by examining the ability of the composition or engineered microorganism to inhibit the growth of a vaginal dysbiosis causing microbe in vitro, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount can be less than the therapeutically effective amount.

Also provided herein are kits comprising compositions as described herein. The kit can include one or more other elements including: instructions for use; other reagents, e.g., a label, a therapeutic agent, or an agent useful for chelating, or otherwise coupling, a therapeutic molecule to a label or other therapeutic agent, or a radioprotective composition; devices or other materials for preparing the therapeutic molecule for administration; pharmaceutically acceptable carriers; and devices or other materials for administration to a subject.

### Methods

In some embodiments, a method for producing a protein is provided. The protein produced may be any protein. In some embodiments, the protein is useful in the treatment of a disease or disorder. In some embodiments, the protein is a therapeutic protein. In some embodiments, the method comprises transfecting a bacterium with a nucleic acid molecule as provided for herein to produce a bacterium comprising the nucleic acid molecule; and culturing the bacteria comprising the nucleic acid molecule under conditions sufficient to grow the bacteria, wherein the bacteria expresses and secretes the payload protein . . .

In some embodiments, the nucleic acid molecule encodes for a polypeptide as provided for herein. In some embodiments, the nucleic acid molecule encodes for a polypeptide comprising the formula X₁-Z₁, wherein X₁ is a pre-protein signal peptide as provided for herein and Z₁ is a payload protein as provided for herein. In some embodiments, X₁ comprises an amino acid sequence having at least 80%, 85%, 90%, 91%, 92%, 93%, 94%, 95%, 96%, 97%, 98%, 99%, or 100% identity to an amino acid sequence selected from SEQ ID NO: 6. SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180 as provided for herein. In some embodiments Z₁ is a payload protein as provided for herein. In some embodiments Z₁ is a payload protein selected from the group consisting of antimicrobial peptides, Catestatin, CRAMP, hBD1, hBD3, β-Cateslytin, Dermaseptin, IL-10, IL-22, ClbS, TIGIT, TFF1, TFF2, TFF3, anti-CCL2 antibody, anti-CCL2 antibody fragments, anti-IL23 antibody, anti-IL23 antibody fragments, anti-TNFα antibody, anti-TNFα antibody fragments, GLP2, IGF1, leptin, and casein as provided for herein. In some embodiments Z₁ is a payload protein selected from the group consisting of anti-TNFα antibody, anti-TNFα antibody fragment, IL-10, IL-22, hBD3, leptin, and beta-casein. In some embodiments Z₁ is an anti-TNFα antibody. In some embodiments Z₁ is an anti-TNFα antibody fragment. In some embodiments Z₁ is IL-10. In some embodiments Z₁ is IL-22. In some embodiments Z₁ is hBD3. In some embodiments Z₁ is leptin. In some embodiments Z₁ is beta-casein.

In some embodiments, the method for producing a protein comprises transfecting a bacterium with a nucleic acid molecule encoding for a polypeptide as provided for herein to produce a bacterium comprising the nucleic acid molecule; and culturing the bacteria comprising the nucleic acid molecule under conditions sufficient to grow the bacteria, wherein the bacteria expresses and secretes the payload protein. In some embodiments, the polypeptide comprises the formula X₁-Z₁, wherein X₁ is a pre-protein signal peptide as provided for herein and Z₁ is a payload protein as provided for herein.

In some embodiments, the method for producing a protein comprises transfecting a bacterium with a nucleic acid molecule encoding for a polypeptide comprising the formula X₁-Z₁, wherein X₁ is a pre-protein signal peptide as provided for herein and Z₁ is a payload protein as provided for herein, to produce a bacterium comprising the nucleic acid molecule; and culturing the bacteria comprising the nucleic acid molecule under conditions sufficient to grow the bacteria, wherein the bacteria expresses and secretes the payload protein.

In some embodiments, the bacterium is as provided for herein. In some embodiments, the bacterium is a Lactobacillus bacterium as provided for herein. In some embodiments, the method for producing a therapeutic protein comprises transfecting a Lactobacillus with a nucleic acid molecule encoding for a polypeptide comprising the formula X₁-Z₁, wherein X₁ is a pre-protein signal peptide as provided for herein and Z₁ is a payload protein as provided for herein, to produce a Lactobacillus comprising the nucleic acid molecule; and culturing the Lactobacillus comprising the nucleic acid molecule under conditions sufficient to grow the Lactobacillus, wherein the Lactobacillus expresses and secretes the payload protein. In some embodiments, the Lactobacillus bacterium is selected from L. gasseri, L. jensenii, L. crispatus, L. rhamnosus, L. casei, L. paracasei, L. acidophilus, L. delbruckii, L. johnsonii, L. fermentum, L. reuteri, and L. salivarus. In some embodiments, the Lactobacillus bacterium is L. gasseri. In some embodiments, the Lactobacillus bacterium is L. jensenii. In some embodiments, the Lactobacillus bacterium is L. crispatus. In some embodiments, the Lactobacillus bacterium is L. rhamnosus. In some embodiments, the Lactobacillus bacterium is L. casei. In some embodiments, the Lactobacillus bacterium is L. paracasei. In some embodiments, the Lactobacillus bacterium is L. acidophilus. In some embodiments, the Lactobacillus bacterium is L. delbruckii. In some embodiments, the Lactobacillus bacterium is L. johnsonii. In some embodiments, the Lactobacillus bacterium is L. fermentum. In some embodiments, the Lactobacillus bacterium is L. reuteri. In some embodiments, the Lactobacillus bacterium is L. salivarus. In some embodiments, the Lactobacillus bacterium is selected from L. gasseri and L. rhamnosus. In some embodiments, the Lactobacillus bacterium is L. gasseri. In some embodiments, the Lactobacillus bacterium is L. rhamnosus . . .

In some embodiments, the bacteria may be cultured for a period of time in an environment effective to maintain the health of the bacteria, thereby generating a desired amount of therapeutic protein. The culturing of bacteria is common practice and well known in the art. In general, bacteria can be grown in nutrient-rich broth, which may comprise amino acids and nitrogen. Engineered bacteria may be grown for any amount of time necessary to generate the desired amount of recombinant payload protein comprising the signal peptide and payload protein. For example, the bacteria may be grown for about 0.5 hours to about 168 hours or longer. In some embodiments, bacteria may be grown for 0.5 h, 1 h, 2 h, 3 h, 4 h, 5 h, 6 h, 12 h, 18 h, 24 h, 30 h, 36 h, 42 h, 48 h, 72 h, 96 h, 120 h, 144 h, or 168 hours, or longer. In some embodiments, bacteria may be grown for any time period within any of the recited time periods or longer. Further, the bacteria may be grown in a continuous culture system, whereby a portion of a bacteria culture is seeded into fresh growth broth and the culture is continued. As such, in some embodiments, the bacteria may be grown for at least 0.5 hours. One of skill in the art will recognize that time of growth is a temperature dependent variable, as different temperatures produce different growth rates of bacteria, and as such growth of the bacteria for any time period is within the scope of the present application. Accordingly, engineered bacteria may be grown at room temperature or, more effectively, at a temperature of about 40° C. to 140° C., though any particular species and/or strain will have an optimal temperature range which will be known to one of ordinary skill in the art. Temperature may be used to control the growth of the bacteria and to control the production of the desired payload protein. Thus, in some embodiments, the bacteria may be cultured at a temperature of about 4° C. to about 140° C. The temperature range used in any of the embodiments herein can be any temperature range within the recited temperature range. Thus, in some embodiments, the bacteria may be cultured at a temperature of about 4° C. to about 140° C., from about 4° C. to about 80° C., from about 4° C. to about 40° C., from about 16° C. to about 40° C., from about 16° C. to about 60° C., from about 22° C. to about 37° C., from about 22° C. to about 45° C., from about 22° C. to about 140° C., and so on. Likewise, the recited temperature ranges include each and every individual temperature within said range. Thus, in some embodiments, the bacteria may be cultured at 4° C. In some embodiments, the bacteria may be cultured at 16° C. In some embodiments, the bacteria may be cultured at 22° C. In some embodiments, the bacteria may be cultured at 25° C. In some embodiments, the bacteria may be cultured at 30° C. In some embodiments, the bacteria may be cultured at 37° C. In some embodiments, the bacteria may be cultured at 4° C., 5° C., 10° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., or any temperature in between the recited temperatures. Further, those skilled in the art will recognize that further modifications to the growth conditions may be necessary depending on the strain of bacteria utilized and the therapeutic protein being produced. Such modifications are within the scope of the present application. Through various intracellular mechanisms, the payload protein will be extracellularly secreted with or without some or all of the synthetic pre-protein signal peptide to which it was fused. In any case, secretion of a payload protein by the host bacteria will result in its accumulation in the surrounding culture medium, where it may then be collected, isolated, and/or quantified. Accordingly, in some embodiments, the method of producing a therapeutic protein further comprises recovering or purifying the payload protein from the culture media. Techniques for the isolation and purification of payload proteins from the culture media are known in the art and any such method is within the scope of the present disclosure.

In some embodiments, the engineered bacteria may be grown in any volume of culture media. One of skill in the art will recognize that the volume of culture media necessary for bacteria growth will depend on the amount of payload protein desired to be produced. Accordingly, in some embodiments, the bacteria are cultured in a volume of about 0.005 L to about 1,000,000 L or more. In some embodiments, the bacteria are cultured in a volume of at least 0.005 L. In some embodiments, the bacteria are cultured in a volume of about 0.005 L, 0.05 L, 0.5 L, 1 L, 2 L, 3 L, 4L, 5 L, 10 L, 20 L, 30 L, 40 L, 50 L, 100 L, 1,000 L, 10,000 L, 100,000 L, or 1,000,000 L or greater. In some embodiments, the bacteria may be cultured at any volume in between any of the recited volumes or greater. Further, the bacteria may be grown in a continuous culture system, whereby a portion of a bacteria culture is seeded into fresh growth broth and the culture is continued. It is to be understood that the volumes recited are in not to be construed as limiting in any way, and that the bacteria may be grown in any volume that is appropriate for payload protein production.

In some embodiments, a method for producing a protein in a subject is provided. In some embodiments, the method comprises administering to the subject a bacterium as provided for herein. In some embodiments, the bacterium comprises a polypeptide as provided for herein. In some embodiments, the polypeptide comprises the formula X₁-Z₁, wherein X₁ is a pre-protein signal peptide as provided for herein and Z₁ is a payload protein as provided for herein. In some embodiments, X₁ comprises an amino acid sequence having at least 80%, 85%, 90%, 91%, 92%, 93%, 94%, 95%, 96%, 97%, 98%, 99%, or 100% identity to an amino acid sequence selected from SEQ ID NO: 6. SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180 as provided for herein. In some embodiments Z₁ is a payload protein as provided for herein. In some embodiments Z₁ is a payload protein selected from the group consisting of antimicrobial peptides, Catestatin, CRAMP, hBD1, hBD3, β-Cateslytin, Dermaseptin, IL-10, IL-22, ClbS, TIGIT, TFF1, TFF2, TFF3, anti-CCL2 antibody, anti-CCL2 antibody fragments, anti-IL23 antibody, anti-IL23 antibody fragments, anti-TNFα antibody, anti-TNFα antibody fragments, GLP2, IGF1, leptin, and casein as provided for herein. In some embodiments Z₁ is a payload protein selected from the group consisting of anti-TNFα antibody, anti-TNFα antibody fragment, IL-10, IL-22, hBD3, leptin, and beta-casein. In some embodiments Z₁ is an anti-TNFα antibody. In some embodiments Z₁ is an anti-TNFα antibody fragment. In some embodiments Z₁ is IL-10. In some embodiments Z₁ is IL-22. In some embodiments Z₁ is hBD3. In some embodiments Z₁ is leptin. In some embodiments Z₁ is beta-casein. In some embodiments, the bacteria are administered orally. In some embodiments, the bacteria are administered topically. The bacteria of the present disclosure are capable of surviving in the environment to which they are administered. Accordingly, the bacteria will continue to produce payload protein once administered to the subject, thereby producing the protein in the subject.

In some embodiments, a method for treating a disease or disorder in a subject in need thereof is provided. In some embodiments, the method comprises administering to the subject a therapeutically effective amount of a bacteria as provided for herein, or a pharmaceutical composition comprising bacteria as provided for herein. The bacteria or pharmaceutical composition comprising the same may be administered via any appropriate route known in the art. In some embodiments, the bacteria are administered orally or topically. In some embodiments the bacteria are administered orally. In some embodiments, the bacteria are administered topically. In some embodiments, the pharmaceutical composition is administered orally or topically. In some embodiments the pharmaceutical composition is administered orally. In some embodiments, the pharmaceutical composition is administered topically. In some embodiments, the bacteria comprises a nucleic acid molecule as provided for herein. In some embodiments, the bacteria comprises a nucleic acid molecule encoding for a polypeptide as provided for herein. In some embodiments, the bacteria comprises a nucleic acid molecule encoding for a polypeptide comprising the formula X₁-Z₁, wherein X₁ is a pre-protein signal peptide as provided for herein and Z₁ is a payload protein as provided for herein. In some embodiments, X₁ comprises an amino acid sequence having at least 80%, 85%, 90%, 91%, 92%, 93%, 94%, 95%, 96%, 97%, 98%, 99%, or 100% identity to an amino acid sequence selected from SEQ ID NO: 6. SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180 as provided for herein. In some embodiments, Z₁ is a payload protein as provided for herein. In some embodiments Z₁ is a payload protein selected from the group consisting of antimicrobial peptides, Catestatin, CRAMP, hBD1, hBD3, β-Cateslytin, Dermaseptin, IL-10, IL-22, ClbS, TIGIT, TFF1, TFF2, TFF3, anti-CCL2 antibody, anti-CCL2 antibody fragments, anti-IL23 antibody, anti-IL23 antibody fragments, anti-TNFα antibody, anti-TNFα antibody fragments, GLP2, IGF1, leptin, and casein as provided for herein. In some embodiments Z₁ is a payload protein selected from the group consisting of anti-TNFα antibody, anti-TNFα antibody fragment, IL-10, IL-22, hBD3, leptin, and beta-casein. In some embodiments Z₁ is an anti-TNFα antibody. In some embodiments Z₁ is an anti-TNFα antibody fragment. In some embodiments Z₁ is IL-10. In some embodiments Z₁ is IL-22. In some embodiments Z₁ is hBD3. In some embodiments Z₁ is leptin. In some embodiments Z₁ is beta-casein. In some embodiments, the bacterium is as provided for herein. In some embodiments, the bacterium is a Lactobacillus bacterium as provided for herein. In some embodiments, the Lactobacillus bacterium is selected from L. gasseri, L. jensenii, L. crispatus, L. rhamnosus, L. casei, L. paracasei, L. acidophilus, L. delbruckii, L. johnsonii, L. fermentum, L. reuteri, and L. salivarus. In some embodiments, the Lactobacillus bacterium is L. gasseri. In some embodiments, the Lactobacillus bacterium is L. jensenii. In some embodiments, the Lactobacillus bacterium is L. crispatus. In some embodiments, the Lactobacillus bacterium is L. rhamnosus. In some embodiments, the Lactobacillus bacterium is L. casei. In some embodiments, the Lactobacillus bacterium is L. paracasei. In some embodiments, the Lactobacillus bacterium is L. acidophilus. In some embodiments, the Lactobacillus bacterium is L. delbruckii. In some embodiments, the Lactobacillus bacterium is L. johnsonii. In some embodiments, the Lactobacillus bacterium is L. fermentum. In some embodiments, the Lactobacillus bacterium is L. reuteri. In some embodiments, the Lactobacillus bacterium is L. salivarus. In some embodiments, the Lactobacillus bacterium is selected from L. gasseri and L. rhamnosus. In some embodiments, the Lactobacillus bacterium is L. gasseri. In some embodiments, the Lactobacillus bacterium is L. rhamnosus.

In some embodiments, the method of treating a disease or disorder in a subject in need thereof comprises administering to the subject a therapeutically effective amount of a therapeutic protein produced by the bacteria provided for herein, or a pharmaceutical composition comprising the same.

In some embodiments, the disease or disorder is inflammation, chronic inflammation, colorectal cancer, periodontits, oral mucositis, infection, pathogenic E coli, inflammatory bowel disease, ulcerative colitis, Crohn's disease, type 2 diabetes, or obesity. In some embodiments, the disease or disorder is inflammation. In some embodiments, the disease or disorder is chronic inflammation. In some embodiments, the disease or disorder is colorectal cancer. In some embodiments, the disease or disorder is periodontits. In some embodiments, the disease or disorder is oral mucositis. In some embodiments, the disease or disorder is an infection. In some embodiments, the disease or disorder is pathogenic E coli. In some embodiments, the disease or disorder is inflammatory bowel disease. In some embodiments, the disease or disorder is ulcerative colitis. In some embodiments, the disease or disorder is Crohn's disease. In some embodiments, the disease or disorder is type 2 diabetes. In some embodiments, the disease or disorder is obesity.

In some embodiments, a method of treating inflammation in a subject in need thereof is provided. In some embodiments, the method comprises administering to the subject a therapeutically effective amount of a bacteria as provided for herein or a pharmaceutical composition as provided for herein. The bacteria or pharmaceutical composition comprising the same may be administered via any appropriate route known in the art. In some embodiments, the bacteria are administered orally or topically. In some embodiments the bacteria are administered orally. In some embodiments, the bacteria are administered topically. In some embodiments, the pharmaceutical composition is administered orally or topically. In some embodiments the pharmaceutical composition is administered orally. In some embodiments, the pharmaceutical composition is administered topically. In some embodiments, the bacteria comprises a nucleic acid molecule as provided for herein. In some embodiments, the bacteria comprises a nucleic acid molecule encoding for a polypeptide as provided for herein. In some embodiments, the bacteria comprises a nucleic acid molecule encoding for a polypeptide comprising the formula X₁-Z₁, wherein X₁ is a pre-protein signal peptide as provided for herein and Z₁ is a payload protein as provided for herein. In some embodiments, X₁ is a pre-protein signal peptide as provided for herein and Z₁ is selected from an anti-TNFα antibody, an anti-TNFα antibody fragment, or IL-10. In some embodiments, the anti-TNFα antibody is as provided for herein. In some embodiments, the anti-TNFα antibody fragment is a fragment of an anti-TNFα antibody is as provided for herein. In some embodiments, X₁ comprises an amino acid sequence having at least 80%, 85%, 86%, 87%, 88%, 89%, 90%, 91%, 92%, 93%, 94%, 95%, 96%, 97%, 98%, 99%, or 100% identity to an amino acid sequence selected from SEQ ID NO: 6, SEQ ID NO: 7, SEQ ID NO: 14, or SEQ ID NO: 20 and Z₁ comprises an amino acid sequence having at least 80%, 85%, 90%, 91%, 92%, 93%, 94%, 95%, 96%, 97%, 98%, 99%, or 100% identity to an amino acid sequence selected from SEQ ID NO: 21, SEQ ID NO: 24, SEQ ID NO: 118, SEQ ID NO: 119, or SEQ ID NO: 120, or a fragment thereof. In some embodiments, X₁ comprises an amino acid sequence having at least 98%, 99%, or 100% identity to an amino acid sequence selected from SEQ ID NO: 6, SEQ ID NO: 7, SEQ ID NO: 14, or SEQ ID NO: 20 and Z₁ comprises an amino acid sequence having at least 98%, 99%, or 100% identity to an amino acid sequence selected from SEQ ID NO: 21, SEQ ID NO: 24, SEQ ID NO: 118, SEQ ID NO: 119, or SEQ ID NO: 120, or a fragment thereof. In some embodiments, X₁ comprises an amino acid sequence selected from SEQ ID NO: 6, SEQ ID NO: 7, SEQ ID NO: 14, or SEQ ID NO: 20 and Z₁ comprises an amino acid sequence selected from SEQ ID NO: 21, SEQ ID NO: 24, SEQ ID NO: 118, SEQ ID NO: 119, or SEQ ID NO: 120, or a fragment thereof.

In some embodiments, X₁ comprises an amino acid sequence having at least 90%, 91%, 92%, 93%, 94%, 95%, 96%, 97%, 98%, 99%, or 100% identity to an amino acid sequence selected from SEQ ID NO: 6, SEQ ID NO: 7, or SEQ ID NO: 14, and Z₁ comprises an amino acid sequence having at least 90%, 91%, 92%, 93%, 94%, 95%, 96%, 97%, 98%, 99%, or 100% identity to an amino acid sequence selected from SEQ ID NO: 21, SEQ ID NO: 118, SEQ ID NO: 119, or SEQ ID NO: 120, or a fragment thereof. In some embodiments, X₁ comprises an amino acid sequence selected from SEQ ID NO: 6, SEQ ID NO: 7, or SEQ ID NO: 14, and Z₁ comprises an amino acid sequence selected from SEQ ID NO: 21, SEQ ID NO: 118, SEQ ID NO: 119, or SEQ ID NO: 120, or a fragment thereof. In some embodiments, X₁ comprises an amino acid sequence having at least 90%, 91%, 92%, 93%, 94%, 95%, 96%, 97%, 98%, 99%, or 100% identity to the amino acid sequence of SEQ ID NO: 20, and Z₁ comprises an amino acid sequence having at least 90%, 91%, 92%, 93%, 94%, 95%, 96%, 97%, 98%, 99%, or 100% identity to the amino acid sequence of SEQ ID NO: 24, or a fragment thereof. In some embodiments, X₁ comprises the amino acid sequence of SEQ ID NO: 20, and Z₁ comprises the amino acid sequence of SEQ ID NO: 24, or a fragment thereof.

In some embodiments, a method of treating obesity in a subject in need thereof is provided. In some embodiments, the method comprises administering to the subject a therapeutically effective amount of a bacteria as provided for herein or a pharmaceutical composition as provided for herein. The bacteria or pharmaceutical composition comprising the same may be administered via any appropriate route known in the art. In some embodiments, the bacteria are administered orally or topically. In some embodiments the bacteria are administered orally. In some embodiments, the bacteria are administered topically. In some embodiments, the pharmaceutical composition is administered orally or topically. In some embodiments the pharmaceutical composition is administered orally. In some embodiments, the pharmaceutical composition is administered topically. In some embodiments, the bacteria comprises a nucleic acid molecule as provided for herein. In some embodiments, the bacteria comprises a nucleic acid molecule encoding for a polypeptide as provided for herein. In some embodiments, the bacteria comprises a nucleic acid molecule encoding for a polypeptide comprising the formula X₁-Z₁, wherein X₁ is a pre-protein signal peptide as provided for herein and Z₁ is a payload protein as provided for herein. In some embodiments, X₁ is a pre-protein signal peptide as provided for herein and Z₁ is leptin or nesfatin. In some embodiments, the amino acid sequences of leptin and nesfatin are as provided for herein. In some embodiments, X₁ comprises an amino acid sequence having at least 80%, 85%, 86%, 87%, 88%, 89%, 90%, 91%, 92%, 93%, 94%, 95%, 96%, 97%, 98%, 99%, or 100% identity to an amino acid sequence selected from SEQ ID NO: 17, SEQ ID NO: 18, SEQ ID NO: 19, SEQ ID NO: 47, SEQ ID NO: 48, SEQ ID NO: 148, or SEQ ID NO: 180, and Z₁ comprises an amino acid sequence having at least 80%, 85%, 90%, 91%, 92%, 93%, 94%, 95%, 96%, 97%, 98%, 99%, or 100% identity to the amino acid sequence of SEQ ID NO: 25, or a fragment thereof. In some embodiments, X₁ comprises an amino acid sequence having at least 98%, 99%, or 100% identity to an amino acid sequence selected from SEQ ID NO: 17, SEQ ID NO: 18, SEQ ID NO: 19, SEQ ID NO: 47, SEQ ID NO: 48, SEQ ID NO: 148, or SEQ ID NO: 180, and Z₁ comprises an amino acid sequence having at least 98%, 99%, or 100% identity to the amino acid sequence of SEQ ID NO: 25, or a fragment thereof. In some embodiments, X₁ comprises an amino acid sequence selected from SEQ ID NO: 17, SEQ ID NO: 18, SEQ ID NO: 19, SEQ ID NO: 47, SEQ ID NO: 48, SEQ ID NO: 148, or SEQ ID NO: 180, and Z₁ comprises the amino acid sequence of SEQ ID NO: 25, or a fragment thereof.

In some embodiments, a method of treating a disease or disorder is provided, the method comprising administering to the subject a therapeutically effective amount of a bacteria as provided for herein or a pharmaceutical composition as provided for herein, wherein the bacteria comprises a nucleic acid molecule encoding for a polypeptide comprising the formula X₁-Z₁, wherein X₁ is a pre-protein signal peptide comprising an amino acid sequence having at least 80%, 85%, 86%, 87%, 88%, 89%, 90%, 91%, 92%, 93%, 94%, 95%, 96%, 97%, 98%, 99%, or 100% identity to an amino acid sequence selected from SEQ ID NO: 17, SEQ ID NO: 18, SEQ ID NO: 19, SEQ ID NO: 47, SEQ ID NO: 48, SEQ ID NO: 148, or SEQ ID NO: 180, and Z₁ is leptin or nesfatin. In some embodiments, Z₁ comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 25. In some embodiments, the disease or disorder is selected from obesity, diabetes, insulin resistance, hyperglycemia, hyperinsulinemia, lipodystrophy, hypertriglyceridemia, systemic lupus erythematosus, hepatic steatosis, low bone density, hypothalamic hypogonadisum, infertility, or hypothalamic amenorrhea.

In some embodiments, a method of treating a disease or disorder is provided, the method comprising administering to the subject a therapeutically effective amount of a bacteria as provided for herein or a pharmaceutical composition as provided for herein, wherein the bacteria comprises a nucleic acid molecule encoding for a polypeptide comprising the formula X₁-Z₁, wherein X₁ is a pre-protein signal peptide comprising an amino acid sequence having at least 80%, 85%, 86%, 87%, 88%, 89%, 90%, 91%, 92%, 93%, 94%, 95%, 96%, 97%, 98%, 99%, or 100% identity to an amino acid sequence selected from SEQ ID NO: 20, and Z₁ is IL-10 In some embodiments, Z₁ comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 24, SEQ ID NO: 283, SEQ ID NO: 284, or SEQ ID NO: 285. In some embodiments, the disease or disorder is selected from an inflammatory disease, autoimmune disease, cancer, non-alcoholic fatty liver disease (NAFLD), non-alcoholic steatohepatits (NASH), infection, sepsis, or a growth hormone disorder.

### Enumerated Embodiments

In some embodiments, the following embodiments are provided:
1. A pre-protein signal peptide comprising an amino acid sequence that has at least 95%, 96%, 97%, 98%, 99%, or 100% identity to an amino acid sequence selected from SEQ ID NO: 6, SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180.
2. The pre-protein signal peptide of embodiment 1, wherein the pre-protein signal peptide has at least 95% identity to an amino acid sequence selected from SEQ ID NO: 6, SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180.
3. The pre-protein signal peptide of embodiment 1, wherein the pre-protein signal peptide has at least 98% identity to an amino acid sequence selected from SEQ ID NO: 6, SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180.
4. The pre-protein signal peptide of embodiment 1, wherein the pre-protein signal peptide has an amino acid sequence selected from SEQ ID NO: 6, SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180.5. A polypeptide comprising a formula of X₁-Z₁ wherein:
   X₁ is a pre-protein signal peptide; and
   Z₁ is a payload protein.
6. The polypeptide of embodiment 5, wherein X₁ comprises an amino acid sequence that has at least 95%, 96%, 97%, 98%, 99%, or 100% identity to an amino acid sequence selected from SEQ ID NO: 6, SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180.
7. The polypeptide of embodiment 5 or embodiment 6, wherein Z₁ is selected from the group consisting of antimicrobial peptides, Catestatin, CRAMP, hBD1, hBD3, β-Cateslytin, Dermaseptin, IL-10, IL-22, ClbS, TIGIT, TFF1, TFF2, TFF3, anti-CCL2 antibody, anti-CCL2 antibody fragments, anti-IL23 antibody, anti-IL23 antibody fragments, anti-TNFαantibody, anti-TNFα antibody fragments, GLP2, IGF1, leptin, and casein.
8. The polypeptide of embodiment 7, wherein Z₁ is selected from the group consisting of anti-TNFα antibody, anti-TNFα antibody fragment, IL-10, IL-22, hBD3, leptin, and beta-casein.
9. The polypeptide of embodiment 5, wherein X₁ comprises an amino acid sequence of SEQ ID NO: 6 and Z₁ comprises an anti-TNFα antibody or an anti-TNFα antibody fragment.
10. The polypeptide of embodiment 9, wherein X₁ comprises an amino acid sequence of SEQ ID NO: 6 and Z₁ comprises an anti-TNFα antibody.
11. The polypeptide of embodiment 9, wherein X₁ comprises an amino acid sequence of SEQ ID NO: 6 and Z₁ comprises an anti-TNFα antibody fragment.
12. The polypeptide of embodiment 9, wherein X₁ comprises an amino acid sequence of SEQ ID NO: 6 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, SEQ ID NO: 120, or fragment thereof.
13. The polypeptide of embodiment 12, wherein X₁ comprises an amino acid sequence of SEQ ID NO: 6 and Z₁ comprises an amino acid sequence having at least 90% identity to SEQ ID NO: 21, SEQ ID NO: 118, SEQ ID NO: 119, SEQ ID NO: 120, or fragment thereof.
14. The polypeptide of embodiment 12, wherein X₁ comprises an amino acid sequence of SEQ ID NO: 6 and Z₁ comprises an amino acid sequence having at least 95% identity to SEQ ID NO: 21, SEQ ID NO: 118, SEQ ID NO: 119, SEQ ID NO: 120, or fragment thereof.
15. The polypeptide of embodiment 12, wherein X₁ comprises an amino acid sequence of SEQ ID NO: 6 and Z₁ comprises an amino acid sequence having at least 99% identity to SEQ ID NO: 21, SEQ ID NO: 118, SEQ ID NO: 119, SEQ ID NO: 120, or fragment thereof.
16. The polypeptide of embodiment 12, wherein X₁ comprises an amino acid sequence of SEQ ID NO: 6 and Z₁ comprises an amino acid sequence of SEQ ID NO: 21, SEQ ID NO: 118, SEQ ID NO: 119, SEQ ID NO: 120, or fragment thereof.
17. The polypeptide of any one of embodiments 12-16, wherein Z₁ comprises an amino acid sequence of SEQ ID NO: 21.
18. The polypeptide of any one of embodiments 12-16, wherein Z₁ comprises an amino acid sequence of SEQ ID NO: 118.
19. The polypeptide of any one of embodiments 12-16, wherein Z₁ comprises an amino acid sequence of SEQ ID NO: 119.
20. The polypeptide of any one of embodiments 12-16, wherein Z₁ comprises an amino acid sequence of SEQ ID NO: 120.
21. The polypeptide of embodiment 5, wherein X₁ comprises an amino acid sequence of SEQ ID NO: 7 and Z₁ comprises an anti-TNFα antibody or an anti-TNFα antibody fragment.
22. The polypeptide of embodiment 21, wherein X₁ comprises an amino acid sequence of SEQ ID NO: 7 and Z₁ comprises an anti-TNFα antibody.
23. The polypeptide of embodiment 21, wherein X₁ comprises an amino acid sequence of SEQ ID NO: 7 and Z₁ comprises an anti-TNFα antibody fragment.
24. The polypeptide of embodiment 21, wherein X₁ comprises an amino acid sequence of SEQ ID NO: 7 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, SEQ ID NO: 120, or fragment thereof.
25. The polypeptide of embodiment 24, wherein X₁ comprises an amino acid sequence of SEQ ID NO: 7 and Z₁ comprises an amino acid sequence having at least 90% identity to SEQ ID NO: 21, SEQ ID NO: 118, SEQ ID NO: 119, SEQ ID NO: 120, or fragment thereof.
26. The polypeptide of embodiment 24, wherein X₁ comprises an amino acid sequence of SEQ ID NO: 7 and Z₁ comprises an amino acid sequence having at least 95% identity to SEQ ID NO: 21, SEQ ID NO: 118, SEQ ID NO: 119, SEQ ID NO: 120, or fragment thereof.
27. The polypeptide of embodiment 24, wherein X₁ comprises an amino acid sequence of SEQ ID NO: 7 and Z₁ comprises an amino acid sequence having at least 99% identity to SEQ ID NO: 21, SEQ ID NO: 118, SEQ ID NO: 119, SEQ ID NO: 120, or fragment thereof.
28. The polypeptide of embodiment 24, wherein X₁ comprises an amino acid sequence of SEQ ID NO: 7 and Z₁ comprises an amino acid sequence of SEQ ID NO: 21, SEQ ID NO: 118, SEQ ID NO: 119, SEQ ID NO: 120, or fragment thereof.
29. The polypeptide of any one of embodiments 24-28, wherein Z₁ comprises an amino acid sequence of SEQ ID NO: 21.
30. The polypeptide of any one of embodiments 24-28, wherein Z₁ comprises an amino acid sequence of SEQ ID NO: 118.
31. The polypeptide of any one of embodiments 24-28, wherein Z₁ comprises an amino acid sequence of SEQ ID NO: 119.
32. The polypeptide of any one of embodiments 24-28, wherein Z₁ comprises an amino acid sequence of SEQ ID NO: 120.
33. The polypeptide of embodiment 5, wherein X₁ comprises an amino acid sequence of SEQ ID NO: 14 and Z₁ comprises an anti-TNFα antibody or an anti-TNFα antibody fragment.
34. The polypeptide of embodiment 33, wherein X₁ comprises an amino acid sequence of SEQ ID NO: 14 and Z₁ comprises an anti-TNFα antibody.
35. The polypeptide of embodiment 33, wherein X₁ comprises an amino acid sequence of SEQ ID NO: 14 and Z₁ comprises an anti-TNFα antibody fragment.
36. The polypeptide of embodiment 33, wherein X₁ comprises an amino acid sequence of SEQ ID NO: 14 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 21, SEQ ID NO: 118, SEQ ID NO: 119, SEQ ID NO: 120, or fragment thereof.
37. The polypeptide of embodiment 36, wherein X₁ comprises an amino acid sequence of SEQ ID NO: 14 and Z₁ comprises an amino acid sequence having at least 90% identity to SEQ ID NO: 21, SEQ ID NO: 118, SEQ ID NO: 119, SEQ ID NO: 120, or fragment thereof.
38. The polypeptide of embodiment 36, wherein X₁ comprises an amino acid sequence of SEQ ID NO: 14 and Z₁ comprises an amino acid sequence having at least 95% identity to SEQ ID NO: 21, SEQ ID NO: 118, SEQ ID NO: 119, SEQ ID NO: 120, or fragment thereof.
39. The polypeptide of embodiment 36, wherein X₁ comprises an amino acid sequence of SEQ ID NO: 14 and Z₁ comprises an amino acid sequence having at least 99% identity to SEQ ID NO: 21, SEQ ID NO: 118, SEQ ID NO: 119, SEQ ID NO: 120, or fragment thereof.
40. The polypeptide of embodiment 36, wherein X₁ comprises an amino acid sequence of SEQ ID NO: 14 and Z₁ comprises an amino acid sequence of SEQ ID NO: 21, SEQ ID NO: 118, SEQ ID NO: 119, SEQ ID NO: 120, or fragment thereof.
41. The polypeptide of any one of embodiments 36-40, wherein Z₁ comprises an amino acid sequence of SEQ ID NO: 21.
42. The polypeptide of any one of embodiments 36-40, wherein Z₁ comprises an amino acid sequence of SEQ ID NO: 118.
43. The polypeptide of any one of embodiments 36-40, wherein Z₁ comprises an amino acid sequence of SEQ ID NO: 119.
44. The polypeptide of any one of embodiments 36-40, wherein Z₁ comprises an amino acid sequence of SEQ ID NO: 120.
45. The polypeptide of embodiment 5, wherein X₁ comprises an amino acid sequence of SEQ ID NO: 6 and Z₁ comprises leptin.
46. The polypeptide of embodiment 45, wherein X₁ comprises an amino acid sequence of SEQ ID NO: 6 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25.
47. The polypeptide of embodiment 45, wherein X₁ comprises an amino acid sequence of SEQ ID NO: 6, and Z₁ comprises an amino acid sequence having at least 90% identity to SEQ ID NO: 25.
48. The polypeptide of embodiment 45, wherein X₁ comprises an amino acid sequence of SEQ ID NO: 6 and Z₁ comprises an amino acid sequence having at least 95% identity to SEQ ID NO: 25.
49. The polypeptide of embodiment 45, wherein X₁ comprises an amino acid sequence of SEQ ID NO: 6, and Z₁ comprises an amino acid sequence having at least 99% identity to SEQ ID NO: 25.
50. The polypeptide of embodiment 45, wherein X₁ comprises an amino acid sequence of SEQ ID NO: 6, and Z₁ comprises the amino acid sequence of SEQ ID NO: 25.
51. The polypeptide of embodiment 5, wherein X₁ comprises an amino acid sequence of SEQ ID NO: 7 and Z₁ comprises leptin.
52. The polypeptide of embodiment 51, wherein X₁ comprises an amino acid sequence of SEQ ID NO: 7 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25.
53. The polypeptide of embodiment 51, wherein X₁ comprises an amino acid sequence of SEQ ID NO: 7, and Z₁ comprises an amino acid sequence having at least 90% identity to SEQ ID NO: 25.
54. The polypeptide of embodiment 51, wherein X₁ comprises an amino acid sequence of SEQ ID NO: 7 and Z₁ comprises an amino acid sequence having at least 95% identity to SEQ ID NO: 25.
55. The polypeptide of embodiment 51, wherein X₁ comprises an amino acid sequence of SEQ ID NO: 7, and Z₁ comprises an amino acid sequence having at least 99% identity to SEQ ID NO: 25.
56. The polypeptide of embodiment 51, wherein X₁ comprises an amino acid sequence of SEQ ID NO: 7, and Z₁ comprises the amino acid sequence of SEQ ID NO: 25.
57. The polypeptide of embodiment 5, wherein X₁ comprises an amino acid sequence of SEQ ID NO: 14 and Z₁ comprises leptin.
58. The polypeptide of embodiment 57, wherein X₁ comprises an amino acid sequence of SEQ ID NO: 14 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID
59. The polypeptide of embodiment 57, wherein X₁ comprises an amino acid sequence of SEQ ID NO: 14, and Z₁ comprises an amino acid sequence having at least 90% identity to SEQ ID NO: 25.
60. The polypeptide of embodiment 57, wherein X₁ comprises an amino acid sequence of SEQ ID NO: 14 and Z₁ comprises an amino acid sequence having at least 95% identity to SEQ ID NO: 25.
61. The polypeptide of embodiment 57, wherein X₁ comprises an amino acid sequence of SEQ ID NO: 14, and Z₁ comprises an amino acid sequence having at least 99% identity to SEQ ID NO: 25.
62. The polypeptide of embodiment 57, wherein X₁ comprises an amino acid sequence of SEQ ID NO: 14, and Z₁ comprises the amino acid sequence of SEQ ID NO: 25.
63. The polypeptide of embodiment 5, wherein X₁ comprises an amino acid sequence of SEQ ID NO: 17 and Z₁ comprises leptin.
64. The polypeptide of embodiment 63, wherein X₁ comprises an amino acid sequence of SEQ ID NO: 17 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25.
65. The polypeptide of embodiment 63, wherein X₁ comprises an amino acid sequence of SEQ ID NO: 17, and Z₁ comprises an amino acid sequence having at least 90% identity to SEQ ID NO: 25.
66. The polypeptide of embodiment 63, wherein X₁ comprises an amino acid sequence of SEQ ID NO: 17 and Z₁ comprises an amino acid sequence having at least 95% identity to SEQ ID NO: 25.
67. The polypeptide of embodiment 63, wherein X₁ comprises an amino acid sequence of SEQ ID NO: 17, and Z₁ comprises an amino acid sequence having at least 99% identity to SEQ ID NO: 25.
68. The polypeptide of embodiment 63, wherein X₁ comprises an amino acid sequence of SEQ ID NO: 17, and Z₁ comprises the amino acid sequence of SEQ ID NO: 25.
69. The polypeptide of embodiment 5, wherein X₁ comprises an amino acid sequence of SEQ ID NO: 18 and Z₁ comprises leptin.
70. The polypeptide of embodiment 69, wherein X₁ comprises an amino acid sequence of SEQ ID NO: 18 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25.
71. The polypeptide of embodiment 69, wherein X₁ comprises an amino acid sequence of SEQ ID NO: 18 and Z₁ comprises an amino acid sequence having at least 90% identity to SEQ ID NO: 25.
72. The polypeptide of embodiment 69, wherein X₁ comprises an amino acid sequence of SEQ ID NO: 18, and Z₁ comprises an amino acid sequence having at least 95% identity to SEQ ID NO: 25.
73. The polypeptide of embodiment 69, wherein X₁ comprises an amino acid sequence of SEQ ID NO: 18 and Z₁ comprises an amino acid sequence having at least 99% identity to SEQ ID NO: 25.
74. The polypeptide of embodiment 69, wherein X₁ comprises an amino acid sequence of SEQ ID NO: 18 and Z₁ comprises the amino acid sequence of SEQ ID NO: 25.
75. The polypeptide of embodiment 5, wherein X₁ comprises an amino acid sequence of SEQ ID NO: 19 and Z₁ comprises leptin.
76. The polypeptide of embodiment 75, wherein X₁ comprises an amino acid sequence of SEQ ID NO: 19 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25.
77. The polypeptide of embodiment 75, wherein X₁ comprises an amino acid sequence of SEQ ID NO: 19 and Z₁ comprises an amino acid sequence having at least 90% identity to SEQ ID NO: 25.
78. The polypeptide of embodiment 75, wherein X₁ comprises an amino acid sequence of SEQ ID NO: 19 and Z₁ comprises an amino acid sequence having at least 95% identity to SEQ ID NO: 25.
79. The polypeptide of embodiment 75, wherein X₁ comprises an amino acid sequence of SEQ ID NO: 19 and Z₁ comprises an amino acid sequence having at least 99% identity to SEQ ID NO: 25.
80. The polypeptide of embodiment 75, wherein X₁ comprises an amino acid sequence of SEQ ID NO: 19 and Z₁ comprises the amino acid sequence of SEQ ID NO: 25.
81. The polypeptide of embodiment 5, wherein X₁ comprises an amino acid sequence of SEQ ID NO: 47 and Z₁ comprises leptin.
82. The polypeptide of embodiment 81, wherein X₁ comprises an amino acid sequence of SEQ ID NO: 47 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25.
83. The polypeptide of embodiment 81, wherein X₁ comprises an amino acid sequence of SEQ ID NO: 47 and Z₁ comprises an amino acid sequence having at least 90% identity to SEQ ID NO: 25.
84. The polypeptide of embodiment 81, wherein X₁ comprises an amino acid sequence of SEQ ID NO: 47 and Z₁ comprises an amino acid sequence having at least 95% identity to SEQ ID NO: 25.
85. The polypeptide of embodiment 81, wherein X₁ comprises an amino acid sequence of SEQ ID NO: 47 and Z₁ comprises an amino acid sequence having at least 99% identity to SEQ ID NO: 25.
86. The polypeptide of embodiment 81, wherein X₁ comprises an amino acid sequence of SEQ ID NO: 47 and Z₁ comprises the amino acid sequence of SEQ ID NO: 25.
87. The polypeptide of embodiment 5, wherein X₁ comprises an amino acid sequence of SEQ ID NO: 48 and Z₁ comprises leptin.
88. The polypeptide of embodiment 87, wherein X₁ comprises an amino acid sequence of SEQ ID NO: 48 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25.
89. The polypeptide of embodiment 87, wherein X₁ comprises an amino acid sequence of SEQ ID NO: 48 and Z₁ comprises an amino acid sequence having at least 90% identity to SEQ ID NO: 25.
90. The polypeptide of embodiment 87, wherein X₁ comprises an amino acid sequence of SEQ ID NO: 48 and Z₁ comprises an amino acid sequence having at least 95% identity to SEQ ID NO: 25.
91. The polypeptide of embodiment 87, wherein X₁ comprises an amino acid sequence of SEQ ID NO: 48 and Z₁ comprises an amino acid sequence having at least 99% identity to SEQ ID NO: 25.
92. The polypeptide of embodiment 87, wherein X₁ comprises an amino acid sequence of SEQ ID NO: 48 and Z₁ comprises the amino acid sequence of SEQ ID NO: 25.
93. The polypeptide of embodiment 5, wherein X₁ comprises an amino acid sequence of SEQ ID NO: 148 and Z₁ comprises leptin.
94. The polypeptide of embodiment 93, wherein X₁ comprises an amino acid sequence of SEQ ID NO: 148 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 25.
95. The polypeptide of embodiment 93, wherein X₁ comprises an amino acid sequence of SEQ ID NO: 148, and Z₁ comprises an amino acid sequence having at least 90% identity to SEQ ID NO: 25.
96. The polypeptide of embodiment 93, wherein X₁ comprises an amino acid sequence of SEQ ID NO: 148 and Z₁ comprises an amino acid sequence having at least 95% identity to SEQ ID NO: 25.
97. The polypeptide of embodiment 93, wherein X₁ comprises an amino acid sequence of SEQ ID NO: 148, and Z₁ comprises an amino acid sequence having at least 99% identity to SEQ ID NO: 25.
98. The polypeptide of embodiment 93, wherein X₁ comprises an amino acid sequence of SEQ ID NO: 148, and Z₁ comprises the amino acid sequence of SEQ ID NO: 25.
99. The polypeptide of embodiment 5, wherein X₁ comprises an amino acid sequence of SEQ ID NO: 180 and Z₁ comprises leptin.
100. The polypeptide of embodiment 99, wherein X₁ comprises an amino acid sequence of SEQ ID NO: 180 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID
101. The polypeptide of embodiment 99, wherein X₁ comprises an amino acid sequence of SEQ ID NO: 180, and Z₁ comprises an amino acid sequence having at least 90% identity to SEQ ID NO: 25.
102. The polypeptide of embodiment 99, wherein X₁ comprises an amino acid sequence of SEQ ID NO: 180 and Z₁ comprises an amino acid sequence having at least 95% identity to SEQ ID NO: 25.
103. The polypeptide of embodiment 99, wherein X₁ comprises an amino acid sequence of SEQ ID NO: 180, and Z₁ comprises an amino acid sequence having at least 99% identity to SEQ ID NO: 25.
104. The polypeptide of embodiment 99, wherein X₁ comprises an amino acid sequence of SEQ ID NO: 180, and Z₁ comprises the amino acid sequence of SEQ ID NO: 25.
105. The polypeptide of embodiment 5, wherein X₁ comprises an amino acid sequence of SEQ ID NO: 20 and Z₁ comprises IL-10.
106. The polypeptide of embodiment 105, wherein X₁ comprises an amino acid sequence of SEQ ID NO: 20 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 24, SEQ ID NO: 283, SEQ ID NO: 284, or SEQ ID NO: 285.
107. The polypeptide of embodiment 105, wherein X₁ comprises an amino acid sequence of SEQ ID NO: 20 and Z₁ comprises an amino acid sequence having at least 90% identity to SEQ ID NO: 24, SEQ ID NO: 283, SEQ ID NO: 284, or SEQ ID NO: 285.
108. The polypeptide of embodiment 105, wherein X₁ comprises an amino acid sequence of SEQ ID NO: 20 and Z₁ comprises an amino acid sequence having at least 95% identity to SEQ ID NO: 24, SEQ ID NO: 283, SEQ ID NO: 284, or SEQ ID NO: 285.
109. The polypeptide of embodiment 105, wherein X₁ comprises an amino acid sequence of SEQ ID NO: 20 and Z₁ comprises an amino acid sequence having at least 99% identity to SEQ ID NO: 24, SEQ ID NO: 283, SEQ ID NO: 284, or SEQ ID NO: 285.
110. The polypeptide of embodiment 105, wherein X₁ comprises an amino acid sequence of SEQ ID NO: 20 and Z₁ comprises an amino acid sequence of SEQ ID NO: 24, SEQ ID NO: 283, SEQ ID NO: 284, or SEQ ID NO: 285.
111. The polypeptide of embodiment 105, wherein X₁ comprises an amino acid sequence of SEQ ID NO: 20 and Z₁ comprises an amino acid sequence of SEQ ID NO: 24.
112. The polypeptide of embodiment 105, wherein X₁ comprises an amino acid sequence of SEQ ID NO: 20 and Z₁ comprises an amino acid sequence of SEQ ID NO: 283.
113. The polypeptide of embodiment 105, wherein X₁ comprises an amino acid sequence of SEQ ID NO: 20 and Z₁ comprises an amino acid sequence of SEQ ID NO: 284.
114. The polypeptide of embodiment 105, wherein X₁ comprises an amino acid sequence of SEQ ID NO: 20 and Z₁ comprises an amino acid sequence of SEQ ID NO: 285.
115. The polypeptide of embodiment 105, wherein X₁ comprises an amino acid sequence of SEQ ID NO: 20 and Z₁ comprises an amino acid sequence of SEQ ID NO: 286.
115. The polypeptide of embodiment 5, wherein X₁ comprises an amino acid sequence of SEQ ID NO: 7 and Z₁ comprises IL-22.
116. The polypeptide of embodiment 115, wherein X₁ comprises an amino acid sequence of SEQ ID NO: 7 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282.
117. The polypeptide of embodiment 115, wherein X₁ comprises an amino acid sequence of SEQ ID NO: 7 and Z₁ comprises an amino acid sequence having at least 90% identity to SEQ ID NO: 22, SEQ ID NO: 59, or SEQ ID NO: 282.
118. The polypeptide of embodiment 115, wherein X₁ comprises an amino acid sequence of SEQ ID NO: 7 and Z₁ comprises an amino acid sequence having at least 95% identity to SEQ ID NO: 22, SEQ ID NO: 59, or SEQ ID NO: 282.
119. The polypeptide of embodiment 115, wherein X₁ comprises an amino acid sequence of SEQ ID NO: 7 and Z₁ comprises an amino acid sequence having at least 99% identity to SEQ ID NO: 22, SEQ ID NO: 59, or SEQ ID NO: 282.
120. The polypeptide of embodiment 115, wherein X₁ comprises an amino acid sequence of SEQ ID NO: 7 and Z₁ comprises an amino acid sequence of SEQ ID NO: 22, SEQ ID NO: 59, or SEQ ID NO: 282.
121. The polypeptide of embodiment 115, wherein X₁ comprises an amino acid sequence of SEQ ID NO: 7 and Z₁ comprises an amino acid sequence of SEQ ID NO: 22.
122. The polypeptide of embodiment 115, wherein X₁ comprises an amino acid sequence of SEQ ID NO: 7 and Z₁ comprises an amino acid sequence of SEQ ID NO: 59.
123. The polypeptide of embodiment 115, wherein X₁ comprises an amino acid sequence of SEQ ID NO: 7 and Z₁ comprises an amino acid sequence of SEQ ID NO: 282.
124. The polypeptide of embodiment 5, wherein X₁ comprises an amino acid sequence of SEQ ID NO: 14 and Z₁ comprises IL-22.
125. The polypeptide of embodiment 124, wherein X₁ comprises an amino acid sequence of SEQ ID NO: 14 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282.
126. The polypeptide of embodiment 124, wherein X₁ comprises an amino acid sequence of SEQ ID NO: 14 and Z₁ comprises an amino acid sequence having at least 90% identity to SEQ ID NO: 22, SEQ ID NO: 59, or SEQ ID NO: 282.
127. The polypeptide of embodiment 124, wherein X₁ comprises an amino acid sequence of SEQ ID NO: 14 and Z₁ comprises an amino acid sequence having at least 95% identity to SEQ ID NO: 22, SEQ ID NO: 59, or SEQ ID NO: 282.
128. The polypeptide of embodiment 124, wherein X₁ comprises an amino acid sequence of SEQ ID NO: 14 and Z₁ comprises an amino acid sequence having at least 99% identity to SEQ ID NO: 22, SEQ ID NO: 59, or SEQ ID NO: 282.
129. The polypeptide of embodiment 124, wherein X₁ comprises an amino acid sequence of SEQ ID NO: 14 and Z₁ comprises an amino acid sequence of SEQ ID NO: 22, SEQ ID NO: 59, or SEQ ID NO: 282.
130. The polypeptide of embodiment 124, wherein X₁ comprises an amino acid sequence of SEQ ID NO: 14 and Z₁ comprises an amino acid sequence of SEQ ID NO: 22.
131. The polypeptide of embodiment 124, wherein X₁ comprises an amino acid sequence of SEQ ID NO: 14 and Z₁ comprises an amino acid sequence of SEQ ID NO: 59.
132. The polypeptide of embodiment 124, wherein X₁ comprises an amino acid sequence of SEQ ID NO: 14 and Z₁ comprises an amino acid sequence of SEQ ID NO: 282.
133. The polypeptide of embodiment 5, wherein X₁ comprises an amino acid sequence of SEQ ID NO: 148 and Z₁ comprises IL-22.
134. The polypeptide of embodiment 133, wherein X₁ comprises an amino acid sequence of SEQ ID NO: 148 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282.
135. The polypeptide of embodiment 133, wherein X₁ comprises an amino acid sequence of SEQ ID NO: 148 and Z₁ comprises an amino acid sequence having at least 90% identity to SEQ ID NO: 22, SEQ ID NO: 59, or SEQ ID NO: 282.
136. The polypeptide of embodiment 133, wherein X₁ comprises an amino acid sequence of SEQ ID NO: 148 and Z₁ comprises an amino acid sequence having at least 95% identity to SEQ ID NO: 22, SEQ ID NO: 59, or SEQ ID NO: 282.
137. The polypeptide of embodiment 133, wherein X₁ comprises an amino acid sequence of SEQ ID NO: 148 and Z₁ comprises an amino acid sequence having at least 99% identity to SEQ ID NO: 22, SEQ ID NO: 59, or SEQ ID NO: 282.
138. The polypeptide of embodiment 133, wherein X₁ comprises an amino acid sequence of SEQ ID NO: 148 and Z₁ comprises an amino acid sequence of SEQ ID NO: 22, SEQ ID NO: 59, or SEQ ID NO: 282.
139. The polypeptide of embodiment 133, wherein X₁ comprises an amino acid sequence of SEQ ID NO: 148 and Z₁ comprises an amino acid sequence of SEQ ID NO: 22.
140. The polypeptide of embodiment 133, wherein X₁ comprises an amino acid sequence of SEQ ID NO: 148 and Z₁ comprises an amino acid sequence of SEQ ID NO: 59.
141. The polypeptide of embodiment 133, wherein X₁ comprises an amino acid sequence of SEQ ID NO: 148 and Z₁ comprises an amino acid sequence of SEQ ID NO: 282.
142. The polypeptide of embodiment 5, wherein X₁ comprises an amino acid sequence of SEQ ID NO: 180 and Z₁ comprises IL-22.
143. The polypeptide of embodiment 142, wherein X₁ comprises an amino acid sequence of SEQ ID NO: 180 and Z₁ comprises an amino acid sequence having at least 85%, 86%, 87%, 88%, 89%, 90%, 91%, 92%, 93%, 94%, 95%, 96%, 97%, 98%, or 99% identity to SEQ ID NO: 22, SEQ ID NO: 59, or SEQ ID NO: 282.
144. The polypeptide of embodiment 142, wherein X₁ comprises an amino acid sequence of SEQ ID NO: 180 and Z₁ comprises an amino acid sequence having at least 90% identity to SEQ ID NO: 22, SEQ ID NO: 59, or SEQ ID NO: 282.
145. The polypeptide of embodiment 142, wherein X₁ comprises an amino acid sequence of SEQ ID NO: 180 and Z₁ comprises an amino acid sequence having at least 95% identity to SEQ ID NO: 22, SEQ ID NO: 59, or SEQ ID NO: 282.
146. The polypeptide of embodiment 142, wherein X₁ comprises an amino acid sequence of SEQ ID NO: 180 and Z₁ comprises an amino acid sequence having at least 99% identity to SEQ ID NO: 22, SEQ ID NO: 59, or SEQ ID NO: 282.
147. The polypeptide of embodiment 142, wherein X₁ comprises an amino acid sequence of SEQ ID NO: 180 and Z₁ comprises an amino acid sequence of SEQ ID NO: 22, SEQ ID NO: 59, or SEQ ID NO: 282.
148. The polypeptide of embodiment 142, wherein X₁ comprises an amino acid sequence of SEQ ID NO: 180 and Z₁ comprises an amino acid sequence of SEQ ID NO: 22.
149. The polypeptide of embodiment 142, wherein X₁ comprises an amino acid sequence of SEQ ID NO: 180 and Z₁ comprises an amino acid sequence of SEQ ID NO: 59.
150. The polypeptide of embodiment 142, wherein X₁ comprises an amino acid sequence of SEQ ID NO: 180 and Z₁ comprises an amino acid sequence of SEQ ID NO: 282.
151. A polypeptide comprising a formula of X₁-Z₁ wherein:
   X₁ is a pre-protein signal peptide comprising the amino acid sequence of SEQ ID NO: 6; and
   Z₁ is a payload protein comprising an anti-TNFα antibody or an anti-TNFα antibody fragment.
152. The polypeptide of embodiment 151, wherein Z₁ comprises an amino acid sequence selected from SEQ ID NO: 21, SEQ ID NO: 118, SEQ ID NO: 119, or SEQ ID NO: 120.
153. A polypeptide comprising a formula of X₁-Z₁ wherein:
   X₁ is a pre-protein signal peptide comprising the amino acid sequence of SEQ ID NO: 7; and
   Z₁ is a payload protein comprising an anti-TNFα antibody or an anti-TNFα antibody fragment.
154. The polypeptide of embodiment 153, wherein Z₁ comprises an amino acid sequence selected from SEQ ID NO: 21, SEQ ID NO: 118, SEQ ID NO: 119, or SEQ ID NO: 120.
155. A polypeptide comprising a formula of X₁-Z₁ wherein:
   X₁ is a pre-protein signal peptide comprising the amino acid sequence of SEQ ID NO: 14; and
   Z₁ is a payload protein comprising an anti-TNFα antibody or an anti-TNFα antibody fragment.
156. The polypeptide of embodiment 155, wherein Z₁ comprises an amino acid sequence selected from SEQ ID NO: 21, SEQ ID NO: 118, SEQ ID NO: 119, or SEQ ID NO: 120.
157. A polypeptide comprising a formula of X₁-Z₁ wherein:
   X₁ is a pre-protein signal peptide comprising the amino acid sequence of SEQ ID NO: 6; and
   Z₁ is a payload protein comprising leptin.
158. The polypeptide of embodiment 157, wherein Z₁ comprises an amino acid sequence of SEQ ID NO: 25.
159. A polypeptide comprising a formula of X₁-Z₁ wherein:
   X₁ is a pre-protein signal peptide comprising the amino acid sequence of SEQ ID NO: 7; and
   Z₁ is a payload protein comprising leptin.
160. The polypeptide of embodiment 159, wherein Z₁ comprises an amino acid sequence
161. A polypeptide comprising a formula of X₁-Z₁ wherein:
   X₁ is a pre-protein signal peptide comprising the amino acid sequence of SEQ ID NO: 14; and
   Z₁ is a payload protein comprising leptin.
162. The polypeptide of embodiment 161, wherein Z₁ comprises an amino acid sequence of SEQ ID NO: 25.
163. A polypeptide comprising a formula of X₁-Z₁ wherein:
   X₁ is a pre-protein signal peptide comprising the amino acid sequence of SEQ ID NO: 17; and
   Z₁ is a payload protein comprising leptin.
164. The polypeptide of embodiment 163, wherein Z₁ comprises an amino acid sequence of SEQ ID NO: 25.
165. A polypeptide comprising a formula of X₁-Z₁ wherein:
   X₁ is a pre-protein signal peptide comprising the amino acid sequence of SEQ ID NO: 18; and
   Z₁ is a payload protein comprising leptin.
166. The polypeptide of embodiment 165, wherein Z₁ comprises an amino acid sequence of SEQ ID NO: 25.
167. A polypeptide comprising a formula of X₁-Z₁ wherein:
   X₁ is a pre-protein signal peptide comprising the amino acid sequence of SEQ ID NO: 19; and
   Z₁ is a payload protein comprising leptin.
168. The polypeptide of embodiment 167, wherein Z₁ comprises an amino acid sequence
169. A polypeptide comprising a formula of X₁-Z₁ wherein:
   X₁ is a pre-protein signal peptide comprising the amino acid sequence of SEQ ID NO: 47; and
   Z₁ is a payload protein comprising leptin.
170. The polypeptide of embodiment 169, wherein Z₁ comprises an amino acid sequence of SEQ ID NO: 25.
171. A polypeptide comprising a formula of X₁-Z₁ wherein:
   X₁ is a pre-protein signal peptide comprising the amino acid sequence of SEQ ID NO: 48; and
   Z₁ is a payload protein comprising leptin.
172. The polypeptide of embodiment 171, wherein Z₁ comprises an amino acid sequence of SEQ ID NO: 25.
173. A polypeptide comprising a formula of X₁-Z₁ wherein:
   X₁ is a pre-protein signal peptide comprising the amino acid sequence of SEQ ID NO: 148; and
   Z₁ is a payload protein comprising leptin.
174. The polypeptide of embodiment 173, wherein Z₁ comprises an amino acid sequence of SEQ ID NO: 25.
175. A polypeptide comprising a formula of X₁-Z₁ wherein:
   X₁ is a pre-protein signal peptide comprising the amino acid sequence of SEQ ID NO: 180; and
   Z₁ is a payload protein comprising leptin.
176. The polypeptide of embodiment 175, wherein Z₁ comprises an amino acid sequence
177. A polypeptide comprising a formula of X₁-Z₁ wherein:
   X₁ is a pre-protein signal peptide comprising the amino acid sequence of SEQ ID NO: 20; and
   Z₁ is a payload protein comprising IL-10.
178. The polypeptide of embodiment 177, wherein Z₁ comprises an amino acid sequence of SEQ ID NO: 24, SEQ ID NO: 283, SEQ ID NO: 284, or SEQ ID NO: 285.
179. A polypeptide comprising a formula of X₁-Z₁ wherein:
   X₁ is a pre-protein signal peptide comprising the amino acid sequence of SEQ ID NO: 7; and
   Z₁ is a payload protein comprising IL-22.
180. The polypeptide of embodiment 179, wherein Z₁ comprises an amino acid sequence of SEQ ID NO: 22, SEQ ID NO: 59, or SEQ ID NO: 282.
181. A polypeptide comprising a formula of X₁-Z₁ wherein:
   X₁ is a pre-protein signal peptide comprising the amino acid sequence of SEQ ID NO: 14; and
   Z₁ is a payload protein comprising IL-22.
182. The polypeptide of embodiment 181, wherein Z₁ comprises an amino acid sequence of SEQ ID NO: 22, SEQ ID NO: 59, or SEQ ID NO: 282.
183. A polypeptide comprising a formula of X₁-Z₁ wherein:
   X₁ is a pre-protein signal peptide comprising the amino acid sequence of SEQ ID NO: 148; and
   Z₁ is a payload protein comprising IL-22.
184. The polypeptide of embodiment 183, wherein Z₁ comprises an amino acid sequence of SEQ ID NO: 22, SEQ ID NO: 59, or SEQ ID NO: 282.
185. A polypeptide comprising a formula of X₁-Z₁ wherein:
   X₁ is a pre-protein signal peptide comprising the amino acid sequence of SEQ ID NO: 180; and
   Z₁ is a payload protein comprising IL-22.
186. The polypeptide of embodiment 185, wherein Z₁ comprises an amino acid sequence of SEQ ID NO: 22, SEQ ID NO: 59, or SEQ ID NO: 282.
187. The polypeptide of any one of embodiments 5-186, wherein X₁ and Z₁ are linked by a linker polypeptide.
188. The polypeptide of embodiment 187, wherein the linker polypeptide is selected from the group consisting of a flexible peptide linker, a rigid peptide linker, a cleavable peptide linker, and a non-cleavable peptide linker.
189. The polypeptide of embodiment 187 or 188, wherein the linker polypeptide is selected from SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, or SEQ ID NO: 45.
190. The polypeptide of embodiment 187 or 188, wherein the linker polypeptide is a flexible peptide linker.
191. The polypeptide of embodiment 190, wherein the linker polypeptide is selected from SEQ ID NO: 27, SEQ ID NO: 28, SEQ ID NO: 29, SEQ ID NO: 30, or SEQ ID NO: 31.
192. A nucleic acid molecule encoding for a polypeptide of any one of embodiments 5-191.
193. A bacterium comprising a heterologous nucleic acid molecule encoding a polypeptide comprising a formula of X₁-Z₁ wherein:
   X₁ is the pre-protein signal peptide of any one of embodiments 1-4; and
   Z₁ is a payload protein.
194. The bacterium of embodiment 193, wherein Z₁ is selected from the group consisting of Catestatin, CRAMP, hBD1, hBD3, β-Cateslytin, Dermaseptin, IL-10, IL-22, ClbS, TIGIT, TFF1, TFF2, TFF3, anti-CCL2 antibody, anti-CCL2 antibody fragments, anti-IL23 antibody, anti-IL23 antibody fragments, anti-TNFα antibody, anti-TNFα antibody fragments, GLP2, IGF1, leptin, and casein.
195. The bacterium of embodiment 193, wherein Z₁ is selected from the group consisting of anti-TNFα antibody, anti-TNFα antibody fragment. IL-10, IL-22, hBD3, leptin, and beta-casein.
196. The bacterium of embodiment 193, wherein the formula X₁-Z₁ comprises the polypeptide of any one of embodiments 5-191.
197. The bacterium of any one of embodiments 193-196, wherein the bacterium is a Lactobacillus.
198. The bacterium of embodiment 197, wherein the Lactobacillus is selected from L. gasseri, L. jensenii, L. crisparus, L. rhamnosus, L. casei, L. paracasei, L. acidophilus, L. delbruckii, L. johnsonii, L. fermentum, L. reuteri, and L. salivarus.
199. The bacterium of embodiment 197, wherein the Lactobacillus is selected from L. gasseri and L. rhamnosus.
200. A pharmaceutical composition comprising the bacterium of any one of embodiments 193-199 and a pharmaceutically acceptable carrier.
201. A method for producing a payload protein, comprising:
   i) transfecting a bacterium with a nucleic acid molecule encoding for the polypeptide of any one of embodiments 5-191 to produce a bacterium comprising the nucleic acid molecule; and
   ii) culturing the bacteria comprising the nucleic acid molecule under conditions sufficient to grow the bacteria; wherein the bacteria expresses and secretes the payload protein.
202. The method of embodiment 201, wherein the bacterium is a Lactobacillus.
203. The method of embodiment 202, wherein the Lactobacillus is selected from L. gasseri, L. jensenii, L. crisparus, L. rhamnosus, L. casei, L. paracasei, L. acidophilus, L. delbruckii, L. johnsonii, L. fermentum, L. reuteri, and L. salivarus.
204. The method of embodiment 202, wherein the Lactobacillus is selected from L. gasseri and L. rhamnosus.
205. The method of any one of embodiments 201-204, wherein the culturing comprises incubating the bacteria in culture media.
206. The method of any one of embodiments 201-205, wherein the method further comprises recovering or purifying the payload protein from the culture media. 207. A method of producing a payload protein in a subject, comprising administering to the subject the bacteria of any one of embodiments 193-199 or the pharmaceutical composition of embodiment 200, wherein the bacteria are live and wherein the live bacteria produce the protein in the subject.
208. A method for treating a disease or disorder in a subject in need thereof comprising administering to the subject a therapeutically effective amount of the bacteria of any one of embodiments 193-199 or the pharmaceutical composition of embodiment 200.
209. The method of embodiment 208, wherein the disease or disorder is selected from inflammation, chronic inflammation, colorectal cancer, periodontits, oral mucositis, infection, pathogenic E coli, inflammatory bowel disease, ulcerative colitis, Crohn's disease, type 2 diabetes, obesity, lipodystrophy, hypertriglyceridemia, systemic lupus erythematosus, hepatic steatosis, low bone density, hypothalamic hypogonadism, infertility, hypothalamic amenorrhea, auto-immune disease, cancer, NAFLD, NASH, infection, sepsis, or a growth hormone disorder.
210. The method of embodiment 208 or 209, wherein the bacteria or pharmaceutical composition is orally or topically administered.
211. A method for treating inflammation in a subject in need thereof comprising administering to the subject a therapeutically effective amount of the bacteria of any one of embodiments 193-199 or the pharmaceutical composition of embodiment 200.
212. A method for treating inflammation in a subject in need thereof comprising administering to the subject a bacteria comprising a heterologous nucleic acid molecule encoding for a polypeptide having the formula X₁-Z₁, wherein X₁ is the pre-protein signal peptide of any one of embodiments 1~4 and Z₁ is a payload protein selected from an anti-TNFα antibody, anti-TNFα antibody fragments, or IL-10.
213. The method of embodiment 212, wherein the X₁ comprises an amino acid sequence having at least 90%, 91%, 92%, 93%, 94%, 95%, 96%, 97%, 98%, 99%, or 100% identity to an amino acid sequence selected from SEQ ID NO: 6, SEQ ID NO: 7, SEQ ID NO: 14, or SEQ ID NO: 20, and wherein Z₁ comprises an amino acid sequence having at least 90%, 91%, 92%, 93%, 94%, 95%, 96%, 97%, 98%, 99%, or 100% identity to an amino acid sequence selected from SEQ ID NO: 21, SEQ ID NO: 24, SEQ ID NO: 118, SEQ ID NO: 119, or SEQ ID NO: 120.
214. The method of embodiment 212, wherein X₁ comprises an amino acid sequence having at least 90%, 91%, 92%, 93%, 94%, 95%, 96%, 97%, 98%, 99%, or 100% identity to an amino acid sequence selected from SEQ ID NO: 6, SEQ ID NO: 7, or SEQ ID NO: 14, and Z₁ comprises an amino acid sequence having at least 90%, 91%, 92%, 93%, 94%, 95%, 96%, 97%, 98%, 99%, or 100% identity to an amino acid sequence selected from SEQ ID NO: 21, SEQ ID NO: 118, SEQ ID NO: 119, or SEQ ID NO: 120.
215. The method of embodiment 212, wherein X₁ comprises an amino acid sequence selected from SEQ ID NO: 6, SEQ ID NO: 7, or SEQ ID NO: 14, and Z₁ comprises an amino acid sequence selected from SEQ ID NO: 21, SEQ ID NO: 118, SEQ ID NO: 119, or SEQ ID NO: 120.
216. The method of embodiment 212, wherein X₁ comprises an amino acid sequence having at least 90%, 91%, 92%, 93%, 94%, 95%, 96%, 97%, 98%, 99%, or 100% identity to the amino acid sequence of SEQ ID NO: 20, and Z₁ comprises an amino acid sequence having at least 90%, 91%, 92%, 93%, 94%, 95%, 96%, 97%, 98%, 99%, or 100% identity to the amino acid sequence of SEQ ID NO: 24.
217. The method of embodiment 212, wherein X₁ comprises the amino acid sequence of SEQ ID NO: 20, and Z₁ comprises the amino acid sequence of SEQ ID NO: 24.
218. A method for treating obesity in a subject in need thereof comprising administering to the subject a therapeutically effective amount of the bacteria of any one of embodiments 193-199 or the pharmaceutical composition of embodiment 200.
219. A method for treating obesity in a subject in need thereof comprising administering to the subject a bacteria comprising a heterologous nucleic acid molecule encoding for a polypeptide having the formula X₁-Z₁, wherein X₁ is the pre-protein signal peptide of any one of embodiments 1~4 and Z₁ is a payload protein comprising leptin.
220. The method of embodiment 219, wherein X₁ comprises an amino acid sequence having at least 90%, 91%, 92%, 93%, 94%, 95%, 96%, 97%, 98%, 99%, or 100% identity to an amino acid sequence selected from SEQ ID NO: 17, SEQ ID NO: 18, SEQ ID NO: 19, SEQ ID NO: 47, or SEQ ID NO: 48, and Z₁ comprises an amino acid sequence having at least 90%, 91%, 92%, 93%, 94%, 95%, 96%, 97%, 98%, 99%, or 100% identity to an amino acid sequence of SEQ ID NO: 25.
221. The method of embodiment 219, wherein X₁ comprises an amino acid sequence selected from SEQ ID NO: 17, SEQ ID NO: 18, SEQ ID NO: 19, SEQ ID NO: 47, or SEQ ID NO: 48, and Z₁ comprises the amino acid of SEQ ID NO: 25.

### EXAMPLES

Although the embodiments presented in the present application have been described in considerable detail, other versions are possible. Therefore, the spirit and scope of the appended claims should not be limited to the description and the embodiments contained within the specification. Various aspects of the present application will be illustrated with reference to the following non-limiting examples:

### Example 1: Generation of Novel Optimal Signal Peptides for Secretion of TNFα

Traditionally, novel signal peptides have been analyzed solely on their capability to increase payload protein yield or secretion, and selection of optimal signal peptides has been based solely on which signal peptides result in the most payload protein production. However, these traditional selection and optimization methods ignore the effect of the signal peptide on the overall health of the host bacteria. For example, if a signal peptide increases protein yield several fold but results in a much slower bacteria doubling time this may indicate that use of the signal peptide is detrimental to the health of the bacteria. Accordingly, an optimal signal peptide may be considered one that increases secretion and has minimal impact on the health or fitness of the bacterial host.

A library of 100,000 unique signal peptides was generated using a known signal peptide SEQ ID NO: 1 as a template. 15 sequences were randomly selected for further analysis. The sequences are recited in Table 5 below, along with their percent similarity to the known signal peptide sequence:

TABLE 5

| SEQ ID | | | % Identity to |
|---|---|---|---|
| NO: | Label | Sequence | SEQ ID NO: 1 |
| 1 | A | MKKLLLLLVVLLGLAALLLSGCGTSVSAAT | 100 |
| 2 | B | MKKLLLLLVVLAGLAALLLSGCGTSVSAAT | 96.7 |
| 3 | C | MKKLLLLLVVLLGLAALLLSGCGTSVSAAK | 96.7 |
| 4 | D | MKKLLILLVVLLGLAALLLSGCGTSVSAAT | 96.7 |
| 5 | E | MKKLLLLLVVLLSLAALLLSGCGTSVSAAK | 93.3 |
| 6 | F | MKKLLLLLVVLAGLAALLLSGAGTSVSAAT | 93.3 |
| 7 | G | MKKLLIGLVVLLGLAALLLSGLGTSVSAAT | 90.0 |
| 8 | H | MKKLLLLLVVLAGLAALLLSGCGTSVSAAD | 93.3 |
| 9 | I | MKKLTLLLVVVLGLAALCLSGCGTSVSADE | 83.3 |
| 10 | J | MHKLLLLLVVLVGLAALLLSGCGTTVSAAT | 90.0 |
| 11 | K | MRKLLLLLVVLLGLAALLLSGAGTPVSAAT | 90.0 |
| 12 | L | MKRLLLGLVGLLGLAILGLSGCGTSVSAQT | 80.0 |
| 13 | M | MKKLLLLLVLVLGLAAMLLSGCGTPTSADT | 80.0 |
| 14 | N | MKKILLLVVVLLGLAALLWSGAGTSVSAST | 83.3 |
| 15 | O | MRRLLLLLVVVLGLAALLLSGCGTSVSAQA | 83.3 |
| 16 | P | MRKLHELLIVLLGISFVLWSGCGTSVSAAG | 66.7 |

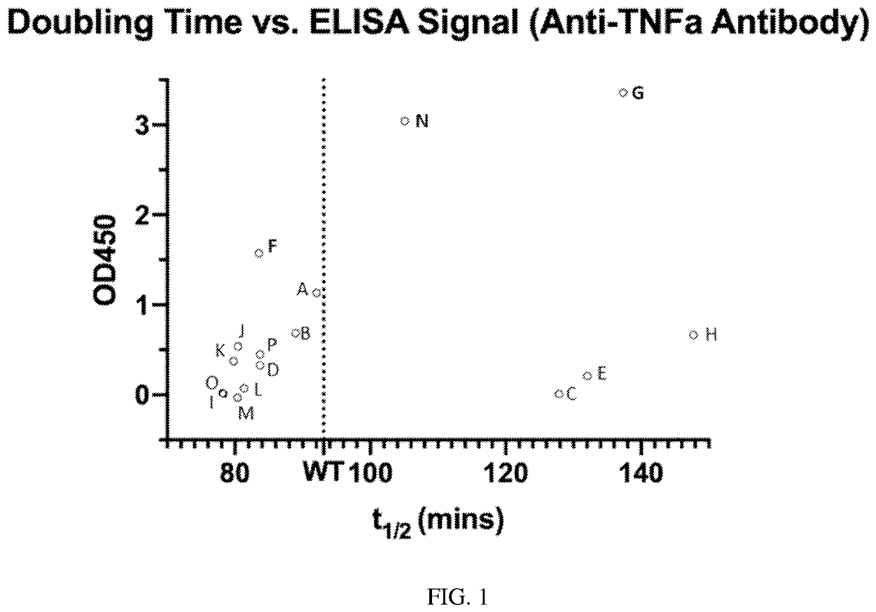

The ability of the randomly selected signal peptides to secrete an anti-TNFαantibody fragment Lactobacillus was then assessed. Bacteria were genetically modified with nucleic acid molecules encoding for the above-recited pre-protein-anti-TNFα constructs and allowed to incubate under conditions sufficient to produce the polypeptides. Supernatant was collected and subject to ELISA analysis. Data are presented as a function of ELISA signal vs. bacterial doubling time (FIG. 1).

Signal peptide sequence F (SEQ ID NO: 6), G (SEQ ID NO: 7), and N (SEQ ID NO: 14) were found to exhibit higher secretion than the known signal peptide SEQ ID NO: 1, with varying influence on doubling time. Examination of the data indicate that % identity to SEQ ID NO: 1 does not predict the doubling time or increased levels of secretion. Signal peptide sequences B, C, and D (SEQ ID NO: 2, SEQ ID NO: 3, and SEQ ID NO: 4, respectively) were the most similar to SEQ ID NO: 1, but all exhibited less TNFα secretion than SEQ ID NO: 1 and had varying influences on doubling time. In contrast, signal peptide sequences F (SEQ ID NO: 6), G (SEQ ID NO: 7), and N (SEQ ID NO: 14) have 93.3, 90.0, and 83.3% identity to SEQ ID NO: 1. These results highlight that sequence similarity does not provide an infallible guideline to generate functional or superior signal peptide alternatives, especially when the signal peptides are analyzed as a function of secretion and strain fitness.

### Example 2: Payload Protein Identity Affects Signal Peptide Performance

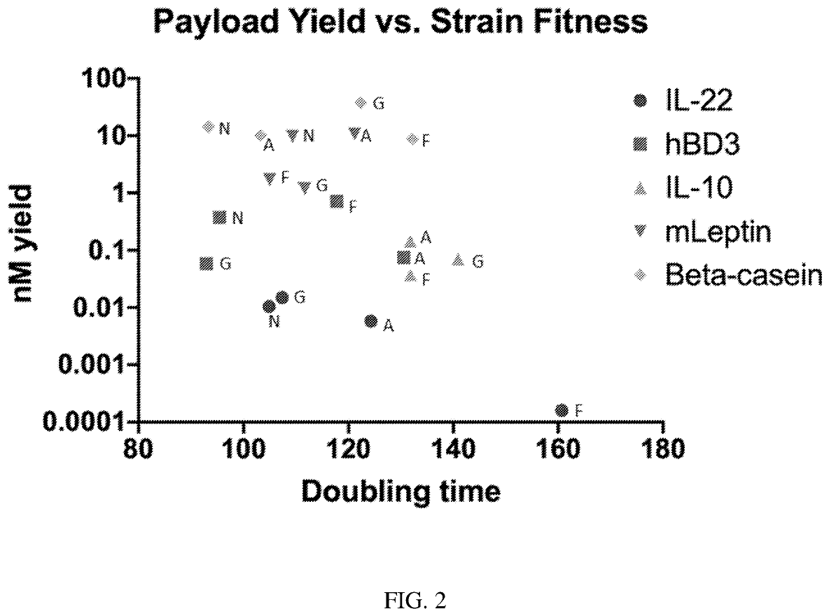

The three signal peptides that resulted in increased secretion of TNFα as compared to the known signal peptide SEQ ID NO: 1 were tested against a larger set of protein payloads. Bacteria were genetically modified with nucleic acid molecules encoding for signal peptides SEQ ID NO: 1, SEQ ID NO: 6, SEQ ID NO: 7, or SEQ ID NO: 14 with payload proteins selected from IL-22, hBD3, IL-10, Leptin, and Beta-casein, and allowed to incubate under conditions sufficient to produce the polypeptides. Supernatant was collected and the concentration of payload protein produced was determined. Data are presented as a function of nM protein yield vs. bacterial doubling time (FIG. 2).

These data illustrate that the effect of a signal peptide on protein yield and strain fitness is dependent on the payload protein being produced. For example, signal peptide sequence F (SEQ ID NO: 6) produces more product than and has a reduced doubling time as compared to the known signal peptide SEQ ID NO: 1 for the payload hBD3. In contrast, the same signal peptide produces almost no product and has a dramatic shift in doubling time for the payload IL-22. Furthermore, the optimal signal peptide varies between each payload. For IL-22, sequence N (SEQ ID NO: 14) and sequence G (SEQ ID NO: 7) performed similarly in terms of both protein yield and doubling time and both were superior to SEQ ID NO: 1. For hBD3, sequence N (SEQ ID NO: 14) and sequence F (SEQ ID NO: 6) both generated more protein and had faster doubling times than SEQ ID NO: 1, but sequence N (SEQ ID NO: 14) had a greater impact on reduced doubling time than sequence F (SEQ ID NO: 6). For Leptin, sequence N (SEQ ID NO: 14) generated similar protein yield to SEQ ID NO: 1, but also had a reduced doubling time as compared to SEQ ID NO: 1. For Beta-casein, sequence N (SEQ ID NO: 14) generated slightly more protein yield to SEQ ID NO: 1 and also had a reduced doubling time as compared to SEQ ID NO: 1. For IL-10, SEQ ID NO: 1 generated the most protein and had the quickest doubling time.

As a whole, these data further highlight that even minor differences in signal peptide sequence result in major differences in secretion. Further, these results highlight the importance of considering both protein yield and strain doubling time for signal peptides. For example, in considering the payload protein Beta-casein, traditional analysis would have identified sequence G (SEQ ID NO: 7) as the “best” signal peptide because it produced the most Beta-casein. However, these results clearly show that sequence G (SEQ ID NO: 7) has a negative impact on strain doubling time. Similarly, in considering the payload protein hBD3, traditional analysis would have identified sequence F (SEQ ID NO: 6) as the “best” signal peptide. However, these results show that sequence N (SEQ ID NO: 14) produces similar amounts of payload protein while having an even greater positive impact on strain doubling time.

### Example 3: Generation of Novel Optimal Signal Peptides for Secretion of Leptin

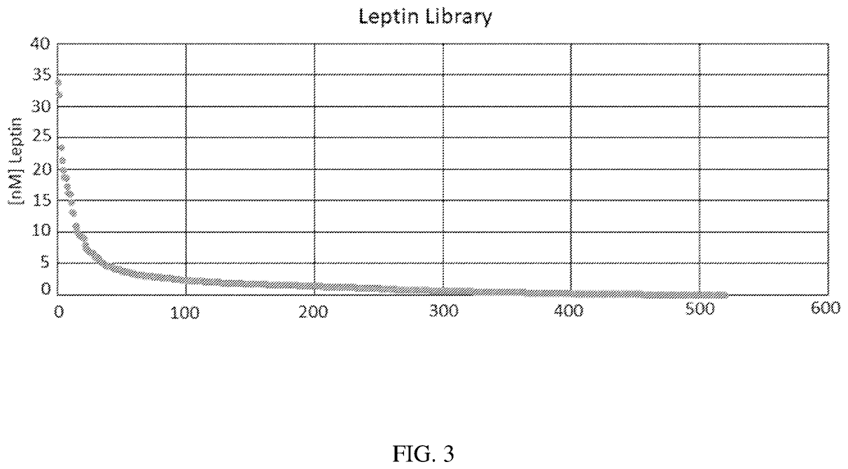

As demonstrated, signal peptide success varies when matched to different payload proteins, accordingly it was determined that a panel of sequences should be examined for each payload protein target. Therefore, a library of 520 signal peptide sequences was created and tested with leptin. The signal peptide sequences of the library ranged from 95% similar to SEQ ID NO: 1 to 63% similar to SEQ ID NO: 1. For the screen, bacteria were genetically modified with nucleic acid molecules encoding for the signal peptides of the library with leptin and allowed to incubate under conditions sufficient to produce the polypeptides. Supernatant was collected and the concentration of payload protein produced was determined. The screening data (FIG. 3) show the distribution of nM leptin generated from the signal peptide-leptin constructs.

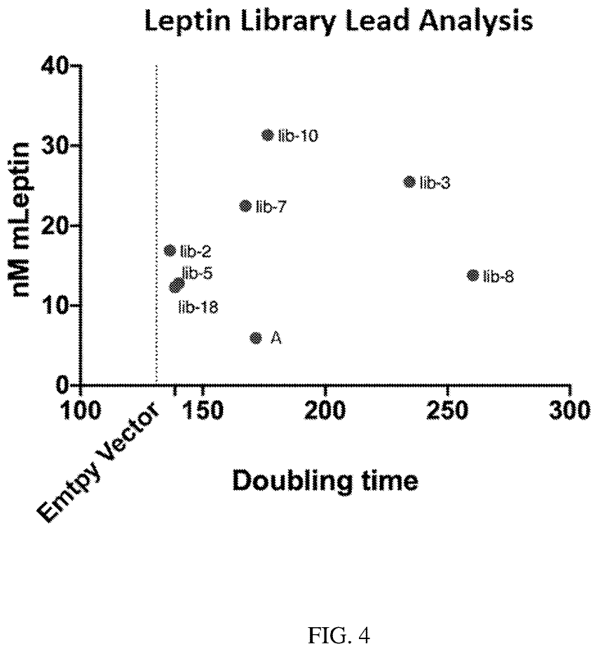

Those signal peptides that resulted in greater protein yield than SEQ ID NO: 1 were selected for further analysis. Signal peptide-Leptin constructs were evaluated for their effect on protein yield and strain doubling time as compared to SEQ ID NO: 1. Five signal peptides were found to result in increased leptin yield while having a reduced or similar strain doubling time as compared to SEQ ID NO: 1 (FIG. 4). Importantly, these signal peptides were all unique compared to those identified in the TNFα screen. The sequences are recited in Table 6 below, along with their percent similarity to the template sequence:

TABLE 6

| SEQ ID | | | % Identity to SEQ |
|---|---|---|---|
| NO: | Label | Sequence | ID NO: 1 |
| 1 | A | MKKLLLLLVVLLGLAALLLSGCGTSVSAAT | 100 |
| 17 | Lib-2 | MKKLLLGLVLLLGLAALALTGCGTPVSAAT | 83.3 |
| 47 | Lib-5 | MKKLTLLLTVSLGMAGFLLSGCGQPVSAAQ | 72.4 |
| 18 | Lib-7 | MKKLLLLLVVLLSVAALLASGCGTFVSAAT | 86.7 |
| 19 | Lib-10 | MKRLLLKAVVLLSLAALLWSGAGTSVSAAT | 80.0 |
| 48 | Lib-18 | MHKLLMLLVVLLGLAGLSLSGCGSSTSAAT | 80.0 |

Similar to the results of the TNFα screen in Example 1, these data indicate that % identity to SEQ ID NO: 1 does not have a bearing on doubling time or increased levels of secretion. Sequences Lib-2 (SEQ ID NO: 17), Lib-5 (SEQ ID NO: 47), and Lib-18 (SEQ ID NO: 48) all produced more leptin than SEQ ID NO: 1 and significantly decreased the strain doubling time, and all had varying degree of similarity to SEQ ID NO: 1 (83.3, 72.4, and 80.0% identity, respectively). Additionally, the results in FIG. 4 show that several signal peptides that were identified in the initial screen to increase overall leptin yield were shown to have a drastic negative effect with respect to doubling time, with each peptide resulting in an increase in doubling time of 50 minutes or more. The signal peptides resulting in increased doubling time all had equal or greater similarity to SEQ ID NO: 1 as compared to the signal peptides Lib-2, Lib-5, and Lib-18. Sequence Lib-8 (MKKALLLLGVATGLAAMLLSGCGTSVSAAT-SEQ ID NO: 141) is 83.3% similar to SEQ ID NO: 1, where sequence Lib-3 (MKKLLLLLVVLLGLAFLLLSGCGTSTSAQN-SEQ ID NO: 142) is 92.9% similar to SEQ ID NO: 1. These results are in agreement with those of the previous examples, illustrating that strain doubling time is an important variable that must be considered when designing signal peptides, and percent identity to a known signal peptide is not a sufficient predictor of success in signal peptide design.

### Example 4: Generation of Novel Optimal Signal Peptides for Secretion of IL-10

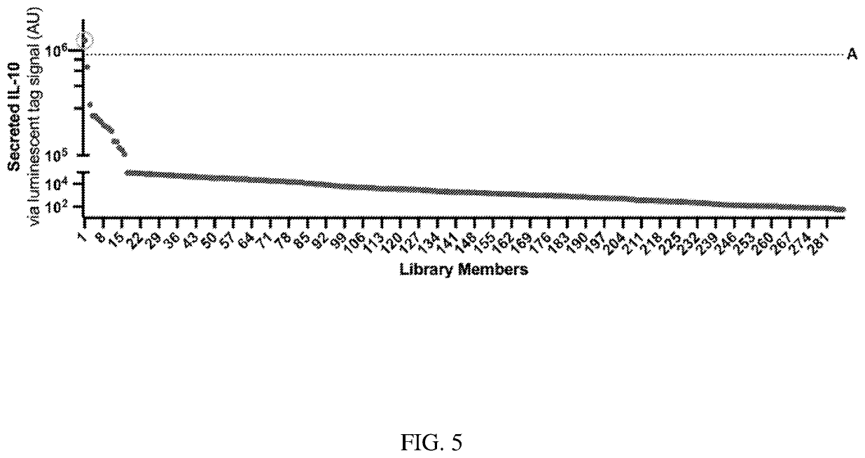

As detailed in Example 2, SEQ ID NO: 1 was identified as the best signal peptide for IL-10 from the initial sequences tested. To generate a novel signal peptide with comparable or better activity than SEQ ID NO: 1, a library of 351 signal peptide sequences was created and tested with IL-10. For the screen, bacteria were genetically modified with nucleic acid molecules encoding for the signal peptides of the library with IL-10 and allowed to incubate under conditions sufficient to produce the polypeptides. Supernatant was collected and the amount of IL-10 was determined using a luminescent tag signal. The screening data (FIG. 5) show the distribution of luminescent signal generated from the signal peptide-IL-10 constructs.

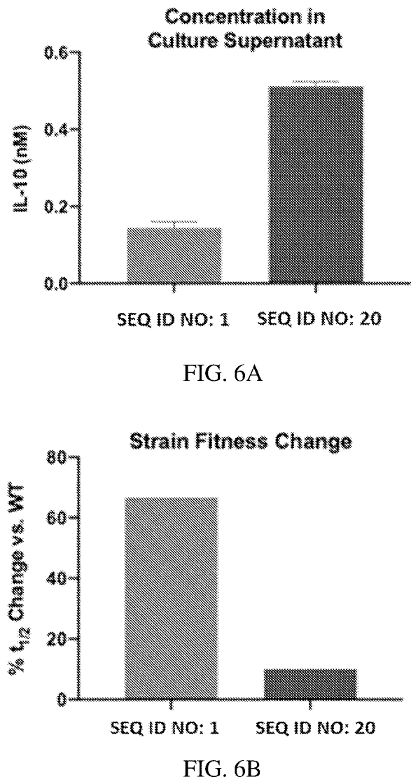

Of the 351 signal peptides tested, only 287 generated detectable IL-10 via the luminescent tag, resulting in a non-functional hit rate of 18%. Only 1 of the remaining 287 hits secreted a greater amount of IL-10 than SEQ ID NO: 1. The hit (SEQ ID NO: 20) was re-tested head-to-head against SEQ ID NO: 1 for both secretory performance and strain fitness, as measured by direct IL-10 concentration measurement via ELISA and strain doubling time (FIG. 6). As illustrated in FIG. 6A, strains utilizing SEQ ID NO: 20 resulted in 3.6× more IL-10 secretion than the strains utilizing SEQ ID NO: 1. Additionally, as illustrated in FIG. 6B, strains utilizing SEQ ID NO: 20 had a 6.7× lower fitness cost as compared to strains utilizing SEQ ID NO: 1. Strains utilizing SEQ ID NO: 20 only grew 10% slower than wild-type bacteria strains, while strains utilizing SEQ ID NO: 1 grew 67% slower.

A comparison of the amino acid sequences of SEQ ID NO: 1 and SEQ ID NO: 20 shows that SEQ ID NO: 20 is only 86.7% similar to SEQ ID NO: 1. Thus, the signal peptide sequence generated for an IL-10 payload follows the same trend observed for TNFα (Examples 1 and 2) and Leptin (Example 3), wherein percent similarity to a known signal peptide is not a sufficient predictor of signal peptide success.

### Example 5: Generation of Novel Optimal Signal Peptides for Secretion of IL-22

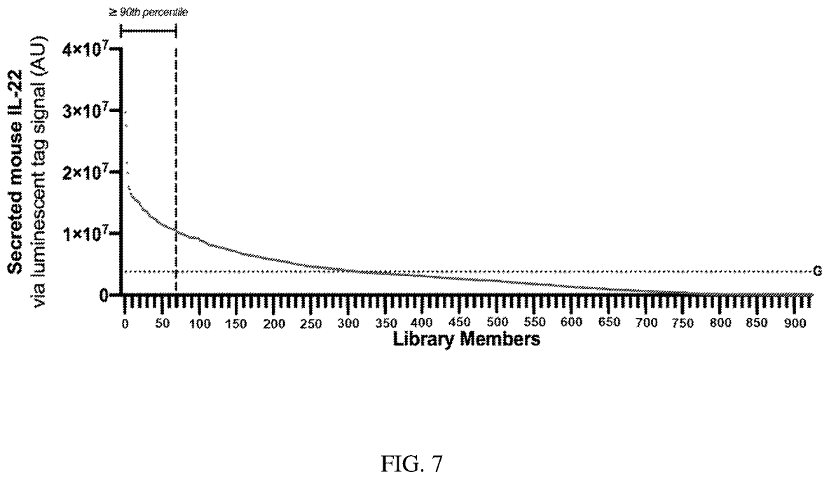

To expand on the IL-22 signal peptides identified in Example 2 above, a library of 925 signal peptide sequences was generated and the ability to secrete mouse IL-22 was assessed. For the screen, bacteria were genetically modified with nucleic acid molecules encoding for the signal peptides of the library with IL-22 and allowed to incubate under conditions sufficient to produce the polypeptides. Supernatant was collected and the amount of IL-22 was determined using a luminescent tag signal. The screening data (FIG. 7) show the distribution of luminescent signal generated from the signal peptide-IL-22 constructs.

Of the 925 signal peptides tested, 313 sequences generated more secreted IL-22 than the “best” IL-22 signal peptide from Example 2 above, i.e. SEQ ID NO: 7. The luminescent signal generated by SEQ ID NO: 7 is shown as the dotted line in FIG. 7. The identities of the peptides in the 90th percentile were determined via sequencing analysis and are provided as SEQ ID NOs: 144-212 in Table 1 above. Table 7 below provides the percent identity of the identified signal peptides both to SEQ ID NO: 1 and SEQ ID NO: 7.

TABLE 7

| | % Identity to | % Identity to |
|---|---|---|
| SEQ ID NO: | SEQ ID NO: 1 | SEQ ID NO: 7 |
| 144 | 86.6667 | 76.6667 |
| 145 | 90 | 83.3333 |
| 146 | 86.6667 | 80 |
| 147 | 90 | 73.3333 |
| 148 | 83.3333 | 83.3333 |
| 149 | 83.3333 | 76.6667 |
| 150 | 86.6667 | 80 |
| 151 | 83.3333 | 73.3333 |
| 152 | 90 | 76.6667 |
| 153 | 86.6667 | 83.3333 |
| 154 | 93.3333 | 83.3333 |
| 155 | 93.3333 | 80 |
| 156 | 93.3333 | 80 |
| 157 | 90 | 80 |
| 158 | 83.3333 | 80 |
| 159 | 90 | 83.3333 |
| 160 | 93.3333 | 80 |
| 161 | 90 | 80 |
| 162 | 86.6667 | 80 |
| 163 | 90 | 83.3333 |
| 164 | 83.3333 | 86.6667 |
| 165 | 90 | 83.3333 |
| 166 | 83.3333 | 80 |
| 167 | 86.6667 | 83.3333 |
| 168 | 77.7778 | 66.6667 |
| 169 | 83.3333 | 83.3333 |
| 170 | 90 | 76.6667 |
| 171 | 90 | 76.6667 |
| 172 | 86.6667 | 83.3333 |
| 173 | 90 | 80 |
| 174 | 86.6667 | 70 |
| 175 | 90 | 80 |
| 176 | 83.3333 | 80 |
| 177 | 90 | 86.6667 |
| 178 | 86.6667 | 80 |
| 179 | 90 | 73.3333 |
| 180 | 83.3333 | 80 |
| 181 | 90 | 76.6667 |
| 182 | 83.3333 | 76.6667 |
| 183 | 80 | 80 |
| 184 | 90 | 83.3333 |
| 185 | 90 | 80 |
| 186 | 80 | 86.6667 |
| 187 | 76.6667 | 76.6667 |
| 188 | 86.6667 | 80 |
| 189 | 86.6667 | 80 |
| 190 | 90 | 86.6667 |
| 191 | 90 | 83.3333 |
| 192 | 76.6667 | 80 |
| 193 | 90 | 73.3333 |
| 194 | 86.6667 | 76.6667 |
| 195 | 90 | 70 |
| 196 | 76.6667 | 80 |
| 197 | 93.3333 | 76.6667 |
| 198 | 86.6667 | 83.3333 |
| 199 | 86.6667 | 80 |
| 200 | 86.6667 | 80 |
| 201 | 90 | 80 |
| 202 | 86.6667 | 76.6667 |
| 203 | 86.6667 | 76.6667 |
| 204 | 86.6667 | 70.3704 |
| 205 | 86.6667 | 80 |
| 206 | 90 | 80 |
| 207 | 90 | 83.3333 |
| 208 | 90 | 76.6667 |
| 209 | 86.6667 | 73.3333 |
| 210 | 80 | 73.3333 |
| 211 | 86.6667 | 73.3333 |
| 212 | 80 | 76.6667 |

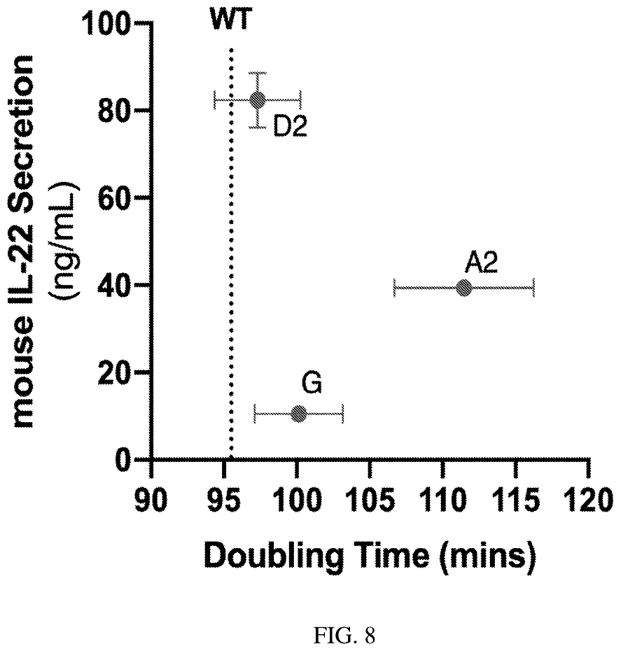

The top two sequences as determined by total IL-22 secreted (SEQ ID NOs: 148 and 180) were selected for further follow up. To confirm the results of FIG. 7 in the absence of the luminescent tag, signal peptide: IL-22 constructs were re-cloned without the luminescent tag, and the generated clones were evaluated for total IL-22 secreted. Clones were also assessed for effect of the constructs on bacteria strain fitness. The results are presented in FIG. 8. IL-22 constructs containing a signal peptide of SEQ ID NO: 148 (A2 in FIG. 8) or SEQ ID NO: 180 (D2 in FIG. 8) both produced more secreted IL-22 than the previously identified SEQ ID NO: 7 (G in FIG. 8). These results confirm the observations detailed above and illustrated in FIG. 7 with respect to IL-22 production and suggest that at least each of the 90th percentile hits from FIG. 7 (i.e., SEQ ID NOs: 144-212) would also promote the secretion IL-22. SEQ ID NO: 148 (A2) generated 3.7× more mouse IL-22 than SEQ ID NO: 7 (G) but also resulted in increased bacteria doubling time. SEQ ID NO: 180 (D2) produced 7.8× more mouse IL-22 than SEQ ID NO: 7 (G) and also demonstrated a slight reduction in doubling time, indicating increased bacteria strain fitness.

### Example 6: Assessment of IL-22 Signal Peptides with Additional Protein Targets

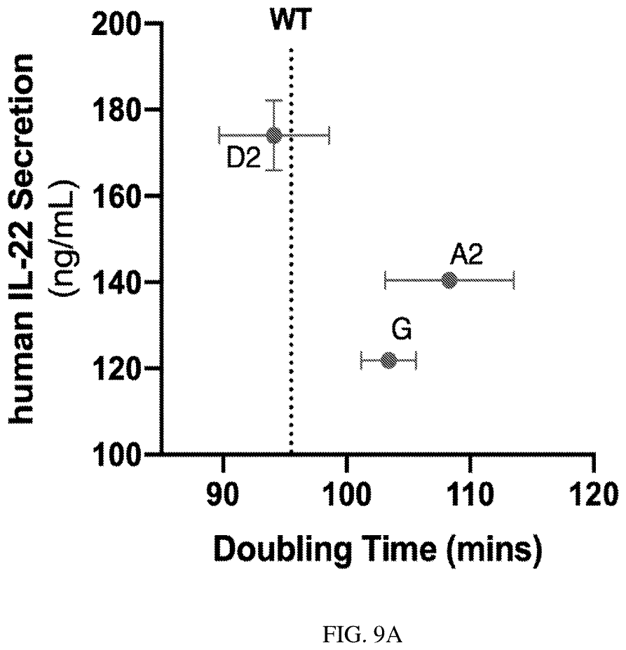

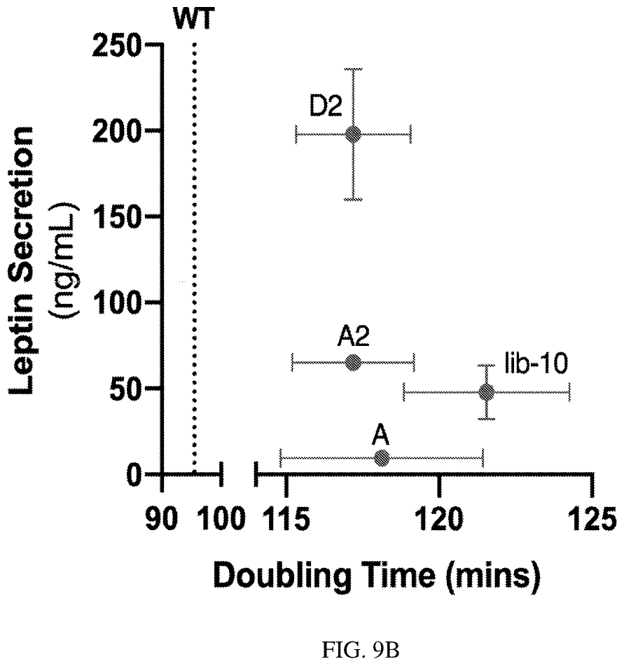

IL-22 signal peptides SEQ ID NO: 148 and SEQ ID NO: 180 were next tested for their ability to promote the secretion of related and unrelated protein targets. The signal peptides were identified in a screen using mouse IL-22, and so the signal peptides were first tested for their ability to promote the secretion of human IL-22. The results are shown in FIG. 9A. Similar to the mouse IL-22 results, both SEQ ID NO: 148 (A2 in FIG. 9A) and SEQ ID NO: 180 (D2 in FIG. 9A) promoted the secretion of more human IL-22 than the previously identified SEQ ID NO: 7 (G in FIG. 9A). These results demonstrate that the signal peptides would be expected to behave similarly between protein homologs (e.g. mouse vs human IL-22) and also suggests that at least each of the 90th percentile hits from FIG. 7 (i.e., SEQ ID NOs: 144-212) would also promote the secretion human IL-22. Also similar to the mouse results, SEQ ID NO: 148 (A2) generated more human IL-22 than SEQ ID NO: 7 (G) but also resulted in increased bacteria doubling time while SEQ ID NO: 180 (D2) generated more human IL-22 than SEQ ID NO: 7 (G) and demonstrated a reduction in doubling time.

The signal peptides were next tested for their ability to promote secretion of a substantially different protein product. Constructs were generated appending SEQ ID NO: 148 and SEQ ID NO: 180 to leptin, and the effects of the signal peptides on leptin secretion were assessed. As compared to IL-22, leptin is roughly half as large, contains half as many di-sulfide bonds, and is composed of a 4-helix bundle rather than a 6-helix bundle. As shown in FIG. 9B, the hits identified in the IL-22 screen surprisingly exhibited increased leptin production and better bacterial strain fitness than the “best” signal peptide identified for leptin in Example 3 above. SEQ ID NO: 148 (A2 in FIG. 9B) and SEQ ID NO: 180 (D2 in FIG. 9B) both demonstrated increase leptin production over SEQ ID NO: 19 (lib-10 in FIG. 9B) and the reference sequence SEQ ID NO: 1 (A in FIG. 9B). SEQ ID NO: 148 (A2) produced roughly 10-20% more leptin than SEQ ID NO: 19 (lib-10) and also demonstrated a decrease in doubling time as compared to SEQ ID NO: 19. SEQ ID NO: 180 (D2) produced roughly 4× more leptin than SEQ ID NO: 19 (lib-10) and also demonstrated a decrease in doubling time as compared to SEQ ID NO: 19. To determine if the apparent promiscuity of the signal peptides was due to sequence similarity, the IL-22 signal peptide sequences were compared to several of the leptin signal peptide sequences identified in Example 3 above. The results are shown in Table 8 below.

TABLE 8

| | | | % ID to | | |
|---|---|---|---|---|---|
| | % ID to SEQ | % ID to SEQ | SEQ ID | % ID to SEQ | % ID to SEQ |
| IL-22 SEQ | ID NO: 17 | ID NO: 47 | NO: 18 (lib- | ID NO: 19 | ID NO: 48 |
| ID NO: | (lib-2) | (lib-5) | 7) | (lib-10) | (lib-18) |
| SEQ ID NO: | 70% | 73.33% | 80% | 70% | 80% |
| 148 | | | | | |
| SEQ ID NO: | 73.33% | 63.33% | 70% | 76.67% | 73.33% |
| 180 | | | | | |

As detailed in Table 8, SEQ ID NO: 148 only has 70% identity to SEQ ID NO: 19 and at most has 80% identity to a leptin signal peptide identified in Example 3. SEQ ID NO: 180 has 76.67% identity to SEQ ID NO: 19, and does not share greater than 77% identity with any leptin signal peptide identified in Example 3.

The results of this example support that signal peptides identified for one homolog of a particular protein target will behave similarly when used with a separate homolog of the protein target. These results also demonstrate the surprising and unexpected finding that SEQ ID NO: 148 and SEQ ID NO: 180 were able to promote secretion of both IL-22 and leptin to a greater degree than previously identified signal peptides.

What is claimed is:

1. A pre-protein signal peptide comprising an amino acid sequence that has at least 95%, 96%, 97%, 98%, 99%, or 100% identity to an amino acid sequence selected from SEQ ID NO: 6, SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180.

2. A polypeptide comprising a formula of X₁-Z₁ wherein:
X₁ is a pre-protein signal peptide; and
Z₁ is a payload protein.

3. The polypeptide of claim 2, wherein X₁ comprises an amino acid sequence that has at least 95%, 96%, 97%, 98%, 99%, or 100% identity to an amino acid sequence selected from SEQ ID NO: 6, SEQ ID NO: 7, SEQ ID NO: 14, SEQ ID NO: 17, SEQ ID NO: 18, SEQ ID NO: 19, SEQ ID NO: 20, SEQ ID NO: 47, SEQ ID NO: 48, SEQ ID NO: 148, or SEQ ID NO: 180.

4. The polypeptide of claim 2 or claim 3, wherein Z₁ is selected from the group consisting of antimicrobial peptides, Catestatin, CRAMP, hBD1, hBD3, β-Cateslytin, Dermaseptin, IL-10, IL-22, ClbS, TIGIT, TFF1, TFF2, TFF3, anti-CCL2 antibody, anti-CCL2 antibody fragments, anti-IL23 antibody, anti-IL23 antibody fragments, anti-TNFαantibody, anti-TNFα antibody fragments, GLP2, IGF1, leptin, and casein.

5. The polypeptide of claim 2, wherein X₁ comprises an amino acid sequence of SEQ ID NO: 6, SEQ ID NO: 7, or SEQ ID NO: 14 and Z₁ comprises an anti-TNFα antibody or an anti-TNFα antibody fragment.

6. The polypeptide of claim 5, wherein X₁ comprises an amino acid sequence of SEQ ID NO: 6, SEQ ID NO: 7, or SEQ ID NO: 14 and Z₁ comprises an amino acid sequence having at least 95%, 96%, 97%, 98%, 99%, or 100% identity to SEQ ID NO: 21, SEQ ID NO: 118, SEQ ID NO: 119, SEQ ID NO: 120, or fragment thereof.

7. The polypeptide of claim 5 or 6, wherein X₁ comprises an amino acid sequence of SEQ ID NO: 6.

8. The polypeptide of claim 5 or 6, wherein X₁ comprises an amino acid sequence of SEQ ID NO: 7.

9. The polypeptide of claim 5 or 6, wherein X₁ comprises an amino acid sequence of SEQ ID NO: 14.

10. The polypeptide of claim 2, wherein X₁ comprises an amino acid sequence of SEQ ID NO: 6, SEQ ID NO: 7, SEQ ID NO: 14, SEQ ID NO: 17, SEQ ID NO: 18, SEQ ID NO: 19, SEQ ID NO: 47, SEQ ID NO: 48, SEQ ID NO: 148, or SEQ ID NO: 180, and Z₁ comprises leptin.

11. The polypeptide of claim 10, wherein X₁ comprises an amino acid sequence of SEQ ID NO: 6, SEQ ID NO: 7, SEQ ID NO: 14, SEQ ID NO: 17, SEQ ID NO: 18, SEQ ID NO: 19, SEQ ID NO: 47, SEQ ID NO: 48, SEQ ID NO: 148, or SEQ ID NO: 180, and Z₁ comprises an amino acid sequence having at least 95%, 96%, 97%, 98%, 99%, or 100% identity to SEQ ID NO: 25.

12. The polypeptide of claim 10 or 11, wherein X₁ comprises an amino acid sequence of SEQ ID NO: 6.

13. The polypeptide of claim 10 or 11, wherein X₁ comprises an amino acid sequence of SEQ ID NO: 7.

14. The polypeptide of claim 10 or 11, wherein X₁ comprises an amino acid sequence of SEQ ID NO: 14.

15. The polypeptide of claim 10 or 11, wherein X₁ comprises an amino acid sequence of SEQ ID NO: 17.

16. The polypeptide of claim 10 or 11, wherein X₁ comprises an amino acid sequence of SEQ ID NO: 18.

17. The polypeptide of claim 10 or 11, wherein X₁ comprises an amino acid sequence of SEQ ID NO: 19.

18. The polypeptide of claim 10 or 11, wherein X₁ comprises an amino acid sequence of SEQ ID NO: 47.

19. The polypeptide of claim 10 or 11, wherein X₁ comprises an amino acid sequence of SEQ ID NO: 48.

20. The polypeptide of claim 10 or 11, wherein X₁ comprises an amino acid sequence of SEQ ID NO: 148.

21. The polypeptide of claim 10 or 11, wherein X₁ comprises an amino acid sequence of SEQ ID NO: 180.

22. The polypeptide of claim 2, wherein X₁ comprises an amino acid sequence of SEQ ID NO: 20 and Z₁ comprises IL-10.

23. The polypeptide of claim 22, wherein X₁ comprises an amino acid sequence of SEQ ID NO: 20 and Z₁ comprises an amino acid sequence having at least 95%, 96%, 97%, 98%, 99%, or 100% identity to SEQ ID NO: 24, SEQ ID NO: 283, SEQ ID NO: 284, or SEQ ID NO: 285.

24. The polypeptide of claim 2, wherein X₁ comprises an amino acid sequence of SEQ ID NO: 7, SEQ ID NO: 14, SEQ ID NO: 148, or SEQ ID NO: 180, and Z₁ comprises IL-22.

25. The polypeptide of claim 24, wherein X₁ comprises an amino acid sequence of SEQ ID NO: 7, SEQ ID NO: 14, SEQ ID NO: 148, or SEQ ID NO: 180 and Z₁ comprises an amino acid sequence having at least 95%, 96%, 97%, 98%, 99%, or 100% identity to SEQ ID NO: 22, SEQ ID NO: 59, or SEQ ID NO: 282.

26. The polypeptide of claim 24 or 25, wherein X₁ comprises an amino acid sequence of SEQ ID NO: 7.

27. The polypeptide of claim 24 or 25, wherein X₁ comprises an amino acid sequence of SEQ ID NO: 14.

28. The polypeptide of claim 24 or 25, wherein X₁ comprises an amino acid sequence of SEQ ID NO: 148.

29. The polypeptide of claim 24 or 25, wherein X₁ comprises an amino acid sequence of SEQ ID NO: 180.

30. A polypeptide comprising a formula of X₁-Z₁ wherein:
X₁ is a pre-protein signal peptide comprising the amino acid sequence of SEQ ID NO: 6, SEQ ID NO: 7, or SEQ ID NO: 14; and
Z₁ is a payload protein comprising an anti-TNFα antibody or an anti-TNFα antibody fragment.

31. The polypeptide of claim 30, wherein Z₁ comprises an amino acid sequence selected from SEQ ID NO: 21, SEQ ID NO: 118, SEQ ID NO: 119, or SEQ ID NO: 120.

32. A polypeptide comprising a formula of X₁-Z₁ wherein:
X₁ is a pre-protein signal peptide comprising the amino acid sequence of SEQ ID NO: 6, SEQ ID NO: 7, SEQ ID NO: 14, SEQ ID NO: 17, SEQ ID NO: 18, SEQ ID NO: 19, SEQ ID NO: 47, SEQ ID NO: 48, SEQ ID NO: 148, or SEQ ID NO: 180; and
Z₁ is a payload protein comprising leptin.

33. The polypeptide of claim 32, wherein Z₁ comprises an amino acid sequence of SEQ ID NO: 25.

34. A polypeptide comprising a formula of X₁-Z₁ wherein:
X₁ is a pre-protein signal peptide comprising the amino acid sequence of SEQ ID NO: 20; and
Z₁ is a payload protein comprising IL-10.

35. The polypeptide of claim 34, wherein Z₁ comprises an amino acid sequence of SEQ ID NO: 24, SEQ ID NO: 283, SEQ ID NO: 284, or SEQ ID NO: 285.

36. A polypeptide comprising a formula of X₁-Z₁ wherein:
X₁ is a pre-protein signal peptide comprising the amino acid sequence of SEQ ID NO: 7, SEQ ID NO: 14, SEQ ID NO: 148, or SEQ ID NO: 180; and
Z₁ is a payload protein comprising IL-22.

37. The polypeptide of claim 36, wherein Z₁ comprises an amino acid sequence of SEQ ID NO: 22, SEQ ID NO: 59, or SEQ ID NO: 282.

38. The polypeptide of any one of claims 2-37, wherein X₁ and Z₁ are linked by a linker polypeptide.

39. A nucleic acid molecule encoding for a polypeptide of any one of claims 2-38.

40. A bacterium comprising a heterologous nucleic acid molecule encoding a polypeptide comprising a formula of X₁-Z₁ wherein:
X₁ is the pre-protein signal peptide of claim 1; and
Z₁ is a payload protein.

41. The bacterium of claim 40, wherein Z₁ is selected from the group consisting of Catestatin, CRAMP, hBD1, hBD3, β-Cateslytin, Dermaseptin, IL-10, IL-22, ClbS, TIGIT, TFF1, TFF2, TFF3, anti-CCL2 antibody, anti-CCL2 antibody fragments, anti-IL23 antibody, anti-IL23 antibody fragments, anti-TNFα antibody, anti-TNFα antibody fragments, GLP2, IGF1, leptin, and casein.

42. The bacterium of claim 40, wherein the formula X₁-Z₁ comprises the polypeptide of any one of claims 2-38.

43. The bacterium of any one of claims 40-42, wherein the bacterium is a Lactobacillus.

44. A pharmaceutical composition comprising the bacterium of any one of claims 40-43 and a pharmaceutically acceptable carrier.

45. A method for producing a payload protein, comprising:
i) transfecting a bacterium with a nucleic acid molecule encoding for the polypeptide of any one of claims 2-38 to produce a bacterium comprising the nucleic acid molecule; and
ii) culturing the bacteria comprising the nucleic acid molecule under conditions sufficient to grow the bacteria; wherein the bacteria expresses and secretes the payload protein.

46. The method of claim 45, wherein the bacterium is a Lactobacillus.

47. A method of producing a payload protein in a subject, comprising administering to the subject the bacteria of any one of claims 40-43 or the pharmaceutical composition of claim 44, wherein the bacteria are live and wherein the live bacteria produce the protein in the subject.

48. A method for treating a disease or disorder in a subject in need thereof comprising administering to the subject a therapeutically effective amount of the bacteria of any one of claims 40-43 or the pharmaceutical composition of claim 44.

49. The method of claim 48, wherein the disease or disorder is selected from inflammation, chronic inflammation, colorectal cancer, periodontits, oral mucositis, infection, pathogenic E coli, inflammatory bowel disease, ulcerative colitis, Crohn's disease, type 2 diabetes, obesity, lipodystrophy, hypertriglyceridemia, systemic lupus erythematosus, hepatic steatosis, low bone density, hypothalamic hypogonadism, infertility, hypothalamic amenorrhea, auto-immune disease, cancer, NAFLD, NASH, infection, sepsis, or a growth hormone disorder.
